(19) United States
(12) Patent Application Publication
(10) Pub. No.: US 2020/0071310 A1
(43) Pub. Date: Mar. 5, 2020

(54) N-ARYL PYRAZOLES AS NRF2 REGULATORS

(71) Applicants: GLAXOSMITHKLINE INTELLECTUAL PROPERTY DEVELOPMENT LIMITED, Brentford, Middlesex (GB); ASTEX THERAPEUTICS LIMITED, Cambridge, Cambridgeshire (GB)

(72) Inventors: Brian W. BUDZIK, King of Prussia, PA (US); Peng LI, King of Prussia, PA (US); Joseph E. PERO, King of Prussia, PA (US); Charlotte Mary GRIFFITHS-JONES, Cambridge, Cambridgeshire (GB); Thomas Daniel HEIGHTMAN, Cambridge, Cambridgeshire (GB); David NORTON, Cambridge, Cambridgeshire (GB); Hendrika Maria Gerarda WILLEMS, Cambridge, Cambridgeshire (GB); Alison Jo-Anne WOOLFORD, Cambridge, Cambridgeshire (GB)

(21) Appl. No.: 16/467,148

(22) PCT Filed: Dec. 11, 2017
(86) PCT No.: PCT/IB2017/057800

§ 371 Date: Jun. 6, 2019

Related U.S. Application Data
(60) Provisional application No. 62/432,887, filed on Dec. 12, 2016.

(51) Int. Cl. C07D 413/08; C07D 413/14; C07D 403/08; C07D 401/14; C07D 417/14; C07D 419/14; A61P 11/00

(52) U.S. Cl. CPC C07D 413/08; C07D 413/14; C07D 403/08; A61P 11/00; C07D 417/14; C07D 419/14; C07D 401/14

(57) ABSTRACT

The present invention relates to N-aryl pyrazole compounds, methods of making them, pharmaceutical compositions containing them and their use as NRF2 regulators. In particular, the compounds of this invention include a compound of Formula (I):

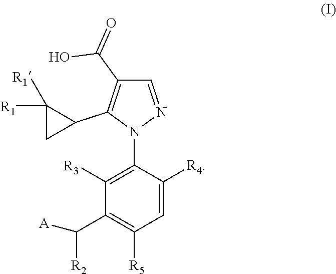

### FIELD OF THE INVENTION

The present invention relates to N-aryl pyrazole compounds, methods of making them, pharmaceutical compositions containing them and their use as NRF2 regulators.

### BACKGROUND OF THE INVENTION

NRF2 (NF-E2 related factor 2) is a member of the cap-n-collar family of transcription factors containing a characteristic basic-leucine zipper motif. Under basal conditions, NRF2 levels are tightly controlled by the cytosolic actin-bound repressor, KEAP1 (Kelch-like ECH associating protein 1), which binds to NRF2 and targets it for ubiquitylation and proteasomal degradation via the Cul3-based E3-ubiquitin ligase complex. Under conditions of oxidative stress, DJ1 (PARK7) is activated and stabilizes NRF2 protein by preventing NRF2 from interacting with KEAP1. Also, modification of reactive cysteines on KEAP1 can cause a conformational change in KEAP1 that alters NRF2 binding and promotes NRF2 stabilization. Thus, the levels of NRF2 in the cell are usually kept low in normal conditions but the system is designed to respond quickly to environmental stress by increasing NRF2 levels and thus downstream NRF2 activity.

Inappropriately low NRF2 activity in the face of on-going oxidative stress appears to be a pathological mechanism underlying chronic obstructive pulmonary disease (COPD). Yamada, K., et al. BMC Pulmonary Medicine, 2016, 16: 27. This may be a result of an altered equilibrium between NRF2 regulators with both inappropriate lack of positive regulators such as DJ1, and overabundance of negative regulators such as Keap1 and Bach1. Therefore, restoration of NRF2 activity in the lungs of COPD patients should result in repair of the imbalance and mitigation of deleterious processes such as apoptosis of structural cells (including alveolar epithelial and endothelial cells) and inflammation. The results of these effects would be enhanced cytoprotection, preservation of lung structure, and structural repair in the COPD lung, thus slowing disease progression. Therefore, NRF2 modulators may treat COPD (Boutten, A., et al. 2011. Trends Mol. Med. 17:363-371) and other respiratory diseases, including asthma, Acute Lung Injury (ALI) (Cho, H. Y., and Kleeberger, S. R., 2015, Arch Toxicol. 89:1931-1957; Zhao, H. et al., 2017, Am J Physiol Lung Clee Mol Physiol 312:L155-L162, first published Nov. 18, 2016; doi:10.1152/ajplung.00449.2016), Acute Respiratory Distress Syndrome (ARDS) and pulmonary fibrosis (Cho, H. Y., and Kleeberger, S. R. 2010. Toxicol. Appl. Pharmacol. 244:43-56).

The therapeutic potential of an NRF2 activator is exemplified in pulmonary macrophages from COPD patients where NRF2 pathway appears maladaptive. These cells have impaired bacterial phagocytosis compared with similar cells from control patients, and this effect is reversed by the addition of NRF2 activators in vitro. Therefore, in addition to the effects mentioned above, restoration of appropriate NRF2 activity could also rescue COPD exacerbations by reducing lung infection.

This is demonstrated by the NRF2 activator, Sulforaphane, which increases the expression of Macrophage Receptor with Collagenous structure (MARCO) by COPD macrophages and alveolar macrophages from cigarette smoke-exposed mice, thereby improving in these cells bacterial phagocytosis (Pseudomonas aeruginosa, non-typable Haemophilus influenzae) and bacterial clearance both ex vivo and in vivo. (Harvey, C. J., et al. 2011. Sci. Transl. Med. 3:78ra32).

The therapeutic potential of targeting NRF2 in the lung is not limited to COPD. Rather, targeting the NRF2 pathway could provide treatments for other human lung and respiratory diseases that exhibit oxidative stress components such as chronic asthma and acute asthma, lung disease secondary to environmental exposures including but not limited to ozone, diesel exhaust and occupational exposures, fibrosis, acute lung infection (e.g., viral (Noah, T. L. et al. 2014. PLoS ONE 9(6): e98671), bacterial or fungal), chronic lung infection, α1 antitrypsin disease, ALI, ARDS and cystic fibrosis (CF, Chen, J. et al. 2008. PLoS One. 2008; 3(10):e3367).

A therapy that targets the NRF2 pathway also has many potential uses outside the lung and respiratory system. Many of the diseases for which an NRF2 activator may be useful are autoimmune diseases (psoriasis, IBD, MS), suggesting that an NRF2 activator may be useful in autoimmune diseases in general.

In the clinic, a drug targeting the NRF2 pathway (bardoxolone methyl) has shown efficacy in diabetic patients with diabetic nephropathy/chronic kidney disease (CKD) (Aleksunes, L. M., et al. 2010. J Pharmacol. Exp. Ther. 335:2-12), though phase III trials with this drug in patients with the most severe stage of CKD were terminated. Furthermore, there is evidence to suspect that such a therapy would be effective in sepsis-induced acute kidney injury, other acute kidney injury (AKI) (Shelton, L. M., et al. 2013. Kidney International. June 19. doi:10.1038/ki.2013.248.), and kidney disease or malfunction seen during kidney transplantation.

In the cardiac area, bardoxolone methyl is currently under investigation in patients 30 with Pulmonary Arterial Hypertension and so a drug targeting NRF2 by other mechanisms may also be useful in this disease area. Oxidative stress is increased in the diseased myocardium, resulting in accumulation of reactive oxygen species (ROS) which impairs cardiac function [Circ (1987) 76(2); 458-468] and increases susceptibility to arrhythmia [J of Mol & Cell Cardio (1991) 23(8); 899-918] by a direct toxic effect of increased necrosis and apoptosis [Circ Res (2000) 87(12); 1172-1179]. In a mouse model of pressure overload (TAC), NRF2 gene and protein expression is increased during the early stage of cardiac adaptive hypertrophy, but decreased in the later stage of maladaptive cardiac remodeling associated with systolic dysfunction [Arterioscler Thromb Vasc Biol (2009) 29(11); 1843-5 1850; PLOS ONE (2012) 7(9); e44899]. In addition, NRF2 activation has been shown to suppress myocardial oxidative stress as well as cardiac apoptosis, fibrosis, hypertrophy, and dysfunction in mouse models of pressure overload [Arterioscler Thromb Vasc Biol (2009) 29(11); J of Mol & Cell Cardio (2014) 72; 305-315; and 1843-1850; PLOS ONE (2012) 7(9); e44899]. NRF2 activation has also been shown to protect against cardiac I/R injury in mice 10 [Circ Res (2009) 105(4); 365-374; J of Mol & Cell Cardio (2010) 49(4); 576-586] and reduce myocardial oxidative damage following cardiac I/R injury in rat. Therefore, a drug targeting NRF2 by other mechanisms may be useful in a variety of cardiovascular diseases including but not limited to atherosclerosis, hypertension, and heart failure (Oxidative Medicine and Cellular Longevity Volume 2013 (2013), Article ID 104308, 10 pages), acute coronary 15 syndrome, myocardial infarction, myocardial repair, cardiac remodeling, cardiac arrhythmias, heart failure with preserved ejection fraction, heart failure with reduced ejection fraction and diabetic cardiomyopathy.

A drug activating the NRF2 pathway could also be useful for treatment of several neurodegenerative diseases including Parkinson's disease (PD), Alzheimer's disease (AD), amyotrophic lateral sclerosis (ALS) (Brain Res. 2012 Mar. 29; 1446:109-18. 2011.12.064. Epub 2012 Jan. 12.) and multiple sclerosis (MS). Multiple in vivo models have shown that NRF2 KO mice are more sensitive to neurotoxic insults than their wild-type counterparts. Treatment of rats with the NRF2 activator tert-butylhydroquinone (tBHQ) reduced cortical damage in rats in a cerebral ischemia-reperfusion model, and cortical glutathione levels were increased in NRF2 wild-type but not KO mice after administration of tBHQ (Shih, A. Y., et al. 2005. J Neurosci. 25: 10321-10335). Tecfidera™ (dimethyl fumarate), which activates NRF2 among other targets, is approved in the U.S. to treat relapsing-remitting multiple sclerosis (MS). Activation of NRF2 may also help treat cases of Friedreich's Ataxia, where increased sensitivity to oxidative stress and impaired NRF2 activation has been reported (Paupe V., et al, 2009. PLoS One; 4(1):e4253. Omaveloxolone (RTA-408) is also in clinical trials for Friedreich's Ataxia.

There is preclinical evidence of the specific protective role of the NRF2 pathway in models of inflammatory bowel disease (IBD, Crohn's Disease and Ulcerative Colitis) and/or colon cancer (Khor, T. O., et al 2008. Cancer Prev. Res. (Phila) 1:187-191).

Age-related macular degeneration (AMD) is a common cause of vision loss in people over the age of 50. Cigarette smoking is a major risk factor for the development of non-neovascular (dry) AMD and perhaps also neovascular (wet) AMD. Findings in vitro and in preclinical species support the notion that the NRF2 pathway is involved in the anti-oxidant response of retinal epithelial cells and modulation of inflammation in pre-clinical models of eye injury (Schimel, et al. 2011. Am. J. Pathol. 178:2032-2043). Fuchs Endothelial Corneal Dystrophy (FECD) is a progressive, blinding disease characterized by corneal endothelial cells apoptosis. It is a disease of aging and increased oxidative stress related to low levels of NRF2 expression and/or function (Bitar, M. S., et al. 2012. Invest Ophthalmol. Vis. Sci. Aug. 24, 2012 vol. 53 no. 9 5806-5813). In addition, an NRF2 activator may be useful in uveitis or other inflammatory eye conditions.

Non-alcoholic steatohepatitis (NASH) is a disease of fat deposition, inflammation, and damage in the liver that occurs in patients who drink little or no alcohol. In pre-clinical models, development of NASH is greatly accelerated in KO mice lacking NRF2 when challenged with a methionine- and choline-deficient diet (Chowdhry S., et al. 2010. Free Rad. Biol. & Med. 48:357-371). Administration of the NRF2 activators oltipraz and NK-252 in rats on a choline-deficient L-amino acid-defined diet significantly attenuated progression ofhistologic abnormalities, especially hepatic fibrosis (Shimozono R. et al. 2012. Molecular Pharmacology. 84:62-70). Other liver diseases that may be amenable to NRF2 modulation are toxin-induced liver disease (e.g., acetaminophen-induced hepatic disease), viral hepatitis, and cirrhosis (Oxidative Medicine and Cellular Longevity Volume 2013 (2013), Article ID 763257, 9 page).

Recent studies have also begun to elucidate the role of ROS in skin diseases such as psoriasis. A study in psoriasis patients showed an increase in serum malondialdehyde and nitric oxide end products and a decrease in erythrocyte-superoxide dismutase activity, catalase activity, and total antioxidant status that correlated in each case with disease severity index (Dipali P. K., et al. Indian J Clin Biochem. 2010 October; 25(4): 388-392). Also, an NRF2 modulator may be useful in treating the dermatitis/topical effects of radiation (Schaifer, M. et al. 2010. Genes & Devl. 24:1045-1058), and the immunosuppression due to radiation exposure (Kim, J. H. et al., J. Clin. Invest. 2014 Feb. 3; 124(2):730-41).

There are also data suggesting that an NRF2 activator may be beneficial in preeclampsia, a disease that occurs in 2-5% of pregnancies and involves hypertension and proteinuria (Annals of Anatomy—Anatomischer Anzeiger Volume 196. Issue 5, September 2014, Pages 268-277).

Preclinical data has shown that compounds with NRF2 activating activity are better at reversing high altitude-induced damage than compounds without NRF2 activity, using animal and cellular models of Acute Mountain Sickness (Lisk C. et al, 2013, Free Radic Biol Med. October 2013; 63: 264-273.)

### SUMMARY OF THE INVENTION

In one aspect, this invention provides for N-aryl pyrazole analogs, or a salt, particularly a pharmaceutically acceptable salt thereof, and pharmaceutical compositions containing them. In particular, the compounds of this invention include a compound of Formula (I):

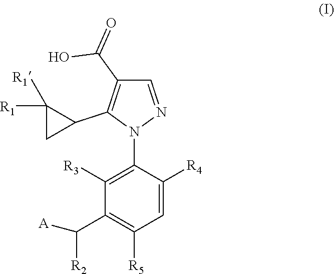

wherein:
R₁ is hydrogen, C₁₋₅alkyl, triazolyl, pyridyl, pyridazinyl, imidazolyl, pyrazolyl, isoxazolyl, halo, —NR₆—C(O)—R₇ or —C(O)R₇, and wherein the triazolyl, pyridyl, pyridazinyl, imidazolyl, pyrazolyl or isoxazolyl is unsubstituted or substituted by one or two substituents independently selected from —C₁₋₃alkyl, —CF₃ and halo;
R₁′ is hydrogen or halo;
R₂ is hydrogen, —C₁₋₅alkyl, —C₃₋₆cycloalkyl, or halo;
R₃ is hydrogen, —C₁₋₅alkyl, —C₃₋₆cycloalkyl, or halo;
or, when R₂ and R₃ are each C₁₋₅alkyl, together they form a 5- to 6-membered cycloalkyl ring fused to the adjacent phenyl ring;
R₄ is hydrogen, —C₁₋₅alkyl, —C₃₋₆cycloalkyl, or halo;
R₅ is hydrogen, —C₁₋₅alkyl, —C₃₋₆cycloalkyl, or halo;
or, when R₂ and R₅ are each C₁₋₅alkyl, together they form a 5- to 6-membered cycloalkyl ring fused to the adjacent phenyl ring;
R₆ and R₇ are independently hydrogen or —C₁₋₅alkyl;

### A is

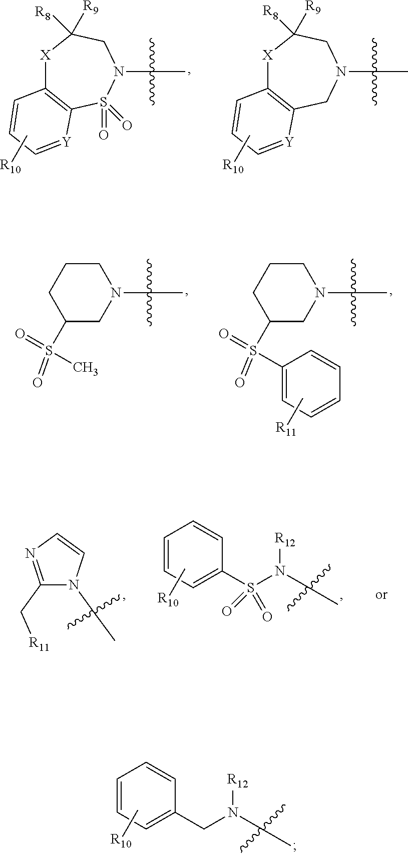

R₈ and R₉ are independently hydrogen or —C₁₋₅alkyl;
Each of R₁₀ is independently hydrogen, —C₁₋₅alkyl, —C₃₋₇cycloalkyl or halo;
R₁₁ is hydrogen or —C₅₋₈cycloalkyl;
R₁₂ is hydrogen, —C₁₋₆alkyl or —C₃₋₆cycloalkyl, wherein —C₁₋₆alkyl is unsubstituted or substituted with C₁₋₃alkyl;

### X is CH₂ or O;

### Y is CH or N;

or a pharmaceutically acceptable salt thereof.

In a second aspect, this invention provides for the use of the compounds of Formula (I) as NRF2 regulators.

Accordingly, the present invention is also directed to a method of regulating NRF2 which method comprises contacting a cell with a compound according to Formula (I), or a salt, particularly a pharmaceutically acceptable salt, thereof.

In another aspect, this invention provides for the use of the compounds of Formula (I) for treating and preventing conditions associated with NRF2 imbalance.

In one aspect, the invention is provides a pharmaceutical composition comprising a compound of the invention according to Formula (I), or a salt, particularly a pharmaceutically acceptable salt thereof, and a pharmaceutically acceptable excipient.

Particularly, this invention is directed to a pharmaceutical composition for the treatment of an NRF2 regulated disease or disorder, wherein the composition comprises a compound according to Formula (I), or a salt, particularly a pharmaceutically acceptable salt thereof, and a pharmaceutically acceptable excipient.

In a further aspect, this invention provides for a method of treating a respiratory or non-respiratory disorder, including COPD, asthma, ALI, ARDS, fibrosis, chronic asthma and acute asthma, lung disease secondary to environmental exposures, acute lung infection, chronic lung infection, α1 antitrypsin disease, cystic fibrosis, autoimmune diseases, diabetic nephropathy, chronic kidney disease, sepsis-induced acute kidney injury, acute kidney injury (AKI), kidney disease or malfunction seen during kidney transplantation, Pulmonary Arterial Hypertension, atherosclerosis, hypertension, heart failure, acute coronary syndrome, myocardial infarction, myocardial repair, cardiac remodelling, cardiac arrhythmias, Parkinson's disease (PD), Alzheimer's disease (AD), Friedreich's Ataxia (FA), amyotrophic lateral sclerosis (ALS), multiple sclerosis (MS), inflammatory bowel disease, colon cancer, neovascular (dry) AMD and neovascular (wet) AMD, eye injury, Fuchs Endothelial Corneal Dystrophy (FECD), uveitis or other inflammatory eye conditions, Non-alcoholic Steatohepatitis (NASH), toxin-induced liver disease (e.g., acetaminophen-induced hepatic disease), viral hepatitis, cirrhosis, psoriasis, dermatitis/topical effects of radiation, immunosuppression due to radiation exposure, Preeclampsia, and high altitude sickness, which comprises administering to a human in need thereof, a compound of Formula (I).

In one aspect, this invention relates to a method of treating COPD, which comprises administering to a human in need thereof, a compound of Formula (I), or a salt, particularly a pharmaceutically acceptable salt thereof.

In one aspect, this invention relates to a method of treating heart failure, which comprises administering to a human in need thereof, a compound of Formula (I), or a salt, particularly a pharmaceutically acceptable salt thereof.

In yet another aspect, this invention provides for the use of a compound of Formula (I), or a salt, particularly a pharmaceutically acceptable salt thereof, for the treatment of a respiratory or non-respiratory disorder, including COPD, asthma, ALI, ARDS, fibrosis, chronic asthma and acute asthma, lung disease secondary to environmental exposures, acute lung infection, chronic lung infection, α1 antitrypsin disease, cystic fibrosis, autoimmune diseases, diabetic nephropathy, chronic kidney disease, sepsis-induced acute kidney injury, acute kidney injury (AKI), kidney disease or malfunction seen during kidney transplantation, Pulmonary Arterial Hypertension, atherosclerosis, hypertension, heart failure, acute coronary syndrome, myocardial infarction, myocardial repair, cardiac remodelling, cardiac arrhythmias, Parkinson's disease (PD), Alzheimer's disease (AD), Friedreich's Ataxia (FA), amyotrophic lateral sclerosis (ALS), multiple sclerosis (MS), inflammatory bowel disease, colon cancer, neovascular (dry) AMD and neovascular (wet) AMD, eye injury, Fuchs Endothelial Corneal Dystrophy (FECD), uveitis or other inflammatory eye conditions, Non-alcoholic Steatohepatitis (NASH), toxin-induced liver disease (e.g., acetaminophen-induced hepatic disease), viral hepatitis, cirrhosis, psoriasis, dermatitis/topical effects of radiation, immunosuppression due to radiation exposure, Preeclampsia, and high altitude sickness.

In one aspect, this invention relates to the use of a compound of Formula (I), or a salt, particularly a pharmaceutically acceptable salt thereof, for the treatment of COPD.

In one aspect, this invention relates to the use of a compound of Formula (I), or a salt, particularly a pharmaceutically acceptable salt thereof, for the treatment of heart failure.

In a further aspect, this invention relates to use of a compound of Formula (I), or a salt, particularly a pharmaceutically acceptable salt thereof, in the manufacture of a medicament for use in the treatment of a respiratory or non-respiratory disorder, including COPD, asthma, ALI, ARDS, fibrosis, chronic asthma and acute asthma, lung disease secondary to environmental exposures, acute lung infection, chronic lung infection, α1 antitrypsin disease, cystic fibrosis, autoimmune diseases, diabetic nephropathy, chronic kidney disease, sepsis-induced acute kidney injury, acute kidney injury (AKI), kidney disease or malfunction seen during kidney transplantation, Pulmonary Arterial Hypertension, atherosclerosis, hypertension, heart failure, acute coronary syndrome, myocardial infarction, myocardial repair, cardiac remodelling, cardiac arrhythmias, Parkinson's disease (PD), Alzheimer's disease (AD), Friedreich's Ataxia (FA), amyotrophic lateral sclerosis (ALS), multiple sclerosis (MS), inflammatory bowel disease, colon cancer, neovascular (dry) AMD and neovascular (wet) AMD, eye injury, Fuchs Endothelial Corneal Dystrophy (FECD), uveitis or other inflammatory eye conditions, Non-alcoholic Steatohepatitis (NASH), toxin-induced liver disease (e.g., acetaminophen-induced hepatic disease), viral hepatitis, cirrhosis, psoriasis, dermatitis/topical effects of radiation, immunosuppression due to radiation exposure, Preeclampsia, and high altitude sickness.

In one aspect, this invention relates to use of a compound of Formula (I), or a salt, particularly a pharmaceutically acceptable salt thereof, in the manufacture of a medicament for the treatment of COPD.

In one aspect, this invention relates to use of a compound of Formula (I), or a salt, particularly a pharmaceutically acceptable salt thereof, in the manufacture of a medicament for the treatment of heart failure.

In a further aspect, this invention relates to a compound of Formula (I), or a salt, particularly a pharmaceutically acceptable salt thereof, for use in medical therapy. This invention relates to a compound of Formula (I), or a salt, particularly a pharmaceutically acceptable salt thereof, for use in therapy, specifically for use in the treatment of a respiratory or non-respiratory disorder, including COPD, asthma, ALI, ARDS, fibrosis, chronic asthma and acute asthma, lung disease secondary to environmental exposures, acute lung infection, chronic lung infection, α1 antitrypsin disease, cystic fibrosis, autoimmune diseases, diabetic nephropathy, chronic kidney disease, sepsis-induced acute kidney injury, acute kidney injury (AKI), kidney disease or malfunction seen during kidney transplantation, Pulmonary Arterial Hypertension, atherosclerosis, hypertension, heart failure, acute coronary syndrome, myocardial infarction, myocardial repair, cardiac remodelling, cardiac arrhythmias, Parkinson's disease (PD), Alzheimer's disease (AD), Friedreich's Ataxia (FA), amyotrophic lateral sclerosis (ALS), multiple sclerosis (MS), inflammatory bowel disease, colon cancer, neovascular (dry) AMD and neovascular (wet) AMD, eye injury, Fuchs Endothelial Corneal Dystrophy (FECD), uveitis or other inflammatory eye conditions, Non-alcoholic Steatohepatitis (NASH), toxin-induced liver disease (e.g., acetaminophen-induced hepatic disease), viral hepatitis, cirrhosis, psoriasis, dermatitis/topical effects of radiation, immunosuppression due to radiation exposure, Preeclampsia, and high altitude sickness.

In one aspect, this invention relates to a compound of Formula (I), or a salt, particularly a pharmaceutically acceptable salt thereof, for use in the treatment of COPD.

In one aspect, this invention relates to a compound of Formula (I), or a salt, particularly a pharmaceutically acceptable salt thereof, for use in the treatment of heart failure.

The compounds of Formula (I) and pharmaceutically acceptable salts thereof may be used in combination with one or more other agents which may be useful in the prevention or treatment of allergic disease, inflammatory disease, autoimmune disease, for example; antigen immunotherapy, anti-histamines, corticosteroids, (e.g., fluticasone propionate, fluticasone furoate, beclomethasone dipropionate, budesonide, ciclesonide, mometasone furoate, triamcinolone, flunisolide), NSAIDs, leukotriene modulators (e.g., montelukast, zafirlukast, pranlukast), iNOS inhibitors, tryptase inhibitors, IKK2 inhibitors, p38 inhibitors, Syk inhibitors, protease inhibitors such as elastase inhibitors, integrin antagonists (e.g., beta-2 integrin antagonists), adenosine A2a agonists, mediator release inhibitors such as sodium chromoglycate, 5-lipoxygenase inhibitors (zyflo), DP1 antagonists, DP2 antagonists, PI3K delta inhibitors, ITK inhibitors, LP (lysophosphatidic) inhibitors or FLAP (5-lipoxygenase activating protein) inhibitors (e.g., sodium 3-(3-(tert-butylthio)-1-(4-(6-ethoxypyridin-3-yl)benzyl)-5-((5-methylpyridin-2-yl)methoxy)-1H-indol-2-yl)-2,2-dimethylpropanoate), bronchodilators (e.g., muscarinic antagonists, beta-2 agonists), methotrexate, and similar agents; monoclonal antibody therapy such as anti-IgE, anti-TNF, anti-IL-5, anti-IL-6, anti-IL-12, anti-IL-1 and similar agents; cytokine receptor therapies e.g. etanercept and similar agents; antigen non-specific immunotherapies (e.g. interferon or other cytokines/chemokines, chemokine receptor modulators such as CCR3, CCR4 or CXCR2 antagonists, other cytokine/chemokine agonists or antagonists, TLR agonists and similar agents).

Suitably, for the treatment of asthma, compounds or pharmaceutical formulations of the invention may be administered together with an anti-inflammatory agent such as, for example, a corticosteroid, or a pharmaceutical formulation thereof. For example, a compound of the invention may be formulated together with an anti-inflammatory agent, such as a corticosteroid, in a single formulation, such as a dry powder formulation for inhalation. Alternatively, a pharmaceutical formulation comprising a compound of the invention may be administered in conjunction with a pharmaceutical formulation comprising an anti-inflammatory agent, such as a corticosteroid, either simultaneously or sequentially. In one embodiment, a pharmaceutical formulation comprising a compound of the invention and a pharmaceutical formulation comprising an anti-inflammatory agent, such as a corticosteroid, may each be held in device suitable for the simultaneous administration of both formulations via inhalation.

Suitable corticosteroids for administration together with a compound of the invention include, but are not limited to, fluticasone furoate, fluticasone propionate, beclomethasone diproprionate, budesonide, ciclesonide, mometasone furoate, triamcinolone, flunisolide and prednisilone. In one embodiment of the invention a corticosteroids for administration together with a compound of the invention via inhalation includes fluticasone furoate, fluticasone propionate, beclomethasone diproprionate, budesonide, ciclesonide, mometasone furoate, and, flunisolide.

Suitably, for the treatment of COPD, compounds or pharmaceutical formulations of the invention may be administered together with one or more bronchodilators, or pharmaceutical formulations thereof. For example, a compound of the invention may be formulated together with one or more bronchodilators in a single formulation, such as a dry powder formulation for inhalation. Alternatively, a pharmaceutical formulation comprising a compound of the invention may be administered in conjunction with a pharmaceutical formulation comprising one or more bronchodilators, either simultaneously or sequentially. In a further alternative, a formulation comprising a compound of the invention and a bronchodilator may be administered in conjunction with a pharmaceutical formulation comprising a further bronchodilator. In one embodiment, a pharmaceutical formulation comprising a compound of the invention and a pharmaceutical formulation comprising one or more bronchodilators may each be held in device suitable for the simultaneous administration of both formulations via inhalation. In a further embodiment, a pharmaceutical formulation comprising a compound of the invention together with a bronchodilator and a pharmaceutical formulation comprising a further bronchodilator may each be held in one or more devices suitable for the simultaneous administration of both formulations via inhalation.

Suitable bronchodilators for administration together with a compound of the invention include, but are not limited to, β₂-adrenoreceptor agonists and anticholinergic agents. Examples of β₂-adrenoreceptor agonists, include, for example, vilanterol, salmeterol, salbutamol, formoterol, salmefamol, fenoterol carmoterol, etanterol, naminterol, clenbuterol, pirbuterol, flerbuterol, reproterol, bambuterol, indacaterol, terbutaline and salts thereof, for example the xinafoate (1-hydroxy-2-naphthalenecarboxylate) salt of salmeterol, the sulphate salt of salbutamol or the fumarate salt of formoterol. Suitable anticholinergic agents include umeclidinium (for example, as the bromide), ipratropium (for example, as the bromide), oxitropium (for example, as the bromide) and tiotropium (for example, as the bromide). In one embodiment of the invention, a compound of the invention may be administered together with a β₂-adrenoreceptor agonist, such as vilanterol, and an anticholinergic agent, such as, umeclidinium.

The compounds may also be used in combination with agents for aiding transplantation including Cyclosporines, Tacrolimus, Mycophenolate mofetil, Prednisone, Azathioprine, Sirolimus, Daclizumab, Basiliximab and OKT3.

They may also be used in combination with agents for Diabetes: metformin (biguanides), meglitinides, sulfonylureas, DPP-4 inhibitors, Thiazolidinediones, Alpha-glucosidase inhibitors, Amylin mimetics, Incretin mimetics and insulin.

The compounds may be used in combination with antihypertensives such as diuretics, ACE inhibitors, ARBS, calcium channel blockers, and beta blockers.

Other aspects and advantages of the present invention are described further in the following detailed description of the preferred embodiments thereof.

### DETAILED DESCRIPTION OF THE INVENTION

The present invention provides for compounds of Formula (I):

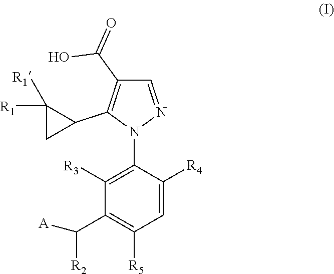

wherein:
R₁ is hydrogen, —C₁₋₅alkyl, triazolyl, pyridyl, pyridazinyl, imidazolyl, pyrazolyl, isoxazolyl, halo, —NR₆—C(O)—R₇ or —C(O)R₇, and wherein the triazolyl, pyridyl, pyridazinyl, imidazolyl, pyrazolyl or isoxazolyl is unsubstituted or substituted by one or two substituents independently selected from —C₁₋₃alkyl, —CF₃ and halo;
R₁′ is hydrogen or halo;
R₂ is hydrogen, —C₁₋₅alkyl, —C₃₋₆cycloalkyl, or halo;
R₃ is hydrogen, —C₁₋₅alkyl, —C₃₋₆cycloalkyl, or halo;
or, when R₂ and R₃ are each C₁₋₅alkyl, together they form a 5- to 6-membered cycloalkyl ring fused to the adjacent phenyl ring;
R₄ is hydrogen, —C₁₋₅alkyl, —C₃₋₆cycloalkyl, or halo;
R₅ is hydrogen, —C₁₋₅alkyl, —C₃₋₆cycloalkyl, or halo;
or, when R₂ and R₅ are each C₁₋₅alkyl, together they form a 5- to 6-membered cycloalkyl ring fused to the adjacent phenyl ring;
R₆ and R₇ are independently hydrogen or —C₁₋₅alkyl;

### A is

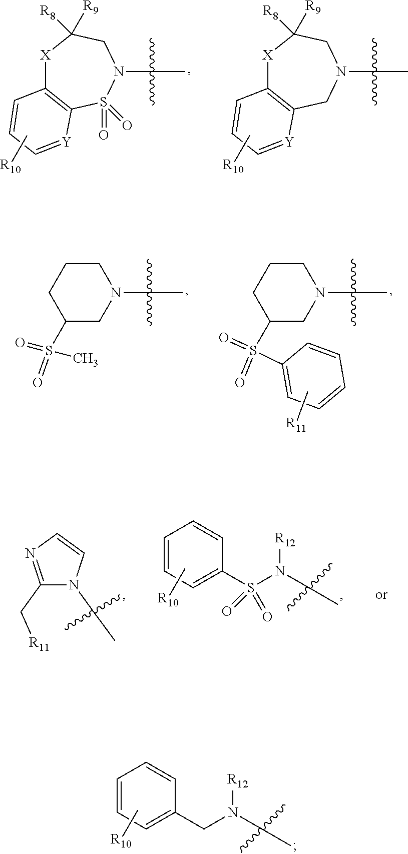

R₈ and R₉ are independently hydrogen or —C₁₋₅alkyl
Each of R₁₀ is independently hydrogen, —C₁₋₅alkyl, —C₃₋₇cycloalkyl or halo;
R₁₁ is hydrogen or —C₅₋₈cycloalkyl;
R₁₂ is hydrogen, —C₁₋₆alkyl or —C₃₋₆cycloalkyl, wherein —C₁₋₆alkyl is unsubstituted or substituted with C₁₋₃alkyl;

### X is CH₂ or O;

### Y is CH or N;

or a pharmaceutically acceptable salt thereof.

“Alkyl” refers to a monovalent saturated hydrocarbon chain having the specified number of carbon member atoms. For example, C₁₋₅alkyl refers to an alkyl group having from 1 to 5 carbon member atoms. Alkyl groups may be straight or branched. Representative branched alkyl groups have one, two, or three branches. Alkyl includes methyl, ethyl, propyl, (n-propyl and isopropyl), butyl (n-butyl, isobutyl, s-butyl, and t-butyl), and pentyl (n-pentyl and isopentyl, etc.).

“Cycloalkyl” refers to a monovalent saturated or unsaturated hydrocarbon ring having the specified number of carbon member atoms. For example, —C₃₋₆cycloalkyl refers to a cycloalkyl group having from 3 to 6 carbon member atoms and —C₅₋₈cycloalkyl refers to a cycloalkyl group having from 5 to 8 carbon member atoms. Unsaturated cycloalkyl groups have one or more carbon-carbon double bonds within the ring. Cycloalkyl groups are not aromatic. Cycloalkyl includes cyclopropyl, cyclopropenyl, cyclobutyl, cyclobutenyl, cyclopentyl, cyclopentenyl, cyclohexyl, and cyclohexenyl.

When used herein, the terms ‘halogen’ and ‘halo’ include fluorine, chlorine, bromine and iodine, and fluoro, chloro, bromo, and iodo, respectively.

“Substituted” in reference to a group indicates that one or more hydrogen atom attached to a member atom within the group is replaced with a substituent selected from the group of defined substituents. It should be understood that the term “substituted” includes the implicit provision that such substitution be in accordance with the permitted valence of the substituted atom and the substituent and that the substitution results in a stable compound (i.e., one that does not spontaneously undergo transformation such as by rearrangement, cyclization, or elimination and that is sufficiently robust to survive isolation from a reaction mixture). When it is stated that a group may contain one or more substituents, one or more (as appropriate) member atoms within the group may be substituted. In addition, a single member atom within the group may be substituted with more than one substituent as long as such substitution is in accordance with the permitted valence of the atom. Suitable substituents are defined herein for each substituted or optionally substituted group.

The term “independently” means that where more than one substituent is selected from a number of possible substituents, those substituents may be the same or different. That is, each substituent is separately selected from the entire group of recited possible substituents.

The invention also includes various isomers of the compounds of Formula (I) and mixtures thereof. “Isomer” refers to compounds that have the same composition and molecular weight but differ in physical and/or chemical properties. The structural difference may be in constitution (geometric isomers) or in the ability to rotate the plane of polarized light (stereoisomers). The compounds according to Formula (I) contain one or more asymmetric centers, also referred to as chiral centers, and may, therefore, exist as individual enantiomers, diastereomers, or other stereoisomeric forms, or as mixtures thereof. All such isomeric forms are included within the present invention, including mixtures thereof.

Chiral centers may also be present in a substituent such as an alkyl group. Where the stereochemistry of a chiral center present in Formula (I), or in any chemical structure illustrated herein, is not specified the structure is intended to encompass any stereoisomer and all mixtures thereof. Thus, compounds according to Formula (I) containing one or more chiral centers may be used as racemic mixtures, enantiomerically enriched mixtures, or as enantiomerically pure individual stereoisomers.

Individual stereoisomers of a compound according to Formula (I) which contain one or more asymmetric centers may be resolved by methods known to those skilled in the art. For example, such resolution may be carried out (1) by formation of diastereoisomeric salts, complexes or other derivatives; (2) by selective reaction with a stereoisomer-specific reagent, for example by enzymatic oxidation or reduction; or (3) by gas-liquid or liquid chromatography in a chiral environment, for example, on a chiral support such as silica with a bound chiral ligand or in the presence of a chiral solvent. The skilled artisan will appreciate that where the desired stereoisomer is converted into another chemical entity by one of the separation procedures described above, a further step is required to liberate the desired form. Alternatively, specific stereoisomers may be synthesized by asymmetric synthesis using optically active reagents, substrates, catalysts or solvents, or by converting one enantiomer to the other by asymmetric transformation.

For compounds falling within the scope of the invention, the structural conventions used in the Examples are as follows: (a) absolute stereochemistry is defined by the structure; (b) when annotated by “or”, then stereochemistry is unknown but resolved; and (c) when annotated by “&” or “and”, then stereochemistry is relative, but racemic.

Preferred compounds of the invention are the trans isomers.

As used herein, “pharmaceutically acceptable” refers to those compounds, materials, compositions, and dosage forms which are, within the scope of sound medical judgment, suitable for use in contact with the tissues of human beings and animals without excessive toxicity, irritation, or other problem or complication, commensurate with a reasonable benefit/risk ratio.

The skilled artisan will appreciate that pharmaceutically acceptable salts of the compounds according to Formula (I) may be prepared. These pharmaceutically acceptable salts may be prepared in situ during the final isolation and purification of the compound, or by separately treating the purified compound in its free acid or free base form with a suitable base or acid, respectively.

In certain embodiments, compounds according to Formula (I) may contain a basic functional group and are therefore capable of forming pharmaceutically acceptable acid addition salts by treatment with a suitable acid. Suitable acids include pharmaceutically acceptable inorganic acids and organic acids. Representative pharmaceutically acceptable acids include hydrogen chloride, hydrogen bromide, nitric acid, sulfuric acid, sulfonic acid, phosphoric acid, acetic acid, hydroxyacetic acid, phenylacetic acid, propionic acid, butyric acid, valeric acid, maleic acid, acrylic acid, fumaric acid, succinic acid, malic acid, malonic acid, tartaric acid, citric acid, salicylic acid, benzoic acid, tannic acid, formic acid, stearic acid, lactic acid, ascorbic acid, methylsulfonic acid, p-toluenesulfonic acid, oleic acid, lauric acid, and the like.

As used herein, the term “a compound of Formula (I)” refers to one or more compounds according to Formula (I). The compound of Formula (I) may exist in solid or liquid form. In the solid state, it may exist in crystalline or noncrystalline form, or as a mixture thereof. The skilled artisan will appreciate that pharmaceutically acceptable solvates may be formed from crystalline compounds wherein solvent molecules are incorporated into the crystalline lattice during crystallization. Solvates may involve non-aqueous solvents such as, but not limited to, ethanol, isopropanol, DMSO, acetic acid, ethanolamine, or ethyl acetate, or they may involve water as the solvent that is incorporated into the crystalline lattice. Solvates wherein water is the solvent incorporated into the crystalline lattice are typically referred to as “hydrates.” Hydrates include stoichiometric hydrates as well as compositions containing variable amounts of water. The invention includes all such solvates.

The skilled artisan will further appreciate that certain compounds of the invention that exist in crystalline form, including the various solvates thereof, may exhibit polymorphism (i.e., the capacity to occur in different crystalline structures). These different crystalline forms are typically known as “polymorphs.” The invention includes all such polymorphs. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. The skilled artisan will appreciate that different polymorphs may be produced, for example, by changing or adjusting the reaction conditions or reagents, used in making the compound. For example, changes in temperature, pressure, or solvent may result in polymorphs. In addition, one polymorph may spontaneously convert to another polymorph under certain conditions.

The subject invention also includes isotopically-labelled compounds, which are identical to those recited in Formula (I) and following, but for the fact that one or more atoms are replaced by an atom having an atomic mass or mass number different from the atomic mass or mass number usually found in nature. Examples of isotopes that can be incorporated into compounds of the invention and pharmaceutically acceptable salts thereof include isotopes of hydrogen, carbon, nitrogen, oxygen, phosphorous, sulphur, fluorine, iodine, and chlorine, such as ²H, ³H, ¹¹C, ¹³C, ¹⁴C, ¹⁵N, ¹⁷O, ¹⁸O, ³¹P, ³²P, ³⁵S, ¹⁸F, ³⁶Cl, ¹²³I and ¹²⁵I.

Compounds of the present invention and pharmaceutically acceptable salts of said compounds that contain the aforementioned isotopes and/or other isotopes of other atoms are within the scope of the present invention. Isotopically-labelled compounds of the present invention, for example those into which radioactive isotopes such as ³H, ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. ¹¹C and ¹⁸F isotopes are particularly useful in PET (positron emission tomography), and ¹²⁵I isotopes are particularly useful in SPECT (single photon emission computerized tomography), all useful in brain imaging. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of Formula (I) and following of this invention can generally be prepared by carrying out the procedures disclosed in the Schemes and/or in the Examples below, by substituting a readily available isotopically labeled reagent for a non-isotopically labeled reagent.

### Representative Embodiments

In one embodiment, the compound of Formula (I) is:

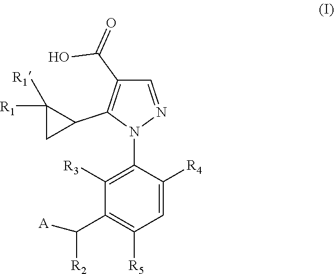

wherein:
R₁ is hydrogen, C₁₋₅alkyl, triazolyl, pyridyl, pyridazinyl, imidazolyl, pyrazolyl, isoxazolyl, halo, —NR₆—C(O)—R₇ or —C(O)R₇, and wherein the triazolyl, pyridyl, pyridazinyl, imidazolyl, pyrazolyl or isoxazolyl is unsubstituted or substituted by one or two substituents independently selected from —C₁₋₃alkyl, —CF₃ and halo;
R₁′ is hydrogen or halo;
R₂ is hydrogen, —C₁₋₅alkyl, —C₃₋₆cycloalkyl, or halo;
R₃ is hydrogen, —C₁₋₅alkyl, —C₃₋₆cycloalkyl, or halo;
or, when R₂ and R₃ are each C₁₋₅alkyl, together they form a 5- to 6-membered cycloalkyl ring fused to the adjacent phenyl ring;
R₄ is hydrogen, —C₁₋₅alkyl, —C₃₋₆cycloalkyl, or halo;
R₅ is hydrogen, —C₁₋₅alkyl, —C₃₋₆cycloalkyl, or halo;
or, when R₂ and R₅ are each C₁₋₅alkyl, together they form a 5- to 6-membered cycloalkyl ring fused to the adjacent phenyl ring;
R₆ and R₇ are independently hydrogen or —C₁₋₅alkyl;

### A is

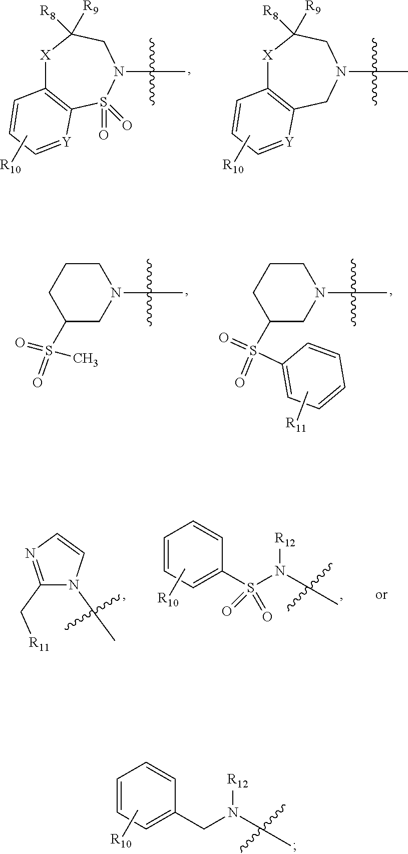

R₈ and R₉ are independently hydrogen or —C₁₋₅alkyl
Each of R₁₀ is independently hydrogen, —C₁₋₅alkyl, —C₃₋₇cycloalkyl or halo;
R₁₁ is hydrogen or —C₅₋₈cycloalkyl;
R₁₂ is hydrogen, —C₁₋₆alkyl or —C₃₋₆cycloalkyl, wherein —C₁₋₆alkyl is unsubstituted or substituted with C₁₋₃alkyl;

### X is CH₂ or O;

### Y is CH or N;

or a pharmaceutically acceptable salt thereof.

In another embodiment, the compound of Formula (I) is substituted as follows:

R₁ is triazolyl, pyridyl, pyridazinyl, imidazolyl, pyrazolyl or isoxazolyl, and wherein the triazolyl, pyridyl, pyridazinyl, imidazolyl, pyrazolyl or isoxazolyl is unsubstituted or substituted by one or two substituents independently selected from —C₁₋₃alkyl, —CF₃ and halo;

R₁′ is hydrogen or halo;

R₂ is hydrogen, —C₁₋₅alkyl, —C₃₋₆cycloalkyl, or halo;

R₃ is hydrogen, —C₁₋₅alkyl, or halo;

R₄ is hydrogen, —C₁₋₅alkyl, or halo;

R₅ is hydrogen, —C₁₋₅alkyl, —C₃₋₆cycloalkyl, or halo;

### A is

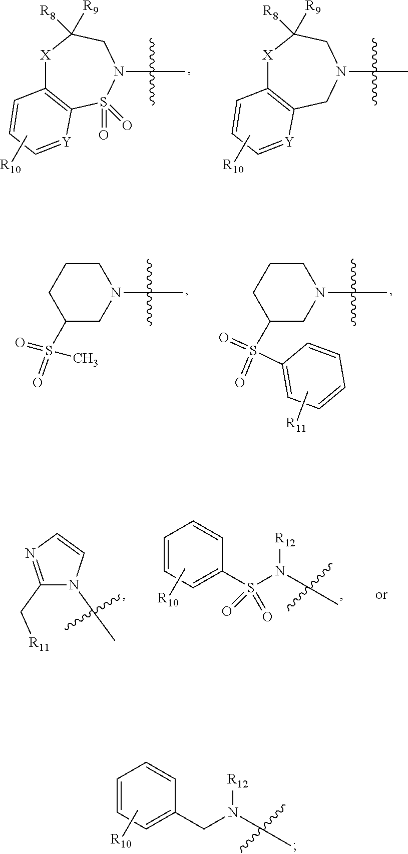

R₈ and R₉ are each hydrogen;
Each of R₁₀ is hydrogen;
R₁₁ is hydrogen;
R₁₂ is hydrogen or —C₁₋₆alkyl, wherein —C₁₋₆alkyl is unsubstituted or substituted with C₁₋₃alkyl;

### X is CH₂ or O;

### Y is CH or N;

or a pharmaceutically acceptable salt thereof.

It is to be understood that the present invention covers all combinations of the embodiments and particular groups described hereinabove

Specific examples of compounds of the present invention include the following:
1-(3-(((R)-4-ethyl-1,1-dioxido-3,4-dihydro-2H-benzo[b][1,4,5]oxathiazepin-2-yl)methyl)phenyl)-5-(trans)-2-(1-methyl-1H-1,2,3-triazol-4-yl)cyclopropyl)-1H-pyrazole-4-carboxylic acid;
1-(3-(((S)-4-Ethyl-1,1-dioxido-4,5-dihydrobenzo[f][1,2]thiazepin-2(3H)-yl)methyl)phenyl)-5-(trans)-2-(1-methyl-1H-1,2,3-triazol-4-yl)cyclopropyl)-1H-pyrazole-4-carboxylic acid.
1-(3-((8-fluoro-4,4-dimethyl-1,1-dioxido-3,4-dihydro-2H-benzo[b][1,4,5]oxathiazepin-2-yl)methyl)phenyl)-5-(trans)-2-(1-methyl-1H-1,2,3-triazol-4-yl)cyclopropyl)-1H-pyrazole-4-carboxylic acid;
1-(3-((R or S)-1-((S)-4-Methyl-1,1-dioxido-8-(trifluoromethyl)-4,5-dihydrobenzo[f][1,2]thiazepin-2(3H)-yl)ethyl)phenyl)-5-((1R,2R)-2-(1-methyl-1H-1,2,3-triazol-4-yl)cyclopropyl)-1H-pyrazole-4-carboxylic acid
1-(3-((4,4-Dimethyl-4,5-dihydro-1H-benzo[c]azepin-2(3H)-yl)methyl)phenyl)-5-(trans)-2-(1-methyl-1H-1,2,3-triazol-4-yl)cyclopropyl)-1H-pyrazole-4-carboxylic acid;
1-(3-((2,2-dimethyl-2,3-dihydrobenzo[f][1,4]oxazepin-4(5H)-yl)methyl)phenyl)-5-(trans)-2-(1-methyl-1H-1,2,3-triazol-4-yl)cyclopropyl)-1H-pyrazole-4-carboxylic acid;
1-(3-((2,2-dimethyl-2,3-dihydropyrido[2,3-f][1,4]oxazepin-4(5H)-yl)methyl)phenyl)-5-(trans)-2-(1-methyl-1H-1,2,3-triazol-4-yl)cyclopropyl)-1H-pyrazole-4-carboxylic acid;
1-(3-(((R)-4-ethyl-4,5-dihydro-1H-benzo[c]azepin-2(3H)-yl)methyl)phenyl)-5-(trans)-2-(1-methyl-1H-1,2,3-triazol-4-yl)cyclopropyl)-1H-pyrazole-4-carboxylic acid;
1-(3-(((S)-4-ethyl-4,5-dihydro-1H-benzo[c]azepin-2(3H)-yl)methyl)phenyl)-5-(trans)-2-(1-methyl-1H-1,2,3-triazol-4-yl)cyclopropyl)-1H-pyrazole-4-carboxylic acid;
1-(3-((7-bromo-2,2-dimethyl-2,3-dihydrobenzo[f][1,4]oxazepin-4(5H)-yl)methyl)phenyl)-5-(trans)-2-(1-methyl-1H-1,2,3-triazol-4-yl)cyclopropyl)-1H-pyrazole-4-carboxylic acid (trifluoroacetate);
1-(3-((5-ethyl-2,2-dimethyl-2,3-dihydrobenzo[f][1,4]oxazepin-4(5H)-yl)methyl)phenyl)-5-(trans)-2-(1-methyl-1H-1,2,3-triazol-4-yl)cyclopropyl)-1H-pyrazole-4-carboxylic acid;
Sodium 1-(3-(((R)-2-ethyl-2,3-dihydrobenzo[f][1,4]oxazepin-4(5H)-yl)methyl)phenyl)-5-(trans)-2-(1-methyl-1H-1,2,3-triazol-4-yl)cyclopropyl)-1H-pyrazole-4-carboxylate;
1-(3-((4-methyl-1,1-dioxido-4,5-dihydrobenzo[f][1,2]thiazepin-2(3H)-yl)methyl)phenyl)-5-((trans)-2-(1-methyl-1H-1,2,3-triazol-4-yl)cyclopropyl)-1H-pyrazole-4-carboxylic acid;
1-(3-((4-methyl-1,1-dioxido-8-(trifluoromethyl)-4,5-dihydrobenzo[f][1,2]thiazepin-2(3H)-yl)methyl)phenyl)-5-((trans)-2-(1-methyl-1H-1,2,3-triazol-4-yl)cyclopropyl)-1H-pyrazole-4-carboxylic acid;
1-(3-((8-bromo-4-methyl-1,1-dioxido-4,5-dihydrobenzo[f][1,2]thiazepin-2(3H)-yl)methyl)phenyl)-5-((trans)-2-(1-methyl-1H-1,2,3-triazol-4-yl)cyclopropyl)-1H-pyrazole-4-carboxylic acid;
1-(3-((4-methyl-1,1-dioxido-7-(trifluoromethyl)-4,5-dihydrobenzo[f][1,2]thiazepin-2(3H)-yl)methyl)phenyl)-5-((trans)-2-(1-methyl-1H-1,2,3-triazol-4-yl)cyclopropyl)-1H-pyrazole-4-carboxylic acid;
1-(3-((4-methyl-1,1-dioxido-7-(trifluoromethyl)-4,5-dihydrobenzo[f][1,2]thiazepin-2(3H)-yl)methyl)phenyl)-5-((trans)-2-(1-methyl-1H-1,2,3-triazol-4-yl)cyclopropyl)-1H-pyrazole-4-carboxylic acid;
1-(3-(((S)-4-ethyl-1,1-dioxido-8-(trifluoromethyl)-4,5-dihydrobenzo[f][1,2]thiazepin-2(3H)-yl)methyl)phenyl)-5-((1R,2R)-2-(1-methyl-1H-1,2,3-triazol-4-yl)cyclopropyl)-1H-pyrazole-4-carboxylic acid;
1-(3-(((S)-4-butyl-1,1-dioxido-8-(trifluoromethyl)-4,5-dihydrobenzo[f][1,2]thiazepin-2(3H)-yl)methyl)phenyl)-5-((1R,2R)-2-(1-methyl-1H-1,2,3-triazol-4-yl)cyclopropyl)-1H-pyrazole-4-carboxylic acid; 1-(3-(((S)-8-bromo-4-ethyl-1,1-dioxido-4,5-dihydrobenzo[f][1,2]thiazepin-2(3H)-yl)methyl)phenyl)-5-((1R,2R)-2-(1-methyl-1H-1,2,3-triazol-4-yl)cyclopropyl)-1H-pyrazole-4-carboxylic acid;
1-(3-((2-(Cycloheptylmethyl)-1H-imidazol-1-yl)methyl)phenyl)-5-(trans)-2-(1-methyl-1H-1,2,3-triazol-4-yl)cyclopropyl)-1H-pyrazole-4-carboxylic acid;
5-(trans)-2-(1-methyl-1H-1,2,3-triazol-4-yl)cyclopropyl)-1-(3-((2-(piperidin-1-ylmethyl)-1H-imidazol-1-yl)methyl)phenyl)-1H-pyrazole-4-carboxylic acid;
1-(3-(((R)-4-Ethyl-1,1-dioxido-3,4-dihydro-2H-benzo[b][1,4,5]oxathiazepin-2-yl)methyl)-4-methylphenyl)-5-(trans)-2-(1-methyl-1H-1,2,3-triazol-4-yl)cyclopropyl)-1H-pyrazole-4-carboxylic acid;
1-((R)-1-((S)-4-Methyl-1,1-dioxido-8-(trifluoromethyl)-4,5-dihydrobenzo[f][1,2]thiazepin-2(3H)-yl)-2,3-dihydro-1H-inden-4-yl)-5-((1R,2R)-2-(1-methyl-1H-1,2,3-triazol-4-yl)cyclopropyl)-1H-pyrazole-4-carboxylic acid;
1-((S)-1-((S)-4-Methyl-1,1-dioxido-8-(trifluoromethyl)-4,5-dihydrobenzo[f][1,2]thiazepin-2(3H)-yl)-2,3-dihydro-1H-inden-4-yl)-5-((1R,2R)-2-(1-methyl-1H-1,2,3-triazol-4-yl)cyclopropyl)-1H-pyrazole-4-carboxylic acid;
1-(3-(2-methoxy-N-methylphenylsulfonamido)-2,3-dihydro-1H-inden-5-yl)-5-((1R,2R)-2-(1-methyl-1H-1,2,3-triazol-4-yl)cyclopropyl)-1H-pyrazole-4-carboxylic acid;
1-(3-(((R)-2-ethyl-2,3-dihydrobenzo[f][1,4]oxazepin-4(5H)-yl)methyl)-4-methylphenyl)-5-(trans)-2-(1-methyl-1H-1,2,3-triazol-4-yl)cyclopropyl)-1H-pyrazole-4-carboxylic acid;
1-(3-(((R)-4-Ethyl-1,1-dioxido-3,4-dihydro-2H-benzo[b][1,4,5]oxathiazepin-2-yl)methyl)phenyl)-5-(trans)-2-(1-(2,2,2-trifluoroethyl)-1H-1,2,3-triazol-4-yl)cyclopropyl)-1H-pyrazole-4-carboxylic acid;
1-(3-(((S)-4-Methyl-1,1-dioxido-7-(trifluoromethyl)-4,5-dihydrobenzo[f][1,2]thiazepin-2(3H)-yl)methyl)phenyl)-5-(trans-2-(1-methyl-1H-pyrazol-4-yl)cyclopropyl)-1H-pyrazole-4-carboxylic acid;
1-(3-(((R)-4-ethyl-1,1-dioxido-3,4-dihydro-2H-benzo[b][1,4,5]oxathiazepin-2-yl)methyl)phenyl)-5-(trans-2-(1-methyl-1H-pyrazol-4-yl)cyclopropyl)-1H-pyrazole-4-carboxylic acid;
1-(3-(((S)-4-methyl-1,1-dioxido-4,5-dihydrobenzo[f][1,2]thiazepin-2(3H)-yl)methyl)phenyl)-5-(trans-2-(1-methyl-1H-pyrazol-4-yl)cyclopropyl)-1H-pyrazole-4-carboxylic acid;
1-(3-(((R)-4-methyl-1,1-dioxido-7-(trifluoromethyl)-4,5-dihydrobenzo[f][1,2]thiazepin-2(3H)-yl)methyl)phenyl)-5-(trans-2-(1-methyl-1H-pyrazol-4-yl)cyclopropyl)-1H-pyrazole-4-carboxylic acid;
1-(3-((N-hexyl-[1,1′-biphenyl]-3-ylsulfonamido)methyl)phenyl)-5-((trans)-2-(1-methyl-1H-1,2,3-triazol-4-yl)cyclopropyl)-1H-pyrazole-4-carboxylic acid;
5-((trans)-2-(1-methyl-1H-1,2,3-triazol-4-yl)cyclo-propyl)-1-(3-((N-methyl-3-phenoxy-phenyl-sulfonamido)-methyl)phenyl)-1H-pyrazole-4-carboxylic acid;
1-(3-((N-hexyl-4-phenoxyphenylsulfonamido)methyl)phenyl)-5-((trans)-2-(1-methyl-1H-1,2,3-triazol-4-yl)-cyclopropyl)-1H-pyrazole-4-carboxylic acid;
1-(3-((N-hexyl-2-methylphenylsulfonamido)methyl)phenyl)-5-((trans)-2-(1-methyl-1H-1,2,3-triazol-4-yl)cyclo-propyl)-1H-pyrazole-4-carboxylic acid;
5-((trans)-2-(1-methyl-1H-1,2,3-triazol-4-yl)cyclo-propyl)-1-(3-((N-methyl-2-phenoxyphenyl-sulfonamido)-methyl)phenyl)-1H-pyrazole-4-carboxylic acid;
5-((trans)-2-(1-methyl-1H-1,2,3-triazol-4-yl)cyclo-propyl)-1-(3-((N-methyl-[1,1′-biphenyl]-4-ylsulfonamido)-methyl)phenyl)-1H-pyrazole-4-carboxylic acid;
1-(3-((5-chloro-2-methoxy-N-methylphenyl-sulfonamido)-methyl)phenyl)-5-((trans)-2-(1-methyl-1H-1,2,3-triazol-4-yl)cyclo-propyl)-1H-pyrazole-4-carboxylic acid;
1-(3-((2-chloro-N-methyl-6-(trifluoro-methyl)phenyl-sulfonamido)-methyl)phenyl)-5-((trans)-2-(1-methyl-1H-1,2,3-triazol-4-yl)cyclo-propyl)-1H-pyrazole-4-carboxylic acid;
1-(3-((N,2-dimethylphenylsulfonamido)methyl)phenyl)-5-((trans)-2-(1-methyl-1H-1,2,3-triazol-4-yl)cyclopropyl)-1H-pyrazole-4-carboxylic acid;
1-(3-((5-bromo-2-methoxy-N-methylphenylsulfonamido)methyl)phenyl)-5-((trans)-2-(1-methyl-1H-1,2,3-triazol-4-yl)cyclopropyl)-1H-pyrazole-4-carboxylic acid;
1-(3-((2-methoxy-N-methyl-4-nitrophenylsulfonamido)methyl)phenyl)-5-((trans)-2-(1-methyl-1H-1,2,3-triazol-4-yl)cyclopropyl)-1H-pyrazole-4-carboxylic acid;
1-(3-((2-ethoxy-N-methylphenylsulfonamido)methyl)phenyl)-5-((trans)-2-(1-methyl-1H-1,2,3-triazol-4-yl)cyclopropyl)-1H-pyrazole-4-carboxylic acid;
5-((trans)-2-(1-methyl-1H-1,2,3-triazol-4-yl)cyclopropyl)-1-(3-((N-methyl-2-(trifluoromethoxy)phenylsulfonamido)methyl)phenyl)-1H-pyrazole-4-carboxylic acid;
1-(3-((2-methoxy-N-methylphenylsulfonamido)methyl)phenyl)-5-((trans)-2-(1-methyl-1H-1,2,3-triazol-4-yl)cyclopropyl)-1H-pyrazole-4-carboxylic acid;
5-((trans)-2-(1-methyl-1H-1,2,3-triazol-4-yl)cyclopropyl)-1-(3-((N-methylphenylsulfonamido)methyl)phenyl)-1H-pyrazole-4-carboxylic acid;
1-(3-((N-hexyl-2-phenoxyphenylsulfonamido)methyl)phenyl)-5-((trans)-2-(1-methyl-1H-1,2,3-triazol-4-yl)cyclopropyl)-1H-pyrazole-4-carboxylic acid;
5-((trans)-2-(1-methyl-1H-1,2,3-triazol-4-yl)cyclopropyl)-1-(3-((2-methyl-N-propylphenylsulfonamido)methyl)phenyl)-1H-pyrazole-4-carboxylic acid;
5-((trans)-2-(1-methyl-1H-1,2,3-triazol-4-yl)cyclopropyl)-1-(3-((N-methylquinoline-8-sulfonamido)methyl)phenyl)-1H-pyrazole-4-carboxylic acid;
1-(3-((2-methoxyphenylsulfonamido)methyl)phenyl)-5-((trans)-2-(1-methyl-1H-1,2,3-triazol-4-yl)cyclopropyl)-1H-pyrazole-4-carboxylic acid;
1-(3-((N-cyclopropyl-2-methoxyphenylsulfonamido)methyl)phenyl)-5-((trans)-2-(1-methyl-1H-1,2,3-triazol-4-yl)cyclopropyl)-1H-pyrazole-4-carboxylic acid; 1-(3-(((5-bromo-2-ethoxybenzyl)(methyl)amino)methyl)phenyl)-5-((trans)-2-(1-methyl-1H-1,2,3-triazol-4-yl)cyclopropyl)-1H-pyrazole-4-carboxylic acid trifluoroacetate; 1-(3-(((5-bromo-2-ethoxybenzyl)(cyclopropyl)amino)methyl)phenyl)-5-((trans)-2-(1-methyl-1H-1,2,3-triazol-4-yl)cyclopropyl)-1H-pyrazole-4-carboxylic acid trifluoroacetate; 1-(3-((N-Hexyl-2-methylphenylsulfonamido)methyl)phenyl)-5-(trans)-2-(1-methyl-1H-pyrazol-4-yl)cyclopropyl)-1H-pyrazole-4-carboxylic acid; and
1-(3-(((S)-4-Methyl-1,1-dioxido-7-(trifluoromethyl)-4,5-dihydrobenzo[f][1,2]thiazepin-2(3H)-yl)methyl)phenyl)-5-(trans-2-(3-methylisoxazol-5-yl)cyclopropyl)-1H-pyrazole-4-carboxylic acid.
   or a pharmaceutically acceptable salt thereof.

### Compound Preparation

The skilled artisan will appreciate that if a substituent described herein is not compatible with the synthetic methods described herein, the substituent may be protected with a suitable protecting group that is stable to the reaction conditions. The protecting group may be removed at a suitable point in the reaction sequence to provide a desired intermediate or target compound. Suitable protecting groups and the methods for protecting and de-protecting different substituents using such suitable protecting groups are well known to those skilled in the art; examples of which may be found in T. Greene and P. Wuts, Protecting Groups in Chemical Synthesis (3rd ed.), John Wiley & Sons, NY (1999). In some instances, a substituent may be specifically selected to be reactive under the reaction conditions used. Under these circumstances, the reaction conditions convert the selected substituent into another substituent that is either useful as an intermediate compound or is a desired substituent in a target compound.

The synthesis of the compounds of the general Formula (I) and pharmaceutically acceptable derivatives and salts thereof may be accomplished as outlined below in Schemes 1-8. In the following description, the groups are as defined above for compounds of Formula (I) unless otherwise indicated. Abbreviations are as defined in the Examples section. Starting materials are commercially available or are made from commercially available starting materials using methods known to those skilled in the art.

### Compound Preparation

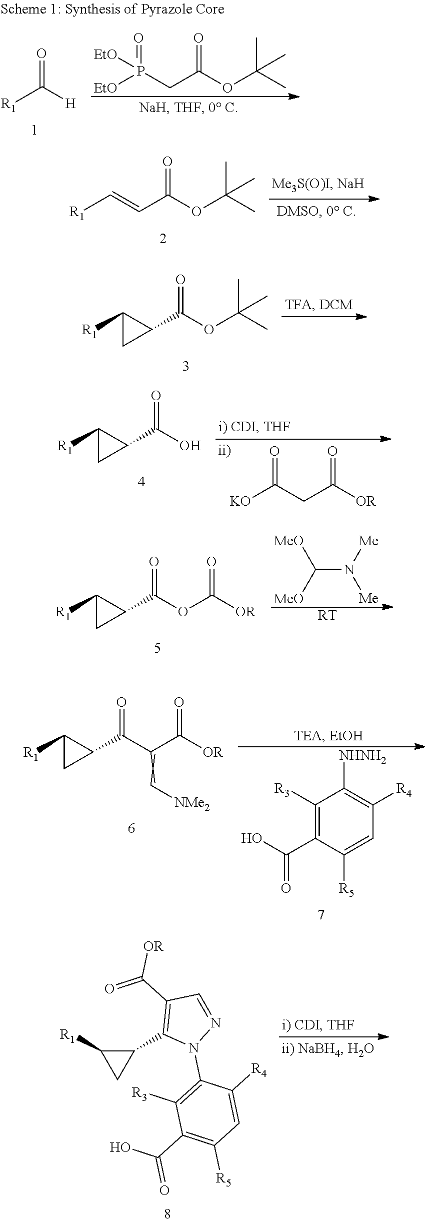

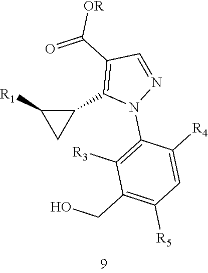

An appropriately-substituted aldehyde (1) is treated with the sodium salt of tert-butyl 2-(diethoxyphosphoryl)acetate in THF to generate olefinated product 2. Subsequent treatment with the sodium salt of trimethylsulfoxonium iodide in DMSO provides trans-cyclopropane 3. Acid-mediated (TFA) hydrolysis of the tert-butyl ester gives acid 4. The carboxylic acid functionality is then activated with CDI, and subjection to potassium-3-alkoxy-3-oxopropanoate provides keto-ester 5, in which “R” is an akyl group (customarily methyl or ethyl) utilized to mask the carboxylic acid functionality. Subsequent exposure to N,N-dimethylformamide dimethyl acetal affords 6, which is treated with a broad range of functionalized aryl hydrazines (7) to assemble substituted pyrazoles (8). Activation of the acid functionality with CDI followed by reduction with sodium borohydride gives alcohol 9, which serves as a veratile intermediate in the work described herein.

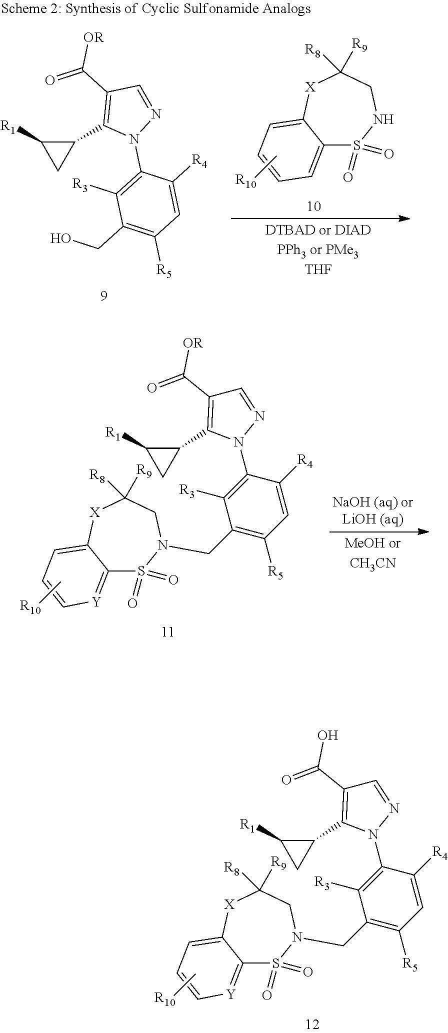

Mitsunobu coupling of alcohol 9 with the described cyclic aryl sulfonamide 10 mediated by di-tert-butyl-azodicarboxylate (DTBAD) or diisopropyl azodicarboxylate (DIAD) and triphenylphosphine or trimethylphosphine affords intermediates such as 11. Subsequent hydrolysis with aqueous lithium hydroxide or sodium hydroxide provides analogs of generic structure 12. This hydrolysis can be conducted in a variety of solvents; most notably methanol, ethanol or acetonitrile.

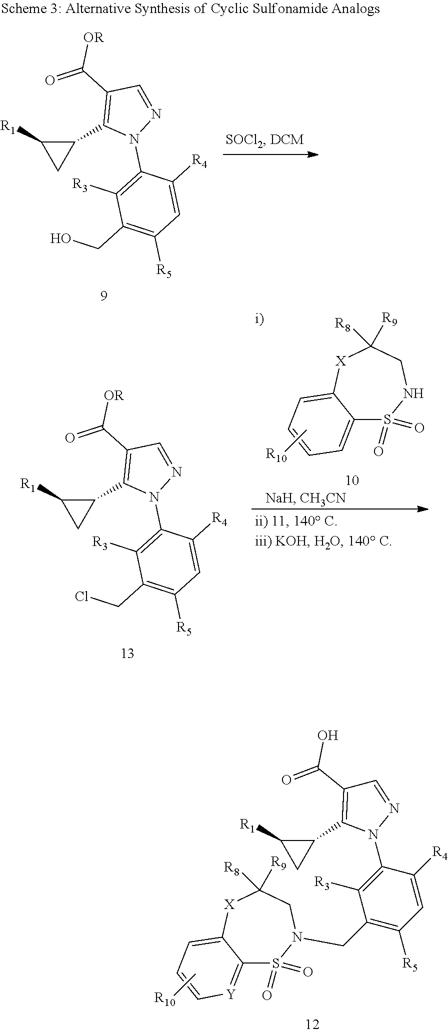

An alternative synthesis of the sulfonamide exemplars involves conversion of alcohol 9 to the corresponding benzyl chloride 13 by subjection to thionyl chloride (SOCl). A one-pot reaction involving displacement of the chloride with the sodium salt of the cyclic aryl sulfonamide 10 followed by ester hydrolysis with aqueous potassium hydroxide affords sulfonamide 12.

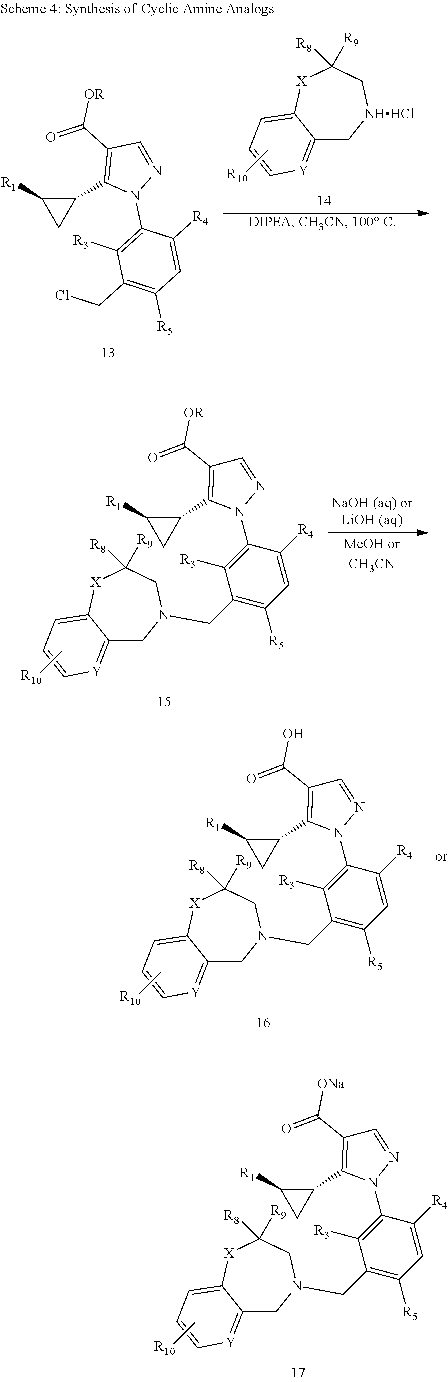

The synthetic strategy providing cyclic benzyl amine analogs is illustrated in Scheme 4. Displacement of the chloride of 13 with cyclic amine 14 affords benzyl amine 15. Subsequent base-mediated (aqueous NaOH or LiOH) hydrolysis gives carboxylic acid 16 or sodium carboxylate 17 depending on the purification procedure (i.e. reverse-phase HPLC can be conducted under either acidic (to give 16) or neutral (to give 17) conditions).

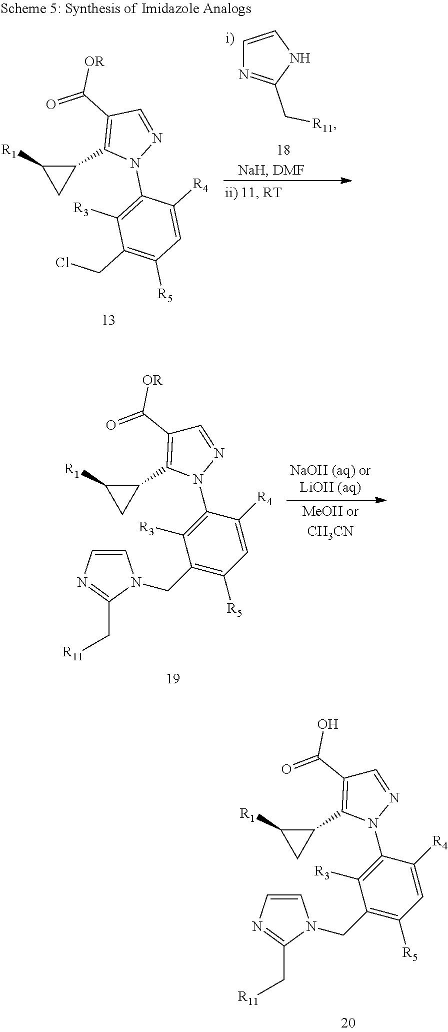

The preparation of the imidazole analogs described herein involves displacement of the chloride of 13 with the sodium salt of functionalized imidazole 18. Subsequent hydrolysis utilizing either aqueous sodium hydroxide or lithium hydroxide gives compounds of generic structure 20.

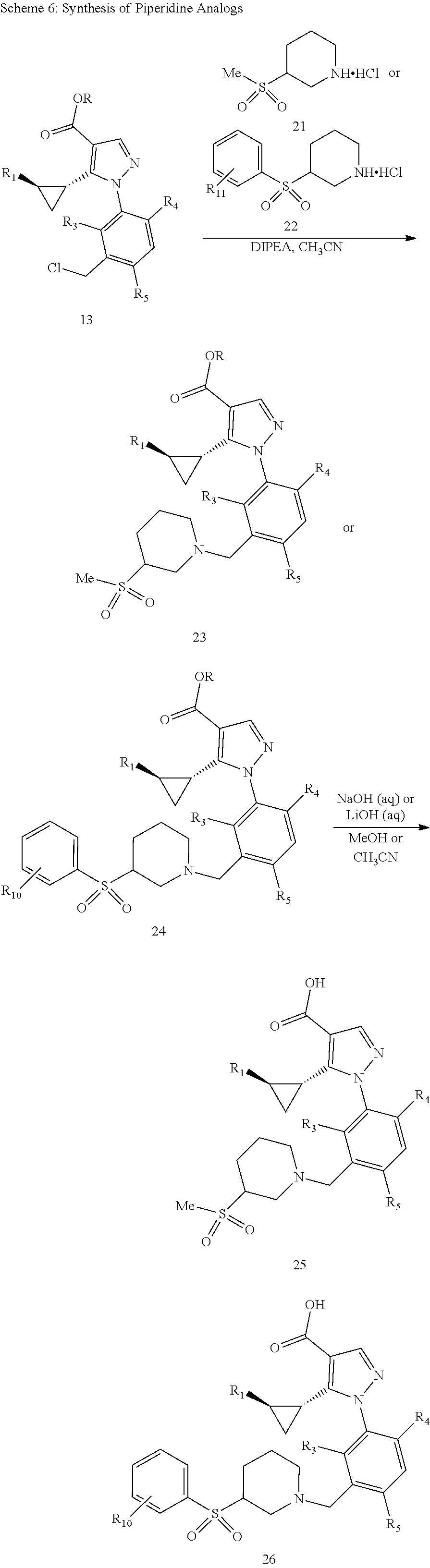

Substituted piperidines 25 and 26 are prepared analogously to cyclic amine 16 and imidazole 20. Displacement of the chloride of 13 with piperidine 21 or 22 affords 23 or 24, respectively. Base-mediated hydrolysis with aqueous sodium hydroxide or lithium hydroxide provides 25 or 26.

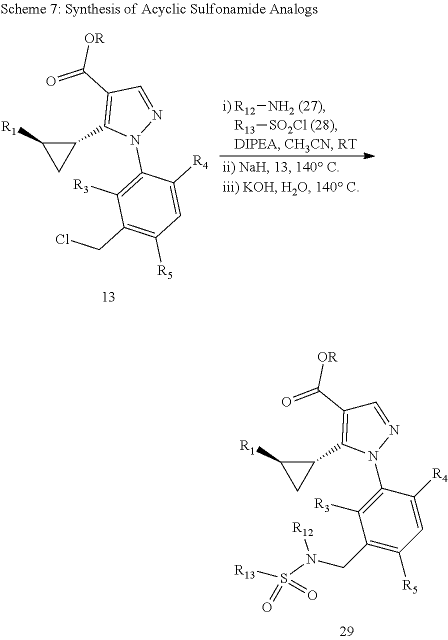

The acyclic sulfonamide analogs described herein are prepared through a one-pot procedure involving multiple transformations. Sulfonylation of amine 27 with sulfonyl chloride 28 prepares the requisite sulfonamide in situ, which is then deprotonated with sodium hydride to furnish the corresponding sodium salt. Subjection to chloride 13 followed by base-mediated ester hydrolysis with aqueous potassium hydroxide generates compounds of generic structure 29.

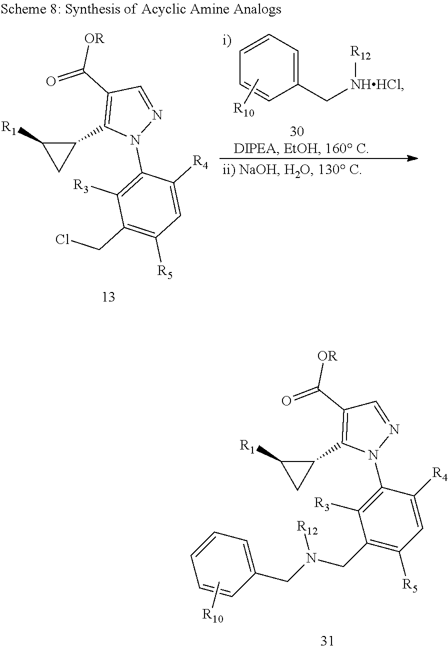

Acyclic amines of general structure 31 are assembled by displacement of the chloride of 13 with benzyl amine 30. Subsequent ester hydrolysis with aqueous sodium hydroxide affords amine 31.

### Biological Activity

As stated above, the compounds according to Formula I are NRF2 regulators, and are useful in the treatment or prevention of human diseases that exhibit oxidative stress components such as respiratory and non-respiratory disorders, including COPD, asthma, ALI, ARDS, fibrosis, chronic asthma and acute asthma, lung disease secondary to environmental exposures, acute lung infection, chronic lung infection, α1 antitrypsin disease, cystic fibrosis, autoimmune diseases, diabetic nephropathy, chronic kidney disease, sepsis-induced acute kidney injury, acute kidney injury (AKI), kidney disease or malfunction seen during kidney transplantation, Pulmonary Arterial Hypertension, atherosclerosis, hypertension, heart failure, acute coronary syndrome, myocardial infarction, myocardial repair, cardiac remodelling, cardiac arrhythmias, Parkinson's disease (PD), Alzheimer's disease (AD), Friedreich's Ataxia (FA), amyotrophic lateral sclerosis (ALS), multiple sclerosis (MS), inflammatory bowel disease, colon cancer, neovascular (dry) AMD and neovascular (wet) AMD, eye injury, Fuchs Endothelial Corneal Dystrophy (FECD), uveitis or other inflammatory eye conditions, Non-alcoholic Steatohepatitis (NASH), toxin-induced liver disease (e.g., acetaminophen-induced hepatic disease), viral hepatitis, cirrhosis, psoriasis, dermatitis/topical effects of radiation, immunosuppression due to radiation exposure, Preeclampsia, and high altitude sickness.

The biological activity of the compounds according to Formula I can be determined using any suitable assay for determining the activity of a candidate compound as a NRF2 antagonist, as well as tissue and in vivo models.

The biological activity of the compounds of Formula (I) are demonstrated by the following tests.

### BEAS-2B NQO1 MTT Assay

NAD(P)H:quinone oxidoreductase 1 (NQO1), also called DT diaphorase, is a homodimeric FAD-containing enzyme that catalyzes obligatory NAD(P)H-dependent two-electron reductions of quinones and protects cells against the toxic and neoplastic effects of free radicals and reactive oxygen species arising from one-electron reductions. The transcription of NQO1 is finely regulated by NRF2, and thus NQO1 activity is a good marker for NRF2 activation. On day one, frozen BEAS-2B cells (ATCC) were thawed in a water bath, counted, and re-suspended at a concentration of 250,000 cells/mL. Fifty microliters of cells were plated in 384 well black clear-bottomed plates. Plates were incubated at 37° C., 5% CO₂ overnight. On day two, plates were centrifuged and 50 nL of compound or controls were added to the cells. Plates were then incubated at 37° C., 5% CO₂ for 48 hours. On day four, medium was aspirated from the plate and crude cell lysates were made by adding 13 uL of 1× Cell Signaling Technologies lysis buffer with 1 Complete, Mini, EDTA-free Protease Inhibitor Tablet (Roche) for each 10 mL of lysis buffer. After lysis plates were incubated for 20 minutes at room temperature. Two microliters of lysate were removed for use in Cell Titer Glo assay (Promega) and MTT cocktail was prepared (Prochaska et. al. 1998) for measurement of NQO1 activity. Fifty microliters of MTT cocktail is added to each well, plate is centrifuged, and analyzed on an Envision plate reader (Perkin Elmer) using Absorbance 570 nm label for 30 minutes. Product formation was measured kinetically and the pEC₅₀ of NQO1 specific activity induction was calculated by plotting the change in absorbance (Delta OD/min) versus the log of compound concentration followed by 3-parameter fitting.

pEC₅₀ is the negative log of the EC₅₀.

### Beas2B NQO1 MTT Assay

All examples described herein possessed NQO1 specific enzyme activity in BEAS-2B cells with EC₅₀s between >10 μM-<1 nM unless otherwise noted (see table below). EC₅₀s<1 nM (+++++), EC₅₀s 1-10 nM (++++), EC₅₀s 10-100 nM (+++), EC₅₀s 100 nM-1 μM (++), EC₅₀s 1-10 μM (+), EC₅₀s>10 μM (−), or were not determined (ND).

| Ex # | EC₅₀ |
|---|---|
| 3 | ++ |
| 4 | ++ |
| 5 | + |
| 6 | ++ |
| 7 | ++ |
| 8 | + |
| 9 | ++ |
| 10 | + |
| 11 | + |
| 12 | + |
| 13 | ++ |
| 14 | + |
| 15 | + |
| 16 | ++ |
| 17 | ++ |
| 18 | +++ |
| 19 | + |
| 20 | ++++ |
| 21 | +++++ |
| 22 | ++++ |
| 23 | ++ |
| 24 | + |
| 25 | + |
| 26a | ++ |
| 26b | + |
| 27 | ++ |
| 28 | ++ |
| 29 | ++ |
| 30 | + |
| 31 | + |
| 32 | + |
| 33 | + |
| 34 | +++ |
| 35 | + |
| 36 | ++ |
| 37 | +++ |
| 38 | + |
| 39 | + |
| 40 | + |
| 41 | + |
| 42 | + |
| 43 | + |
| 44 | + |
| 45 | + |
| 46 | + |
| 47 | +++ |
| 48 | + |
| 49 | ++ |
| 50 | +++ |
| 51 | ++ |
| 52 | ++ |
| 53 | ++ |
| 54 | ++ |
| 55 | ++ |
| 56 | ++ |
| 57 | ++ |

*in some determinations IC₅₀ values were >10 μM

### NRF2-Keap1 FP Assay

One model for the NRF2-Keap1 interaction is through two binding sites in the Neh2 domain on NRF2. The two sites are referred to as the DLG binding motif (latch domain, uM affinity) and the ETGE binding motif (hinge domain, nM affinity). The Keap1 protein consists of an N-terminal region (NTR), a broad complex, tramtrack, and brick a' brac domain (BTB), an intervening region (IVR), a double glycine repeat domain (DGR or Kelch), and a C-terminal region. The DLG and ETGE motifs of NRF2's Neh2 domain bind to the Kelch domain of Keap1 at different affinities. In the Keap1 Kelch fluorescence polarization (FP) assay, a TAMRA-labeled 16mer peptide (AFFAQLQLDEETGEFL) containing the ETGE motif of NRF2 and the Kelch domain (321-609) of Keap1 is used. The assay determines if a compound interferes with the binding between Keap1 (361-609) and the TAMRA-labeled peptide. Binding of TAMRA-labeled NRF2 peptide to Keap1 (321-609) results in a high FP signal. If a compound interferes with the binding between the peptide and the protein, it will cause the assay signal to decrease. Thus, assay signal is inversely proportional to binding inhibition.

### FP Assay:

100 nl of 100× compound dose response curves (serial 3-fold dilutions) in DMSO were stamped using an Echo liquid handling system (Labcyte) into 384-well low volume black assay plates (Greiner, #784076), with DMSO in columns 6 and 18. The top concentration of compound was located in columns 1 and 13. Keap1 (321-609) was diluted to 40 nM (2×) in 1× assay buffer (50 mM Tris, pH 8.0, 100 mM NaCl, 5 mM MgCl₂, 1 mM DTT, 2 mM CHAPS, and 0.005% BSA) and 5 ul was added using a Multidrop Combi (Thermo Electron Corporation) equipped with a metal tip dispenser to all wells of the compound plate, except column 18. Column 18 received only 5 ul of assay buffer. Immediately, 5 uL of 16 nM (2×) of Tamra labeled peptide (AFFAQLQLDEETGEFL, 21^{st} Century Biochemicals) was added to all wells of the plate. The plates were spun at 500 rpm for 1 min, incubated for 1 hr at room temperature, and read on an Analyst GT (Molecular Devices) equipped with excitation (530/25 nm) and emission (580/10 nm) filters designed for Tamra probes. A 561 nm dichroic mirror was also used in the Analyst. The final assay concentrations of Keap1l (321-609) and Tamra labeled peptide were 20 nM and 8 nM, respectively. Fluorescence measurements, represented as mP, were used in the transformation of the data. Compound activity was calculated based on percent inhibition, normalized against controls in the assay (Control 1 contains the Tamra peptide and Keap1 (321-609) together (0% response) and control 2 contains the Tamra peptide alone (100% response)). Data analysis was handled using the software package Abase XE (Surrey, United Kingdom. The % inhibition values were calculated by the equation:

100−(100*((compound response−average control 2)/(average control 1−average control2))).

For calculation of pIC₅₀s, Abase XE used a four parameter equation.

All examples described herein possessed activity in the Keap1/NRF2 FP assay as listed (see table below) unless otherwise noted. IC₅₀s<1 nM (+++++), IC₅₀s 1 nM-10 nM (++++), IC₅₀s 10-100 nM (+++), IC₅₀s 100 nM-1 μM (++), IC₅₀s 1-10 μM (+), IC₅₀s>10 μM (−), or were not determined (ND).

| Ex # | EC₅₀ |
|---|---|
| 3 | ++++ |
| 4 | ++++ |
| 5 | ++++ |
| 6 | ++++ |
| 7 | +++ |
| 8 | +++ |
| 9 | +++ |
| 10 | +++ |
| 11 | +++ |
| 12 | + |
| 13 | ++++ |
| 14 | ++ |
| 15 | +++ |
| 16 | ++++ |
| 17 | ++++ |
| 18 | ++++ |
| 19 | ++ |
| 20 | +++++ |
| 21 | +++++ |
| 22 | +++++ |
| 23 | ++++ |
| 24 | +++ |
| 25 | ++++ |
| 26a | +++ |
| 26b | ++ |
| 27 | + |
| 28 | ++ |
| 29 | ++++ |
| 30 | +++ |
| 31 | ++ |
| 32 | ++ |
| 33 | + |
| 34 | ++++ |
| 35 | +++ |
| 36 | +++ |
| 37 | +++ |
| 38 | +++ |
| 39 | +++ |
| 40 | ++ |
| 41 | ++ |
| 42 | ++ |
| 43 | ++ |
| 44 | ++ |
| 45 | ++ |
| 46 | ++ |
| 47 | ++ |
| 48 | ++ |
| 49 | ++ |
| 50 | ++ |
| 51 | ++ |
| 52 | ++ |
| 53 | + |
| 54 | ++ |
| 55 | + |
| 56 | + |
| 57 | ++ |

### NRF2-Keap1 TR-FRET Low Protein Assay

In the NRF2-Keap1 TR-FRET (time-resolved fluorescence resonance energy transfer) low protein assay, full length NRF2 protein and full length Keap1 protein (Keap1 exists a dimer) are used. The assay detects a compound's ability to displace the binding of Keap1 FlagHis with biotinylated Avi-NRF2 protein. Biotin-NRF2 binds to streptavidin-europium (a component of the detection mix) and Keap1 FlagHis is recognized by anti-Flag APC (allophycocyanin) antibody (also a component of the detection mix). If binding occurs between the two proteins, there will be an energy transfer from the Eu+3 (donor) at 615 nm to the APC (acceptor) at 665 nm. A potential NRF2 inhibitor will cause a reduction in the TR-FRET signal by interfering with the binding of Keap1 to NRF2.

Ten nanoliters of 100× compound dose response curves (serial 3-fold dilutions) in DMSO were stamped using an Echo liquid handling system (Labcyte) into 384-well, low volume, black assay plates (Greiner, #784076), with DMSO in columns 6 and 18. An additional 90 nl DMSO was added to each well, to bring the total volume to 100 nl per well. The top concentration of compound was located in columns 1 and 13, with the serial dilutions going across the row. All reagents were diluted in assay buffer (50 mM Tris, pH 8.0, 5 mM MgCl₂, 100 mM NaCl, 0.005% BSA, 1 mM DTT, and 2 mM CHAPS. The BSA, DTT, and CHAPS were added to the assay buffer on the day of assay. Using a Multidrop Combi (Thermo Electron Corporation) equipped with a metal tip dispenser, 5 ul of 1.25 nM Keap1 FlagHis protein was added to all wells of the compound plate, with the exception of the wells in column 18. Wells in column 18 received 5 ul of assay buffer instead. Plates were centrifuged at 500 rpm for 1 minute, covered with a plate lid, and incubated at 37° C. for 2.25 hours. Plates were then removed from the incubator and allowed to cool to RT for 15 minutes. Five microliters of 2.5 nM biotin-NRF2 protein was then added to all wells of the plates and the plates were spun at 500 rpm for 1 minute, followed by incubating at 4° C. for 1.25 hours. The plates were then allowed to warm to RT for 15 minutes, followed by the addition of 10 ul of detection mix (1 nM Streptavidin Eu+W1024 and 5 ug/ml mouse anti-DYKDDDDK IgG conjugated to SureLight APC antibody; both from Columbia Biosciences) to all wells. Plates were spun at 500 rpm for 1 minute, incubated for 1 hour at RT, and read on an Envision plate reader using a 320 nm excitation filter and 615 nm and 665 nm emission filters. Compound response (% inhibition) and potency (pIC50) were calculated based on the ratio of the two emissions (665 nm/615 nm) and then the transformed data was normalized against controls in the assay (control 1=1% DMSO in the presence of NRF2 and Keap1 protein and control 2=1% DMSO in the presence of only the NRF2 protein). Data analysis was handled using the software package Abase XE (Surrey, United Kingdom). The % inhibition values were calculated from the ratio (transformed) data by the equation:

100−(100*(compound response−average control 2)/(average control 1−average control2)).

For calculation of pIC₅₀s, Abase XE used a four parameter equation.

All examples described herein possessed activity in the NRF2/Keap1 Low Protein TR-FRET assay as listed (see table below) unless otherwise noted. IC₅₀s<10 nM (+++++), IC₅₀s 10-100 nM (++++), IC₅₀s 100 nM-1 uM (+++), IC₅₀s 1-10 uM (++). and IC₅₀s 10 uM-100 uM (+), IC₅₀s>100 uM (−), or were not determined (ND).

| Ex # | EC₅₀ |
|---|---|
| 3 | ++++ |
| 4 | ++++ |
| 5 | ++++ |
| 6 | ++++ |
| 7 | +++ |
| 8 | +++ |
| 9 | +++ |
| 10 | +++ |
| 11 | +++ |
| 12 | + |
| 13 | ++++ |
| 14 | ++ |
| 15 | +++ |
| 16 | ++++ |
| 17 | ++++ |
| 18 | ++++ |
| 19 | ++ |
| 20 | +++++ |
| 21 | +++++ |
| 22 | +++++ |
| 23 | ++++ |
| 24 | +++ |
| 25 | ++++ |
| 26a | +++ |
| 26b | ++ |
| 27 | + |
| 28 | ++ |
| 29 | ++++ |
| 30 | +++ |
| 31 | ++ |
| 32 | ++ |
| 33 | + |
| 34 | ++++ |
| 35 | +++ |
| 36 | +++ |
| 37 | +++ |
| 38 | +++ |
| 39 | +++ |
| 40 | ++ |
| 41 | ++ |
| 42 | ++ |
| 43 | ++ |
| 44 | ++ |
| 45 | ++ |
| 46 | ++ |
| 47 | ++ |
| 48 | ++ |
| 49 | ++ |
| 50 | ++ |
| 51 | ++ |
| 52 | ++ |
| 53 | + |
| 54 | ++ |
| 55 | + |
| 56 | + |
| 57 | ++ |

### Methods of Use

The compounds of the invention are NRF2 regulators, and are useful in the treatment or prevention of respiratory disorders, including COPD, asthma, ALI, ARDS, fibrosis, lung infection, diabetic nephropathy/chronic kidney disease, autoimmune diseases (e.g., multiple sclerosis and inflammatory bowel disease), eye diseases (e.g., AMD, Fuchs, and uveitis), cardiovascular diseases, Non-alcoholic steatohepatitis (NASH), Parkinson's, Alzheimer's, psoriasis, acute kidney injury, topical effects of radiation, and kidney transplant.

Accordingly, in another aspect the invention is directed to methods of treating such conditions.

The methods of treatment of the invention comprise administering a safe and effective amount of a compound according to Formula I or a pharmaceutically-acceptable salt thereof to a patient in need thereof.

As used herein, “treat” in reference to a condition means: (1) to ameliorate or prevent the condition or one or more of the biological manifestations of the condition, (2) to interfere with (a) one or more points in the biological cascade that leads to or is responsible for the condition or (b) one or more of the biological manifestations of the condition, (3) to alleviate one or more of the symptoms or effects associated with the condition, or (4) to slow the progression of the condition or one or more of the biological manifestations of the condition.

The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof.

As used herein, “safe and effective amount” in reference to a compound of the invention or other pharmaceutically-active agent means an amount of the compound sufficient to treat the patient's condition but low enough to avoid serious side effects (at a reasonable benefit/risk ratio) within the scope of sound medical judgment. A safe and effective amount of a compound will vary with the particular compound chosen (e.g. consider the potency, efficacy, and half-life of the compound); the route of administration chosen; the condition being treated; the severity of the condition being treated; the age, size, weight, and physical condition of the patient being treated; the medical history of the patient to be treated; the duration of the treatment; the nature of concurrent therapy; the desired therapeutic effect; and like factors, but can nevertheless be routinely determined by the skilled artisan.

As used herein, “patient” refers to a human or other animal.

The compounds of the invention may be administered by any suitable route of administration, including both systemic administration and topical administration. Systemic administration includes oral administration, parenteral administration, transdermal administration, rectal administration, and administration by inhalation. Parenteral administration refers to routes of administration other than enteral, transdermal, or by inhalation, and is typically by injection or infusion. Parenteral administration includes intravenous, intramuscular, and subcutaneous injection or infusion. Inhalation refers to administration into the patient's lungs whether inhaled through the mouth or through the nasal passages. Topical administration includes application to the skin as well as intraocular, otic, intravaginal, and intranasal administration.

The compounds of the invention may be administered once or according to a dosing regimen wherein a number of doses are administered at varying intervals of time for a given period of time. For example, doses may be administered one, two, three, or four times per day. Doses may be administered until the desired therapeutic effect is achieved or indefinitely to maintain the desired therapeutic effect. Suitable dosing regimens for a compound of the invention depend on the pharmacokinetic properties of that compound, such as absorption, distribution, and half-life, which can be determined by the skilled artisan. In addition, suitable dosing regimens, including the duration such regimens are administered, for a compound of the invention depend on the condition being treated, the severity of the condition being treated, the age and physical condition of the patient being treated, the medical history of the patient to be treated, the nature of concurrent therapy, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change.

Typical daily dosages may vary depending upon the particular route of administration chosen. Typical dosages for oral administration range from 1 mg to 1000 mg per person per day. Preferred dosages are 1-500 mg once daily, more preferred is 1-100 mg per person per day. IV dosages range form 0.1-000 mg/day, preferred is 0.1-500 mg/day, and more preferred is 0.1-100 mg/day. Inhaled daily dosages range from 10 ug-10 mg/day, with preferred 10 ug-2 mg/day, and more preferred 50 uug-500 ug/day.

Additionally, the compounds of the invention may be administered as prodrugs. As used herein, a “prodrug” of a compound of the invention is a functional derivative of the compound which, upon administration to a patient, eventually liberates the compound of the invention in vivo. Administration of a compound of the invention as a prodrug may enable the skilled artisan to do one or more of the following: (a) modify the onset of the compound in vivo; (b) modify the duration of action of the compound in vivo; (c) modify the transportation or distribution of the compound in vivo; (d) modify the solubility of the compound in vivo; and (e) overcome a side effect or other difficulty encountered with the compound. Typical functional derivatives used to prepare prodrugs include modifications of the compound that are chemically or enzymatically cleaved in vivo. Such modifications, which include the preparation of phosphates, amides, ethers, esters, thioesters, carbonates, and carbamates, are well known to those skilled in the art.

### Compositions

The compounds of the invention will normally, but not necessarily, be formulated into pharmaceutical compositions prior to administration to a patient. Accordingly, in another aspect the invention is directed to pharmaceutical compositions comprising a compound of the invention and one or more pharmaceutically-acceptable excipient.

The pharmaceutical compositions of the invention may be prepared and packaged in bulk form wherein a safe and effective amount of a compound of the invention can be extracted and then given to the patient such as with powders or syrups. Alternatively, the pharmaceutical compositions of the invention may be prepared and packaged in unit dosage form wherein each physically discrete unit contains a safe and effective amount of a compound of the invention. When prepared in unit dosage form, the pharmaceutical compositions of the invention typically contain from 1 mg to 1000 mg.

The pharmaceutical compositions of the invention typically contain one compound of the invention. However, in certain embodiments, the pharmaceutical compositions of the invention contain more than one compound of the invention. For example, in certain embodiments the pharmaceutical compositions of the invention contain two compounds of the invention. In addition, the pharmaceutical compositions of the invention may optionally further comprise one or more additional pharmaceutically active compounds.

As used herein, “pharmaceutically-acceptable excipient” means a pharmaceutically acceptable material, composition or vehicle involved in giving form or consistency to the pharmaceutical composition. Each excipient must be compatible with the other ingredients of the pharmaceutical composition when commingled such that interactions which would substantially reduce the efficacy of the compound of the invention when administered to a patient and interactions which would result in pharmaceutical compositions that are not pharmaceutically acceptable are avoided. In addition, each excipient must of course be of sufficiently high purity to render it pharmaceutically-acceptable.

The compound of the invention and the pharmaceutically-acceptable excipient or excipients will typically be formulated into a dosage form adapted for administration to the patient by the desired route of administration. For example, dosage forms include those adapted for (1) oral administration such as tablets, capsules, caplets, pills, troches, powders, syrups, elixers, suspensions, solutions, emulsions, sachets, and cachets; (2) parenteral administration such as sterile solutions, suspensions, and powders for reconstitution; (3) transdermal administration such as transdermal patches; (4) rectal administration such as suppositories; (5) inhalation such as dry powders, aerosols, suspensions, and solutions; and (6) topical administration such as creams, ointments, lotions, solutions, pastes, sprays, foams, and gels.

Suitable pharmaceutically-acceptable excipients will vary depending upon the particular dosage form chosen. In addition, suitable pharmaceutically-acceptable excipients may be chosen for a particular function that they may serve in the composition. For example, certain pharmaceutically-acceptable excipients may be chosen for their ability to facilitate the production of uniform dosage forms. Certain pharmaceutically-acceptable excipients may be chosen for their ability to facilitate the production of stable dosage forms. Certain pharmaceutically-acceptable excipients may be chosen for their ability to facilitate the carrying or transporting of the compound or compounds of the invention once administered to the patient from one organ, or portion of the body, to another organ, or portion of the body. Certain pharmaceutically-acceptable excipients may be chosen for their ability to enhance patient compliance.

Suitable pharmaceutically-acceptable excipients include the following types of excipients: diluents, fillers, binders, disintegrants, lubricants, glidants, granulating agents, coating agents, wetting agents, solvents, co-solvents, suspending agents, emulsifiers, sweeteners, flavoring agents, flavor masking agents, coloring agents, anticaking agents, hemectants, chelating agents, plasticizers, viscosity increasing agents, antioxidants, preservatives, stabilizers, surfactants, and buffering agents. The skilled artisan will appreciate that certain pharmaceutically-acceptable excipients may serve more than one function and may serve alternative functions depending on how much of the excipient is present in the formulation and what other ingredients are present in the formulation.

Skilled artisans possess the knowledge and skill in the art to enable them to select suitable pharmaceutically-acceptable excipients in appropriate amounts for use in the invention. In addition, there are a number of resources that are available to the skilled artisan which describe pharmaceutically-acceptable excipients and may be useful in selecting suitable pharmaceutically-acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company).

In one aspect, the invention is directed to a solid oral dosage form such as a tablet or capsule comprising a safe and effective amount of a compound of the invention and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (e.g. corn starch, potato starch, and pre-gelatinized starch), gelatin, acacia, sodium alginate, alginic acid, tragacanth, guar gum, povidone, and cellulose and its derivatives (e.g. microcrystalline cellulose). The oral solid dosage form may further comprise a disintegrant. Suitable disintegrants include crospovidone, sodium starch glycolate, croscarmelose, alginic acid, and sodium carboxymethyl cellulose. The oral solid dosage form may further comprise a lubricant. Suitable lubricants include stearic acid, magnesium stearate, calcium stearate, and talc.

In another aspect, the invention is directed to a dosage form adapted for administration to a patient parenterally including subcutaneous, intramuscular, intravenous or intradermal. Pharmaceutical formulations adapted for parenteral administration include aqueous and non-aqueous sterile injection solutions which may contain anti-oxidants, buffers, bacteriostats, and solutes that render the formulation isotonic with the blood of the intended recipient; and aqueous and non-aqueous sterile suspensions which may include suspending agents and thickening agents. The formulations may be presented in unit-dose or multi-dose containers, for example sealed ampules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets.

In another aspect, the invention is directed to a dosage form adapted for administration to a patient by inhalation. For example, the compound of the invention may be inhaled into the lungs as a dry powder, an aerosol, a suspension, or a solution.

Dry powder compositions for delivery to the lung by inhalation typically comprise a compound of the invention as a finely divided powder together with one or more pharmaceutically acceptable excipients as finely divided powders. Pharmaceutically acceptable excipients particularly suited for use in dry powders are known to those skilled in the art and include lactose, starch, mannitol, and mono-, di-, and polysaccharides.

The dry powder compositions for use in accordance with the present invention are administered via inhalation devices. As an example, such devices can encompass capsules and cartridges of for example gelatin, or blisters of, for example, laminated aluminum foil. In various embodiments, each capsule, cartridge or blister may contain doses of composition according to the teachings presented herein. Examples of inhalation devices can include those intended for unit dose or multi-dose delivery of composition, including all of the devices set forth herein. As an example, in the case of multi-dose delivery, the formulation can be pre-metered (e.g., as in Diskus®, see GB2242134, U.S. Pat. Nos. 6,032,666, 5,860,419, 5,873,360, 5,590,645, 6,378,519 and 6,536,427 or Diskhaler, see GB 2178965, 2129691 and 2169265, U.S. Pat. Nos. 4,778,054, 4,811,731, 5,035,237) or metered in use (e.g., as in Turbuhaler, see EP 69715, or in the devices described in U.S. Pat. No. 6,321,747). An example of a unit-dose device is Rotahaler (see GB 2064336). In one embodiment, the Diskus® inhalation device comprises an elongate strip formed from a base sheet having a plurality of recesses spaced along its length and a lid sheet peelably sealed thereto to define a plurality of containers, each container having therein an inhalable formulation containing the compound optionally with other excipients and additive taught herein. The peelable seal is an engineered seal, and in one embodiment the engineered seal is a hermetic seal. Preferably, the strip is sufficiently flexible to be wound into a roll. The lid sheet and base sheet will preferably have leading end portions which are not sealed to one another and at least one of the leading end portions is constructed to be attached to a winding means. Also, preferably the engineered seal between the base and lid sheets extends over their whole width. The lid sheet may preferably be peeled from the base sheet in a longitudinal direction from a first end of the base sheet.

A dry powder composition may also be presented in an inhalation device which permits separate containment of two different components of the composition. Thus, for example, these components are administrable simultaneously but are stored separately, e.g., in separate pharmaceutical compositions, for example as described in WO 03/061743 A1 WO 2007/012871 A1 and/or WO2007/068896, as well as U.S. Pat. Nos. 8,113,199, 8,161,968, 8,511,304, 8,534,281, 8,746,242 and 9,333,310.

In one embodiment an inhalation device permitting separate containment of components is an inhaler device having two peelable blister strips, each strip containing pre-metered doses in blister pockets arranged along its length, e.g., multiple containers within each blister strip, e.g., as found in ELLIPTA®. Said device has an internal indexing mechanism which, each time the device is actuated, peels opens a pocket of each strip and positions the blisters so that each newly exposed dose of each strip is adjacent to the manifold which communicates with the mouthpiece of the device. When the patient inhales at the mouthpiece, each dose is simultaneously drawn out of its associated pocket into the manifold and entrained via the mouthpiece into the patient's respiratory tract. A further device that permits separate containment of different components is DUOHALER™ of Innovata. In addition, various structures of inhalation devices provide for the sequential or separate delivery of the pharmaceutical composition(s) from the device, in addition to simultaneous delivery.

Aerosols may be formed by suspending or dissolving a compound of the invention in a liquefied propellant. Suitable propellants include halocarbons, hydrocarbons, and other liquefied gases. Representative propellants include: trichlorofluoromethane (propellant 11), dichlorofluoromethane (propellant 12), dichlorotetrafluoroethane (propellant 114), tetrafluoroethane (HFA-134a), 1,1-difluoroethane (HFA-152a), difluoromethane (HFA-32), pentafluoroethane (HFA-12), heptafluoropropane (HFA-227a), perfluoropropane, perfluorobutane, perfluoropentane, butane, isobutane, and pentane. Aerosols comprising a compound of the invention will typically be administered to a patient via a metered dose inhaler (MDI). Such devices are known to those skilled in the art.

The aerosol may contain additional pharmaceutically acceptable excipients typically used with multiple dose inhalers such as surfactants, lubricants, cosolvents and other excipients to improve the physical stability of the formulation, to improve valve performance, to improve solubility, or to improve taste.

Suspensions and solutions comprising a compound of the invention may also be administered to a patient via a nebulizer. The solvent or suspension agent utilized for nebulization may be any pharmaceutically acceptable liquid such as water, aqueous saline, alcohols or glycols, e.g., ethanol, isopropyl alcohol, glycerol, propylene glycol, polyethylene glycol, etc. or mixtures thereof. Saline solutions utilize salts which display little or no pharmacological activity after administration. Both organic salts, such as alkali metal or ammonium halogen salts, e.g., sodium chloride, potassium chloride or organic salts, such as potassium, sodium and ammonium salts or organic acids, e.g., ascorbic acid, citric acid, acetic acid, tartaric acid, etc. may be used for this purpose.

Other pharmaceutically acceptable excipients may be added to the suspension or solution. The compound of the invention may be stabilized by the addition of an inorganic acid, e.g., hydrochloric acid, nitric acid, sulfuric acid and/or phosphoric acid; an organic acid, e.g., ascorbic acid, citric acid, acetic acid, and tartaric acid, etc., a complexing agent such as EDTA or citric acid and salts thereof; or an antioxidant such as antioxidant such as vitamin E or ascorbic acid. These may be used alone or together to stabilize the compound of the invention. Preservatives may be added such as benzalkonium chloride or benzoic acid and salts thereof. Surfactant may be added particularly to improve the physical stability of suspensions. These include lecithin, disodium dioctylsulphosuccinate, oleic acid and sorbitan esters.

The compounds of Formula (I) and pharmaceutically acceptable salts thereof may be used in combination with one or more other agents which may be useful in the prevention or treatment of allergic disease, inflammatory disease, autoimmune disease, for example; antigen immunotherapy, anti-histamines, corticosteroids, (eg fluticasone propionate, fluticasone furoate, beclomethasone dipropionate, budesonide, ciclesonide, mometasone furoate, triamcinolone, flunisolide), NSAIDs, leukotriene modulators (e.g. montelukast, zafirlukast, pranlukast), iNOS inhibitors, tryptase inhibitors, IKK2 inhibitors, p38 inhibitors, Syk inhibitors, protease inhibitors such as elastase inhibitors, integrin antagonists (e.g., beta-2 integrin antagonists), adenosine A2a agonists, mediator release inhibitors such as sodium chromoglycate, 5-lipoxygenase inhibitors (zyflo), DP1 antagonists, DP2 antagonists, PI3K delta inhibitors, ITK inhibitors, LP (lysophosphatidic) inhibitors or FLAP (5-lipoxygenase activating protein) inhibitors (e.g. sodium 3-(3-(tert-butylthio)-1-(4-(6-ethoxypyridin-3-yl)benzyl)-5-((5-methylpyridin-2-yl)methoxy)-1H-indol-2-yl)-2,2-dimethylpropanoate), bronchodilators (e.g., muscarinic antagonists, beta-2 agonists), methotrexate, and similar agents; monoclonal antibody therapy such as anti-IgE, anti-TNF, anti-IL-5, anti-IL-6, anti-IL-12, anti-IL-1 and similar agents; cytokine receptor therapies e.g. etanercept and similar agents; antigen non-specific immunotherapies (e.g. interferon or other cytokines/chemokines, chemokine receptor modulators such as CCR3, CCR4 or CXCR2 antagonists, other cytokine/chemokine agonists or antagonists, TLR agonists and similar agents).

The compounds may also be used in combination with agents for aiding transplantation including Cyclosporines, Tacrolimus, Mycophenolate mofetil, Prednisone, Azathioprine, Sirolimus, Daclizumab, Basiliximab, or OKT3.

They may also be used in combination with agents for Diabetes: metformin (biguanides), meglitinides, sulfonylureas, DPP-4 inhibitors, Thiazolidinediones, Alpha-glucosidase inhibitors, Amylin mimetics, Incretin mimetics, insulin.

The compounds may be used in combination with antihypertensives such as diuretics, ACE inhibitors, ARBS, calcium channel blockers, and beta blockers.

One embodiment of the invention encompasses combinations comprising one or two other therapeutic agents. It will be clear to a person skilled in the art that, where appropriate, the other therapeutic ingredient(s) may be used in the form of salts, for example as alkali metal or amine salts or as acid addition salts, or prodrugs, or as esters, for example lower alkyl esters, or as solvates, for example hydrates to optimize the activity and/or stability and/or physical characteristics, such as solubility, of the therapeutic ingredient. It will be clear also that, where appropriate, the therapeutic ingredients may be used in optically pure form.

The combinations referred to above may conveniently be presented for use in the form of a pharmaceutical formulation and thus pharmaceutical formulations comprising a combination as defined above together with a pharmaceutically acceptable diluent or carrier represent a further aspect of the invention.

The individual compounds of such combinations may be administered either sequentially or simultaneously in separate or combined pharmaceutical formulations. In one embodiment, the individual compounds will be administered simultaneously in a combined pharmaceutical formulation. Appropriate doses of known therapeutic agents will readily be appreciated by those skilled in the art.

The invention thus provides, in a further aspect, a pharmaceutical composition comprising a combination of a compound of the invention together with another therapeutically active agent.

### EXAMPLES

The following examples illustrate the invention. These examples are not intended to limit the scope of the present invention, but rather to provide guidance to the skilled artisan to prepare and use the compounds, compositions, and methods of the present invention. While particular embodiments of the present invention are described, the skilled artisan will appreciate that various changes and modifications can be made without departing from the spirit and scope of the invention.

All temperatures are given in degrees Celsius, all solvents are highest available purity and all reactions run under anhydrous conditions in an argon (Ar) or nitrogen (N₂) atmosphere where necessary.

Analtech Silica Gel GF and E. Merck Silica Gel 60 F-254 thin layer plates were used for thin layer chromatography. Both flash and gravity chromatography were carried out on E. Merck Kieselgel 60 (230-400 mesh) silica gel. The CombiFlash® system used for purification in this application was purchased from Isco, Inc. CombiFlash® purification was carried out using prepacked silica gel columns, a detector with UV wavelength at 254 nm and a variety of solvents or solvent combinations.

Preparative HPLC was performed using a Gilson Preparative System with variable wavelength UV detection or an Agilent Mass Directed AutoPrep (MDAP) system with both mass and variable wavelength UV detection or Waters Preparative System with UV/PDA detection or an Shimadzu PREP LC 20AP. A variety of reverse phase columns, e.g., Luna 5 m C18(2) 100A, SunFire C18, XBridge C18, Atlantics T3 were used in the purification with the choice of column support dependent upon the conditions used in the purification. The compounds are eluted using a gradient of CH₃CN and water. Neutral conditions used an CH₃CN and water gradient with no additional modifier, acidic conditions used an acid modifier, 0.1% TFA (added to both the CH₃CN and water) or 0.1% formic acid and basic conditions used a basic modifier, 0.1% NH₄OH (added to the water) or 10 mM ammonium bicarbonate.

Analytical HPLC was run using an Agilent system, Shimadzu/Sciex LCMS with variable wavelength UV detection using reverse phase chromatography with a CH₃CN and water gradient with a 0.02 or 0.1% TFA modifier (added to each solvent). LC-MS was determined using either a PE Sciex Single Quadrupole 150EX LC-MS, or Waters ZQ Single Quadrupole LC-MS or Agilent 1200 series SL (dectectors: Agilent 6140 single quadrupole and Agilent 1200 MWD SL) instruments. The compound is analyzed using a reverse phase column, e.g., Thermo Hypersil Gold C18, eluted using a gradient of CH₃CN and water with a low percentage of an acid modifier such as 0.02% TFA or 0.1% formic acid or a base modifier such as 5 mM ammonium bicarbonate (adjusted to pH 10 with aqueous ammonia). When specified “acid method” refers to 0.1% formic acid in water and CH₃CN gradient (1.8 min. 0.9 mL/min flow) with a Waters Acquity UPLC HSS C18; 1.8μ; 2.1×50 mm at 50° C.; “basic method” refers to 95:5 H₂O+0.1% NH₄OH:CH₃CN (pH=9.4) and water gradient (1.8 min. 0.9 mL/min flow) with a Waters Acquity UPLC BEH C18; 1.7μ; 2.1×50 mm at 50° C. and “overnight basic method” refers to 95:5 H₂O+0.1% NH₄OH:CH₃CN (pH=9.4) and water gradient (16 min. 0.8 mL/min flow) with a Waters Acquity UPLC BEH C18; 1.7μ; 2.1×50 mm at 50° C.

Preparative Chiral SFC was performed using a Thar/Waters Preparative SFC System with single wavelength UV detection system or PDA detector. A variety of chiral SFC columns, e.g. Chiralpak IA, IC, AY, AD. OD, OJ, C2 were used in the purification. The compounds are eluted using supercritical fluid CO₂ and co-solvents, such as MeOH, EtOH, IPA, and combination of these solvent in different ratio based on the compound selectivity. Modifiers (0.1% of TFA, NH₄OH, DEA) would be used as needed.

Analytical Chiral SFC was run using a Thar/Waters SFC system with variable wavelength UV detection or PDA detector. A variety of chiral SFC columns, e.g. Chiralpak IA, IB, IC, ID, AY, AD, AS, CCL4 were used in the purification. The compounds are eluted using supercritical fluid CO₂ and co-solvents, such as MeOH, EtOH, IPA, and combination of these solvent in different ratio based on the compound selectivity. Modifiers (0.1% of TFA, NH₄OH, DEA) would be used as needed.

Celite® is a filter aid composed of acid-washed diatomaceous silica, and is a registered trademark of Manville Corp., Denver, Colo. Isolute® is a functionalized silica gel based sorbent, and is a registered trademark of Biotage AB Corp., Sweden.

Nuclear magnetic resonance spectra were recorded at 400 MHz using a Bruker AVANCE 400 or Brucker DPX400 or Varian MR400 400 MHz spectrometer. CDCl₃ is deuteriochloroform, DMSO-D₆ is hexadeuteriodimethylsulfoxide, and MeOD is tetradeuteriomethanol, CD₂Cl₂ is deuteriodichloromethane. Chemical shifts are reported in parts per million (δ) downfield from the internal standard tetramethylsilane (TMS) or calibrated to the residual proton signal in the NMR solvent (e.g., CHCl₃ in CDCl₃). Abbreviations for NMR data are as follows: s=singlet, d=doublet, t=triplet, q=quartet, m=multiplet, dd=doublet of doublets, dt=doublet of triplets, app=apparent, br=broad. J indicates the NMR coupling constant measured in Hertz.

Heating of reaction mixtures with microwave irradiations was carried out on a Biotage Initiator® or CEM microwave reactor, typically employing the high absorbance setting.

Cartridges or columns containing polymer based functional groups (acid, base, metal chelators, etc) can be used as part of compound workup. The “amine” columns or cartridges are used to neutralize or basify acidic reaction mixtures or products. These include NH₂ Aminopropyl SPE-ed SPE Cartridges available from Applied Separations and diethylamino SPE cartridges available from United Chemical Technologies, Inc.

### General Methods Used in Examples:

### Acidic Method (Analytical)

HPLC System: Agilent 1200 series SL
Mass Spec Detector: Agilent 6140 single quadrupole

### Second Detector: Agilent 1200 MWD SL

### Eluent A: 0.1% Formic Acid in Water

### Eluent B: CH₃CN

Flow Rate: 0.9 ml/min

### Column: Waters Acquity UPLC HSS C18; 1.8μ; 2.1×50 mm

### Column T: 50° C.

| Time (mins) | % B |
|---|---|
| 0.0 | 5 |
| 0.1 | 5 |
| 1.11 | 95 |
| 1.67 | 95 |
| 1.68 | 5 |
| 1.80 | 5 |

Capillary voltage: 3000V on ES pos (2700V on ES Neg)
Fragmentor/Gain: 190 on ES pos (160 on ES neg)

### Gain: 1

Drying gas flow: 12.0 L/min

### Gas Temperature: 345° C.

Nebuliser Pressure: 60 psig

### Scan Range: 125-1000 amu

Ionisation Mode: ElectroSpray Positive-Negative switching

### Basic Method (Analytical)

HPLC System: Agilent 1200 series SL
Mass Spec Detector: Agilent 6140 single quadrupole

### Second Detector: Agilent 1200 MWD SL

Eluent A: 95:5 H₂O+0.1% NH₄OH:CH₃CN (pH=9.4)

### Eluent B: CH₃CN

Flow Rate: 0.9 ml/min

### Column: Waters Acquity UPLC BEH C18; 1.7μ; 2.1×50 mm

### Column T: 50° C.

| Time (mins) | % B |
|---|---|
| 0.0 | 5 |
| 0.1 | 5 |
| 1.11 | 95 |
| 1.67 | 95 |
| 1.68 | 5 |
| 1.80 | 5 |

Capillary voltage: 3000V on ES pos (2700V on ES Neg)
Fragmentor/Gain: 190 on ES pos (160 on ES neg)

### Gain: 1

Drying gas flow: 12.0 L/min

### Gas Temperature: 345° C.

Nebuliser Pressure: 60 psig

### Scan Range: 125-1000 amu

Ionisation Mode: ElectroSpray Positive-Negative switching

### Overnight Basic Method (Analytical)

HPLC System: Agilent 1200 series SL
Mass Spec Detector: Agilent 6140 single quadrupole

### Second Detector: Agilent 1200 MWD SL

Eluent A: 95:5 H₂O+0.1% NH₄OH:CH₃CN (pH=9.4)

### Eluent B: CH₃CN

Flow Rate: 0.8 ml/min

### Column: Waters Acquity UPLC BEH C18; 1.7μ; 2.1×50 mm

### Column T: 50° C.

| Time (mins) | % B |
|---|---|
| 0.0 | 5 |
| 0.6 | 5 |
| 11.0 | 95 |
| 14.1 | 95 |
| 14.2 | 5 |
| 16 | 5 |

Capillary voltage: 3000V on ES pos (2700V on ES Neg)
Fragmentor/Gain: 190 on ES pos (160 on ES neg)

### Gain: 1

Drying gas flow: 12.0 L/min

### Gas Temperature: 345° C.

Nebuliser Pressure: 60 psig

### Scan Range: 125-800 amu

Ionisation Mode: ElectroSpray Positive-Negative switching

Abbreviations are listed in the table below. All other abbreviations are as described in

the ACS Style Guide (American Chemical Society, Washington, D.C., 1986).

Table of Abbreviations
[Rh(cod)Cl]₂ or [RhCl(cod)]₂: di-μ-chlorido-
bis[η²,η²-(cycloocta-1,5-diene)rhodium
® T3P: 2,4,6-triporopyl-1,3,5,2,4,6-trioxatriphosphorinane 2,4,6-trioxide
□C: degree Celcius
AcOH: acetic acid
ADDP: (E)-diazene-1,2-diylbis(piperidin-1-ylmethanone)
aq = aqueous
BINAP: 2,′-bis(diphenylphosphino)-1,1′-binaphthalene
CDI: Carbonyl dimidazole
CH₂Cl₂: dichloromethane
CH₃CN: acetonitrile
CHCl₃: chloroform
Cs₂CO₃: cesium carbonate
DBU: 1.8-diazabicyclo[5.4.0]undec-7-ene
DCE: dichloromethane
DCM: dichloromethane
DIPEA or DIEA: diisopropylethyl amine
DME: dimethyl ether
DMF: N,N-dimethylformamide
DMF-DMA or DMF-0dimethyl acetal: N,N-dimethylformamide-dimethyl
acetal
DMSO: dimethyl sulfoxide
EDC: 1-ethyl-3-(3-dimethylaminopropyl)carbodiimide
Et₂O: diethyl ether
Et₃N: triethylamine
EtOAc: ethyl acetate
EtOH: ethanol
g: gram(s)
h: hour(s)
HATU: O-(7-azabenzotriazol-1-yl)-N,N,N′,N′-tetramethyluronium
hexafluorophosphate
HBTU: N,N,N′,N′-tetramethyl-O-(1H-benzotriazol-1-yl)uronium
hexafluorophosphate
HCl: hydrochloric acid
HOAt: 1-hydroxy-7-azabenzotriazole
HPLC: high performance liquid chromatography
IPA: isopropyl alcohol
K₂CO₃: potassium carbonate
KOAc: potassium acetate
LAH: lithium aluminum hydride
LC: liquid chromatography
LC-MS: liquid chromatography-mass spectroscopy
LiBH₄: lithium borohydride
LiHMDS: lithium hexamethyldisilazane
LiOH: lithium hydroxide
M: molar
MeCN: acetonitrile
MeI: methyl iodide
MeOH: methanol
mg: milligram(s)
MgCl₂: magnesium chloride
MgSO₄: magnesium sulfate
MHz: megahertz
min: minute(s)
mL: milliliter(s)
mmol: millimole(s)
MS: mass spectroscopy
N₂: nitrogen gas
Na₂CO₃: sodium carbonate
Na₂SO₄: sodium sulfate
NaBH₃CN or NaCNBH₃: sodium cyanoborohydride
NaCl: sodium chloride
NaH: sodium hydride
NaHCO₃: sodium bicarbonate
NaHMDS: sodium hexamethyldisilazane
NaHSO₄: sodium bisulfate
NaOAc: sodium acetate
NaOH: sodium hydroxide
NBS: N-bromosuccinimide
nBuLi: n-butyl lithium
NH₄Cl: ammonium chloride
NMR: nuclear magnetic resonanc
P(tBu)₃: tri-t-butyl phosphine
Pd(PhP₃)₄: tetrakistriphenylphosphine palladium
Pd/C: palladium on carbon
Pd₂(dba)₃: tris(dibenzylideneacetone)-dipalladium(0)
PdCl₂(dppf) or Pd(dppf)Cl₂: [1,1′-bis(diphenylphosphino)-ferrocene]
dichloropalladium(II)
Petrol: petroleum ether
PS-PPh₃: polymer supported triphenylphosphine
PtO₂: platinum(IV) oxide
RT: room temperature
T3P: 2,4,6-tripropyl-1,3,5,2,4,6-trioxatriphosphorinane-2,4,6-trioxide
solution
TEA: triethylamine
TFA: trifluoroacetic acid
TFFH: tetrafluoroformamidinium hexafluorophosphate
THF: tetrahydrofuran
triflic anhydride: trifluoromethanesulfonic anhydride
TsOH: p-tolunesulfonic acid
wt %: weight percent

### Intermediates

### Intermediate 1

### 2-Bromo-N-(2-methylallyl)benzenesulfonamide

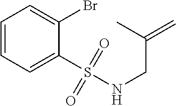

To a solution of 2-bromobenzene-1-sulfonyl chloride (25 g, 98 mmol) in dichloromethane (DCM) (250 mL) at 0° C. was added TEA (13.64 mL, 98 mmol) and 2-methylprop-2-en-1-amine (6.96 g, 98 mmol) and stirred for 10 min. Then it was stirred at RT for 16 h. The reaction mixture was quenched with ice cold water and extracted with DCM (2×200 mL). The combined organic layer washed with ice cold water (2×100 mL), washed with brine solution (100 mL), dried over anhydrous Na₂SO₄. It was filtered and concentrated to give the title compound (20 g, 68.3 mmol, 69.8% yield). LC-MS m/z 289.81 (M+H)⁺, 2.20 min (ret. time).

### Intermediate 2

### 4-Methyl-2,3,4,5-tetrahydrobenzo[f][1,2]thiazepine 1,1-dioxide

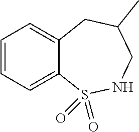

To a solution of 2-bromo-N-(2-methylallyl)benzenesulfonamide (16 g, 55.1 mmol) in toluene (160 mL) at RT was added AIBN (1.811 g, 11.03 mmol). The reaction mixture was heated to 75° C. and added tri-n-butyltin hydride (29.4 mL, 110 mmol). It was heated at 110° C. for 18 h. The reaction mixture was cooled to RT and diluted with ice water (500 mL) and extracted with EtOAc (2×300 mL). The combined organic layer was washed with chilled brine solution (200 mL) and dried over anhydrous Na₂SO₄, filtered and concentrated. The crude residue was purified on flash column chromatography eluting with 15% ethyl acetate in hexane. Desired fractions were concentrated to give the title compound (8.51 g, 39.9 mmol, 72.3% yield) as a white solid. LC-MS m/z 211.11 (M+H)⁺, 1.826 min (ret. time).

### Intermediate 3

### (S)-4-Methyl-2,3,4,5-tetrahydrobenzo[f][1,2]thiazepine 1,1-dioxide and (R)-4-methyl-2,3,4,5-tetrahydrobenzo[f][1,2]thiazepine 1,1-dioxide

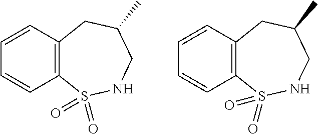

4-Methyl-2,3,4,5-tetrahydrobenzo[f][1,2]thiazepine 1,1-dioxide (4000 mg, 18.93 mmol) was resolved by Chiral SFC (Column: Chiralpak AY 20×250 mm, 5u; Co-solvent: 20% EtOH; Flow rate: 50 mg/min; Back pressure: 100 Bar) to give single enantiomerically pure (S)-4-methyl-2,3,4,5-tetrahydrobenzo[f][1,2]thiazepine 1,1-dioxide (2.2996 g, 10.88 mmol, 57.5% yield) (chiral SFC ret. time: 1.85 min) LC-MS m/z 211.9 (M+H)⁺, 0.72 min (ret. time) and single enantiomerically pure (R)-4-methyl-2,3,4,5-tetrahydrobenzo[f][1,2]thiazepine 1,1-dioxide (2.2195 g, 10.50 mmol, 55.5% yield) (chiral SFC ret. time: 2.5 min) LC-MS m/z 211.9 (M+H)⁺, 0.72 min (ret. time).

### Intermediate 4

### N-(4-Methoxybenzyl)-2-methylenebutan-1-amine

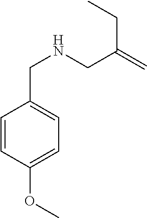

To a solution of (4-methoxyphenyl)methanamine (24.46 g, 178 mmol) in toluene (100 mL) was added 2-methylenebutanal (15 g, 178 mmol). It was heated at 95° C. for 48 h. The reaction mixture was concentrated and dissolved in ethanol (100.0 mL), NaBH₄ (6.75 g, 178 mmol) was added at 0° C. and stirred at RT for 4 h. The reaction mixture was concentrated, quenched with water, extracted with DCM twice. The organic layer was dried under anhydrous Na₂SO₄, filtered and concentrated. The crude product was purified on flash column chromatography eluting with EtOAc:ether (18:72). Desired fractions were concentrated to give the title compound (13 g, 30.1 mmol, 16.87% yield) as pale yellow liquid. LCMS m/z 206.05 (M+H)⁺, 3.70 min (ret. time).

### Intermediate 5

### 2-Bromo-N-(4-methoxybenzyl)-N-(2-methylenebutyl)benzenesulfonamide

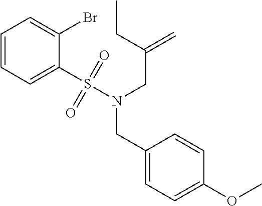 [SMILES: C=C(CC)CN(Cc1ccc(OC)cc1)S(=O)(=O)c1ccccc1Br]

To a solution of 2-bromobenzene-1-sulfonyl chloride (7.60 g, 29.8 mmol) in dichloromethane (DCM) (130 mL) at 0° C. was added N-(4-methoxybenzyl)-2-methylenebutan-1-amine (13 g, 29.8 mmol) and TEA (8.30 mL, 59.5 mmol). The reaction mixture was stirred at RT for 6 h. The reaction mixture was quenched with cold water, extracted with twice DCM, brine solution. The organic layer was dried under anhydrous Na₂SO₄, filtered and concentrated. The crude residue was purified by column chromatography eluting with EtOAC:hexane (5:95). Desired fractions were concentrated to give the title compound (9 g, 21.21 mmol, 71.3% yield) as white solid. LCMS m/z 426.05 (M+H)⁺, 4.18 min (ret. time).

### Intermediate 6

### 2-Bromo-N-(2-methylenebutyl)benzenesulfonamide

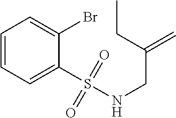 [SMILES: C=C(CC)CNS(=O)(=O)c1ccccc1Br]

To a solution of 2-bromo-N-(4-methoxybenzyl)-N-(2-methylenebutyl)benzenesulfonamide (9 g, 19.67 mmol) in acetonitrile (90 mL) and water (30 mL) at 0° C. was added CAN (43.1 g, 79 mmol). It was stirred at RT for 6 h. The reaction mixture was concentrated, quenched with ice water, extracted with DCM twice. The organic layer was dried under anhydrous Na₂SO₄, filtered and concentrated. The crude compound was purified by column chromatography eluting with EtOAC:hexane (15:85). Desired fractions were concentrated. It was then dissolved in methanol (50 mL). NaBH₄ (0.744 g, 19.67 mmol) was added at 0° C. The reaction mixture was stirred at ambient temperature for 4 h. The reaction mixture was concentrated, quenched with ice water, extracted with DCM twice. The organic layer was dried over anhydrous Na₂SO₄, filtered and concentrated. The crude compound was purified by column chromatography eluting with EtOAC:hexane (06:94) to give the title compound (3 g, 9.14 mmol, 46.5% yield) as white solid. LCMS m/z 303.96 (M+H)⁺, 2.31 min (ret. time).

### Intermediate 7

### 4-Ethyl-2,3,4,5-tetrahydrobenzo[f][1,2]thiazepine 1,1-dioxide

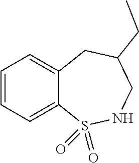

To a solution of 2-bromo-N-(2-methylenebutyl)benzenesulfonamide (3 g, 9.07 mmol) in benzene (30 mL) was added AIBN (0.447 g, 2.72 mmol) and heated at 65° C., tributylstannane (2.90 g, 9.98 mmol) was added at this temperature. The reaction was stirred at 85° C. for 16 h. The reaction mixture was concentrated. The crude product was purified on grace column chromatography with 100-200 silica gel mesh by using EtOAc:hexane (23:77) as solvent. The eluted fractions were concentrated. The product was washed with n-pentane (23 mL) and diethyl ether (10 mL) to give the title compound (980 mg, 4.21 mmol, 46.4% yield) as white solid. LCMS m/z 226.11 (M−H)⁺, 2.04 min (ret. time) ¹H NMR (400 MHz, DMSO-d6) δ ppm: 7.77 (dd, J=7.78, 1.21 Hz, 1H) 7.55 (br t, J=6.47 Hz, 1H) 7.45-7.51 (m, 1H) 7.34-7.42 (m, 2H) 3.37 (br s, 1H) 3.04-3.28 (m, 3H) 1.56 (br s, 1H) 1.15 (br s, 2H) 0.87 (t, J=7.34 Hz, 3H).

### Intermediate 8

### (S)-4-Ethyl-2,3,4,5-tetrahydrobenzo[f][1,2]thiazepine 1,1-dioxide and (R)-4-ethyl-2,3,4,5-tetrahydrobenzo[f][1,2]thiazepine 1,1-dioxide

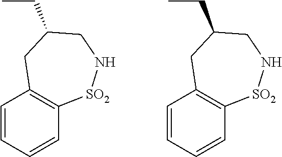

4-Ethyl-2,3,4,5-tetrahydrobenzo[f][1,2]thiazepine 1,1-dioxide (1 g, 4.44 mmol) was resolved by Chiral SFC (Column: Chiralpak AY 20×250 mm, 5u; Co-solvent: 25% EtOH; Flowrate: 50 g/min; Back pressure: 100 Bar) to give single enantiomerically pure (S)-4-ethyl-2,3,4,5-tetrahydrobenzo[f][1,2]thiazepine 1,1-dioxide (471 mg, 2.090 mmol, 47.1% yield) (chiral SFC ret. time: 1.86 min) LC-MS m/z 226.1 (M+H)⁺, 0.92 min (ret. time) and (R)-4-ethyl-2,3,4,5-tetrahydrobenzo[f][1,2]thiazepine 1,1-dioxide (472 mg, 2.095 mmol, 47.2% yield) (chiral SFC ret. time: 2.47 min) LC-MS m/z 226.1 (M+H)⁺, 0.92 min (ret. time).

### Intermediate 9

### 2,5-Dibromo-N-(2-methylallyl)benzenesulfonamide

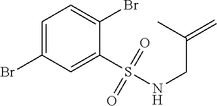 [SMILES: C=C(C)CNS(=O)(=O)c1cc(Br)ccc1Br]

To a solution of 2,5-dibromobenzene-1-sulfonyl chloride (5 g, 14.95 mmol) in dichloromethane (50 mL) at 0° C. was added 2-methylprop-2-en-1-amine (1.063 g, 14.95 mmol) and TEA (2.084 mL, 14.95 mmol). It was stirred for 10 min and then stirred at RT for 16 h. The reaction mixture was quenched with ice cold water and extracted with DCM (2×50 mL). The combined organic layer washed with ice cold water (2×35 mL), washed with brine solution (50 mL), dried over anhydrous Na₂SO₄, filtered and concentrated to give the title compound (3.4 g, 8.58 mmol, 57.4% yield) LC-MS m/z 367.8 (M+H)⁺, 2.58 min (ret. time).

### Intermediate 10

### 8-Bromo-4-methyl-2,3,4,5-tetrahydrobenzo[f][1,2]thiazepine 1,1-dioxide

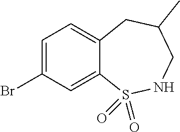

To a solution of 2,5-dibromo-N-(2-methylallyl)benzenesulfonamide (3.4 g, 9.21 mmol) in toluene (35 mL) at RT was added AIBN (0.303 g, 1.842 mmol). The reaction mixture was heated at 75° C. and tri-n-butyltin hydride (4.92 mL, 18.42 mmol) was added. It was stirred at 110° C. for 18 h. The crude residue was diluted with ethyl acetate (100 mL) and washed with brine solution (100 mL). The organic layer was dried over anhydrous Na₂SO₄, filtered and concentrated. The crude residue was purified on flash column chromatography eluting with 15% ethyl acetate in hexane. Desired fractions were concentrated to give the title compound (600 mg, 1.991 mmol, 21.61% yield) as an off-white solid. LC-MS m/z 287.8 (M+H)⁺, 2.29 min (ret. time).

### Intermediate 11

### rel-(R or S)-8-Bromo-4-methyl-2,3,4,5-tetrahydrobenzo[f][1,2]thiazepine 1,1-dioxide and rel-(R or S)-8-bromo-4-methyl-2,3,4,5-tetrahydrobenzo[f][1,2]thiazepine 1,1-dioxide

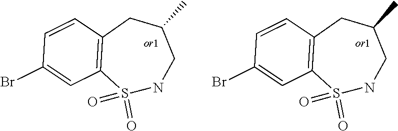

8-Bromo-4-methyl-2,3,4,5-tetrahydrobenzo[f][1,2]thiazepine 1,1-dioxide was resolved by Chiral SFC (Column: Chiralpak AY 20×250 mm, 5u; Co-solvent: 20% EtOH; Flow rate: 50 g/min; Back pressure: 100 Bar) to give single enantiomerically pure rel-(R or S)-8-bromo-4-methyl-2,3,4,5-tetrahydrobenzo[f][1,2]thiazepine 1,1-dioxide (195 mg, 0.672 mmol, 32.5% yield) (chiral SFC ret. time: 3.06 min) LC-MS m/z 289.8 (M+H)⁺, 0.94 min (ret. time) and single enantiomerically pure rel-(R or S)-8-bromo-4-methyl-2,3,4,5-tetrahydrobenzo[f][1,2]thiazepine 1,1-dioxide (190 mg, 0.655 mmol, 31.7% yield) (chiral SFC ret. time: 4.03 min) LC-MS m/z 289.8 (M+H)⁺, 0.95 min (ret. time).

### Intermediate 12

### 2-Bromo-N-(2-methylallyl)-5-(trifluoromethyl)benzenesulfonamide

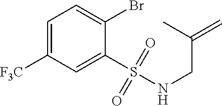 [SMILES: C=C(C)CNS(=O)(=O)c1cc(C(F)(F)F)ccc1Br]

To a suspension of 2-bromo-5-(trifluoromethyl)benzene-1-sulfonyl chloride (5 g, 15.46 mmol) in dichloromethane (DCM) (50 mL) at RT was added 2-methylprop-2-en-1-amine (1.231 g, 17.31 mmol) and triethylamine (4.31 mL, 30.9 mmol). It was stirred for 20 h. The reaction mixture was poured into ice-cold water and extracted with ethyl acetate (2×100 mL). The combined organic layer was washed with brine (100 mL) and dried over Na₂SO₄, filtered and concentrated to give the title compound (5 g, 13.88 mmol, 90% yield) as gummy liquid. LC-MS m/z 355.9 (M+H)⁺, 2.61 min (ret. time).

### Intermediate 13

### 4-Methyl-8-(trifluoromethyl)-2,3,4,5-tetrahydrobenzo[f][1,2]thiazepine 1,1-dioxide

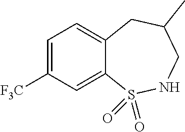 [SMILES: CC1CNS(=O)(=O)c2cc(C(F)(F)F)ccc2C1]

To a solution of 2-bromo-N-(2-methylallyl)-5-(trifluoromethyl)benzenesulfonamide (5 g, 13.96 mmol) in toluene (50 mL) was added AIBN (0.458 g, 2.79 mmol). The reaction mixture was heated to 60° C. and then tributylstannane (8.13 g, 27.9 mmol) was added. It was stirred at 100° C. for 20 h. The reaction mixture was cooled and concentrated. The crude residue was purified on flash column chromatography eluting with 25% EtOAc in hexane. Desired fractions were concentrated to give the title compound (720 mg, 2.52 mmol, 18.02% yield) as a white solid. LC-MS m/z 278.01 (M+H)⁺, 2.37 min (ret. time).

### Intermediate 14

### (S)-4-Methyl-8-(trifluoromethyl)-2,3,4,5-tetrahydrobenzo[f][1,2]thiazepine 1,1-dioxide and (R)-4-methyl-8-(trifluoromethyl)-2,3,4,5-tetrahydrobenzo[f][1,2]thiazepine 1,1-dioxide

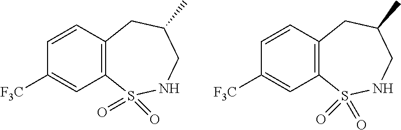 [SMILES: C[C@@H]1CNS(=O)(=O)c2cc(C(F)(F)F)ccc2C1 ; C[C@H]1CNS(=O)(=O)c2cc(C(F)(F)F)ccc2C1]

4-Methyl-8-(trifluoromethyl)-2,3,4,5-tetrahydrobenzo[f][1,2]thiazepine 1,1-dioxide (650 mg, 2.327 mmol) was resolved by Chiral SFC (Column: Chiralpak AD 20×250 mm, 5 u; Co-solvent: 5% IPA in Hexane; Flowrate: 10 mL/min; Back pressure: 100 Bar) to give single enantiomerically pure (S)-4-methyl-8-(trifluoromethyl)-2,3,4,5-tetrahydrobenzo[f][1,2]thiazepine 1,1-dioxide (219 mg, 0.784 mmol, 33.7% yield) (chiral HPLC ret. time: 15.171 min) LC-MS m/z 279.9 (M+H)⁺, 0.95 min (ret. time) and single enantiomerically pure (R)-4-methyl-8-(trifluoromethyl)-2,3,4,5-tetrahydrobenzo[f][1,2]thiazepine 1,1-dioxide (126 mg, 0.451 mmol, 19.38% yield) (chiral HPLC ret. time: 17.076 min) LC-MS m/z 279.9 (M+H)⁺, 0.96 min (ret. time).

### Intermediate 15

### 2-Bromo-N-(2-methylallyl)-4-(trifluoromethyl)benzenesulfonamide

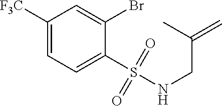 [SMILES: C=C(C)CNS(=O)(=O)c1ccc(C(F)(F)F)cc1Br]

To a solution of 2-bromo-4-(trifluoromethyl)benzene-1-sulfonyl chloride (5 g, 15.46 mmol) in dichloromethane (DCM) (50 mL) at 0° C. was added 2-methylprop-2-en-1-amine (1.209 g, 17.00 mmol) and TEA (4.31 mL, 30.9 mmol). The reaction mixture was stirred at RT for 16 h. The reaction mixture was quenched with cold water, extracted with DCM twice. The combined organic layer was washed with brine solution, dried over anhydrous Na₂SO₄, filtered and concentrated to give the title compound (4.2 g, 11.64 mmol, 75% yield). LC-MS m/z 357.98 (M+H)⁺, 2.254 min (ret. time).

### Intermediate 16

### 4-Methyl-7-(trifluoromethyl)-2,3,4,5-tetrahydrobenzo[f][1,2]thiazepine 1,1-dioxide

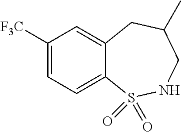 [SMILES: CC1CNS(=O)(=O)c2ccc(C(F)(F)F)cc2C1]

To a solution of 2-bromo-N-(2-methylallyl)-4-(trifluoromethyl)benzenesulfonamide (4.2 g, 11.73 mmol) in toluene (40 mL) was added AIBN (0.385 g, 2.345 mmol) and heated to 75° C. Tributyltin hydride (3.75 g, 12.90 mmol) was added at 75° C. and the reaction mixture was stirred at 110° C. for 16 h. The reaction mixture was cool and concentrated. The crude residue was purified on flash column chromatography eluting with EtOAC:hexane (11:89). Desired fractions were concentrated to give the title compound (1.6 g, 5.61 mmol, 47.8% yield). LC-MS m/z 278.09 (M+H)⁺, 2.08 min (ret. time).

### Intermediate 17

### (R)-4-Methyl-7-(trifluoromethyl)-2,3,4,5-tetrahydrobenzo[f][1,2]thiazepine 1,1-dioxide and (S)-4-methyl-7-(trifluoromethyl)-2,3,4,5-tetrahydrobenzo[f][1,2]thiazepine 1,1-dioxide

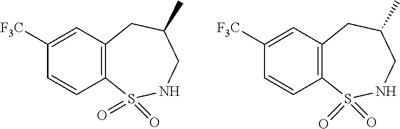 [SMILES: C[C@H]1CNS(=O)(=O)c2ccc(C(F)(F)F)cc2C1 ; C[C@@H]1CNS(=O)(=O)c2ccc(C(F)(F)F)cc2C1]

4-Methyl-7-(trifluoromethyl)-2,3,4,5-tetrahydrobenzo[f][1,2]thiazepine 1,1-dioxide (1500 mg, 5.37 mmol) was resolved by Chiral SFC (Column: Chiralpak AD 20×250 mm, 5u; Co-solvent: 4% IPA/Hexane; Flow rate: 10 mL/min; Back pressure: 30 Bar) to give single enantiomerically pure (R)-4-methyl-7-(trifluoromethyl)-2,3,4,5-tetrahydrobenzo[f][1,2]thiazepine 1,1-dioxide (685 mg, 2.453 mmol, 45.7% yield) (chiral HPLC ret. time: 22.284 min) LC-MS m/z 280.0 (M+H)⁺, 0.98 min (ret. time) and single enantiomerically pure (S)-4-methyl-7-(trifluoromethyl)-2,3,4,5-tetrahydrobenzo[f][1,2]thiazepine 1,1-dioxide (660 mg, 2.363 mmol, 44.0% yield) (chiral HPLC ret. time: 27.803 min) LC-MS m/z 280.0 (M+H)⁺, 0.98 min (ret. time).

### Intermediate 18

### (R)-4-Ethyl-3,4-dihydro-2H-benzo[b][1,4,5]oxathiazepine 1,1-dioxide

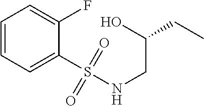 [SMILES: CC[C@@H](O)CNS(=O)(=O)c1ccccc1F]

To a solution of (R)-1-aminobutan-2-ol (14.66 g, 164 mmol) in tetrahydrofuran (THF) (200 mL) and water (60 mL) at RT was added K₂CO₃ (14.20 g, 103 mmol) and 2-fluorobenzene-1-sulfonyl chloride (20 g, 103 mmol). It was stirred for 16 h. The reaction mixture was diluted with water (100 mL) and extracted with EtOAc (2×100 mL). The combined organic layer was washed with brine solution (200 mL) and dried over anhydrous Na₂SO₄, filtered and concentrated to give the title compound (14 g, 53.8 mmol, 52.3% yield) as a gammy liquid. LC-MS m/z 494.83 (2M−H)⁺, 1.660 min (ret. time).

### Intermediate 19

### (R)-4-Ethyl-3,4-dihydro-2H-benzo[b][1,4,5]oxathiazepine 1,1-dioxide

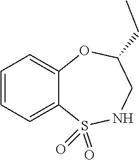

To a solution of (R)-2-fluoro-N-(2-hydroxybutyl)benzenesulfonamide (14 g, 56.6 mmol) in dimethyl sulfoxide (DMSO) (140 mL) at 0° C. was added potassium tert-butoxide (6.35 g, 56.6 mmol). It was then heated at 80° C. for 4 h. The reaction mixture was cooled and neutralized with 1N HCl, diluted with ice water (500 mL) and extracted with EtOAc (2×400 mL). The combined organic layer was washed with chilled brine solution (200 mL) and dried over anhydrous Na₂SO₄, filtered and concentrated. The crude residue was purified on flash column chromatography eluting with 50% EtOAc in hexane. Desired fractions were concentrated to give the title compound (11.12 g, 48.9 mmol, 86% yield) as a white solid. LC-MS m/z 228.05 (M+H)⁺, 1.84 min (ret. time).

### Intermediate 20

### 2,5-Difluoro-N-(2-hydroxy-2-methylpropyl)benzenesulfonamide

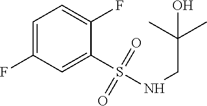 [SMILES: CC(C)(O)CNS(=O)(=O)c1cc(F)ccc1F]

To a solution of 2,5-difluorobenzene-1-sulfonyl chloride (10 g, 47.0 mmol) in tetrahydrofuran (THF) (10 mL) at 0° C. was added 1-amino-2-methylpropan-2-ol (4.19 g, 47.0 mmol) and potassium carbonate (6.50 g, 47.0 mmol). The reaction mixture was stirred at ambient temperature for 4 h. The reaction mixture was extracted with EtOAc (2×100 mL). The combined organic layer washed with brine solution (50 mL), dried over anhydrous Na₂SO₄, filtered and concentrated. The crude product was purified on flash column chromatography eluting with 60% ethyl acetate in n-hexane. Desired fractions were concentrated to give the title compound (7 g, 25.5 mmol, 54.2% yield) as off white solid. LCMS m/z 264 (M−H)⁺, 4.13 min (ret. time).

### Intermediate 21

### 8-Fluoro-4,4-dimethyl-3,4-dihydro-2H-benzo[b][1,4,5]oxathiazepine 1,1-dioxide

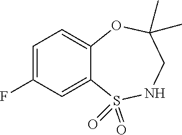 [SMILES: CC1(C)CNS(=O)(=O)c2cc(F)ccc2O1]

To a solution of 2,5-difluoro-N-(2-hydroxy-2-methylpropyl)benzenesulfonamide (7 g, 26.4 mmol) in DMSO (50 mL) at 0° C. was added potassium tert-butoxide (5.92 g, 52.8 mmol). The reaction mixture was stirred at 100° C. for 6 h. The reaction mixture was neutralized with 1N HCl at pH 6-7. It was extracted with ethyl acetate (3×100 mL). The combined organic layer was washed with brine solution (80 mL) and dried over anhydrous Na₂SO₄, filtered and concentrated. The crude residue was purified on flash column chromatography eluting with EtOAc:Hexane (6:4). Desired fractions were concentrated to give the title compound (2.9 g, 10.93 mmol, 41.4% yield) as an off-white solid. LCMS m/z 244.12 (M−H)⁺, 2.28 min (ret. time).

### Intermediate 22

### N-(2,4-Dimethoxybenzyl)-2-methylenebutan-1-amine

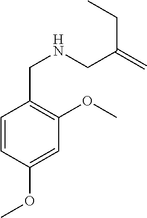 [SMILES: C=C(CC)CNCc1ccc(OC)cc1OC]

To a solution of 2-methylenebutanal (100 g, 1189 mmol) in toluene (135 mL) was added (2,4-dimethoxyphenyl)methanamine (199 g, 1189 mmol) and stirred at 110° C. for 48 hr. The reaction mixture was concentrated and dissolved in ethanol (82 mL). NaBH₄ (90 g, 2378 mmol) was added at 0° C. and the reaction stirred at ambient temperature for 6 h. The reaction mixture was evaporated under reduced pressure, quenched with water (200 mL) and extracted with DCM (2×200 mL). The organic layer was dried over anhydrous Na₂SO₄ and filtered. The filtrate was evaporated under reduced pressure and the residue was purified by flash chromatography eluting with 1:9 EtOAc:Hexane. To provide the title compound. (68 g, 16.53% yield). LC/MS m/z 236 (M+H)⁺, 3.62 min (ret. time).

### Intermediate 23

### 2-Bromo-N-(2,4-dimethoxybenzyl)-N-(2-methylenebutyl)-5-(trifluoromethyl)benzenesulfonamide

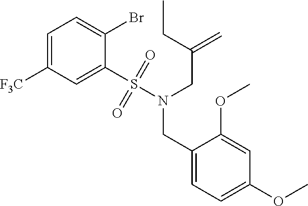 [SMILES: C=C(CC)CN(Cc1ccc(OC)cc1OC)S(=O)(=O)c1cc(C(F)(F)F)ccc1Br]

To a solution of N-(2,4-dimethoxybenzyl)-2-methylenebutan-1-amine (15 g, 43.3 mmol) in dichloromethane (DCM) (300 mL) was added Et₃N (12.08 mL, 87 mmol) at 0° C. followed by addition of 2-bromo-5-(trifluoromethyl)benzene-1-sulfonyl chloride (14.02 g, 43.3 mmol) and the reaction allowed to stir at ambient temperature for 16 h. The reaction mixture was evaporated under reduced pressure, quenched with water (300 mL) and extracted with DCM (2×300 mL). The organic layer was dried over anhydrous Na₂SO₄ and filtered. The filtrate was evaporated under reduced pressure and the residue was purified by flash chromatography eluting with 2%, 4% then 8% petroleum ether/ethyl acetate to provide the title compound. (20 g, 81% yield). GC/MS m/z 521/523 (M+H)⁺, 10.66 min (ret. time).

### Intermediate 24

### 2-Bromo-N-(2-methylenebutyl)-5-(trifluoromethyl)benzenesulfonamide

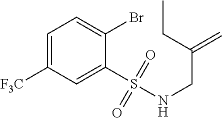 [SMILES: C=C(CC)CNS(=O)(=O)c1cc(C(F)(F)F)ccc1Br]

To a solution of 2-bromo-N-(2,4-dimethoxybenzyl)-N-(2-methylenebutyl)-5-(trifluoromethyl)benzenesulfonamide (39 g, 38.1 mmol) in dichloromethane (DCM) (300 mL) was added TFA (32 mL, 415 mmol) at 0° C. Anisole (10 mL, 92 mmol) was added and the reaction stirred at ambient temperature for 16 h. The reaction mixture was evaporated under reduced pressure, quenched with water (200 mL) and extracted with DCM (2×200 mL). The organic layer was dried over anhydrous Na₂SO₄ and filtered. The filtrate was evaporated under reduced pressure and the residue was purified by flash chromatography eluting with 2%, 4% then 8% petroleum ether/ethyl acetate to provide the title compound. (17 g, 96% yield). LC/MS m/z 369/371 (M−H)(M), 2.67 min (ret. time).

### Intermediate 25

### (S)-4-Ethyl-8-(trifluoromethyl)-2,3,4,5-tetrahydrobenzo[f][1,2]thiazepine 1,1-dioxide

### (R)-4-ethyl-8-(trifluoromethyl)-2,3,4,5-tetrahydrobenzo[f][1,2]thiazepine 1,1-dioxide

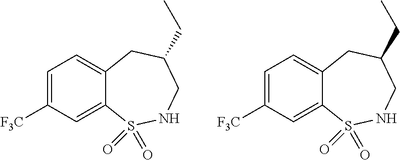 [SMILES: CC[C@@H]1CNS(=O)(=O)c2cc(C(F)(F)F)ccc2C1 ; CC[C@H]1CNS(=O)(=O)c2cc(C(F)(F)F)ccc2C1]

To a solution of 2-bromo-N-(2-methylenebutyl)-5-(trifluoromethyl)benzenesulfonamide (17.5 g, 45.1 mmol) in toluene (200 mL) was added AIBN (3.71 g, 22.57 mmol) and the reaction was heated to 70° C. Tri-n-butyltin hydride (36.4 mL, 135 mmol) was added and the reaction stirred at 110° C. for 16 h. The reaction mixture was cooled to ambient temperature and concentrated under reduced pressure. The residue was purified by flash chromatography eluting with EtOAc:Hexane (15:85) to provide the title compound as a racemate. (11.5 g, 79% yield). LC/MS m/z 292 (M−H), 2.54 min (ret. time). The compound was resolved by chiral SFC (Column: Lux Cellulose-2 30×250 mm, 5u; Co-solvent: 20% (100% IPA); 80% CO₂, Flowrate: 90 g/min; Back pressure: 90 Bar) to provide (S)-4-ethyl-8-(trifluoromethyl)-2,3,4,5-tetrahydrobenzo[f][1,2]thiazepine 1,1-dioxide (4.2 g, 36% yield). m/z 294 (M+H)⁺, 3.29 min (ret. time), (chiral SFC ret. time: 4.91 min) and (R)-4-ethyl-8-(trifluoromethyl)-2,3,4,5-tetrahydrobenzo[f][1,2]thiazepine 1,1-dioxide (3.8 g, 32% yield). LCMS m/z 294 (M+H)⁺, 3.29 min (ret. time), (chiral SFC ret. time: 6.71 min).

The intermediate in Table 1 was prepared in an analogous manner:

TABLE 1

| | | LCMS | Retention | |
|---|---|---|---|---|
| | | [M + | Time | |
| Structure | Name | H]⁺ | (min) | ¹HNMR |
| | (S)-4-butyl-8- | 322.1 | 7.57 | 1H NMR (400 |
| | (trifluoromethyl)- | | | MHz, DMSO-d6) |
| | 2,3,4,5-tetrahydro- | | | δ ppm 0.80-0.88 |
| | benzo[f][1,2] | | | (m, 3 H), 1.08- |
| | thiazepine 1,1- | | | 1.35 (m, 6 H) |
| | dioxide | | | 1.68 (br s, 1 H) |
| | | | | 3.14-3.28 (m, 3 |
| | | | | H) 3.43 (br s, 1 |
| | | | | H) 7.69 (d, |
| | | | | J = 7.89 Hz, 1 H) |
| | | | | 7.78 (br s, 1 H) |
| | | | | 7.90 (dd, |
| | | | | J = 7.89, 1.53 Hz, |
| | | | | 1 H), 7.97 (d, |
| | | | | J 1.53 Hz, 1 H) |

### Intermediate 26

### 8-Bromo-4-ethyl-2,3,4,5-tetrahydrobenzo[f][1,2]thiazepine 1,1-dioxide

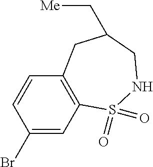 [SMILES: CCC1CNS(=O)(=O)c2cc(Br)ccc2C1]

To 4-ethyl-2,3,4,5-tetrahydrobenzo[f][1,2]thiazepine 1,1-dioxide (1.2 g, 1.791 mmol) was added NBS (0.319 g, 1.791 mmol) followed by H₂SO₄ (0.095 ml, 1.791 mmol). The resulting mixture was allowed to stir at RT for 3 h. Following this duration, the reaction mixture was poured into crushed ice and extracted with EtOAc (2×10 mL). The organic layer was washed with 0.1 N aqueous NaOH (2×10 mL), dried over anhydrous Na₂SO₄ and filtered. The filtrate was evaporated under reduced pressure to give a brown oil. Purification by reverse-phase HPLC provided the title compound as a white solid. LC-MS m/z 303.9 (M+H)⁺, 3.56 min (ret. time).

### Intermediate 27

### (R)-8-Bromo-4-ethyl-2,3,4,5-tetrahydrobenzo[f][1,2]thiazepine 1,1-dioxide (S)-8-Bromo-4-ethyl-2,3,4,5-tetrahydrobenzo[f][1,2]thiazepine 1,1-dioxide

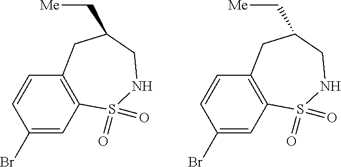 [SMILES: CC[C@H]1CNS(=O)(=O)c2cc(Br)ccc2C1 ; CC[C@@H]1CNS(=O)(=O)c2cc(Br)ccc2C1]

8-Bromo-4-ethyl-2,3,4,5-tetrahydrobenzo[f][1,2]thiazepine 1,1-dioxide (4.0 g, 13.15 mmol) was purified through chiral SFC (Column: Lux Cellulose-2 30×250 mm, 5u; Co-solvent: 20% (100% IPA); 80% CO₂, Flowrate: 90 g/min; Back pressure: 90 Bar) to provide (R)-8-bromo-4-ethyl-2,3,4,5-tetrahydrobenzo[f][1,2]thiazepine 1,1-dioxide (1.3 g, 4.4 mmol, 33% yield; LC-MS m/z 304/306 (M+H)⁺, 4.53 min (ret. time); ¹H NMR (400 MHz, DMSO-d6) δ ppm 0.87 (3H, t, J=7.34 Hz), 1.14 (2H, br s), 1.56 (1H, br s), 3.04-3.25 (3H, m), 3.31-3.42 (1H, m), 7.39 (1H, d, J=8.11 Hz), 7.70 (2H, dd, J=8.00, 2.08 Hz), 7.82 (1H, d, J=1.97 Hz)) and (S)-8-bromo-4-ethyl-2,3,4,5-tetrahydrobenzo[f][1,2]thiazepine 1,1-dioxide (1.5 g, 4.5 mmol, 34% yield; LC-MS m/z 304/306 (M+H)⁺, 4.57 min (ret. time); ¹H NMR (400 MHz, DMSO-d6) δ ppm 0.87 (3H, t, J=7.34 Hz), 1.14 (2H, br s), 1.56 (1H, br s), 3.08-3.26 (3H, m), 3.40 (1H, br s), 7.39 (1H, d, J=8.11 Hz), 7.70 (2H, dd, J=8.00, 2.08 Hz), 7.82 (1H, d, J=2.19 Hz)), with the absolute stereochemistry of each enantiomer confirmed by VCD analysis.

### Intermediate 28

### 1-((1-(2-Fluorophenyl)propyl)amino)-2-methylpropan-2-ol

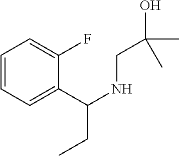 [SMILES: CCC(NCC(C)(C)O)c1ccccc1F]

To a solution of 1-amino-2-methylpropan-2-ol (10.54 g, 118 mmol) in toluene (400 mL) was added 1-(2-fluorophenyl)propan-1-one (15 g, 99 mmol). It was heated at 120° C. for 48 h. NaBH₄ (7.46 g, 197 mmol) was added and stirred at RT for 48 h. The reaction mixture was concentrated and quenched with 1 N NaOH solution (80 mL) solution and extracted with ethyl acetate (2×100 mL). The organic layer was concentrated and purified on flash column chromatography eluting with EtOAc:hexane (4:6). Desired fractions were concentrated under vacuum to give the title compound (3.8 g, 10.69 mmol, 10.85% yield) as gummy liquid. LC-MS m/z 226.1 (M+H)⁺, 3.872 min (ret. time).

### Intermediate 29

### 5-Ethyl-2,2-dimethyl-2,3,4,5-tetrahydrobenzo[f][1,4]oxazepine

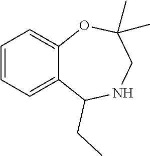

To a solution of 1-((1-(2-fluorophenyl)propyl)amino)-2-methylpropan-2-ol (3.5 g, 15.53 mmol) in dimethyl sulfoxide (DMSO) (20 mL) at 10° C. was added potassium tert-butoxide (8.72 g, 78 mmol). It was heated to 90° C. for 12 h. The reaction mixture was cooled to room temperature and poured in ice water (50 mL), then extracted with ethyl acetate (2×50 mL). The combined organic layer was concentrated. The crude residue was purified on flash column chromatography eluting with EtOAc:hexane (4:6). Desired fractions were concentrated to give the title compound (1.5 g, 4.79 mmol, 30.9% yield) as a gummy liquid. LC-MS m/z 206.2 (M+H)⁺, 3.255 min (ret. time).

### Intermediate 30

### Ethyl 3-(2-cyanophenyl)-2,2-dimethylpropanoate

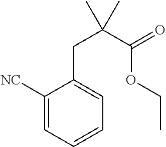 [SMILES: CCOC(=O)C(C)(C)Cc1ccccc1C#N]

To a solution of ethyl isobutyrate (4.74 g, 40.8 mmol) in tetrahydrofuran (THF) (80 mL) at −78° C. was added LDA (30.6 mL, 61.2 mmol). It was stirred at that temperature for 45 min, then a solution of 2-(bromomethyl)benzonitrile (8 g, 40.8 mmol) in tetrahydrofuran (THF) (30 mL) was added slowly and stirred for at −78° C. for 1 h. The reaction was then allowed to warm to ambient temperature for 3 h. The reaction mixture was quenched with saturated NH₄C₁ solution and extracted with DCM (2×30 mL). The combined organic layer was washed with brine solution (50 mL). The organic layer was dried over anhydrous Na₂SO₄, filtered and concentrated. The crude residue was purified by column chromatography eluting with 12% ethyl acetate in n-hexane. Desired fractions were concentrated to give the title compound (6 g, 24.85 mmol, 60.9% yield). ¹H NMR (400 MHz, chloroform-d) δ ppm: 1.11-1.31 (m, 9H) 3.10-3.23 (m, 2H) 4.15 (q, J=7.02 Hz, 2H) 7.26-7.37 (m, 2H) 7.44-7.53 (m, 1H) 7.62 (d, J=7.67 Hz, 1H).

### Intermediate 31

### 3-(2-(Aminomethyl)phenyl)-2,2-dimethylpropan-1-ol

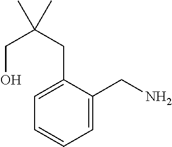

To a solution of ethyl 3-(2-cyanophenyl)-2,2-dimethylpropanoate (6 g, 25.9 mmol) in tetrahydrofuran (THF) (60 mL) at 0° C. was added LAH (78 mL, 78 mmol). It was stirred at 25° C. for 16 h. The reaction mixture was quenched with saturated Na₂SO₄ solution (15 mL), filtered and the filtrate was extracted with ethyl acetate (3×50 mL). The combined organic layer dried over anhydrous Na₂SO₄, filtered and concentrated to give the title compound (3 g, 14.88 mmol, 57.3% yield). LC-MS m/z 194.0 (M+H)⁺, 3.73 min (ret. time).

### Intermediate 32

### Ethyl 3-(2-cyanophenyl)-2,2-dimethylpropanoate

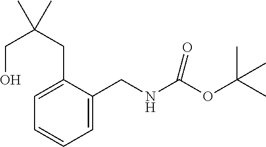 [SMILES: CC(C)(CO)Cc1ccccc1CNC(=O)OC(C)(C)C]

To a solution of 3-(2-(aminomethyl)phenyl)-2,2-dimethylpropan-1-ol (3 g, 15.52 mmol) in dichloromethane (DCM) (30 mL) was added Boc₂O (3.60 mL, 15.52 mmol). It was stirred at ambient temperature for 16 h. The reaction mixture was concentrated and purified by column chromatography eluting with 25% ethyl acetate in n-hexane. Desired fractions were concentrated to give the title compound (6 g, 24.85 mmol, 60.9% yield). LC-MS m/z 294.34 (M+H)⁺, 3.78 min (ret. time).

### Intermediate 33

### 3-(2-(((tert-Butoxycarbonyl)amino)methyl)phenyl)-2,2-dimethylpropyl methanesulfonate

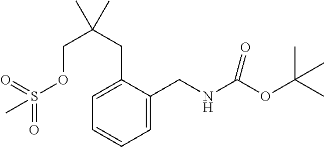 [SMILES: CC(C)(COS(C)(=O)=O)Cc1ccccc1CNC(=O)OC(C)(C)C]

To a solution of tert-butyl 2-(3-hydroxy-2,2-dimethylpropyl)benzylcarbamate (3 g, 10.22 mmol) in dichloromethane (DCM) (35 mL) at 0° C. was added TEA (3.56 mL, 25.6 mmol) and mesyl chloride (1.594 mL, 20.45 mmol). It was stirred at ambient temperature for 2 h. The reaction mixture was quenched with water (20 mL) and extracted with DCM (2×30 mL). The combined organic layer was washed with brine solution (50 mL). The organic layer was dried over anhydrous Na₂SO₄, filtered and concentrated. The crude residue was purified by column chromatography eluting with 20% ethyl acetate in n-hexane. Desired fractions were concentrated under reduced pressure to give the title compound (3 g, 7.70 mmol, 75% yield). LC-MS m/z 372.21 (M+H)⁺, 2.48 min (ret. time).

### Intermediate 34

### tert-Butyl 4,4-dimethyl-4,5-dihydro-1H-benzo[c]azepine-2(3H)-carboxylate

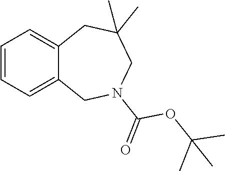 [SMILES: CC1(C)Cc2ccccc2CN(C(=O)OC(C)(C)C)C1]

To a solution of 3-(2-(((tert-butoxycarbonyl)amino)methyl)phenyl)-2,2-dimethylpropyl methanesulfonate (3 g, 8.08 mmol) in isopropanol (50 mL) was added Cs₂CO₃ (7.89 g, 24.23 mmol) and copper(I) iodide (0.154 g, 0.808 mmol). The reaction mixture was heated to 95° C. for 72 h. The reaction mixture was filtered through celite pad and washed with 10% MeOH in DCM (80 mL). The filtrate was concentrated to afford crude residue. The crude residue was purified by column chromatography eluting with 4% ethyl acetate in n-hexane. Desired fractions were concentrated to give the title compound (1.5 g, 4.56 mmol, 56.5% yield). LC-MS m/z 276.62 (M+H)⁺, 5.55 min (ret. time).

### Intermediate 35

### 4,4-Dimethyl-2,3,4,5-tetrahydro-1H-benzo[c]azepine hydrochloride

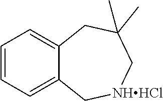

To a solution of tert-butyl 4,4-dimethyl-4,5-dihydro-1H-benzo[c]azepine-2(3H)-carboxylate (1.5 g, 5.45 mmol) in 1,4-dioxane (5 mL) at 0° C. was added 4M HCl in 1,4-dioxane (4 mL, 16.00 mmol). It was stirred at ambient temperature for 2 h. The reaction mixture was concentrated. Diethyl ether (20 mL) was added to the crude residue and stirred for 30 min. It was filtered and dried to give the title compound (1.05 g, 4.93 mmol, 90% yield). LC-MS m/z 176.19 (M+H)⁺, 1.26 min (ret. time).

### Intermediate 36

### Ethyl 2-(2-cyanobenzyl)butanoate

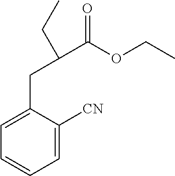 [SMILES: CCOC(=O)C(CC)Cc1ccccc1C#N]

To a solution of ethyl butyrate (0.681 mL, 5.10 mmol) in tetrahydrofuran (THF) (10 mL) at −78° C., was added lithium diisopropylamide (2M in THF) (3.83 mL, 7.65 mmol) slowly. After 30 min, a solution of 2-(bromomethyl)benzonitrile (1 g, 5.10 mmol) in THF (2 mL) was added slowly. It was stirred at −78° C. for 3 h. The reaction mixture was quenched with ammonium chloride solution (50 mL) and extracted with EtOAc (2×20 mL). The combined organic layer was washed with brine solution (20 mL) and dried over Na₂SO₄, filtered and concentrated. The crude residue was purified by silica gel chromatography to give the title compound (600 mg, 2.360 mmol, 46.3% yield) as a colorless liquid. LCMS m/z: 232.17 (M+H)⁺, 3.716 min (ret. time).

### Intermediate 37

### 2-(2-(Aminomethyl)benzyl)butan-1-ol

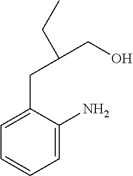

To a solution of ethyl 2-(2-cyanobenzyl)butanoate (600 mg, 2.59 mmol) in tetrahydrofuran (THF) (10 mL) at ambient temperature was added LAH (7.78 mL, 7.78 mmol) slowly. The reaction mixture was stirred for 3 h. The reaction mixture was quenched with ammonium chloride solution and extracted with EtOAc (2×50 mL). The combined organic layer was dried over Na₂SO₄, filtered and concentrated. The crude residue was purified by silica gel chromatography to give the title compound (400 mg, 2.069 mmol, 80% yield). LCMS m/z: 194 (M+H)⁺, 3.036 min (ret. time).

### Intermediate 38

### (2-(2-(Chloromethyl)butyl)phenyl)methanamine

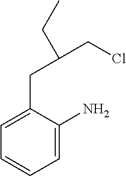

To a solution of 2-(2-(aminomethyl)benzyl)butan-1-ol (400 mg, 2.069 mmol) in 1,2-dichloroethane (DCE) (10 mL) at 5° C. was added sulfurous dichloride (0.302 mL, 4.14 mmol) slowly. The reaction mixture was allowed to stir at ambient temperature for 15 h. It was concentrated and quenched with saturated sodium bicarbonate and extracted with DCM (2×25 mL). The combined organic layer was washed with brine solution (20 mL), dried over Na₂SO₄, filtered and concentrated. The crude residue was purified by silica gel chromatography to give the title compound (300 mg, 1.417 mmol, 68.5% yield). LCMS m/z: 212 (M+H)⁺, 1.94 min (ret. time).

### Intermediate 39

### 4-Ethyl-2,3,4,5-tetrahydro-1H-benzo[c]azepine

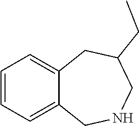

To a solution of (2-(2-(chloromethyl)butyl)phenyl)methanamine (300 mg, 1.417 mmol) in acetonitrile (2 mL) was added DIPEA (1.237 mL, 7.08 mmol). The reaction mixture was stirred at ambient temperature for 16 h. It was concentrated and extracted with DCM (2×50 mL). The organic layer was dried over Na₂SO₄, filtered, and concentrated. The crude residue was purified by silica gel chromatography to give the title compound (180 mg, 1.027 mmol, 72.5% yield). LCMS m/z: 176.22 (M+H)⁺, 1.33 min (ret. time).

### Intermediate 40

### 1-((2-Bromobenzyl)amino)-2-methylpropan-2-ol

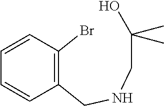

To a solution of 2-bromobenzaldehyde (25 g, 135 mmol) in methanol (250 mL) was added 1-amino-2-methylpropan-2-ol (12.04 g, 135 mmol) and NaOH (13.51 mL, 13.51 mmol). The reaction mixture was stirred under nitrogen atmosphere for 1 h. Then NaBH₄ (4.09 g, 108 mmol) was added portion wise for 10 min. It was stirred at 25° C. for 40 h. The reaction mixture was concentrated under reduced pressure. The residue was dissolved in ethyl acetate (500 mL) and washed with brine solution (300 mL). The organic layer was dried over anhydrous Na₂SO₄, filtered and concentrated to give the title compound (28 g, 108 mmol, 80% yield). ¹H NMR (DMSO-d6, 400 MHz): δ=7.55 (ddd, J=19.7, 7.7, 1.1 Hz, 2H), 7.33-7.39 (m, 1H), 7.18 (td, J=7.6, 1.6 Hz, 1H), 4.19 (s, 1H), 3.78 (s, 2H), 2.40 (s, 2H), 1.99 (s, 1H), 1.03-1.13 ppm (m, 6H).

### Intermediate 41

### 2,2-Dimethyl-2,3,4,5-tetrahydrobenzo[f][1,4]oxazepine

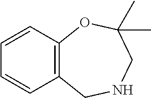

To a solution of 2-((2-bromobenzyl)amino)propan-2-ol (2.0 g, 8.19 mmol) in isopropanol (12 mL) was added Cs₂CO₃ (5.34 g, 16.38 mmol) and copper(I) iodide (0.156 g, 0.819 mmol). It was heated in microwave at 130° C. for 1 h. The reaction mixture was filtered through celite pad and washed with ethyl acetate. The filtrate was concentrated and the crude residue was purified by column chromatography eluting with 2% MeOH in DCM. Desired fractions were concentrated under reduced pressure to give the title compound (1.14 g, 4.97 mmol, 60.7% yield). LCMS m/z 178.19 (M+H)⁺, 2.82 min (ret. time).

### Intermediate 42

### tert-Butyl 2,2-dimethyl-2,3-dihydrobenzo[f][1,4]oxazepine-4(5H)-carboxylate

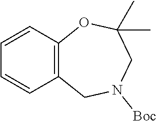 [SMILES: CC(C)(C)OC(=O)N1Cc2ccccc2OC(C)(C)C1]

To a solution of 2,2-dimethyl-2,3,4,5-tetrahydrobenzo[f][1,4]oxazepine (8 g, 45.1 mmol) in dichloromethane (DCM) (80 mL) was added TEA (9.44 mL, 67.7 mmol) and added Boc₂O (15.72 mL, 67.7 mmol) slowly. The reaction mixture was stirred at 25° C. for 16 h. The reaction mixture was diluted with water and extracted with DCM (2×100 mL). The combined organic layer was washed with brine solution (150 mL). The organic layer was dried over anhydrous Na₂SO₄, filtered and concentrated. The crude residue was purified by column chromatography eluting with 4% ethyl acetate in n-hexane. Desired fractions were concentrated to give the title compound (9 g, 32.4 mmol, 71.9% yield) ¹H NMR (CDCl₃, 400 MHz): δ=7.11-7.22 (m, 2H), 6.99-7.06 (m, 1H), 6.90-6.97 (m, 1H), 4.36-4.46 (m, 2H), 3.55-3.64 (m, 2H), 1.38-1.45 (m, 9H), 1.20 ppm (s, 6H).

### Intermediate 43

### 2,2-Dimethyl-2,3,4,5-tetrahydrobenzo[f][1,4]oxazepine hydrochloride

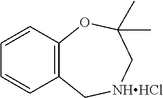

To a solution of tert-butyl 2,2-dimethyl-2,3-dihydrobenzo[f][1,4]oxazepine-4(5H)-carboxylate (9 g, 32.4 mmol) in 1,4-dioxane (50 mL) at 0° C. was added 4M HCl in 1,4-dioxane (16.22 mL, 64.9 mmol) slowly. The reaction mixture was allowed to stir at 25° C. for 2 h. The reaction mixture was concentrated. Diethyl ether was added to the residue and stirred for 30 min. Solid was precipitated out. The solid was filtered and dried to give the title compound (6.2 g, 28.2 mmol, 87% yield). LCMS m/z 178.32 (M+H)⁺, 2.78 min (ret. time).

### Intermediate 44

### 1-((5-Bromo-2-fluorobenzyl)amino)-2-methylpropan-2-ol

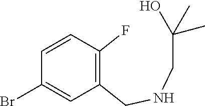

To a solution of 5-bromo-2-fluorobenzaldehyde (1 g, 4.93 mmol) in methanol (50 mL) was added 1-amino-2-methylpropan-2-ol (0.439 g, 4.93 mmol) and 1N sodium hydroxide (0.493 mL, 0.493 mmol). It was stirred for 4 h; sodium tetrahydroborate (0.186 g, 4.93 mmol) was added and stirred for 16 h. The reaction mixture was concentrated, quenched with ice cold water (50 mL) and extracted with ethyl acetate (3×30 mL). The combined organic layer was washed with brine solution (50 mL). The organic layer was dried over anhydrous Na₂SO₄, filtered and concentrated. The crude residue was purified by column chromatography eluting with 50% ethyl acetate in n-hexane. Desired fractions were concentrated to give the title compound (820 mg, 2.89 mmol, 58.6% yield). LCMS m/z 275.97 (M+H)⁺, 1.97 min (ret. time).

### Intermediate 45

### 7-Bromo-2,2-dimethyl-2,3,4,5-tetrahydrobenzo[f][1,4]oxazepine

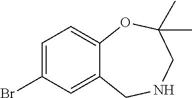

To a solution of 1-((5-bromo-2-fluorobenzyl)amino)-2-methylpropan-2-ol (6 g, 21.73 mmol) in dimethyl sulfoxide (DMSO) (40 mL) was added potassium tert-butoxide (6.10 g, 54.3 mmol). It was heated at 90° C. for 1 h. The reaction mixture was cooled and quenched with ice (10 g). It was extracted with ethyl acetate (3×20 mL). The combined organic layer was washed with ice cold water (3×30 mL) and brine solution (30 mL). The organic layer was dried over anhydrous Na₂SO₄, filtered and concentrated. The crude residue was purified by column chromatography eluting with 70% ethyl acetate in n-hexane. Desired fractions were concentrated to give the title compound (2.4 g, 3.87 mmol, 17.82% yield). LCMS m/z 257.91 (M+2H)⁺, 3.42 min (ret. time).

### tert-Butyl 7-bromo-2,2-dimethyl-2,3-dihydrobenzo[f][1,4]oxazepine-4(5H)-carboxylate

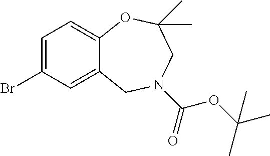 [SMILES: CC(C)(C)OC(=O)N1Cc2cc(Br)ccc2OC(C)(C)C1]

To a solution of 7-bromo-2,2-dimethyl-2,3,4,5-tetrahydrobenzo[f][1,4]oxazepine (7.2 g, 28.1 mmol) in dichloromethane (DCM) (50 mL) was added TEA (5.88 mL, 42.2 mmol) and Boc-anhydride (6.53 mL, 28.1 mmol). It was stirred at ambient temperature for 30 min. The reaction mixture was quenched with water (10 mL) and extracted with DCM (2×20 mL). The combined organic layer washed with brine solution (20 mL). The organic layer was dried over anhydrous Na₂SO₄, filtered and concentrated. The crude residue was purified by column chromatography eluting with 5% ethyl acetate in n-hexane. Desired fractions were concentrated to give the title compound (3.3 g, 9.08 mmol, 32.3% yield). LCMS m/z 299.91 (M-57)⁺, 4.26 min (ret. time).

### Intermediate 46

### 7-Bromo-2,2-dimethyl-2,3,4,5-tetrahydrobenzo[f][1,4]oxazepine hydrochloride

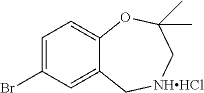

To a solution of tert-butyl 7-bromo-2,2-dimethyl-2,3-dihydrobenzo[f][1,4]oxazepine-4(5H)-carboxylate (3.3 g, 9.26 mmol) in 1,4-dioxane (40 mL) at 0° C. was added 4M HCl in 1,4-dioxane (6.95 mL, 27.8 mmol). It was then stirred at ambient temperature for 2 h. The reaction mixture was concentrated. Diethyl ether (20 mL) was added and stirred for 30 min. Solid was filtered, washed with hexane (5 mL) and dried to give the title compound (2.1 g, 7.08 mmol, 76% yield) as off-white solid. LCMS m/z 256.04 (M−HCl)⁺, 1.48 min (ret. time).

### Intermediate 47

### 1-(((3-Fluoropyridin-2-yl)methyl)amino)-2-methylpropan-2-ol

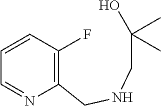

To a solution of 3-fluoropicolinaldehyde (10 g, 80 mmol) in methanol (100 mL) was added 1-amino-2-methylpropan-2-ol (7.13 g, 80 mmol) and Indium(III) trifluoromethanesulfonate (8.99 g, 15.99 mmol). It was stirred under nitrogen atmosphere for 1 h, then NaCNBH₄ (5.53 g, 88 mmol) was added portion wise for 10 min. It was stirred at RT for 24 h. It was concentrated and purified on flash column chromatography (Neutral alumina) by using MeOH:DCM (1:9) as solvent. Desired fractions were concentrated to give the title compound (4 g, 13.58 mmol, 16.99% yield) as a colorless liquid. LC-MS m/z 198.91 (M+H)⁺, 1.592 min (ret. time).

### Intermediate 48

### 2,2-Dimethyl-2,3,4,5-tetrahydropyrido[2,3-f][1,4]oxazepine

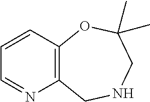

To a solution of 1-(((3-fluoropyridin-2-yl)methyl)amino)-2-methylpropan-2-ol (4 g, 20.18 mmol) in dimethyl sulfoxide (DMSO) (10 mL) was added potassium tert-butoxide (2.264 g, 20.18 mmol). The reaction mixture was heated at 90° C. for 1 h. The reaction mixture was poured in ice water (50 mL) and extracted with ethyl acetate (2×50 mL). The combined organic layer was washed with water (2×40 mL), brine (20 mL), dried over Na₂SO₄, filtered and concentrated. The crude residue was purified on flash column chromatography (Neutral alumina) using 45% ethyl acetate in hexane. The collected the fractions were concentrated to give the title compound (3.2 g, 15.59 mmol, 77% yield) a gummy liquid. LC-MS m/z 178.92 (M+H)⁺, 1.057 min (ret. time).

### Intermediate 49

### tert-Butyl 2,2-dimethyl-2,3-dihydropyrido[2,3-f][1,4]oxazepine-4(5H)-carboxylate

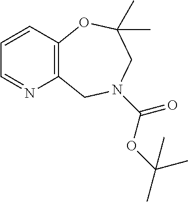 [SMILES: CC(C)(C)OC(=O)N1Cc2ncccc2OC(C)(C)C1]

To a solution of 2,2-dimethyl-2,3,4,5-tetrahydropyrido[2,3-f][1,4]oxazepine (3.2 g, 17.95 mmol) in dichloromethane (DCM) (5 mL) at 0° C. was added TEA (5.00 mL, 35.9 mmol) and Boc-anhydride (5.42 mL, 23.34 mmol). It was stirred at RT for 3 h. The crude residue was diluted with water (10 mL) and extracted with ethyl acetate (2×20 mL). The combined organic layer was washed with brine solution (10 mL), dried over anhydrous Na₂SO₄, filtered and concentrated. The crude residue was purified on flash column chromatography eluting with 3% ethyl acetate in hexane. Desired fractions were concentrated to give the title compound (3.1 g, 9.45 mmol, 52.6% yield) as a colorless liquid. LC-MS m/z 279.13 (M+H)⁺, 3.583 min (ret. time).

### Intermediate 50

### 2,2-Dimethyl-2,3,4,5-tetrahydropyrido[2,3-f][1,4]oxazepine hydrochloride

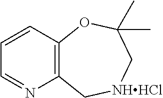

To a solution of tert-butyl 2,2-dimethyl-2,3-dihydropyrido[2,3-f][1,4]oxazepine-4(5H)-carboxylate (3.1 g, 11.14 mmol) in dichloromethane (DCM) (20 mL) at 10° C. was added 4 M HCl in 1,4-dioxane (3.34 mL, 13.36 mmol). It was stirred for 1 h. The obtained precipitation was filtered and the solid was washed with hexane, diethyl ether and dried to give the title compound (2.16 g, 9.85 mmol, 88% yield) as a brown solid. LC-MS m/z 179.1 (M−HCl)⁺, 4.040 min (ret. time)

### Intermediate 51

### (R)-1-((2-Bromobenzyl)amino)butan-2-ol

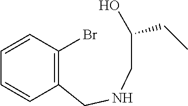

To a solution of 2-bromobenzaldehyde (10 g, 54.0 mmol) in methanol (100 mL) was added (R)-1-aminobutan-2-ol (4.82 g, 54.0 mmol) and NaOH (5.40 mL, 5.40 mmol). It was stirred under nitrogen atmosphere for 1 h. NaBH₄ (0.818 g, 21.62 mmol) was added portion wise for 10 min and stirred at RT for 72 h. Solvent was concentrated. The crude product was purified on flash column chromatography (Neutral alumina) eluting with EtOAc:hexane (3:7). Combined fractions were concentrated to give the title compound (10 g, 28.9 mmol, 53.5% yield). as an off-white solid. LC-MS m/z 258.12 (M+H)⁺, 1.302 min (ret. time)

### Intermediate 52

### (R)-2-Ethyl-2,3,4,5-tetrahydrobenzo[f][1,4]oxazepine

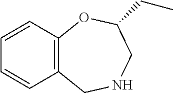

To a solution of (R)-1-((2-bromobenzyl)amino)butan-2-ol (3 g, 11.62 mmol) in isopropanol (30 mL) was added Cs₂CO₃ (7.57 g, 23.24 mmol) and copper(I) iodide (0.443 g, 2.324 mmol). The reaction mixture was heated in microwave reactor at 130° C. for 1 h. The reaction mixture was filtered through celite, washed with isopropanol and concentrated. The crude residue was purified on flash column chromatography eluting with 2.5% MeOH in DCM. Desired fractions were concentrated to give the title compound (1.7 g, 8.77 mmol, 75% yield) as a gummy liquid. LC-MS m/z 178.18 (M+H)⁺, 1.27 min (ret. time).

### Intermediate 53

### (R)-2-Ethyl-2,3,4,5-tetrahydrobenzo[f][1,4]oxazepine hydrochloride

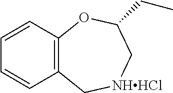

To a solution of (R)-2-ethyl-2,3,4,5-tetrahydrobenzo[f][1,4]oxazepine (5.5 g, 31.0 mmol) in dichloromethane (DCM) (20 mL) at 10° C. was added 4M HCl in 1,4-dioxane (9.31 mL, 37.2 mmol). It was stirred for 1 h. The obtained precipitates were filtered and the solid was washed with hexane and dried to give the title compound (5.1 g, 23.82 mmol, 77% yield) as an off-white solid. LC-MS m/z 178.1 (M+H)⁺, 1.563 min (ret. time).

### Intermediate 54

### Methyl 2-cycloheptylacetate

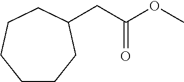

To a solution of 2-cycloheptylacetic acid (4.76 g, 30.5 mmol) in methanol (50 mL) was added sulfuric acid (2.99 g, 30.5 mmol) slowly. Then it was stirred at 70° C. for 16 h. After it was cooled to ambient temperature, the reaction mixture was added to 50 mL of water and extracted with ethyl acetate (3×50 mL), washed with brine, concentrated to obtain the title compound methyl 2-cycloheptylacetate (5.18 g, 28.3 mmol, 92.9% yield). LCMS m/z 171.2 (M+H)⁺, 1.82 min (Ret. time).

### Intermediate 55

### 2-Cycloheptylethanol

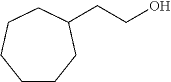

To a solution of methyl 2-cycloheptylacetate (4.33 g, 25.4 mmol) in tetrahydrofuran (THF) (20 mL) was added lithium aluminum hydride (1.931 g, 50.9 mmol) slowly under nitrogen at 0° C. and stirred for 1 hour. Then the reaction mixture was stirred at 25° C. for 16 hours. Then 30 mL of HCl (3 M) was added, extracted with EtOAc (3×30 mL), washed with brine, dried over MgSO₄ and concentrated to obtain the title compound 2-cycloheptylethanol (3.28 g, 20.75 mmol, 82% yield) as a white oil. ¹H NMR (400 MHz, DMSO-d₆) δ: 4.26 (t, J=4.9 Hz, 1H), 3.41 (q, J=5.9 Hz, 2H), 1.73-1.29 (m, 13H), 1.22-1.08 (m, 2H).

### Intermediate 56

### 2-Cycloheptylacetaldehyde

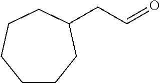

To a solution of 2-cycloheptylethanol (3.28 g, 23.06 mmol) in dichloromethane (DCM) (50 mL) was added PCC (7.46 g, 34.65 mmol) and silica gel (15 g). The reaction mixture was stirred at 25° C. for 16 h. Then it was filtered through a pad of celite. The filtrate was concentrated under vacuum. The crude product was purified by silica gel chromatography (EtOAC:Hexane=1:5) to obtain the title compound 2-cycloheptylacetaldehyde (1.30 g, 8.81 mmol, 38.2% yield) as a yellow oil. H NMR (400 MHz, CDCl₃) δ: 9.74 (s, 1H), 2.32-1.28 (m, 15H).

### Intermediate 57

### 2-(Cycloheptylmethyl)-1H-imidazole

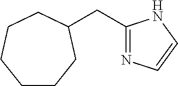 [SMILES: c1c[nH]c(CC2CCCCCC2)n1]

To a solution of 2-cycloheptylacetaldehyde (1.2 g, 8.56 mmol) in methanol (36 mL) and water (36 mL) was added oxaldehyde (0.993 g, 17.12 mmol) and ammonia hydrate (2.189 g, 62.5 mmol). The reaction mixture was stirred at 0° C. for 2 h, then it was stirred at ambient temperature for 18 h. The solid was filtered and dried under vacuum to obtain the title compound 2-(cycloheptylmethyl)-1H-imidazole (680 mg, 3.43 mmol, 40.1% yield) as a white solid. LCMS m/z 179.2 (M+H)⁺, 1.22 min (ret. time).

### Intermediate 58

### 1-((1H-Imidazol-2-yl)methyl)piperidine

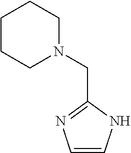 [SMILES: c1c[nH]c(CN2CCCCC2)n1]

To a solution of 1H-imidazole-2-carbaldehyde (2 g, 20.81 mmol) in 1,2-dichloroethane (DCE) (100 mL), piperidine (1.772 g, 20.81 mmol) and acetic acid (0.5 mL) were added. After it was stirred at ambient temperature for 16 h, NaBH(OAc)₃ (8.82 g, 41.6 mmol) was added. The reaction mixture was stirred at 25° C. for a further 2 h. The solvent was removed and the residue was purified by reverse-phase HPLC (0.05% NH₄HCO₃/H₂O:CH₃CN=5%-95%) to give the title compound 1-((1H-imidazol-2-yl)methyl)piperidine (1.6 g, 9.68 mmol, 46.5% yield) as a yellow solid. LC-MS m/z 166.2 (M+H)⁺, 1.27 min (ret. time).

### Example 1. Methyl 3-(dimethylamino)-2-(trans)-2-(1-methyl-1H-1,2,3-triazol-4-yl)cyclo-propanecarbonyl)acrylate

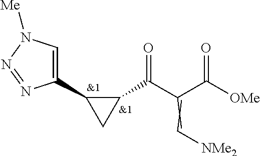 [SMILES: COC(=O)C(=CN(C)C)C(=O)[C@@H]1C[C@H]1c1cn(C)nn1]

### 1a) 4-(Diethoxymethyl)-1-methyl-1H-1,2,3-triazole

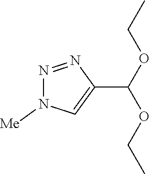

A solution of iodomethane (166 g, 1170 mmol) in tert-butanol (500 mL) was added to NaHCO₃ (98 g, 1170 mmol), copper(II) sulfate (12.45 g, 78 mmol), sodium azide (76 g, 1170 mmol) and sodium (R)-2-((S)-1,2-dihydroxyethyl)-4-hydroxy-5-oxo-2,5-dihydrofuran-3-olate (30.9 g, 156 mmol) in water (500 mL) slowly at room temperature. Then 3,3-diethoxyprop-1-yne (50 g, 390 mmol) was added. The reaction mixture was stirred at 60° C. for 16 h. The reaction mixture was extracted with ethyl acetate (3×1000 mL). The combined organic layer was dried with MgSO₄ and concentrated to obtain the title compound 4-(diethoxymethyl)-1-methyl-1H-1,2,3-triazole (46 g, 236 mmol, 60.5% yield) which was carried over to next step without further purification. LC-MS m/z 186.1 (M+H)⁺, 1.46 min (ret. time).

### 1b) 1-Methyl-1H-1,2,3-triazole-4-carbaldehyde

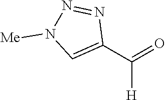

To a solution of 4-(diethoxymethyl)-1-methyl-1H-1,2,3-triazole (46 g, 248 mmol) in water (200 mL), TFA (100 mL, 649 mmol) was added. The reaction mixture was stirred at room temperature for 1 h. The water was evaporated and dried under vacuum to get the title compound, 1-methyl-1H-1,2,3-triazole-4-carbaldehyde (26 g, 234 mmol, 94% yield) as a yellow solid. LC-MS m/z 112.2 (M+H)⁺, 0.51 min (ret. time).

### 1c) (E)-tert-Butyl 3-(1-methyl-1H-1,2,3-triazol-4-yl)acrylate

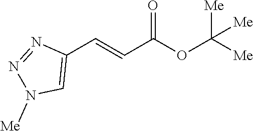 [SMILES: Cn1cc(/C=C/C(=O)OC(C)(C)C)nn1]

To a solution of tert-butyl 2-(diethoxyphosphoryl)acetate (62.4 g, 248 mmol) in tetrahydrofuran (500 mL), sodium hydride (10.80 g, 270 mmol, 60%) was added at 0° C. The reaction mixture was stirred at 0° C. under N₂ for 10 min. Then a solution of 1-methyl-1H-1,2,3-triazole-4-carbaldehyde (25 g, 225 mmol) in THF (500 mL) was added dropwise and the reaction mixture was stirred at 0° C. for 15 min. Water (500 mL) was added and extracted with ethyl acetate (3×300 mL). The combined organic layer was washed with water (2×100 mL) and brine (2×100 mL), dried over Na₂SO₄ and concentrated. The crude product was purified by Combiflash chromatography (hexane:ethyl acetate=1:5) to give the title compound (E)-tert-butyl 3-(1-methyl-1H-1,2,3-triazol-4-yl)acrylate (40 g, 184 mmol, 82% yield) as an oil. LC-MS m/z 210.1 (M+H)⁺, 1.73 min (ret. time).

### 1d) (trans)-tert-Butyl 2-(1-methyl-1H-1,2,3-triazol-4-yl)cyclopropanecarboxylate

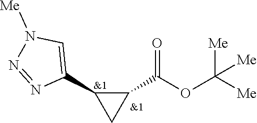 [SMILES: Cn1cc([C@@H]2C[C@H]2C(=O)OC(C)(C)C)nn1]

To a solution of trimethylsulfoxonium iodide (126 g, 573 mmol) in dimethyl sulfoxide (300 mL), sodium hydride (16.06 g, 401 mmol) was added at 0° C. The reaction mixture was stirred at room temperature under N₂ for 1 h. A solution of (E)-tert-butyl 3-(1-methyl-1H-1,2,3-triazol-4-yl)acrylate (40 g, 191 mmol) in tetrahydrofuran (300 mL) was subsequently added dropwise. The reaction mixture was stirred at room temperature for 1 hr and heated to 50° C. for another 1 h. The reaction mixture was cooled to RT and partitioned with 200 mL of ethyl acetate and 250 mL of water. The water layer was extracted with ethyl acetate (3×250 mol), the combined organic layer was dried with Na₂SO₄ and concentrated to afford (trans)-tert-butyl 2-(1-methyl-1H-1,2,3-triazol-4-yl)cyclopropanecarboxylate (36 g, 144 mmol, 75% yield). LC-MS m/z 224.1 (M+H)⁺, 1.69 min (ret. time).

### 1e) (trans)-2-(1-Methyl-1H-1,2,3-triazol-4-yl)cyclopropanecarboxylic acid

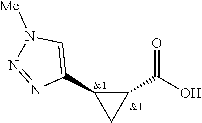 [SMILES: Cn1cc([C@@H]2C[C@H]2C(=O)O)nn1]

A solution of (trans)-tert-butyl 2-(1-methyl-1H-1,2,3-triazol-4-yl)cyclopropanecarboxylate (36 g, 161 mmol) in dichloromethane (400 mL), TFA (200 mL, 2596 mmol) was added slowly under nitrogen at room temperature. The reaction mixture was stirred at room temperature for 4 h Then it was concentrated. 100 mL of ethyl acetate and 100 mL of water were added to residue. The water layer was extracted with ethyl acetate (3×100 mL). The combined organic phase was dried with MgSO₄ and concentrated to get title compound 2-(1-methyl-1H-1,2,3-triazol-4-yl)cyclopropanecarboxylic acid (24 g, 134 mmol, 83% yield) as a white solid. LC-MS m/z 168.1 (M+H)⁺, 1.16 min (ret. time).

### 1f) Methyl 3-((trans)-2-(1-methyl-1H-1,2,3-triazol-4-yl)cyclopropyl)-3-oxopropanoate

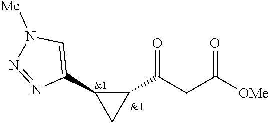 [SMILES: COC(=O)CC(=O)[C@@H]1C[C@H]1c1cn(C)nn1]

To a solution of (trans)-2-(1-methyl-1H-1,2,3-triazol-4-yl)cyclopropanecarboxylic acid (24 g, 144 mmol) in tetrahydrofuran (700 mL), was added CDI (30.3 g, 215 mmol). The reaction mixture was stirred at room temperature for 2 h. Potassium 3-methoxy-3-oxopropanoate (67.3 g, 431 mmol) was subsequently added. The reaction mixture was stirred at room temperature for 18 h. The solvent was evaporated and re-dissolved in ethyl acetate (200 mL). It was then washed with 1 M KHSO₄ (150 mL), saturated NaHCO₃ (150 mL) and brine (150 mL). The organic layer was dried with Na₂SO₄ and concentrated to obtain the title compound methyl 3-(2-(1-methyl-1H-1,2,3-triazol-4-yl)cyclopropyl)-3-oxopropanoate as an oil (20 g, 85 mmol, 59.3% yield). LC-MS m/z 224.1 (M+H)⁺, 1.39 min (ret. time).

### 1g) Methyl 3-(dimethylamino)-2-(trans)-2-(1-methyl-1H-1,2,3-triazol-4-yl)cyclopropane-carbonyl)acrylate

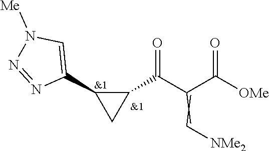 [SMILES: COC(=O)C(=CN(C)C)C(=O)[C@@H]1C[C@H]1c1cn(C)nn1]

A mixture of methyl 3-(2-(1-methyl-1H-1,2,3-triazol-4-yl)cyclopropyl)-3-oxopropanoate (19 g, 85 mmol) 1,1-dimethoxy-N,N-dimethylmethanamine (11.16 g, 94 mmol) was stirred at 25° C. for 12 h. The reaction mixture was concentrated to obtain the title compound methyl 3-(dimethylamino)-2-(2-(1-methyl-1H-1,2,3-triazol-4-yl)cyclopropanecarbonyl)acrylate (24 g, 77 mmol, 90% yield) as an oil which was used in next step without further purification. LC-MS m/z 279.1 (M+H)⁺, 1.61 min (ret. time).

The example in Table 2 was prepared in an analogous manner:

TABLE 2

| | | | LCMS | Retention |
|---|---|---|---|---|
| Ex # | Structure | Name | [M + H]⁺ | Time (min) |
| Example 2 | 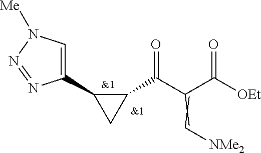 | ethyl 3-(dimethylamino)- 2-(trans)-2-(1-methyl-1H- 1,2,3-triazol-4- yl)cyclopropanecarbonyl) acrylate | 292.9 | 2.74 |

### Example 3. 1-(3-(((R)-4-ethyl-1,1-dioxido-3,4-dihydro-2H-benzo[b][1,4,5]oxathiazepin-2-yl)methyl)phenyl)-5-(trans)-2-(1-methyl-1H-1,2,3-triazol-4-yl)cyclopropyl)-1H-pyrazole-4-carboxylic acid

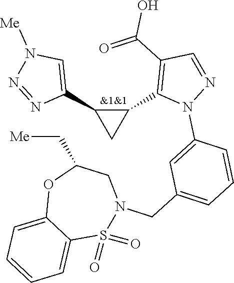 [SMILES: CC[C@@H]1CN(Cc2cccc(-n3ncc(C(=O)O)c3[C@@H]3C[C@H]3c3cn(C)nn3)c2)S(=O)(=O)c2ccccc2O1]

### 3a) 3-(4-(Ethoxycarbonyl)-5-(trans)-2-(1-methyl-1H-1,2,3-triazol-4-yl)cyclopropyl)-1H-pyrazol-1-yl)benzoic acid

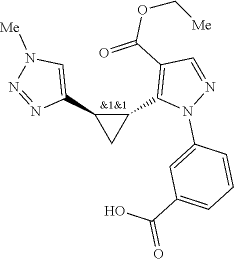 [SMILES: CCOC(=O)c1cnn(-c2cccc(C(=O)O)c2)c1[C@@H]1C[C@H]1c1cn(C)nn1]

To a suspension of 3-hydrazinylbenzoic acid (72.9 mg, 0.479 mmol) in ethanol (4789 μl) was added ethyl 3-(dimethylamino)-2-(trans)-2-(1-methyl-1H-1,2,3-triazol-4-yl)cyclopropanecarbonyl)acrylate (140 mg, 0.479 mmol) and triethylamine (66.7 μl, 0.479 mmol) sequentially. Warned to 65° C. After 50 min, cooled to RT, added an additional amount of 3-hydrazinylbenzoic acid (30 mg, 0.197 mmol) and warmed to 65° C. After 10 min, cooled to RT. Partitioned with 40 mL EtOAc and 20 mL aqueous 1M HCl, and separated layers. Back-extracted aqueous with 3×10 mL EtOAc. Dried combined organics over Na₂SO₄, filtered, and concentrated in vacuo to give an orange solid. Purified by normal-phase CombiFlash ISCO (12 g Gold column, 0-50% (3:1 EtOAc:EtOH):Hexane) to give 3-(4-(ethoxycarbonyl)-5-(trans)-2-(1-methyl-1H-1,2,3-triazol-4-yl)cyclopropyl)-1H-pyrazol-1-yl)benzoic acid as an orange solid (110 mg, 0.288 mmol, 60% yield). LC-MS m/z 382.1 (M+H)⁺, 0.71 min (ret. time).

### 3b) Ethyl 1-(3-(hydroxymethyl)phenyl)-5-(trans)-2-(1-methyl-1H-1,2,3-triazol-4-yl)cyclopropyl)-1H-pyrazole-4-carboxylate

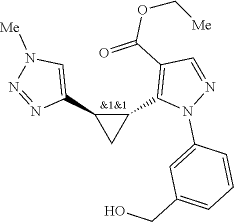 [SMILES: CCOC(=O)c1cnn(-c2cccc(CO)c2)c1[C@@H]1C[C@H]1c1cn(C)nn1]

To a cloudy mixture of 3-(4-(ethoxycarbonyl)-5-(trans)-2-(1-methyl-1H-1,2,3-triazol-4-yl)cyclopropyl)-1H-pyrazol-1-yl)benzoic acid (187.7 mg, 0.492 mmol was added CDI (160 mg, 0.984 mmol, giving a clear, orange solution. Stirred for 2 h, then cautiously added to a vigorously stirring solution of NaBH₄ (93 mg, 2.461 mmol in water (2355 μl at RT. After 3 min at RT, quenched with a dropwise addition of 1M aqueous hydrochloric acid until pH=2. Partitioned with 50 mL EtOAc and 10 mL H₂O. Separated layers, back-extracted aqueous with 3×10 mL EtOAc. Dried combined organics over Na₂SO₄, filtered, and concentrated in vacuo to give an orange oil. Purified by normal-phase CombiFlash ISCO (24 g column, 0-70% (3:1 EtOAc:EtOH):Hexane) to give ethyl 1-(3-(hydroxymethyl)phenyl)-5-(trans)-2-(1-methyl-1H-1,2,3-triazol-4-yl)cyclopropyl)-1H-pyrazole-4-carboxylate as a pale yellow oil (67 mg, 0.182 mmol, 37% yield). LC-MS m/z 368.0 (M+H)⁺, 0.76 min (ret. time). ¹H NMR (400 MHz, CHLOROFORM-d) δ ppm 1.35-1.41 (m, 3H) 1.41-1.47 (m, 1H) 1.50-1.61 (m, 1H) 2.18-2.28 (m, 1H) 2.43-2.59 (m, 1H) 4.05 (s, 3H) 4.25-4.42 (m, 2H) 4.72 (s, 2H) 7.28 (br. s., 1H) 7.39 (d, J=4.27 Hz, 1H) 7.45 (d, J=4.52 Hz, 2H) 7.63 (s, 1H) 8.06 (s, 1H).

### 3c) Ethyl 1-(3-(((R)-4-ethyl-1,1-dioxido-3,4-dihydro-2H-benzo[b][1,4,5]oxathiazepin-2-yl)methyl)phenyl)-5-(trans)-2-(1-methyl-1H-1,2,3-triazol-4-yl)cyclopropyl)-1H-pyrazole-4-carboxylate

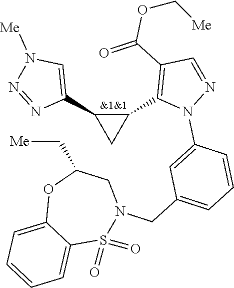 [SMILES: CCOC(=O)c1cnn(-c2cccc(CN3C[C@@H](CC)Oc4ccccc4S3(=O)=O)c2)c1[C@@H]1C[C@H]1c1cn(C)nn1]

To a solution of ethyl 1-(3-(hydroxymethyl)phenyl)-5-(trans)-2-(1-methyl-1H-1,2,3-triazol-4-yl)cyclopropyl)-1H-pyrazole-4-carboxylate (55.0 mg, 0.150 mmol) in tetrahydrofuran (THF) (2994 μl) was added sequentially (R)-4-ethyl-3,4-dihydro-2H-benzo[b][1,4,5]oxathiazepine 1,1-dioxide (68.0 mg, 0.299 mmol), DTBAD (68.9 mg, 0.299 mmol) and triphenylphosphine (79 mg, 0.299 mmol). After 1 h, added additional amounts of triphenylphosphine (160 mg, 0.598 mmol) and DTBAD (140 mg, 0.598 mmol). After 40 min, concentrated to give a pale yellow oil. Purified by normal-phase CombiFlash ISCO (24 g Gold column, 0-70% EtOAc:Hexane) to give a white solid. LC-MS consistent with ethyl 1-(3-(((R)-4-ethyl-1,1-dioxido-3,4-dihydro-2H-benzo[b][1,4,5]oxathiazepin-2-yl)methyl)phenyl)-5-(trans)-2-(1-methyl-1H-1,2,3-triazol-4-yl)cyclopropyl)-1H-pyrazole-4-carboxylate (m/z 577.1 (M+H)⁺, 1.08 min (ret. time)) as well as triphenylphosphine oxide (m/z 279.0 (M+H)⁺, 0.88 min (ret. time)). Carried mixture forward to ester hydrolysis step.

### 3d) 1-(3-(((R)-4-Ethyl-1,1-dioxido-3,4-dihydro-2H-benzo[b][1,4,5]oxathiazepin-2-yl)methyl)phenyl)-5-(trans)-2-(1-methyl-1H-1,2,3-triazol-4-yl)cyclopropyl)-1H-pyrazole-4-carboxylic acid

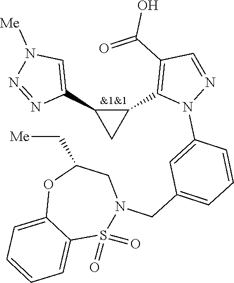 [SMILES: CC[C@@H]1CN(Cc2cccc(-n3ncc(C(=O)O)c3[C@@H]3C[C@H]3c3cn(C)nn3)c2)S(=O)(=O)c2ccccc2O1]

To a solution of ethyl 1-(3-(((R)-4-ethyl-1,1-dioxido-3,4-dihydro-2H-benzo[b][1,4,5]oxathiazepin-2-yl)methyl)phenyl)-5-(trans)-2-(1-methyl-1H-1,2,3-triazol-4-yl)cyclopropyl)-1H-pyrazole-4-carboxylate (86 mg, 0.149 mmol in methanol (497 μl was added an aqueous solution of NaOH (1491 μl, 1.491 mmol, 1M. The resulting reaction mixture was warmed to 50° C. After 4 h, cooled to room temperature. Partitioned with 15 mL EtOAc and 5 mL H₂O, separated layers. Back-extracted aqueous with 3×5 mL EtOAc. Acidified aqueous layer to pH=2 with 1M aqueous HCl, partitioned with 10 mL EtOAc, separated layers. Back-extracted aqueous with 3×5 mL EtOAc. Dried over Na₂SO₄, filtered and concentrated in vacuo to give 1-(3-(((R)-4-ethyl-1,1-dioxido-3,4-dihydro-2H-benzo[b][1,4,5]oxathiazepin-2-yl)methyl)phenyl)-5-(trans)-2-(1-methyl-1H-1,2,3-triazol-4-yl)cyclopropyl)-1H-pyrazole-4-carboxylic acid as a white solid (24.6 mg, 0.045 mmol, 30% yield). LC-MS m/z 549.0 (M+H)⁺, 0.93 min (ret. time). ¹H NMR (400 MHz, CHLOROFORM-d) δ ppm 1.14 (t, J=7.28 Hz, 3H) 1.46 (d, J=2.76 Hz, 1H) 1.56 (d, J=3.76 Hz, 1H) 1.66 (br. s., 1H) 1.71-1.82 (m, 1H) 2.14 (s, 3H) 2.26-2.39 (m, 1H) 2.43-2.56 (m, 1H) 3.14 (d, J=15.06 Hz, 1H) 3.76-3.96 (m, 2H) 4.01 (d, J=4.52 Hz, 1H) 4.07 (d, J=1.00 Hz, 3H) 4.43-4.57 (m, 1H) 7.23 (d, J=8.03 Hz, 1H) 7.30 (br. s., 1H) 7.42-7.58 (m, 5H) 7.88 (d, J=7.03 Hz, 1H) 8.13 (s, 1H).

### Example 4. 1-(3-(((S)-4-Ethyl-1,1-dioxido-4,5-dihydrobenzo[f][1,2]thiazepin-2(3H)-yl)methyl)phenyl)-5-(trans)-2-(1-methyl-1H-1,2,3-triazol-4-yl)cyclopropyl)-1H-pyrazole-4-carboxylic acid

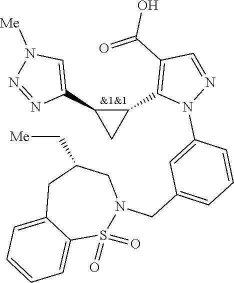 [SMILES: CC[C@H]1Cc2ccccc2S(=O)(=O)N(Cc2cccc(-n3ncc(C(=O)O)c3[C@@H]3C[C@H]3c3cn(C)nn3)c2)C1]

### 4a) 3-(4-(Methoxycarbonyl)-5-(trans)-2-(1-methyl-1H-1,2,3-triazol-4-yl)cyclopropyl)-1H-pyrazol-1-yl)benzoic acid

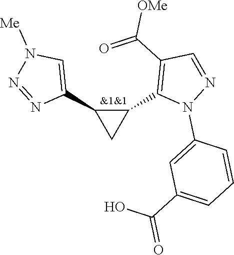 [SMILES: COC(=O)c1cnn(-c2cccc(C(=O)O)c2)c1[C@@H]1C[C@H]1c1cn(C)nn1]

To a suspension of 3-hydrazinylbenzoic acid (1.203 g, 7.90 mmol) in ethanol (79 ml) was added methyl 3-(dimethylamino)-2-(trans)-2-(1-methyl-1H-1,2,3-triazol-4-yl)cyclo-propanecarbonyl)acrylate (2.2 g, 7.90 mmol) and triethylamine (1.102 ml, 7.90 mmol) sequentially. Warmed to 65° C. After 1 h, cooled to RT. Partitioned with 400 mL EtOAc and 200 mL 1M aqueous HCl, separated layers. Back-extracted aqueous with 3×50 mL EtOAc. Dried combined organics over Na₂SO₄, filtered, and concentrated in vacuo to give an orange solid. Purified by normal-phase CombiFlash Torrent (220 g Gold column, 0-15% MeOH:DCM) to give 3-(4-(methoxycarbonyl)-5-(trans)-2-(1-methyl-1H-1,2,3-triazol-4-yl)cyclopropyl)-1H-pyrazol-1-yl)benzoic acid as a yellow solid (1.8 g, 4.9 mmol, 62% yield). LC-MS m/z 368.1 (M+H)⁺, 0.64 min (ret. time). ¹H NMR (400 MHz, DMSO-d₆) δ ppm 1.09 (d, J=4.02 Hz, 1H) 1.27-1.41 (m, 1H) 2.12 (d, J=8.03 Hz, 1H) 2.53-2.57 (m, 1H) 3.76 (s, 3H) 3.97 (s, 3H) 7.58-7.64 (m, 1H) 7.66 (s, 1H) 7.87 (d, J=8.03 Hz, 1H) 8.01 (d, J=7.78 Hz, 1H) 8.06 (s, 1H) 8.07 (s, 1H), 12.62-13.18 (m, 1H).

### 4b) Methyl 1-(3-(hydroxymethyl)phenyl)-5-(trans)-2-(1-methyl-1H-1,2,3-triazol-4-yl)cyclopropyl)-1H-pyrazole-4-carboxylate

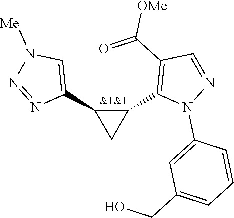 [SMILES: COC(=O)c1cnn(-c2cccc(CO)c2)c1[C@@H]1C[C@H]1c1cn(C)nn1]

To a yellow suspension of 3-(4-(methoxycarbonyl)-5-(trans)-2-(1-methyl-1H-1,2,3-triazol-4-yl)cyclopropyl)-1H-pyrazol-1-yl)benzoic acid (1.8 g, 4.90 mmol) in tetrahydrofuran (88 ml) at RT was added CDI (2.384 g, 14.70 mmol), giving a clear, yellow solution. After 2 h, cautiously added the solution to a mixture of NaBH₄ (0.927 g, 24.50 mmol) in water (23.44 ml) at RT. After 3 min, partitioned with 300 mL EtOAc and 150 mL H₂O. Separated layers, back-extracted aqueous with 3×40 mL EtOAc. Dried combined organics over Na₂SO₄, filtered, and concentrated in vacuo to give a yellow oil (4.7 g). Purified by normal-phase CombiFlash ISCO (220 g Gold column, 0-10% MeOH:DCM) to give methyl 1-(3-(hydroxymethyl)phenyl)-5-(trans)-2-(1-methyl-1H-1,2,3-triazol-4-yl)cyclopropyl)-1H-pyrazole-4-carboxylate as a yellow solid (1.6 g, 4.5 mmol, 92% yield). LC-MS m/z 354.1 (M+H)⁺, 0.59 min (ret. time). ¹H NMR (400 MHz, METHANOL-d₄) δ ppm 1.22-1.30 (m, 1H) 1.41-1.51 (m, 1H) 2.12-2.25 (m, 1H) 2.43-2.52 (m, 1H) 3.83 (s, 3H) 4.06 (s, 3H) 4.63 (s, 2H) 7.38-7.46 (m, 1H) 7.49 (d, J=1.00 Hz, 1H) 7.49-7.51 (m, 1H) 7.52 (d, J=0.75 Hz, 1H) 7.58 (s, 1H) 8.05 (s, 1H).

### 4c) Methyl 1-(3-(((S)-4-ethyl-1,1-dioxido-4,5-dihydrobenzo[f][1,2]thiazepin-2(3H)-yl)methyl)phenyl)-5-(trans)-2-(1-methyl-1H-1,2,3-triazol-4-yl)cyclopropyl)-1H-pyrazole-4-carboxylate

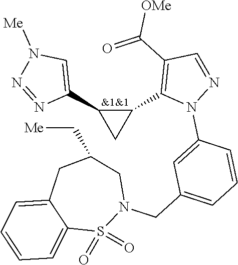 [SMILES: CC[C@H]1Cc2ccccc2S(=O)(=O)N(Cc2cccc(-n3ncc(C(=O)OC)c3[C@@H]3C[C@H]3c3cn(C)nn3)c2)C1]

To a solution of methyl 1-(3-(hydroxymethyl)phenyl)-5-(trans)-2-(1-methyl-1H-1,2,3-triazol-4-yl)cyclopropyl)-1H-pyrazole-4-carboxylate (41.6 mg, 0.118 mmol), (S)-4-ethyl-2,3,4,5-tetrahydrobenzo[f][1,2]thiazepine 1,1-dioxide (29.2 mg, 0.129 mmol) and trimethylphosphine (235 μl, 0.235 mmol, 1M in THF) in tetrahydrofuran (THF) (589 μl) was added DIAD (45.8 μl, 0.235 mmol) at RT. After 45 min, partitioned with 10 mL EtOAc and 5 mL water, separated layers. Back-extracted aqueous with 1×5 mL EtOAc. Washed combined organics with 4×5 mL water, 1×5 mL brine. Dried combined organics over Na₂SO₄, filtered, and concentrated in vacuo to give a yellow oil. Purified by normal-phase CombiFlash ISCO (12 g Gold column, 0-70% EtOAc:Hexanes) to give methyl 1-(3-(((S)-4-ethyl-1,1-dioxido-4,5-dihydrobenzo[f][1,2]thiazepin-2(3H)-yl)methyl)phenyl)-5-(trans)-2-(1-methyl-1H-1,2,3-triazol-4-yl)cyclopropyl)-1H-pyrazole-4-carboxylate as a white solid (23.8 mg, 0.042 mmol, 36%). LC-MS m/z 561.1 (M+H)⁺, 1.13 min (ret. time).

### 4d) 1-(3-(((S)-4-Ethyl-1,1-dioxido-4,5-dihydrobenzo[f][1,2]thiazepin-2(3H)-yl)methyl)phenyl)-5-(trans)-2-(1-methyl-1H-1,2,3-triazol-4-yl)cyclopropyl)-1H-pyrazole-4-carboxylic acid

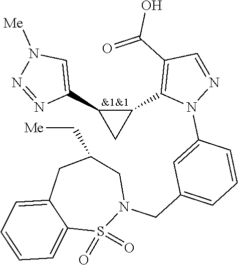 [SMILES: CC[C@H]1Cc2ccccc2S(=O)(=O)N(Cc2cccc(-n3ncc(C(=O)O)c3[C@@H]3C[C@H]3c3cn(C)nn3)c2)C1]

To a suspension of methyl 1-(3-(((S)-4-ethyl-1,1-dioxido-4,5-dihydrobenzo[f][1,2]thiazepin-2(3H)-yl)methyl)phenyl)-5-(trans)-2-(1-methyl-1H-1,2,3-triazol-4-yl)cyclopropyl)-1H-pyrazole-4-carboxylate (23.8 mg, 0.042 mmol) in methanol (424 μl) was added aqueous NaOH (424 μl, 0.424 mmol, 1M). Warmed to 100° C. After 1 h, 40 min, cooled to RT, added 2 drops of DMSO, and injected directly onto reverse-phase HPLC (Mega-Gilson, 10 min run, 10-90% CH₃CN:H₂O, acidic conditions) to give (following concentration in vacuo of product-containing fractions) 1-(3-(((S)-4-ethyl-1,1-dioxido-4,5-dihydrobenzo[f][1,2]thiazepin-2(3H)-yl)methyl)phenyl)-5-(trans)-2-(1-methyl-1H-1,2,3-triazol-4-yl)cyclopropyl)-1H-pyrazole-4-carboxylic acid as a white solid (13.1 mg, 0.024 mmol, 57% yield). LC-MS m/z 547.1 (M+H)⁺, 1.01 min (ret. time). ¹H NMR (400 MHz, METHANOL-d₄) δ ppm 0.96 (d, J=5.02 Hz, 3H) 1.30-1.35 (m, 2H) 1.44-1.51 (m, 1H) 1.69-1.83 (m, 1H) 2.14-2.31 (m, 1H) 2.37-2.52 (m, 1H) 2.83-2.98 (m, 1H) 3.40-3.49 (m, 1H) 3.64-3.89 (m, 2H) 4.05 (d, J=1.25 Hz, 3H) 4.17-4.30 (m, 1H) 7.41-7.57 (m, 8H) 7.87-7.96 (m, 1H) 8.05 (s, 1H).

The example in Table 3 was prepared in an analogous manner:

TABLE 3

| | | | | Retention | |
|---|---|---|---|---|---|
| | | | LCMS | Time | |
| Ex # | Structure | Name | [M + H]⁺ | (min) | ¹HNMR |
| Example 5 | 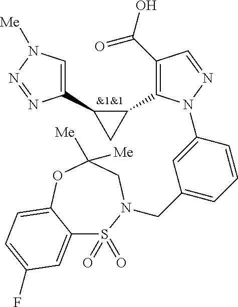 | 1-(3-((8-fluoro-4,4- dimethyl-1,1-dioxido- 3,4-dihydro-2H- benzo[b][1,4,5]- oxathiazepin-2- yl)methyl)phenyl)-5- (trans)-2-(1-methyl- 1H-1,2,3-triazol-4- yl)cyclopropyl)-1H- pyrazole-4- carboxylic acid | 567.1 | 0.94 | ¹H NMR (400 MHz, METHANOL- d₄) δ ppm 1.32 (s, 7 H) 1.45- 1.55 (m, 1H) 2.20-2.31 (m, 1 H) 2.41-2.51 (m, 1H) 3.57- 3.76 (m, 2 H) 4.06 (s, 3 H) 4.45-4.55 (m, 2 H) 7.17-7.24 (m, 1 H) 7.28- 7.36 (m, 1 H) 7.46-7.58 (m, 6 H) 8.06 (s, 1 H) |

### Example 6. 1-(3-((R or S)-1-((S)-4-Methyl-1,1-dioxido-8-(trifluoromethyl)-4,5-dihydrobenzo[f][1,2]thiazepin-2(3H)-yl)ethyl)phenyl)-5-((1R,2R)-2-(1-methyl-1H-1,2,3-triazol-4-yl)cyclopropyl)-1H-pyrazole-4-carboxylic acid

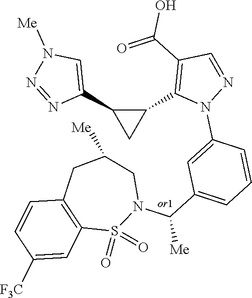 [SMILES: C[C@H]1Cc2ccc(C(F)(F)F)cc2S(=O)(=O)N([C@@H](C)c2cccc(-n3ncc(C(=O)O)c3[C@@H]3C[C@H]3c3cn(C)nn3)c2)C1]

### 6a) Methyl 1-(3-formylphenyl)-5-(trans)-2-(1-methyl-1H-1,2,3-triazol-4-yl-cyclopropyl)-1H-pyrazole-4-carboxylate

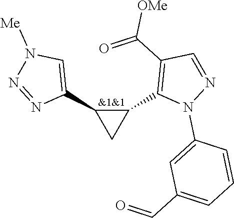 [SMILES: COC(=O)c1cnn(-c2cccc(C=O)c2)c1[C@@H]1C[C@H]1c1cn(C)nn1]

To a solution of methyl 1-(3-(hydroxymethyl)phenyl)-5-(trans)-2-(1-methyl-1H-1,2,3-triazol-4-yl)cyclopropyl)-1H-pyrazole-4-carboxylate (5 mg, 0.014 mmol) in dichloromethane (DCM) (0.7 mL) at RT was added 3-oxo-115-benzo[d][1,2]iodaoxole-1,1,1(3H)-triyl triacetate (8.40 mg, 0.020 mmol). Stirred for 30 min. Upon completion, diluted with Et₂O, and a white precipitate formed. Extracted with aqueous NaOH (1N) and washed twice with Et₂O. The organic layers were combined, dried with Na₂SO₄, filtered, and concentrated to give methyl 1-(3-formylphenyl)-5-(trans)-2-(1-methyl-1H-1,2,3-triazol-4-yl-cyclopropyl)-1H-pyrazole-4-carboxylate (148.7 mg, 0.415 mmol, 89% yield), which was used without further purification. LC-MS m/z 352.1 (M+H)⁺, 0.66 min (ret. Time). ¹H NMR (DMSO-d₆) δ ppm 9.96 (s, 1H), 8.08 (s, 1H), 7.88-8.01 (m, 3H), 7.66-7.75 (m, 1H), 7.62 (s, 1H), 3.95 (s, 3H), 3.76 (s, 3H), 2.55-2.61 (m, 1H), 2.02-2.14 (m, 1H), 1.32-1.42 (m, 1H), 1.05-1.15 (m, 1H).

### 6b) Methyl 1-(3-(1-hydroxyethyl)phenyl)-5-(trans)-2-(1-methyl-1H-1,2,3-triazol-4-yl)cyclopropyl)-1H-pyrazole-4-carboxylate

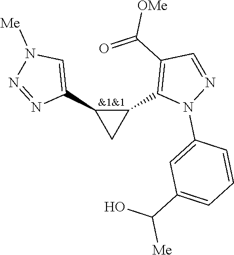 [SMILES: COC(=O)c1cnn(-c2cccc(C(C)O)c2)c1[C@@H]1C[C@H]1c1cn(C)nn1]

To a solution of methyl 1-(3-formylphenyl)-5-(trans)-2-(1-methyl-1H-1,2,3-triazol-4-yl)cyclopropyl)-1H-pyrazole-4-carboxylate (148.7 mg, 0.423 mmol in tetrahydrofuran (THF) (8266 μl) was added methylmagnesium bromide (0.198 mL, 0.592 mmol) as a 3.0 M solution in Et₂O. After 5 min, removed from dry ice bath and allowed to warm to RT over 60 min. Upon completion, quenched excess methylmagnesium bromide by addition of H₂O. Partitioned with EtOAc. Separated layers, and washed aqueous layer with EtOAc. Dried the combined organic layers with Na₂SO₄, filtered, and concentrated. The residue was dissolved in minimal DCM/MeOH, loaded onto Celite, and purified by silica gel chromatography (12 g, 0-15% MeOH/DCM) to provide methyl 1-(3-(1-hydroxyethyl)phenyl)-5-(trans)-2-(1-methyl-1H-1,2,3-triazol-4-yl)cyclopropyl)-1H-pyrazole-4-carboxylate (56.4 mg, 0.140 mmol, 33% yield) as a white solid. LC-MS m/z 368.2 m/z (M+H)⁺, 0.56 min (ret. Time). ¹H NMR (DMSO-d₆) δ ppm 8.04 (s, 1H), 7.60-7.70 (m, 1H), 7.48-7.54 (m, 1H), 7.34-7.48 (m, 3H), 5.22-5.35 (m, 1H), 4.65-4.79 (m, 1H), 3.97 (s, 3H), 3.74 (s, 3H), 2.30-2.73 (m, 1H), 2.10-2.26 (m, 1H), 1.04-1.40 (m, 5H).

### 6c) Methyl 1-(3-(1-chloroethyl)phenyl)-5-(trans)-2-(1-methyl-1H-1,2,3-triazol-4-yl)cyclopropyl)-1H-pyrazole-4-carboxylate

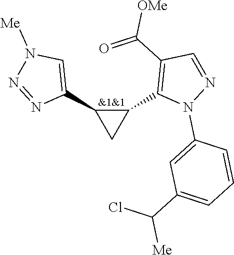 [SMILES: COC(=O)c1cnn(-c2cccc(C(C)Cl)c2)c1[C@@H]1C[C@H]1c1cn(C)nn1]

To a stirred solution of methyl 1-(3-(1-hydroxyethyl)phenyl)-5-(trans)-2-(1-methyl-1H-1,2,3-triazol-4-yl)cyclopropyl)-1H-pyrazole-4-carboxylate (50.0 mg, 0.136 mmol) in dichloromethane (DCM) (1361 μl) was added thionyl chloride (49.2 μl, 0.680 mmol) at RT. Stirred for 40 min. Upon completion, quenched with saturated aq. NaHCO₃ and extracted with EtOAc. Organic layer was dried with Na₂SO₄, filtered, and concentrated. The residue was purified by silica gel chromatography (12 g, 0-10% MeOH in DCM) to provide methyl 1-(3-(1-chloroethyl)phenyl)-5-(trans)-2-(1-methyl-1H-1,2,3-triazol-4-yl)cyclopropyl)-1H-pyrazole-4-carboxylate (46.4 mg, 0.103 mmol, 76% yield) as a white solid. LC-MS m/z 386.1 m/z (M+H)⁺, 0.90 min (ret. time). ¹H NMR (DMSO-d₆) δ ppm 8.06 (s, 1H), 7.39-7.73 (m, 5H), 5.26-5.41 (m, 1H), 3.97 (s, 3H), 3.75 (s, 3H), 2.27-2.74 (m, 1H), 2.06-2.22 (m, 1), 1.69-1.77 (m, 3H), 1.29-1.41 (m, 1H), 1.04-1.18 (m, 1H).

### 6d) Methyl 1-(3-((R or S)-1-((S)-4-methyl-1,1-dioxido-8-(trifluoromethyl)-4,5-dihydrobenzo[f][1,2]thiazepin-2(3H)-yl)ethyl)phenyl)-5-((1R,2R)-2-(1-methyl-1H-1,2,3-triazol-4-yl)cyclopropyl)-1H-pyrazole-4-carboxylate

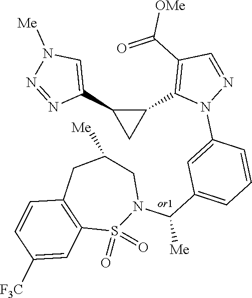 [SMILES: COC(=O)c1cnn(-c2cccc([C@H](C)N3C[C@@H](C)Cc4ccc(C(F)(F)F)cc4S3(=O)=O)c2)c1[C@@H]1C[C@H]1c1cn(C)nn1]

To a solution of (S)-4-methyl-8-(trifluoromethyl)-2,3,4,5-tetrahydrobenzo[f][1,2]thiazepine 1,1-dioxide (37.4 mg, 0.134 mmol) and methyl 1-(3-(1-chloroethyl)phenyl)-5-(trans)-2-(1-methyl-1H-1,2,3-triazol-4-yl)cyclopropyl)-1H-pyrazole-4-carboxylate (47.0 mg, 0.122 mmol) in N,N-dimethylformamide (DMF) (2436 μl) was added K₂CO₃ (18.52 mg, 0.134 mmol). Heated at 85° C. for 3 hr. Cooled and diluted with H₂O. The aqueous layer was washed with EtOAc. The organic layer was dried with Na₂SO₄, filtered, and concentrated. The mixture of diastereomers was resolved by reverse-phase HPLC (0-100% H₂O/MeCN with 0.1% TFA) to afford 1-(3-((R or S)-1-((S)-4-methyl-1,1-dioxido-8-(trifluoromethyl)-4,5-dihydrobenzo[f][1,2]thiazepin-2(3H)-yl)ethyl)phenyl)-5-((1R,2R)-2-(1-methyl-1H-1,2,3-triazol-4-yl)cyclopropyl)-1H-pyrazole-4-carboxylate (20.8 mg, 0.033 mmol, 27% yield) as a white solid. LC-MS m/z 629.4 m/z (M+H)⁺, 1.25 min (ret. time). ¹H NMR (METHANOL-d₄) δ ppm 8.12 (s, 1H), 8.07 (s, 1H), 7.85 (d, J=7.5 Hz, 1H), 7.64 (d, J=8.0 Hz, 1H), 7.46-7.60 (m, 5H), 5.08-5.23 (m, 1H), 4.07 (s, 3H), 3.84 (s, 3H), 3.45-3.70 (m, 2H), 2.85-3.18 (m, 3H), 2.44-2.58 (m, 1H), 2.15-2.30 (m, 1H), 1.91-2.07 (m, 2H), 1.43-1.53 (m, 1H), 1.21 (d, J=7.0 Hz, 4H), 0.99 (br. s., 3H).

### 6e) 1-(3-((R or S)-1-((S)-4-Methyl-1,1-dioxido-8-(trifluoromethyl)-4,5-dihydrobenzo[f][1,2]thiazepin-2(3H)-yl)ethyl)phenyl)-5-((1R,2R)-2-(1-methyl-1H-1,2,3-triazol-4-yl)cyclopropyl)-1H-pyrazole-4-carboxylic acid

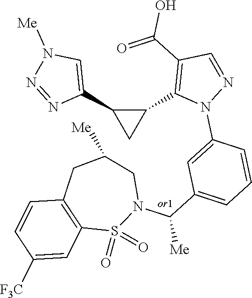 [SMILES: C[C@H]1Cc2ccc(C(F)(F)F)cc2S(=O)(=O)N([C@@H](C)c2cccc(-n3ncc(C(=O)O)c3[C@@H]3C[C@H]3c3cn(C)nn3)c2)C1]

To a suspension of methyl 1-(3-((R or S)-1-((S)-4-methyl-1,1-dioxido-8-(trifluoromethyl)-4,5-dihydrobenzo[f][1,2]thiazepin-2(3H)-yl)ethyl)phenyl)-5-((1R,2R)-2-(1-methyl-1H-1,2,3-triazol-4-yl)cyclopropyl)-1H-pyrazole-4-carboxylate (20.8 mg, 0.033 mmol) in methanol (350 μl) was added sodium hydroxide (14.0 mg, 0.35 mmol). Heated to 60° C. for 3 hr. The reaction mixture was concentrated and purified by reverse-phase HPLC (0-100% H₂O/MeCN with 0.1% TFA), providing 1-(3-((R or S)-1-((S)-4-methyl-1,1-dioxido-8-(trifluoromethyl)-4,5-dihydrobenzo[f][1,2]thiazepin-2(3H)-yl)ethyl)phenyl)-5-((1R,2R)-2-(1-methyl-1H-1,2,3-triazol-4-yl)cyclopropyl)-1H-pyrazole-4-carboxylic acid (4.0 mg, 6.5 μmol, 19% yield) as a white solid after lyophilization. LC-MS m/z 629.4 (M+H)⁺, 1.11 min (ret. time). ¹H NMR (METHANOL-d₄) δ ppm 8.12 (s, 1H), 8.05 (s, 1H), 7.85 (d, J=7.5 Hz, 1H), 7.64 (d, J=7.8 Hz, 1H), 7.43-7.59 (m, 5H), 5.07-5.20 (m, 1H), 4.05 (s, 3H), 3.45-3.72 (m, 2H), 2.82-3.20 (m, 2H), 2.68 (s, 1H), 2.39-2.54 (m, 1H), 2.17-2.33 (m, 1H), 1.92-2.05 (m, 2H), 1.43-1.56 (m, 1H), 1.27-1.40 (m, 1H), 1.20 (d, J=6.8 Hz, 3H), 0.99 (br. s., 3H).

### Example 7. 1-(3-((4,4-Dimethyl-4,5-dihydro-1H-benzo[c]azepin-2(3H)-yl)methyl)phenyl)-5-(trans)-2-(1-methyl-1H-1,2,3-triazol-4-yl)cyclopropyl)-1H-pyrazole-4-carboxylic acid

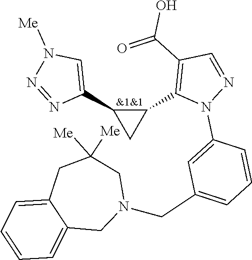 [SMILES: Cn1cc([C@@H]2C[C@H]2c2c(C(=O)O)cnn2-c2cccc(CN3Cc4ccccc4CC(C)(C)C3)c2)nn1]

### 7a) Methyl 1-(3-(chloromethyl)phenyl)-5-(trans)-2-(1-methyl-1H-1,2,3-triazol-4-yl)cyclopropyl)-1H-pyrazole-4-carboxylate

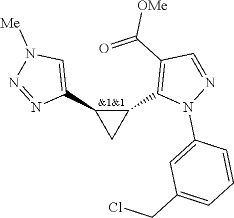 [SMILES: COC(=O)c1cnn(-c2cccc(CCl)c2)c1[C@@H]1C[C@H]1c1cn(C)nn1]

Methyl 1-(3-(hydroxymethyl)phenyl)-5-(trans)-2-(1-methyl-1H-1,2,3-triazol-4-yl)cyclopropyl)-1H-pyrazole-4-carboxylate (1.5 g, 4.11 mmol) was dissolved in anhydrous dichloromethane (20 mL) and treated with thionyl chloride (0.60 mL, 8.21 mmol). After 10 min at RT, concentrated in vacuo to give methyl 1-(3-(chloromethyl)phenyl)-5-(trans)-2-(1-methyl-1H-1,2,3-triazol-4-yl)cyclopropyl)-1H-pyrazole-4-carboxylate as a light pink solid (1.7 g, 4.03 mmol, 98% yield). LC-MS m/z 372.0 (M+H)⁺, 0.81 min (ret. time). Carried forward without further purification.

### 7b) Methyl 1-(3-((4,4-dimethyl-4,5-dihydro-1H-benzo[c]azepin-2(3H)-yl)methyl)phenyl)-5-(trans)-2-(1-methyl-1H-1,2,3-triazol-4-yl)cyclopropyl)-1H-pyrazole-4-carboxylate

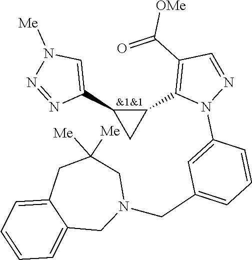 [SMILES: COC(=O)c1cnn(-c2cccc(CN3Cc4ccccc4CC(C)(C)C3)c2)c1[C@@H]1C[C@H]1c1cn(C)nn1]

To a solution of methyl 1-(3-(chloromethyl)phenyl)-5-(trans)-2-(1-methyl-1H-1,2,3-triazol-4-yl)cyclopropyl)-1H-pyrazole-4-carboxylate (100 mg, 0.269 mmol) in acetonitrile (2689 μl) was added DIPEA (94 μl, 0.538 mmol) and 4,4-dimethyl-2,3,4,5-tetrahydro-1H-benzo[c]azepine hydrochloride (62.6 mg, 0.296 mmol) sequentially. Warmed to 100° C. After 2 h, concentrated in vacuo to give a tan solid. LC-MS m/z 511.1 (M+H)⁺, 0.78 min (ret. time). Carried forward without further purification.

### 7c) 1-(3-((4,4-Dimethyl-4,5-dihydro-1H-benzo[c]azepin-2(3H)-yl)methyl)phenyl)-5-(trans)-2-(1-methyl-1H-1,2,3-triazol-4-yl)cyclopropyl)-1H-pyrazole-4-carboxylic acid

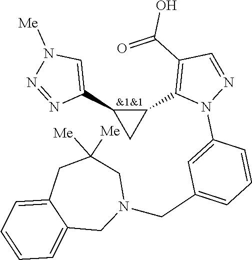 [SMILES: Cn1cc([C@@H]2C[C@H]2c2c(C(=O)O)cnn2-c2cccc(CN3Cc4ccccc4CC(C)(C)C3)c2)nn1]

To a suspension of methyl 1-(3-((4,4-dimethyl-4,5-dihydro-1H-benzo[c]azepin-2(3H)-yl)methyl)phenyl)-5-(trans)-2-(1-methyl-1H-1,2,3-triazol-4-yl)cyclopropyl)-1H-pyrazole-4-carboxylate (137 mg, 0.268 mmol) in methanol (1341 μl) was added aqueous NaOH (805 μl, 0.805 mmol, 1M). Heated to 80° C. After 31 h, LCMS was consistent with desired product plus unreacted starting material. Added an additional amount of aqueous NaOH (805 μl, 0.805 mmol, 1M) and heated back up to 80° C. After an additional 24 h, LC-MS suggested complete consumption of starting material. Removed MeOH solvent in vacuo to give an orange semi-solid. Dissolved in 2 mL acetonitrile plus 2 drops DMSO, injected directly onto reverse-phase HPLC (10-70% CH₃CN:H₂O, acidic conditions) to give (following concentration of product-containing fractions in vacuo) 1-(3-((4,4-dimethyl-4,5-dihydro-1H-benzo[c]azepin-2(3H)-yl)methyl)phenyl)-5-(trans)-2-(1-methyl-1H-1,2,3-triazol-4-yl)cyclopropyl)-1H-pyrazole-4-carboxylic acid (trifluoroacetate) as a white solid (172.6 mg, 0.266 mmol, 99% yield). LC-MS m/z 497.2 (M+H)⁺, 0.68 min (ret. time). ¹H NMR (400 MHz, DMSO-d₆) δ ppm 0.73 (br. s., 3H) 0.81-0.91 (m, 2H) 1.04 (d, J=9.79 Hz, 3H) 1.26 (br. s., 2H) 1.33-1.41 (m, 1H) 2.08-2.16 (m, 1H) 3.17-3.24 (m, 2H) 3.98-4.05 (m, 2H) 4.51 (br. s., 4H) 4.58-4.67 (m, 1H) 7.18-7.25 (m, 1H) 7.28-7.33 (m, 1H) 7.34-7.38 (m, 1H) 7.39-7.44 (m, 1H) 7.52-7.61 (m, 1H) 7.70 (d, J=7.53 Hz, 3H) 7.87-7.93 (m, 1H) 8.03 (s, 1H).

Examples in Table 4 were prepared in an analogous manner:

TABLE 4

| | | | | Retention | |
|---|---|---|---|---|---|
| | | | LCMS | Time | |
| Ex # | Structure | Name | [M + H]⁺ | (min) | ¹HNMR |
| Example 8 | 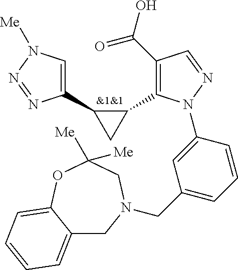 | 1-(3-((2,2-dimethyl- 2,3-dihydrobenzo- [f][1,4]oxazepin- 4(5H)-yl)methyl)- phenyl)-5-(trans)-2- (1-methyl-1H-1,2,3- triazol-4- yl)cyclopropyl)-1H- pyrazole-4- carboxylic acid (trifluoroacetate) | 499.2 | 0.68 | ¹H NMR (400 MHz, METHANOL-d₄) δ ppm 1.26-1.34 (m, 1H) 1.37 (br. s., 6H) 1.43- 1.50 (m, 1 H) 2.21-2.33 (m, 1 H) 2.44-2.53 (m, 1 H) 3.49 (br. s., 2 H) 4.04 (s, 3 H) 4.55 (br. s., 2 H) 4.59 (br. s., 2 H) 7.09 (d, J = 8.03 Hz, 1 H) 7.23 (s, 1 H) 7.37- 7.48 (m, 2 H) |
| | | | | | 7.63-7.68 (m, 2 |
| | | | | | H) 7.72 (d, |
| | | | | | J = 8.28 Hz, 2 H) |
| | | | | | 7.85 (s, 1 H) 8.09 |
| | | | | | (s, 1 H) |
| Example 9 | 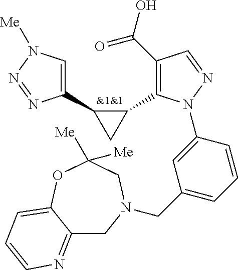 | 1-(3-((2,2-dimethyl- 2,3-dihydropyrido- [2,3-f][1,4]oxazepin- 4(5H)-yl)methyl)- phenyl)-5-(trans)-2- (1-methyl-1H-1,2,3- triazol-4-yl)cyclo- propyl)-1H- pyrazole-4- carboxylic acid (trifluoroacetate) | 500.2 | 0.54 min | ¹H NMR (400 MHz, METHANOL-d₄) δ ppm 1.38 (s, 7 H) 1.42-1.50 (m, 1 H) 2.20- 2.31 (m, 1 H) 2.40-2.53 (m, 1 H) 3.27 (br. s., 2 H) 4.05 (s, 3 H) 4.11-4.23 (m, 2 H) 4.28 (br. s., 2 H) 7.58 (t, J = 8.16 Hz, 3 H) 7.64 (s, 2 H) 7.68 (br. s., 1 H) 7.78-7.86 (m, 1 H) 8.06 (s, |
| | | | | | 1 H) 8.38 (d, |
| | | | | | J = 5.02 Hz, 1 H) |
| Example 10 | 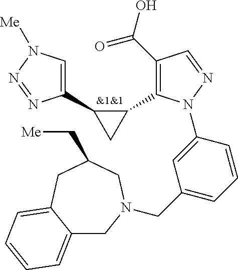 | 1-(3-(((R)-4-ethyl- 4,5-dihydro-1H- benzo[c]azepin- 2(3H)-yl)methyl)- phenyl)-5-(trans)-2- (1-methyl)-1H-1,2,3- triazol-4-yl)cyclo- propyl)-1H- pyrazole-4- carboxylic acid (trifluoroacetate) | 497.2 | 0.60 | ¹H NMR (400 MHz, METHANOL-d₄) δ ppm 0.93-1.08 (m, 3 H) 1.29- 1.37 (m, 1 H) 1.42-1.55 (m, 3 H) 2.24-2.33 (m, 1 H) 2.47- 2.56 (m, 1 H) 2.83-2.98 (m, 1 H) 2.99-3.11 (m, 2 H) 3.59- 3.72 (m, 1 H) 4.03 (s, 3 H) 4.42- 4.52 (m, 1 H) 4.55-4.62 (m, 2 |
| | | | | | H) 7.22-7.46 |
| | | | | | (m, 4 H) 7.63- |
| | | | | | 7.69 (m, 2 H) |
| | | | | | 7.71-7.81 (m, 2 |
| | | | | | H) 7.82-7.90 |
| | | | | | (m, 1 H) 8.09 (s, |
| | | | | | 1 H) |
| Example 11 | 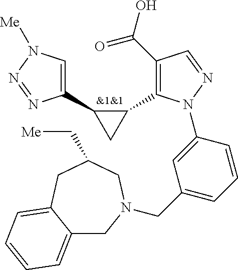 | 1-(3-(((S)-4-ethyl- 4,5-dihydro-1H- benzo[c]azepin- 2(3H)- yl)methyl)phenyl)-5- (trans)-2-(1-methyl- 1H-1,2,3-triazol-4- yl)cyclopropyl)-1H- pyrazole-4- carboxylic acid (trifluoroacetate) | 497.2 | 0.60 | ¹H NMR (400 MHz, METHANOL-d₄) δ ppm 0.96-1.07 (m, 3 H) 1.28- 1.38 (m, 1 H) 1.41-1.56 (m, 3 H) 2.22-2.34 (m, 1 H) 2.43- 2.53 (m, 1 H) 2.86-2.99 (m, 1 H) 2.99-3.13 (m, 2 H) 3.58- 3.75 (m, 1 H) 4.04 (s, 3 H) 4.41- 4.67 (m, 3 H) 7.19-7.45 (m, 4 |
| | | | | | H) 7.68 (s, 2 H) |
| | | | | | 7.70-7.79 (m, 2 |
| | | | | | H) 7.81-7.88 |
| | | | | | (m, 1 H) 8.09 (s, |
| | | | | | 1 H) |
| Example 12 | 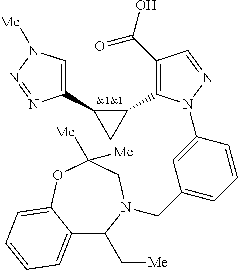 | 1-(3-((5-ethyl-2,2- dimethyl-2,3- dihydrobenzo[f][1,4] oxazepin-4(5H)- yl)methyl)phenyl)-5- (trans)-2-(1-methyl- 1H-1,2,3-triazol-4- yl)cyclopropyl)-1H- pyrazole-4-carboxylic acid (trifluoroacetate) | 527.1 | 0.77 | ¹H NMR (400 MHz, METHANOL- d₄) δ ppm 0.80 (t, J = 6.90 Hz, 3 H) 1.22 (br. s., 3 H) 1.34 (br. s., 1 H) 1.44 (br. s., 1 H) 1.53 (d, J = 3.26 Hz, 3 H) 2.26 (br. s., 2 H) 2.35-2.43 (m, 1 H) 2.48 (d, J = 3.26 Hz, 1 H) 3.25-3.31 (m, 1 H) 3.62-3.76 (m, 1 H) 4.05 (s, |
| | | | | | 3 H) 4.25-4.41 |
| | | | | | (m, 1 H) 4.48- |
| | | | | | 4.74 (m, 2 H) |
| | | | | | 7.08 (d, J = 8.03 |
| | | | | | Hz, 1 H) 7.22 |
| | | | | | (d, J = 7.53 Hz, 1 |
| | | | | | H) 7.27 (br. s., 1 |
| | | | | | H) 7.43 (s, 1 H) |
| | | | | | 7.59-7.67 (m, 2 |
| | | | | | H) 7.71 (br. s., 2 |
| | | | | | H) 7.85 (br. s., 1 |
| | | | | | H) 8.08 (s, 1 H) |
| Example 13 | 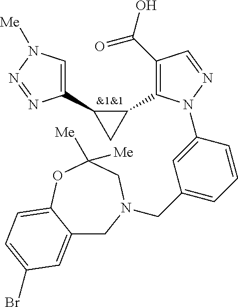 | 1-(3-((7-bromo-2,2- dimethyl-2,3- dihydrobenzo[f][1,4] oxazepin-4(5H)- yl)methyl)phenyl)-5- (trans)-2-(1-methyl- 1H-1,2,3-triazol-4- yl)cyclopropyl)-1H- pyrazole-4-carboxylic acid (trifluoroacetate) | 577.1 | 0.78 | ¹H NMR (400 MHz, METHANOL- d₄) δ ppm 1.32 (d, J = 7.28 Hz, 1 H) 1.37 (br. s., 6 H) 1.42-1.52 (m, 1 H) 2.17- 2.31 (m, 1 H) 2.42-2.54 (m, 1 H) 3.49 (s, 2 H) 4.04 (s, 3 H) 4.51 (br. s., 2 H) 4.54-4.64 (m, 2 H) 7.00 (d, J = 8.28 Hz, 1 H) 7.57 (d, J = 8.53 Hz, 1 H) 7.60 (br. s., 1 H) 7.62- |
| | | | | | 7.67 (m, 2 H) |
| | | | | | 7.72 (t, J = 6.40 |
| | | | | | Hz, 2 H) 7.84 |
| | | | | | (br. s., 1 H) 8.09 |
| | | | | | (s, 1 H) |

### Example 14. Sodium 1-(3-(((R)-2-ethyl-2,3-dihydrobenzo[f][1,4]oxazepin-4(5H)-yl)methyl)phenyl)-5-(trans)-2-(1-methyl-1H-1,2,3-triazol-4-yl)cyclopropyl)-1H-pyrazole-4-carboxylate

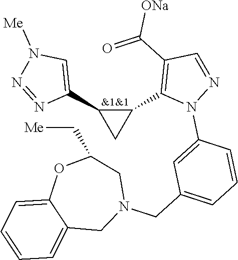

### 14a) Ethyl 1-(3-(chloromethyl)phenyl)-5-(trans)-2-(1-methyl-1H-1,2,3-triazol-4-yl)cyclopropyl)-1H-pyrazole-4-carboxylate

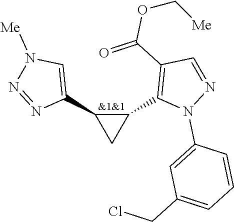 [SMILES: CCOC(=O)c1cnn(-c2cccc(CCl)c2)c1[C@@H]1C[C@H]1c1cn(C)nn1]

To a solution of ethyl 1-(3-(hydroxymethyl)phenyl)-5-(trans)-2-(1-methyl-1H-1,2,3-triazol-4-yl)cyclopropyl)-1H-pyrazole-4-carboxylate (96.5 mg, 0.263 mmol) in anhydrous dichloromethane (876 μl) was added thionyl chloride (38.3 μl, 0.525 mmol). After 10 min at RT, concentrated in vacuo to give ethyl 1-(3-(chloromethyl)phenyl)-5-(trans)-2-(1-methyl-1H-1,2,3-triazol-4-yl)cyclopropyl)-1H-pyrazole-4-carboxylate as a dark red oil (134 mg). LC-MS m/z 386.0 (M+H)⁺, 0.89 min (ret. time). Carried forward without further purification.

### 14b) Ethyl 1-(3-(((R)-2-ethyl-2,3-dihydrobenzo[f][1,4]oxazepin-4(5H)-yl)methyl)phenyl)-5-(trans)-2-(1-methyl-1H-1,2,3-triazol-4-yl)cyclopropyl)-1H-pyrazole-4-carboxylate

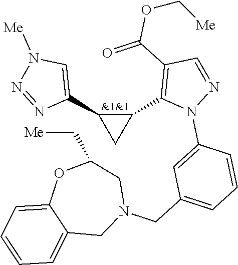 [SMILES: CCOC(=O)c1cnn(-c2cccc(CN3Cc4ccccc4O[C@H](CC)C3)c2)c1[C@@H]1C[C@H]1c1cn(C)nn1]

To a solution of ethyl 1-(3-(chloromethyl)phenyl)-5-(trans)-2-(1-methyl-1H-1,2,3-triazol-4-yl)cyclopropyl)-1H-pyrazole-4-carboxylate (101 mg, 0.262 mmol) in acetonitrile (2618 μl) was added DIPEA (274 μl, 1.571 mmol) and (R)-2-ethyl-2,3,4,5-tetrahydrobenzo[f][1,4]oxazepine hydrochloride (112 mg, 0.524 mmol) sequentially. Warmed to 100° C. After 2.5 h, concentrated in vacuo to give a brown oil. Purified by normal-phase CombiFlash ISCO (24 g Gold column, 0-50% (3:1 EtOAc:EtOH):Hexanes) to give ethyl 1-(3-(((R)-2-ethyl-2,3-dihydrobenzo[f][1,4]oxazepin-4(5H)-yl)methyl)phenyl)-5-(trans)-2-(1-methyl-1H-1,2,3-triazol-4-yl)cyclopropyl)-1H-pyrazole-4-carboxylate as a tan solid (68.6 mg, 0.130 mmol, 50% yield). LC-MS m/z 527.1 (M+H)⁺, 0.80 min (ret. time).

### 14c) Sodium 1-(3-(((R)-2-ethyl-2,3-dihydrobenzo[f][1,4]oxazepin-4(5H)-yl)methyl)phenyl)-5-(trans)-2-(1-methyl-1H-1,2,3-triazol-4-yl)cyclopropyl)-1H-pyrazole-4-carboxylate

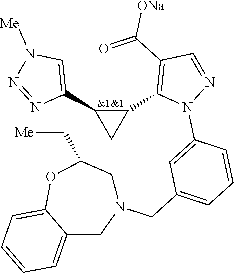

To a solution of ethyl 1-(3-(((R)-2-ethyl-2,3-dihydrobenzo[f][1,4]oxazepin-4(5H)-yl)methyl)phenyl)-5-(trans)-2-(1-methyl-1H-1,2,3-triazol-4-yl)cyclopropyl)-1H-pyrazole-4-carboxylate (93.4 mg, 0.177 mmol) in methanol (591 μl) was added an aqueous solution of NaOH (1774 μl, 1.774 mmol, 1 M) and the resulting mixture was heated to 100° C. After 21 h, cooled to RT. Acidified aqueous layer to pH=2 with 1M aqueous HCl, partitioned with 10 mL EtOAc and separated the resulting layers. Back-extracted aqueous layer with 3×5 mL EtOAc. Added 1M aqueous NaOH to aqueous layer until pH=7. Back-extracted with 3×5 mL EtOAc. Concentrated combined organics to give a white solid. Re-dissolved in 1M NaOH, purified directly by reverse-phase HPLC (0-60% CH₃CN:H₂O) to give (followed concentration of product-containing fractions in vacuo) sodium 1-(3-(((R)-2-ethyl-2,3-dihydrobenzo[f][1,4]oxazepin-4(5H)-yl)methyl)phenyl)-5-(trans)-2-(1-methyl-1H-1,2,3-triazol-4-yl)cyclopropyl)-1H-pyrazole-4-carboxylate as a white solid (27 mg, 0.052 mmol, 29% yield). LC-MS m/z 521.2 (M+H)⁺, 0.68 min (ret. time). ¹H NMR (400 MHz, DMSO-d₆) δ ppm 1.00 (t, J=7.28 Hz, 3H) 1.26-1.48 (m, 3H) 1.49-1.62 (m, 1H) 2.28-2.35 (m, 1H) 2.37-2.44 (m, 1H) 2.76-2.85 (m, 1H) 2.91-2.99 (m, 1H) 3.58 (s, 2H) 3.61-3.65 (m, 1H) 3.71-3.79 (m, 1H) 3.80-3.87 (m, 1H) 3.94 (s, 3H) 6.91-7.04 (m, 3H) 7.16-7.24 (m, 1H) 7.29-7.38 (m, 1H) 7.43 (br. s., 3H) 7.68 (d, J=2.51 Hz, 1H) 7.90 (s, 1H).

### Example 15. 1-(3-(((S)-4-methyl-1,1-dioxido-4,5-dihydrobenzo[f][1,2]thiazepin-2(3H)-yl)methyl)phenyl)-5-((trans)-2-(1-methyl-1H-1,2,3-triazol-4-yl)cyclopropyl)-1H-pyrazole-4-carboxylic acid

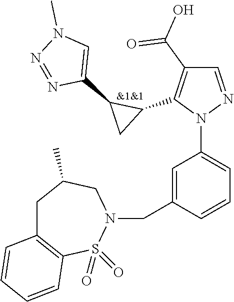 [SMILES: C[C@H]1Cc2ccccc2S(=O)(=O)N(Cc2cccc(-n3ncc(C(=O)O)c3[C@@H]3C[C@H]3c3cn(C)nn3)c2)C1]

(S)-4-Methyl-2,3,4,5 tetrahydrobenzo[f][1,2]thiazepine 1,1-dioxide (28.8 mg, 0.136 mmol) was dissolved in dry CH₃CN (619 μl), followed by the addition of NaH, 60 wt %, (7.42 mg, 0.186 mmol). Methyl 1-(3-(chloromethyl)phenyl)-5-((trans)-2-(1-methyl-1H-1,2,3-triazol-4-yl)cyclopropyl)-1H-pyrazole-4-carboxylate (46 mg, 0.124 mmol) was then added to the reaction, and the reaction was heated in a microwave reactor to 140 C for 1 min. KOH, 0.5 N aqueous solution, (742 μl, 0.371 mmol) was then added to the reaction and the reaction was then heated again in microwave reactor to 140 C for 1 min to obtain a homogeneous yellow solution which was then directly injected onto reverse-phase HPLC and purified, to obtain after evaporation 1-(3-(((S)-4-methyl-1,1-dioxido-4,5-dihydrobenzo[f][1,2]thiazepin-2(3H)-yl)methyl)phenyl)-5-((trans)-2-(1-methyl-1H-1,2,3-triazol-4-yl)cyclopropyl)-1H-pyrazole-4-carboxylic acid (39.7 mg, 60%). LC-MS m/z 533 (M+H)⁺, 0.97 (ret. time), acidic method. ¹H NMR (400 MHz, DMSO-d6): δ ppm 7.98 (s, 1H), 7.84 (m, 1H), 7.53-7.64 (m, 2H), 7.36-7.52 (m, 6H), 4.11 (m, 1H), 3.92 (m, 3H), 3.58 (m, 1H), 3.34 (m, 1H), 2.92 (m, 2H), 2.41 (m, 1H), 1.95-2.23 (m, 3H), 1.34 (m, 1H), 1.20 (m, 1H), 0.91 (m, 3H).

Examples in Table 5 were prepared in an analogous manner:

TABLE 5

| | | | | Retention | |
|---|---|---|---|---|---|
| | | | LCMS | Time | |
| Ex # | Structure | Name | [M + H]⁺ | (min) | ¹HNMR |
| Example 16 | 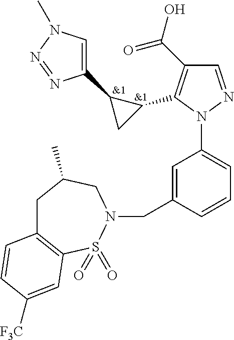 | 1-(3-((4-methyl-1,1- dioxido-8- (trifluoromethyl)-4,5- dihydro- benzo[f][1,2]thiazepin- 2(3H)- yl)methyl)phenyl)-5- ((trans)-2-(1-methyl- 1H-1,2,3-triazol-4- yl)cyclopropyl)-1H- pyrazole-4-carboxylic acid | 601.1 | 1.06 | ¹H NMR (400 MHz, DMSO-d6): δ ppm 8.05 (br. s., 1H), 7.92-8.02 (m, 2H), 7.76 (d, J = 7.78 Hz, 1H), 7.54-7.64 (m, 1H), 7.36-7.52 (m, 4H), 4.10-4.29 (m, 1H), 3.92 (m, 3H), 3.61 (m, 1H), 3.38 (m, 1H), 3.04 (m, 2H), 2.41 (m, 1H), 1.98-2.22 (m, 3H), 1.34 (m, 1H), 1.20 (m, 1H), 0.94 (m, 3H) |
| Example 17 | 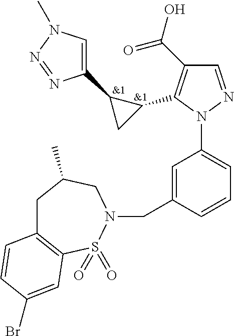 | 1-(3-((8-bromo-4- methyl-1,1-dioxido-4,5- dihydrobenzo[f][1,2] thiazepin-2(3H)- yl)methyl)phenyl)-5- ((trans)-2-(1-methyl- 1H-1,2,3-triazol-4- yl)cyclopropyl)-1H- pyrazole-4-carboxylic acid | 611.0 | 1.04 | ¹H NMR (400 MHz, DMSO-d6): δ ppm 7.98 (s, 1H), 7.90 (m, 1H), 7.78 (d, J = 8.03 Hz, 1H), 7.60 (m, 1H), 7.35-7.52 (m, 5H), 4.18 (m, 1H), 3.93 (m, 3H), 3.58 (m, 1H), 3.26 (m, 1H), 2.95 (m, 2H), 2.41 (m, 1H), 1.95- 2.22 (m, 3H), 1.34 (m, 1H), 1.20 (m, 1H), 0.94 (m, 3H) |
| Example 18 | 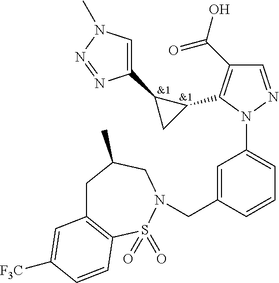 | 1-(3-((4-methyl-1,1- dioxido-7- (trifluoromethyl)-4,5- dihydro- benzo[f][1,2]thiazepin- 2(3H)- yl)methyl)phenyl)-5- ((trans)-2-(1-methyl- 1H-1,2,3-triazol-4- yl)cyclopropyl)-1H- pyrazole-4-carboxylic acid | 601.1 | 1.09 | ¹H NMR (400 MHz, DMSO-d6): δ ppm 8.01-8.10 (m, 1H), 7.98 (s, 1H), 7.93 (s, 1H), 7.85 (d, J = 7.53 Hz, 1H), 7.60 (m, 1H), 7.33-7.53 (m, 4H), 4.16 (m, 1H), 3.93 (m, 3H), 3.60 (m, 1H), 3.25-3.43 (m, 1H), 3.08 (m, 2H), 2.41 (m, 1H), 1.97-2.23 (m, 3H), 1.34 (m, 1H), 1.20 (m, 1H), 0.93 (m, 3H) |
| Example 19 | 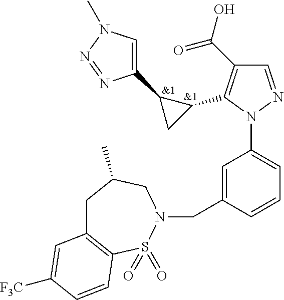 | 1-(3-((4-methyl-1,1- dioxido-7- (trifluoromethyl)-4,5- dihydro- benzo[f][1,2]thiazepin- 2(3H)- yl)methyl)phenyl)-5- ((trans)-2-(1-methyl- 1H-1,2,3-triazol-4-yl) cyclopropyl)-1H- pyrazole-4-carboxylic acid | 601.0 | 1.09 | ¹H NMR (400 MHz, DMSO-d6): δ ppm 8.01-8.10 (m, 1H), 7.98 (s, 1H), 7.93 (s, 1H), 7.84 (m, 1H), 7.60 (m, 1H), 7.34- 7.53 (m, 4H), 4.16 (m, 1H), 3.93 (m, 3H), 3.61 (m, 1H), 3.36 (m, 1H), 3.07 (m, 2H), 2.41 (m, 1H), 1.97-2.24 (m, 3H), 1.34 (m, 1H), 1.20 (m, 1H), 0.93 (m, 3H) |
| Example 20 | 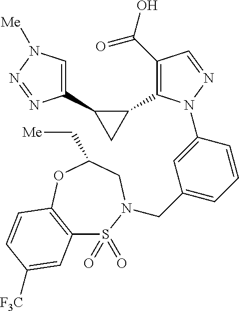 [SMILES: CC[C@@H]1CN(Cc2cccc(-n3ncc(C(=O)O)c3[C@@H]3C[C@H]3c3cn(C)nn3)c2)S(=O)(=O)c2cc(C(F)(F)F)ccc2O1] | 1-(3-(((S)-4-ethyl-1,1- dioxido-8- (trifluoromethyl)-4,5- dihydrobenzo[f][1,2] thiazepin-2(3H)- yl)methyl)phenyl)-5- ((1R,2R)-2-(1-methyl- 1H-1,2,3-triazol-4- yl)cyclopropyl)-1H- pyrazole-4-carboxylic acid | 615.3 | 1.14 | ¹H NMR (400 MHz, DMSO-d6) δ ppm 0.88 (t, J = 7.40 Hz, 3 H) 1.20 (m, 1 H) 1.24- 1.37 (m, 3 H) 1.78 (m, 1 H) 2.06-2.15 (m, 1 H) 2.39-2.47 (m, 2 H) 3.07 (m, 2 H) 3.66 (m, 1 H) 3.77- 3.90 (m, 1 H) 3.92 (s, 3 H) 4.18 (d, J = 15.56 Hz, 1 H) 7.37-7.53 (m, 4 H) 7.60 (s, 1 H) 7.80 (d, J = 7.78 Hz, 1 H) 7.98 (s, 1 H) 8.00 (dd, J = 8.03, 1.51 Hz, 1 H) 8.06 (s, 1 H) 12.48 (br. s.. 1 H) |
| Example 21 | 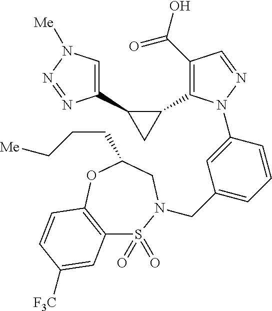 [SMILES: CCCC[C@@H]1CN(Cc2cccc(-n3ncc(C(=O)O)c3[C@@H]3C[C@H]3c3cn(C)nn3)c2)S(=O)(=O)c2cc(C(F)(F)F)ccc2O1] | 1-(3-(((S)-4-butyl-1,1- dioxido-8- (trifluoromethyl)-4,5- dihydrobenzo[f][1,2] thiazepin-2(3H)- yl)methyl)phenyl)-5- ((1R,2R)-2-(1-methyl- 1H-1,2,3-triazol-4- yl)cyclopropyl)-1H- pyrazole-4-carboxylic acid | 643.4 | 1.28 | ¹H NMR (400 MHz, DMSO-d6) δ ppm 0.78-0.85 (m, 3 H) 1.15-1.27 (m, 6 H) 1.28-1.38 (m, 2 H) 1.78-1.90 (m, 1 H) 2.11 (dt, J = 8.72, 5.43 Hz, 1 H) 2.40-2.47 (m, 1 H) 2.96-3.16 (m, 2 H) 3.30-3.42 (m, 1 H) 3.64 (d, J = 11.29 Hz, 1 H) 3.82-3.95 (m, 1 H) 3.92 (s, 3 H) 4.19 (d, J = 15.31 Hz, 1 H) 7.36-7.53 (m, 4 H) 7.58 (s, 1 H) 7.78 (d, J = 8.03 Hz, 1 H) 7.95- 8.01 (m, 1 H) 7.98 |
| | | | | | (s, 1 H) 8.05 (s, 1 H) |
| | | | | | 12.38 (br. s., 1 H) |
| Example 22 | 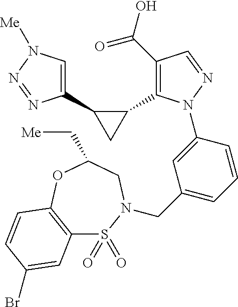 [SMILES: CC[C@@H]1CN(Cc2cccc(-n3ncc(C(=O)O)c3[C@@H]3C[C@H]3c3cn(C)nn3)c2)S(=O)(=O)c2cc(Br)ccc2O1] | 1-(3-(((S)-8-bromo-4- ethyl-1,1-dioxido-4,5- dihydrobenzo[f][1,2] thiazepin-2(3H)- yl)methyl)phenyl)-5- ((1R,2R)-2-(1-methyl- 1H-1,2,3-triazol-4- yl)cyclopropyl)-1H- pyrazole-4-carboxylic acid | 625.3 | 1.16 | ¹H NMR (400 MHz, DMSO-d6) δ ppm 0.86 (t, J = 7.28 Hz, 3 H) 1.16-1.30 (m, 3 H) 1.34 (dt, J = 10.29, 4.64 Hz, 1 H) 1.68- 1.78 (m, 1 H) 2.12 (dt, J = 8.72, 5.43 Hz, 1 H) 2.41-2.48 (m, 1 H) 2.85-3.15 (m, 2 H) 3.18-3.34 (m, 1 H) 3.58-3.66 (m, 1 H) 3.80-3.90 (m, 1 H) 3.94 (s, 3 H) 4.17 (d, J = 15.56 Hz, 1 H) 7.38-7.53 (m, 5 H) 7.59 (s, 1 H) 7.79 (dd, J = 8.16, 2.13 Hz, 1 H) 7.92 (d, J = 2.01 |
| | | | | | Hz, 1 H) 7.98 (s, 1 H) |
| | | | | | 12.39 (br. s., 1 H) |

### Example 23. 1-(3-((2-(Cycloheptylmethyl)-1H-imidazol-1-yl)methyl)phenyl)-5-(trans)-2-(1-methyl-1H-1,2,3-triazol-4-yl)cyclopropyl)-1H-pyrazole-4-carboxylic acid

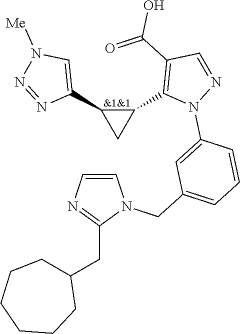 [SMILES: Cn1cc([C@@H]2C[C@H]2c2c(C(=O)O)cnn2-c2cccc(Cn3ccnc3CC3CCCCCC3)c2)nn1]

### 23a) Ethyl 1-(3-(chloromethyl)phenyl)-5-(trans)-2-(1-methyl-1H-1,2,3-triazol-4-yl)cyclopropyl)-1H-pyrazole-4-carboxylate

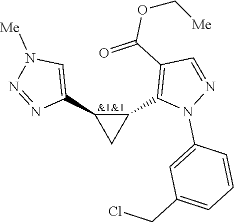 [SMILES: CCOC(=O)c1cnn(-c2cccc(CCl)c2)c1[C@@H]1C[C@H]1c1cn(C)nn1]

To a solution of ethyl 1-(3-(hydroxymethyl)phenyl)-5-(trans)-2-(1-methyl-1H-1,2,3-triazol-4-yl)cyclopropyl)-1H-pyrazole-4-carboxylate (469 mg, 1.277 mmol) in anhydrous dichloromethane (4255 μl) was added thionyl chloride (186 μl, 2.55 mmol). After 10 min at RT, concentrated in vacuo to give ethyl 1-(3-(chloromethyl)phenyl)-5-(trans)-2-(1-methyl-1H-1,2,3-triazol-4-yl)cyclopropyl)-1H-pyrazole-4-carboxylate as a dark red oil (483.3 mg, 1.25 mmol, 98% yield). LC-MS m/z 386.1 (M+H)⁺, 0.89 min (ret. time). Carried forward without further purification.

### 23b) Ethyl 1-(3-((2-(cycloheptylmethyl)-1H-imidazol-1-yl)methyl)phenyl)-5-(trans)-2-(1-methyl-1H-1,2,3-triazol-4-yl)cyclopropyl)-1H-pyrazole-4-carboxylate

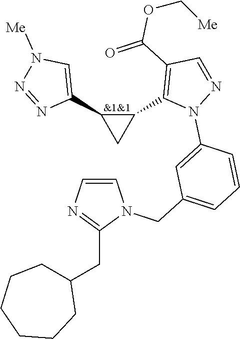 [SMILES: CCOC(=O)c1cnn(-c2cccc(Cn3ccnc3CC3CCCCCC3)c2)c1[C@@H]1C[C@H]1c1cn(C)nn1]

To a solution of 2-(cycloheptylmethyl)-1H-imidazole (102 mg, 0.570 mmol) in N,N-dimethylformamide (1425 μl) was added NaH (45.6 mg, 1.140 mmol, 60% dispersion in mineral oil) at RT. After 30 min, added ethyl 1-(3-(chloromethyl)phenyl)-5-(trans)-2-(1-methyl-1H-1,2,3-triazol-4-yl)cyclopropyl)-1H-pyrazole-4-carboxylate (110 mg, 0.285 mmol) in N,N-dimethylformamide (1425 μl) dropwise. After 30 min at RT, cautiously quenched with 5 mL H₂O, partitioned with 10 mL EtOAc and separated layers. Back-extracted aqueous with 1×5 mL EtOAc. Washed combined organics with 5×5 mL water, 1×5 mL brine. Dried combined organics over Na₂SO₄, filtered, and concentrated in vacuo to give an orange oil. Purified by normal-phase CombiFlash ISCO (24 g Gold column, 0-80% (3:1 EtOAc:EtOH):Hexane) to give ethyl 1-(3-((2-(cycloheptylmethyl)-1H-imidazol-1-yl)methyl)phenyl)-5-(trans)-2-(1-methyl-1H-1,2,3-triazol-4-yl)cyclopropyl)-1H-pyrazole-4-carboxylate as a yellow solid (91 mg, 0.172 mmol, 61% yield). LC-MS m/z 528.2 (M+H)⁺, 0.88 min (ret. time). ¹H NMR (400 MHz, CHLOROFORM-d) δ ppm 1.16-1.30 (m, 5H) 1.31-1.42 (m, 6H) 1.47 (d, J=10.04 Hz, 2H) 1.51-1.65 (m, 7H) 1.67-1.78 (m, 4H) 2.00 (d, J=3.26 Hz, 1H) 2.29-2.38 (m, 1H) 2.45-2.51 (m, 1H) 2.54 (d, J=7.28 Hz, 3H) 4.07 (s, 3H) 4.25-4.40 (m, 2H) 5.12 (s, 2H) 6.80 (s, 1H) 6.99-7.07 (m, 2H) 7.36 (s, 1H) 7.40-7.46 (m, 1H) 7.49 (d, J=7.53 Hz, 1H) 8.05 (s, 1H).

### 23c) 1-(3-((2-(Cycloheptylmethyl)-1H-imidazol-1-yl)methyl)phenyl)-5-(trans)-2-(1-methyl-1H-1,2,3-triazol-4-yl)cyclopropyl)-1H-pyrazole-4-carboxylic acid

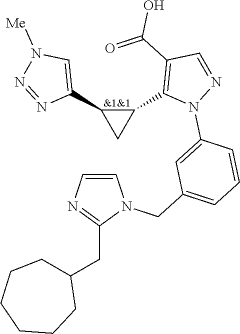 [SMILES: Cn1cc([C@@H]2C[C@H]2c2c(C(=O)O)cnn2-c2cccc(Cn3ccnc3CC3CCCCCC3)c2)nn1]

To a solution of ethyl 1-(3-((2-(cycloheptylmethyl)-1H-imidazol-1-yl)methyl)phenyl)-5-(trans)-2-(1-methyl-1H-1,2,3-triazol-4-yl)cyclopropyl)-1H-pyrazole-4-carboxylate (91 mg, 0.172 mmol) in methanol (575 μl) was added an aqueous solution of NaOH (1725 μl, 1.725 mmol, 1 M). Warmed to 70° C. After 1 h, cooled to RT, acidified aqueous layer to pH=1-2 with 1M aqueous hydrochloric acid. Concentrated in vacuo to give a pink semi-solid. Dissolved in 3 mL water, injected directly onto reverse-phase HPLC (0-80% CH₃CN:H₂O, acidic conditions) to give (following concentration of product-containing fraction in vacuo) the desired compound as a TFA salt. Treated with 3 mL of 1.25 M HCl in MeOH solution, concentrated in vacuo. Repeated 2 more times, then concentrated in vacuo to give 1-(3-((2-(cycloheptylmethyl)-1H-imidazol-1-yl)methyl)phenyl)-5-(trans)-2-(1-methyl-1H-1,2,3-triazol-4-yl)cyclopropyl)-1H-pyrazole-4-carboxylic acid (hydrochloride salt) as a white solid (62.9 mg, 0.117 mmol, 68%). LC-MS m/z 500.1 (M+H)⁺, 0.73 min (ret. time). ¹H NMR (400 MHz, DMSO-d₆) δ ppm 1.09-1.33 (m, 7H) 1.51 (br. s., 7H) 1.82-1.90 (m, 1H) 2.16-2.23 (m, 1H) 2.42-2.47 (m, 1H) 2.90-2.95 (m, 2H) 3.98 (s, 3H) 5.47 (br. s., 2H) 7.35-7.42 (m, 1H) 7.51-7.55 (m, 1H) 7.56 (s, 1H) 7.58-7.63 (m, 1H) 7.66-7.69 (m, 1H) 7.71 (d, J=4.77 Hz, 2H) 8.00 (s, 1H).

The example in Table 6 was prepared in an analogous manner:

TABLE 6

| | | | LCMS | Retention | |
|---|---|---|---|---|---|
| Ex # | Structure | Name | [M + H]⁺ | Time (min) | ¹HNMR |
| Example 24 | 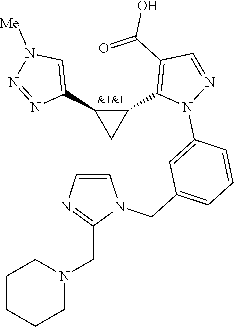 [SMILES: Cn1cc([C@@H]2C[C@H]2c2c(C(=O)O)cnn2-c2cccc(Cn3ccnc3CN3CCCCC3)c2)nn1] | 5-(trans)-2-(1-methyl- 1H-1,2,3-triazol-4- yl)cyclo-propyl)-1-(3- ((2-(piperidin-1- ylmethyl)-1H- imidazol-1- yl)methyl)phenyl)-1H- pyrazole-4-carboxylic acid | 487.2 | 0.50 | ¹H NMR (400 MHz, DMSO- d₆) δ ppm 1.81 (br. s., 6 H) 2.15-2.21 (m, 1 H) 2.42- 2.48 (m, 1 H) 3.06-3.14 (m, 2 H) 3.41- 3.57 (m, 2 H) 3.99 (s, 3 H) 4.66 (br. s., 2 H) 5.61 (br. s., 2 H) 7.47 (br. s., 1 H) 7.53 (s, 1 H) 7.57- 7.63 (m, 4 H) 7.64-7.67 (m, 1 H) 7.71 (s, 1 H) 7.99 (s, 1 |
| | | | | | H) 11.18- |
| | | | | | 11.80 (m, 1 H) |

### Example 25. 1-(3-(((R)-4-Ethyl-1,1-dioxido-3,4-dihydro-2H-benzo[b][1,4,5]oxathiazepin-2-yl)methyl)-4-methylphenyl)-5-(trans)-2-(1-methyl-1H-1,2,3-triazol-4-yl)cyclopropyl)-1H-pyrazole-4-carboxylic acid

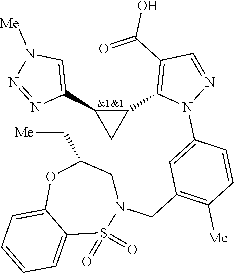

### 25a) 5-(4-(Methoxycarbonyl)-5-(trans)-2-(1-methyl-1H-1,2,3-triazol-4-yl)cyclopropyl)-1H-pyrazol-1-yl)-2-methylbenzoic acid

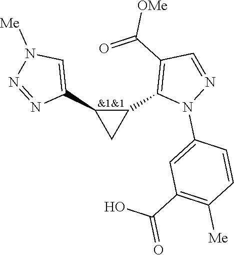

To a suspension of 5-hydrazinyl-2-methylbenzoic acid, hydrochloride (4.6 g, 22.70 mmol) in ethanol (56.8 ml) was added methyl 3-(dimethylamino)-2-(trans)-2-(1-methyl-1H-1,2,3-triazol-4-yl)cyclopropanecarbonyl)acrylate (1.579 g, 5.68 mmol) and triethylamine (3.16 ml, 22.70 mmol) sequentially. Warmed to 65° C. After 30 min, cooled to RT. Partitioned with 300 mL EtOAc and 100 mL 1M aqueous HCl, separated layers, and washed organics with 1×50 mL aqueous 1M HCl. Back-extracted combined aqueous with 2×40 mL EtOAc. Dried combined organics over Na₂SO₄, filtered, and concentrated in vacuo to give an orange oil (7.2 g). LC-MS m/z 382.1 (M+H)⁺, 0.69 min (ret. time). Carried forward without further purification.

### 25b) Methyl 1-(3-(hydroxymethyl)-4-methylphenyl)-5-(trans)-2-(1-methyl-1H-1,2,3-triazol-4-yl)cyclopropyl)-1H-pyrazole-4-carboxylate

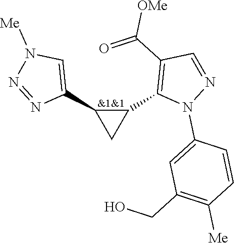

To a cloudy mixture of 5-(4-(methoxycarbonyl)-5-(trans)-2-(1-methyl-1H-1,2,3-triazol-4-yl)cyclopropyl)-1H-pyrazol-1-yl)-2-methylbenzoic acid (1 g, 2.62 mmol) in anhydrous tetrahydrofuran (47.0 ml) at RT was added CDI (1.275 g, 7.87 mmol), giving a clear, orange solution. After 2 h, added to a vigorously stirred mixture of NaBH₄ (0.496 g, 13.11 mmol) in water (12.55 ml) at RT. After 8 min at RT, partitioned with 100 mL EtOAc and 20 mL H₂O. Separated layers, washed organics with 2×40 mL aqueous 1M NaOH. Dried organics over Na₂SO₄, filtered, and concentrated in vacuo to give a yellow oil. Purified by normal-phase CombiFlash ISCO (24 g Gold column, 0-100% (3:1 EtOAc:EtOH):Hexanes) to give methyl 1-(3-(hydroxymethyl)-4-methylphenyl)-5-(trans)-2-(1-methyl-1H-1,2,3-triazol-4-yl)cyclopropyl)-1H-pyrazole-4-carboxylate as a pale yellow oil (184.3 mg, 0.502 mmol, 19% yield). LC-MS m/z 368.1 (M+H)⁺, 0.66 min (ret. time).

### 25c) Methyl 1-(3-(((R)-4-ethyl-1,1-dioxido-3,4-dihydro-2H-benzo[b][1,4,5]oxathiazepin-2-yl)methyl)-4-methylphenyl)-5-(trans)-2-(1-methyl-1H-1,2,3-triazol-4-yl)cyclopropyl)-1H-pyrazole-4-carboxylate

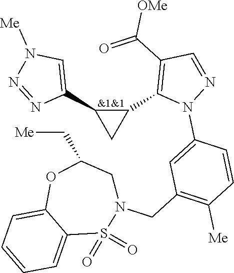

To a solution of methyl 1-(3-(hydroxymethyl)-4-methylphenyl)-5-(trans)-2-(1-methyl-1H-1,2,3-triazol-4-yl)cyclopropyl)-1H-pyrazole-4-carboxylate (81 mg, 0.220 mmol) in tetrahydrofuran (1102 μl) was added sequentially (R)-4-ethyl-3,4-dihydro-2H-benzo[b][1,4,5]oxathiazepine 1,1-dioxide (100 mg, 0.441 mmol), DTBAD (203 mg, 0.882 mmol) and tributylphosphine (220 μl, 0.882 mmol). After 30 min at RT, concentrated in vacuo to give a pale yellow oil. Purified by normal-phase CombiFlash ISCO (24 g Gold column, 0-100% EtOAc:Hexanes) to give methyl 1-(3-(((R)-4-ethyl-1,1-dioxido-3,4-dihydro-2H-benzo[b][1,4,5]oxathiazepin-2-yl)methyl)-4-methylphenyl)-5-(trans)-2-(1-methyl-1H-1,2,3-triazol-4-yl)cyclopropyl)-1H-pyrazole-4-carboxylate as a white solid (62.8 mg, 0.109 mmol, 49% yield). LC-MS m/z 577.1 (M+H)⁺, 1.06 min (ret. time). ¹H NMR (400 MHz, CHLOROFORM-d) δ ppm 1.08-1.15 (m, 3H) 1.51-1.58 (m, 3H) 1.74 (dd, J=14.31, 7.03 Hz, 1H) 2.39 (d, J=2.76 Hz, 3H) 2.42-2.50 (m, 2H) 3.03-3.17 (m, 1H) 3.77 (dt, J=15.00, 10.07 Hz, 1H) 3.83-3.87 (m, 3H) 3.92 (d, J=14.05 Hz, 1H) 4.06 (d, J=4.52 Hz, 4H) 4.49 (d, J=15.06 Hz, 1H) 7.23 (d, J=8.03 Hz, 1H) 7.36 (s, 2H) 7.38-7.42 (m, 2H) 7.48 (d, J=4.77 Hz, 1H) 7.55 (t, J=7.65 Hz, 1H) 7.89 (d, J=7.78 Hz, 1H) 8.05 (s, 1H).

### 25d) 1-(3-(((R)-4-Ethyl-1,1-dioxido-3,4-dihydro-2H-benzo[b][1,4,5]oxathiazepin-2-yl)methyl)-4-methylphenyl)-5-(trans)-2-(1-methyl-1H-1,2,3-triazol-4-yl)cyclopropyl)-1H-pyrazole-4-carboxylic acid

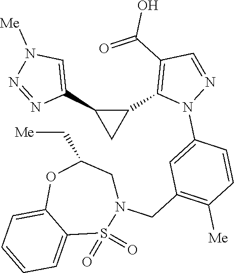

To a suspension of methyl 1-(3-(((R)-4-ethyl-1,1-dioxido-3,4-dihydro-2H-benzo[b][1,4,5]oxathiazepin-2-yl)methyl)-4-methylphenyl)-5-(trans)-2-(1-methyl-1H-1,2,3-triazol-4-yl)cyclopropyl)-1H-pyrazole-4-carboxylate (37.6 mg, 0.065 mmol) in methanol (217 μl) was added an aqueous solution of NaOH (652 μl, 0.652 mmol, 1 M). Warmed to 70° C. After 30 min, cooled to RT. Partitioned with 15 mL EtOAc and 5 mL H₂O, separated layers. Acidified aqueous layer to pH=2 with 1M aqueous HCl, partitioned with 10 mL EtOAc, and separated layers. Back-extracted aqueous with 3×5 mL EtOAc. Dried combined organics over Na₂SO₄, filtered, concentrated in vacuo to give (after trituration with DCM/Hexane) 1-(3-(((R)-4-ethyl-1,1-dioxido-3,4-dihydro-2H-benzo[b][1,4,5]oxathiazepin-2-yl)methyl)-4-methylphenyl)-5-(trans)-2-(1-methyl-1H-1,2,3-triazol-4-yl)cyclopropyl)-1H-pyrazole-4-carboxylic acid as a white solid (27.0 mg, 0.048 mmol, 74%). LC-MS m/z 563.1 (M+H)⁺, 0.96 min (ret. time). ¹H NMR (400 MHz, DMSO-d₆) δ ppm 1.05 (t, J=7.15 Hz, 3H) 1.15-1.25 (m, 2H) (d, J=9.29 Hz, 2H) 2.12-2.21 (m, 1H) 2.34 (s, 3H) 2.39-2.48 (m, 1H) 3.30 (s, 2H) 3.63-3.82 (m, 1H) 3.94 (s, 4H) 4.03 (s, 1H) 4.06-4.14 (m, 1H) 4.30-4.41 (m, 1H) 7.32 (d, J=7.78 Hz, 2H) 7.39 (d, J=13.80 Hz, 2H) 7.50 (s, 1H) 7.60 (d, J=6.02 Hz, 1H) 7.67 (s, 1H) 7.79 (d, J=7.78 Hz, 1H) 7.98 (s, 1H).

### Example 26. 1-((R)-1-((S)-4-Methyl-1,1-dioxido-8-(trifluoromethyl)-4,5-dihydrobenzo[f][1,2]thiazepin-2(3H)-yl)-2,3-dihydro-1H-inden-4-yl)-5-((1R,2R)-2-(1-methyl-1H-1,2,3-triazol-4-yl)cyclopropyl)-1H-pyrazole-4-carboxylic acid

### Example 26a: 1-((S)-1-((S)-4-Methyl-1,1-dioxido-8-(trifluoromethyl)-4,5-dihydrobenzo[f][1,2]thiazepin-2(3H)-yl)-2,3-dihydro-1H-inden-4-yl)-5-((1R,2R)-2-(1-methyl-1H-1,2,3-triazol-4-yl)cyclopropyl)-1H-pyrazole-4-carboxylic acid

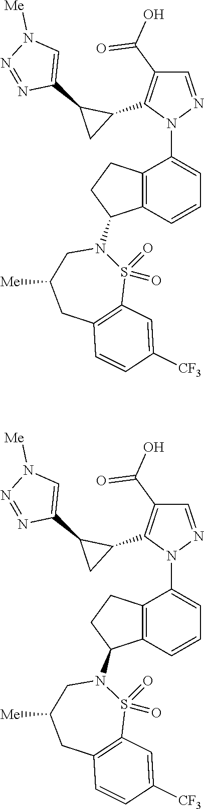 [SMILES: C[C@H]1Cc2ccc(C(F)(F)F)cc2S(=O)(=O)N([C@@H]2CCc3c2cccc3-n2ncc(C(=O)O)c2[C@@H]2C[C@H]2c2cn(C)nn2)C1 ; C[C@H]1Cc2ccc(C(F)(F)F)cc2S(=O)(=O)N([C@H]2CCc3c2cccc3-n2ncc(C(=O)O)c2[C@@H]2C[C@H]2c2cn(C)nn2)C1]

### 26(i) 1 4-Iodo-2,3-dihydro-1H-inden-1-ol

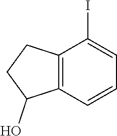 [SMILES: OC1CCc2c(I)cccc21]

To a suspension of 4-iodo-2,3-dihydro-1H-inden-1-one (1000 mg, 3.88 mmol) in methanol (19.4 mL) at RT was added NaBH₄ (293 mg, 7.75 mmol). After 45 min, the reaction mixture was added dropwise to 20 mL water and diluted with 50 mL EtOAc. The layers were separated and the aqueous layer was extracted with 3×10 mL EtOAc. The combined organics were dried

over Na₂SO₄, filtered and concentrated to give the title compound as a yellow solid (1.0 g, 3.9 mmol, 100% yield) which was carried forward without further purification. LC-MS m/z 243.0 (M−OH)⁺, 0.85 min (ret. time).

### 26(ii) Benzyl 1-(1-hydroxy-2,3-dihydro-1H-inden-4-yl)hydrazine-1-carboxylate

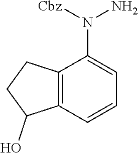 [SMILES: NN(C(=O)OCc1ccccc1)c1cccc2c1CCC2O]

4-Iodo-2,3-dihydro-1H-inden-1-ol (586.1 mg, 2.254 mmol) was combined with benzyl hydrazinecarboxylate (899 mg, 5.41 mmol), cesium carbonate (2056 mg,

6.31 mmol), (1S,2S)—N1,N2-dimethylcyclohexane-1,2-diamine (70.9 μl, 0.451 mmol) and copper(I) iodide (42.9 mg, 0.225 mmol) under an atmosphere of N₂. To this was added degassed N,N-dimethylformamide (DMF) (2504 μl) and the resulting reaction mixture was warmed to 80° C. After 18 h, the reaction mixture was cooled to RT, filtered through Celite, washed with 50 mL EtOAc and partitioned with 25 mL water. The layers were separated and the aqueous layer was extracted with 2×15 mL EtOAc. The combined organics were washed with 3×20 mL water and 3×25 mL brine. The combined organics were dried over Na₂SO₄, filtered and concentrated to give an orange oil. Purification by silica gel chromatography (40 g column, 0-10% MeOH:DCM) afforded the title compound as a clear, orange semi-solid (190 mg, 0.64 mmol, 28% yield). LC-MS m/z 299.1 (M+H)⁺, 0.66 min (ret. time). ¹H NMR (400 MHz, DMSO-d6) δ ppm 1.71 (dd, J=12.55, 7.03 Hz, 1H) 2.22-2.34 (m, 1H) 2.55-2.63 (m, 1H) 2.75-2.87 (m, 1H) 3.29 (s, 1H) 4.99-5.08 (m, 1H) 5.12 (s, 2H) 5.17 (s, 1H) 5.25 (d, J=5.77 Hz, 1H) 7.14 (br. s., 1H) 7.20 (d, J=5.77 Hz, 2H) 7.25-7.45 (m, 5H).

### 26(iii) 4-Hydrazinyl-2,3-dihydro-1H-inden-1-ol

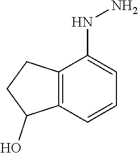 [SMILES: NNc1cccc2c1CCC2O]

To a mixture of benzyl 1-(1-hydroxy-2,3-dihydro-1H-inden-4-yl)hydrazine-1-carboxylate (258.6 mg, 0.867 mmol) in triethylsilane (3184 μl, 19.94 mmol) was added triethylamine (242 μl, 1.734 mmol) and palladium(II) chloride (154 mg, 0.867 mmol). Sonicated the reaction mixture until most of the starting material dissolved. After stirring for 1 h, 20 min, the reaction contents were filtered through Celite, washed with EtOAc and concentrated to give an orange oil (142 mg) which was carried forward without further purification. LC-MS m/z 299.0 (M+H)⁺, 0.66 min (ret. time).

### 26(iv) Methyl 1-(1-hydroxy-2,3-dihydro-1H-inden-4-yl)-5-((1R,2R)-2-(1-methyl-1H-1,2,3-triazol-4-yl)cyclopropyl)-1H-pyrazole-4-carboxylate

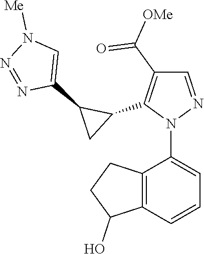 [SMILES: COC(=O)c1cnn(-c2cccc3c2CCC3O)c1[C@@H]1C[C@H]1c1cn(C)nn1]

To a solution of 4-hydrazinyl-2,3-dihydro-1H-inden-1-ol (142 mg, 0.865 mmol) in ethanol (8648 μl) was added methyl (Z)-3-(dimethylamino)-2-((1R,2R)-2-(1-methyl-1H-1,2,3-triazol-4-yl)cyclopropane-1-carbonyl)acrylate (241 mg, 0.865 mmol) followed by triethylamine (121 μl, 0.865 mmol). The resulting reaction mixture was warmed to 65° C. After 30 min, the reaction contents were cooled to RT and concentrated in vacuo to give an orange oil. Purification by silica gel chromatography (24 g column, 0-10% MeOH:DCM) afforded the title compound (diastereomeric mixture) as a white solid (29.2 mg, 0.08 mmol, 8.9% yield). LC-MS m/z 402.1 (M+Na)⁺, 0.61, 0.64 min (ret. time).

### 26(v) Methyl 1-(1-chloro-2,3-dihydro-1H-inden-4-yl)-5-((1R,2R)-2-(1-methyl-1H-1,2,3-triazol-4-yl)cyclopropyl)-1H-pyrazole-4-carboxylate

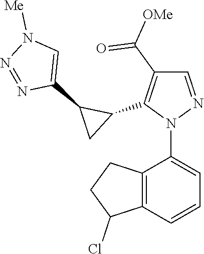 [SMILES: COC(=O)c1cnn(-c2cccc3c2CCC3Cl)c1[C@@H]1C[C@H]1c1cn(C)nn1]

To a solution of methyl 1-(1-hydroxy-2,3-dihydro-1H-inden-4-yl)-5-((1R,2R)-2-(1-methyl-1H-1,2,3-triazol-4-yl)cyclopropyl)-1H-pyrazole-4-carboxylate (29.2 mg, 0.077 mmol) in dichloromethane (DCM) (385 μl) was added thionyl chloride (16.85 μl, 0.231 mmol) to give a clear, pale yellow solution. The resulting reaction mixture was allowed to stir at 0° C. for 25 min. Following this duration, the reaction mixture was concentrated in vacuo to give a tan solid (34.6 mg, diastereomeric mixture) which was carried forward without further purification. LC-MS m/z 362.2 (M+H)⁺, 0.89, 0.91 min (ret. time).

### 26(vi) Methyl 1-(1-((S)-4-methyl-1,1-dioxido-8-(trifluoromethyl)-4,5-dihydrobenzo[f][1,2]thiazepin-2(3H)-yl)-2,3-dihydro-1H-inden-4-yl)-5-((1R,2R)-2-(1-methyl-1H-1,2,3-triazol-4-yl)cyclopropyl)-1H-pyrazole-4-carboxylate

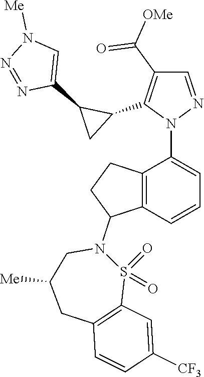 [SMILES: COC(=O)c1cnn(-c2cccc3c2CCC3N2C[C@@H](C)Cc3ccc(C(F)(F)F)cc3S2(=O)=O)c1[C@@H]1C[C@H]1c1cn(C)nn1]

To a solution of (S)-4-methyl-8-(trifluoromethyl)-2,3,4,5-tetrahydrobenzo[f][1,2]thiazepine 1,1-dioxide (43.0 mg, 0.154 mmol) in N,N-dimethylformamide (DMF) (342 μl) at 0° C. was added NaH (6.15 mg, 0.154 mmol, 60% dispersion in mineral oil). After 30 min at 0° C., a solution of methyl 1-(1-chloro-2,3-dihydro-1H-inden-4-yl)-5-((1R,2R)-2-(1-methyl-1H-1,2,3-triazol-4-yl)cyclopropyl)-1H-pyrazole-4-carboxylate (30.6 mg, 0.077 mmol) and TBAI (2.84 mg, 7.69 μmol) in N,N-dimethylformamide (DMF) (171 μl) was added and the resulting mixture was warmed to 80° C. After 50 min, the reaction contents were cooled to RT and partitioned with 15 mL EtOAc and 10 mL saturated aqueous NaHCO₃. The resulting layers were separated and the aqueous layer was extracted with 1×5 mL EtOAc. The combined organics were washed with 4×5 mL water and 1×5 mL brine. The combined organics were dried over Na₂SO₄, filtered and concentrated to give a brown oil (90.4 mg, diastereomeric mixture), which was carried forward without further purification. LC-MS m/z 641.4 (M+H)⁺, 1.24 min (ret. time).

### 26(vii) 1-((R)-1-((S)-4-Methyl-1,1-dioxido-8-(trifluoromethyl)-4,5-dihydrobenzo[f][1,2]thiazepin-2(3H)-yl)-2,3-dihydro-1H-inden-4-yl)-5-((1R,2R)-2-(1-methyl-1H-1,2,3-triazol-4-yl)cyclopropyl)-1H-pyrazole-4-carboxylic acid

### 26a: 1-((S)-1-((S)-4-Methyl-1,1-dioxido-8-(trifluoromethyl)-4,5-dihydrobenzo[f][1,2]thiazepin-2(3H)-yl)-2,3-dihydro-1H-inden-4-yl)-5-((1R,2R)-2-(1-methyl-1H-1,2,3-triazol-4-yl)cyclopropyl)-1H-pyrazole-4-carboxylic acid

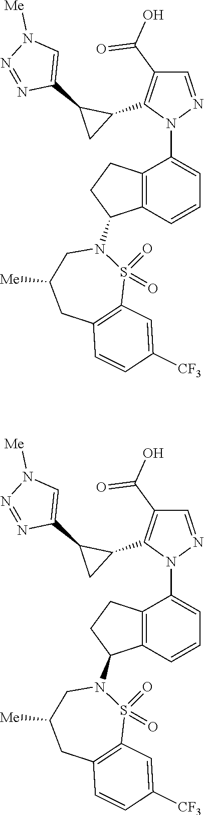 [SMILES: C[C@H]1Cc2ccc(C(F)(F)F)cc2S(=O)(=O)N([C@@H]2CCc3c2cccc3-n2ncc(C(=O)O)c2[C@@H]2C[C@H]2c2cn(C)nn2)C1 ; C[C@H]1Cc2ccc(C(F)(F)F)cc2S(=O)(=O)N([C@H]2CCc3c2cccc3-n2ncc(C(=O)O)c2[C@@H]2C[C@H]2c2cn(C)nn2)C1]

To a suspension of methyl 1-(1-((S)-4-methyl-1,1-dioxido-8-(trifluoromethyl)-4,5-dihydrobenzo[f][1,2]thiazepin-2(3H)-yl)-2,3-dihydro-1H-inden-4-yl)-5-((1R,2R)-2-(1-methyl-1H-1,2,3-triazol-4-yl)cyclopropyl)-1H-pyrazole-4-carboxylate (49.3 mg, 0.077 mmol) in methanol (769 μl) was added aqueous NaOH (769 μl, 0.769 mmol, 1.0 M). The resulting reaction mixture was heated to 100° C. After 50 min, the reaction contents were cooled to RT and purified by reverse-phase HPLC (10-90% CH₃CN+0.1% TFA:H₂O+0.1% TFA) to give

an orange oil (36.2 mg, diastereomeric mixture). Subsequent purification by chiral SFC (Chiralpak IG) afforded the following:

1-((R)-1-((S)-4-Methyl-1,1-dioxido-8-(trifluoromethyl)-4,5-dihydrobenzo[f][1,2]thiazepin-2(3H)-yl)-2,3-dihydro-1H-inden-4-yl)-5-((1R,2R)-2-(1-methyl-1H-1,2,3-triazol-4-yl)cyclopropyl)-1H-pyrazole-4-carboxylic acid (white solid, 3.0 mg, 4.8 μmol, 6% yield): LC-MS m/z 627.4 (M+H)⁺, 1.14 min (ret. time); ¹H NMR (400 MHz, METHANOL-d4) δ ppm 0.99 (d, J=6.53 Hz, 3H) 1.37-1.44 (m, 1H) 1.46-1.53 (m, 1H) 1.65-1.75 (m, 1H) 1.78-1.85 (m, 1H) 1.87-1.97 (m, 1H) 2.26-2.33 (m, 2H) 2.46-2.59 (m, 1H) 2.61-2.76 (m, 1H) 2.91-3.10 (m, 2H) 3.62-3.74 (m, 2H) 4.00-4.05 (m, 3H) 5.34-5.43 (m, 1H) 7.30-7.37 (m, 1H) 7.44 (s, 3H) 7.59-7.66 (m, 1H) 7.82-7.87 (m, 1H) 8.01 (s, 1H) 8.06-8.11 (m, 1H).

1-((S)-1-((S)-4-Methyl-1,1-dioxido-8-(trifluoromethyl)-4,5-dihydrobenzo[f][1,2]thiazepin-2(3H)-yl)-2,3-dihydro-1H-inden-4-yl)-5-((1R,2R)-2-(1-methyl-1H-1,2,3-triazol-4-yl)cyclopropyl)-1H-pyrazole-4-carboxylic acid (white solid, 4.1 mg, 6.5 μmol, 9% yield): LC-MS m/z 627.4 (M+H)⁺, 1.13 min (ret. time); ¹H NMR (400 MHz, METHANOL-d4) δ ppm 0.83 (d, J=6.78 Hz, 3H) 0.87-0.93 (m, 1H) 0.94-1.03 (m, 1H) 1.41-1.50 (m, 1H) 1.54-1.67 (m, 1H) 1.98-2.11 (m, 1H) 2.24-2.32 (m, 1H) 2.35-2.47 (m, 2H) 2.54-2.70 (m, 1H) 2.88-2.99 (m, 1H) 3.00-3.11 (m, 1H) 3.16-3.26 (m, 1H) 3.51-3.59 (m, 1H) 4.04 (s, 3H) 5.52-5.61 (m, 1H) 6.42-6.56 (m, 1H) 7.09-7.19 (m, 1H) 7.27-7.33 (m, 1H) 7.52-7.58 (m, 1H) 7.61-7.67 (m, 1H) 7.86-7.93 (m, 1H) 8.02 (s, 1H) 8.15-8.21 (m, 1H).

The example in Table 7 was prepared in an analogous manner:

TABLE 7

| | | | LCMS | Retention | |
|---|---|---|---|---|---|
| Ex # | Structure | Name | [M + H]⁺ | Time (min) | ¹HNMR |
| Example 27 | 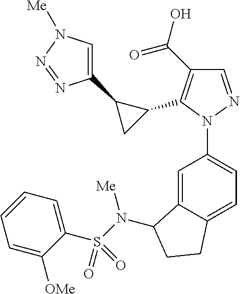 [SMILES: COc1ccccc1S(=O)(=O)N(C)C1CCc2ccc(-n3ncc(C(=O)O)c3[C@@H]3C[C@H]3c3cn(C)nn3)cc21] | 1-(3-(2-methoxy-N- methylphenylsulfonamido)- 2,3-dihydro- 1H-inden-5-yl)-5- ((1R,2R)-2-(1-methyl- 1H-1,2,3-triazol-4- yl)cyclopropyl)-1H- pyrazole-4-carboxylic acid | 549.1 | 0.84 | ¹H NMR (400 MHz, CHLOROFORM- d) δ ppm 1.27 (d, J = 16.31 Hz, 2 H) 1.50 (br. s., 2 H) 1.90- 2.09 (m, 2 H) 2.22 (d, J = 9.29 Hz, 2 H) 2.43 (br. s., 4 H) 2.64 (s, 6 H) 2.84-2.96 (m, 2 H) 3.01 (br. s., 2 H) 3.98 (d, J = 10.54 Hz, 6 H) 4.14 (d, |
| | | | | | J = 10.54 Hz, 6 |
| | | | | | H) 5.42 (t, |
| | | | | | J = 7.40 Hz, 1 |
| | | | | | H) 5.62 (t, |
| | | | | | J = 7.91 Hz, 1 |
| | | | | | H) 7.00-7.12 |
| | | | | | (m, 4 H) 7.26- |
| | | | | | 7.29 (m, 2 |
| | | | | | H) 7.31-7.38 |
| | | | | | (m, 4 H) 7.39- |
| | | | | | 7.51 (m, 2 |
| | | | | | H) 7.56 (q, |
| | | | | | J = 7.19 Hz, 2 |
| | | | | | H) 7.98 (dd, |
| | | | | | J = 12.42, 7.91 |
| | | | | | Hz, 2 H) 8.17 |
| | | | | | (br. s., 2 H) |

### Example 28. 1-(3-(((R)-2-ethyl-2,3-dihydrobenzo[f][1,4]oxazepin-4(5H)-yl)methyl)-4-methylphenyl)-5-(trans)-2-(1-methyl-1H-1,2,3-triazol-4-yl)cyclopropyl)-1H-pyrazole-4-carboxylic acid

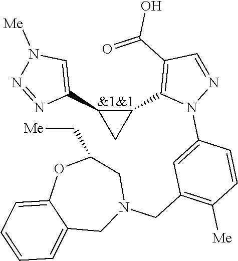 [SMILES: CC[C@@H]1CN(Cc2cc(-n3ncc(C(=O)O)c3[C@@H]3C[C@H]3c3cn(C)nn3)ccc2C)Cc2ccccc2O1]

### 28a) Methyl 1-(3-(chloromethyl)-4-methylphenyl)-5-(trans)-2-(1-methyl-1H-1,2,3-triazol-4-yl)cyclopropyl)-1H-pyrazole-4-carboxylate

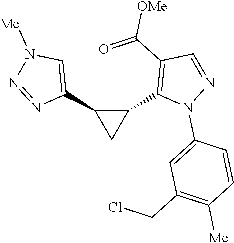 [SMILES: COC(=O)c1cnn(-c2ccc(C)c(CCl)c2)c1[C@@H]1C[C@H]1c1cn(C)nn1]

To an orange solution of methyl 1-(3-(hydroxymethyl)-4-methylphenyl)-5-(trans)-2-(1-methyl-1H-1,2,3-triazol-4-yl)cyclopropyl)-1H-pyrazole-4-carboxylate (93.2 mg, 0.254 mmol) in dichloromethane (846 μl) was added thionyl chloride (37.0 μl, 0.507 mmol). After 10 min at RT, concentrated in vacuo to give an orange oil (118.1 mg). LC-MS m/z 385.9 (M+H)⁺, 0.95 min (ret. time). Carried forward without further purification.

### 28b) Methyl 1-(3-(((R)-2-ethyl-2,3-dihydrobenzo[f][1,4]oxazepin-4(5H)-yl)methyl)-4-methylphenyl)-5-(trans)-2-(1-methyl-1H-1,2,3-triazol-4-yl)cyclopropyl)-1H-pyrazole-4-carboxylate

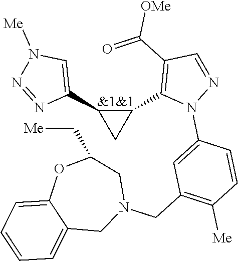 [SMILES: CC[C@@H]1CN(Cc2cc(-n3ncc(C(=O)OC)c3[C@@H]3C[C@H]3c3cn(C)nn3)ccc2C)Cc2ccccc2O1]

To an orange solution of methyl 1-(3-(chloromethyl)-4-methylphenyl)-5-(trans)-2-(1-methyl-1H-1,2,3-triazol-4-yl)cyclopropyl)-1H-pyrazole-4-carboxylate (98 mg, 0.254 mmol) in acetonitrile (2540 μl) was added DIPEA (266 μl, 1.524 mmol) and (R)-2-ethyl-2,3,4,5-tetrahydrobenzo[f][1,4]oxazepine hydrochloride (109 mg, 0.508 mmol) sequentially. Warmed to 100° C. After 30 min, concentrated in vacuo to give a brown oil. Purified by normal-phase CombiFlash ISCO (24 g Gold column, 0-100% (3:1 EtOAc:EtOH):Hexanes) to give methyl 1-(3-(((R)-2-ethyl-2,3-dihydrobenzo[f][1,4]oxazepin-4(5H)-yl)methyl)-4-methylphenyl)-5-(trans)-2-(1-methyl-1H-1,2,3-triazol-4-yl)cyclopropyl)-1H-pyrazole-4-carboxylate as a light brown solid (43 mg, 0.082 mmol, 32% yield). LC-MS m/z 527.1 (M+H)⁺, 0.78 min (ret. time). ¹H NMR (400 MHz, METHANOL-d₄) δ ppm 1.09 (t, J=7.28 Hz, 3H) 1.23-1.34 (m, 1H) 1.39-1.46 (m, 1H) 1.47-1.55 (m, 1H) 1.59-1.70 (m, 1H) 2.25-2.31 (m, 1H) 2.38 (s, 3H) 2.41-2.47 (m, 1H) 2.82-2.96 (m, 1H) 2.98-3.07 (m, 1H) 3.62 (s, 3H) 3.83 (s, 4H) 3.88-3.95 (m, 1H) 4.02 (s, 3H) 6.98 (d, J=6.78 Hz, 3H) 7.16-7.23 (m, 1H) 7.33 (s, 2H) 7.41 (s, 1H) 7.57 (s, 1H) 8.04 (s, 1H).

### 28c) 1-(3-(((R)-2-Ethyl-2,3-dihydrobenzo[f][1,4]oxazepin-4(5H)-yl)methyl)-4-methylphenyl)-5-(trans)-2-(1-methyl-1H-1,2,3-triazol-4-yl)cyclopropyl)-1H-pyrazole-4-carboxylic acid

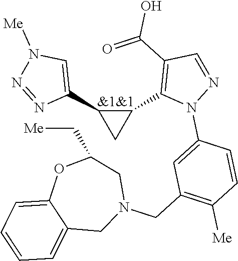 [SMILES: CC[C@@H]1CN(Cc2cc(-n3ncc(C(=O)O)c3[C@@H]3C[C@H]3c3cn(C)nn3)ccc2C)Cc2ccccc2O1]

To a suspension of methyl 1-(3-(((R)-2-ethyl-2,3-dihydrobenzo[f][1,4]oxazepin-4(5H)-yl)methyl)-4-methylphenyl)-5-(trans)-2-(1-methyl-1H-1,2,3-triazol-4-yl)cyclopropyl)-1H-pyrazole-4-carboxylate (26.3 mg, 0.050 mmol) in methanol (166 μl) was added an aqueous solution of NaOH (499 μl, 0.499 mmol, 1 M). Warmed to 70° C. After 60 min, concentrated in vacuo to give a brown semi-solid. Dissolved in 200 uL 1,4-dioxane, acidified with 2 mL 1M aqueous HCl. Added 3 drops DMSO and purified by reverse-phase HPLC (10-70% CH₃CN:H₂O, acidic conditions) to give (following concentration of product-containing fractions in vacuo) 1-(3-(((R)-2-ethyl-2,3-dihydrobenzo[f][1,4]oxazepin-4(5H)-yl)methyl)-4-methylphenyl)-5-(trans)-2-(1-methyl-1H-1,2,3-triazol-4-yl)cyclopropyl)-1H-pyrazole-4-carboxylic acid (TFA salt) as a white solid (31.2 mg, 0.046 mmol, 91% yield). LC-MS m/z 513.1 (M+H)⁺, 0.72 min (ret. time). ¹H NMR (400 MHz, DMSO-d₆) δ ppm 0.81-0.92 (m, 1H) 1.00-1.10 (m, 3H) 1.14-1.22 (m, 1H) 1.28-1.39 (m, 1H) 1.55-1.70 (m, 2H) 2.11-2.25 (m, 1H) 2.59 (s, 3H) 3.43-3.52 (m, 2H) 3.65-3.73 (m, 2H) 3.74 (s, 2H) 3.98 (br. s., 3H) 4.36-4.48 (m, 1H) 7.08-7.18 (m, 1H) 7.38-7.45 (m, 2H) 7.53-7.62 (m, 1H) 7.64-7.74 (m, 2H) 7.78-7.88 (m, 1H) 7.92-7.96 (m, 1H) 8.00 (s, 1H).

### Example 29. 1-(3-(((R)-4-Ethyl-1,1-dioxido-3,4-dihydro-2H-benzo[b][1,4,5]oxathiazepin-2-yl)methyl)phenyl)-5-(trans)-2-(1-(2,2,2-trifluoroethyl)-1H-1,2,3-triazol-4-yl)cyclopropyl)-1H-pyrazole-4-carboxylic acid

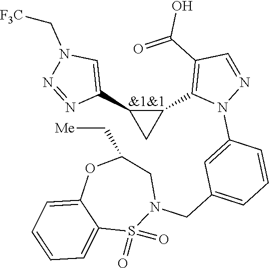 [SMILES: CC[C@@H]1CN(Cc2cccc(-n3ncc(C(=O)O)c3[C@@H]3C[C@H]3c3cn(CC(F)(F)F)nn3)c2)S(=O)(=O)c2ccccc2O1]

### 29a) Ethyl 2-(2,2-dibromovinyl)cyclopropanecarboxylate

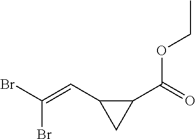

To a solution of triphenylphosphine (258 g, 985 mmol) in dichloromethane (DCM) (300 mL) was added a solution of carbon tetrabromide (163 g, 492 mmol) in dichloromethane (DCM) (300 mL) dropwise at 0° C. After addition, the mixture was stirred for 10 min, a solution of ethyl 2-formylcyclopropanecarboxylate (35 g, 246 mmol) in dichloromethane (DCM) (50 mL) was then added dropwise. After addition, the reaction mixture was stirred for 30 min at 0° C. The ice bath was removed and the reaction mixture was stirred for additional 30 min. The mixture was filtered and the filtrate was concentrated. The residue was triturated with hexanes and filtered through celite. The filtrate was filtered again through celite. To the filtrate was added isolute and concentrated. The isolute was loaded onto a cartridge and purified on the Torrent column chromatography (750 g silica gel, gold) eluting with a gradient of 0-5% ethyl acetate in hexanes. The title compound was obtained as clear colorless oil (43.7 g, 147 mmol, 59.6% yield). LC-MS m/z 296.9 (M+H)⁺, 1.14 min (ret. time).

### 29b) 2-(2,2-Dibromovinyl)cyclopropanecarboxylic acid

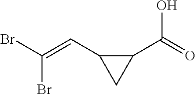

To a solution of ethyl 2-(2,2-dibromovinyl)cyclopropanecarboxylate (43.7 g, 147 mmol) in tetrahydrofuran (THF) (70 mL) and methanol (70 mL) was added 6.0 N NaOH (73.3 mL, 440 mmol). The reaction mixture was stirred for 1.0 hour at room temperature. The mixture was acidified with 6.0 N HCl (aq) and extracted with ethyl acetate. The organic extract was washed with water and dried over anhydrous magnesium sulfate. It was filtered and the filtrated was concentrated to give the title compound (37.6 g, 139 mmol, 95% yield) as white solid used in the next step without further purification. LC-MS m/z 268.5 (M+H)⁺, 0.86 min (ret. time).

### 29c) Methyl 3-(2-(2,2-dibromovinyl)cyclopropyl)-3-oxopropanoate

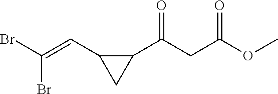

To a solution of 2-(2,2-dibromovinyl)cyclopropanecarboxylic acid (37.6 g, 139 mmol) in tetrahydrofuran (THF) (350 mL) was added CDI (33.9 g, 209 mmol). The mixture was stirred for 2 hours at room temperature, potassium 3-methoxy-3-oxopropanoate (65.3 g, 418 mmol) was added, followed by magnesium chloride (15.92 g, 167 mmol). The resulting reaction mixture was stirred overnight at room temperature. The mixture was neutralized with 6.0 N HCl and diluted with water, extracted with ethyl acetate. The organic extract was washed with brine and dried over anhydrous magnesium sulfate. It was filtered and the filtrate was concentrated. The residue was dissolved in DCM and purified on the Torrent flash column chromatography (750 g silica gel) eluting with a gradient of 0-25% ethyl acetate in hexanes. The title compound was obtained as clear yellow oil (45.4 g, 139 mmol, 100% yield). LC-MS m/z 324.8 (M+H)⁺, 1.0 min (ret. time).

### 29d) Methyl 2-(2-(2,2-dibromovinyl)cyclopropanecarbonyl)-3-(dimethylamino)acrylate

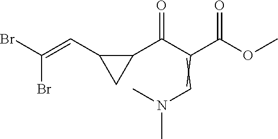 [SMILES: COC(=O)C(=CN(C)C)C(=O)C1CC1C=C(Br)Br]

A mixture of methyl 3-(2-(2,2-dibromovinyl)cyclopropyl)-3-oxopropanoate (45.4 g, 139 mmol) and 1,1-dimethoxy-N,N-dimethylmethanamine (24 ml, 180 mmol) in 1,4-Dioxane (24 mL) was stirred overnight at room temperature. The mixture was concentrated by rotavap. 1,4-dioxane (25 mL) was added and concentrated by rotavap (repeated one more time) to give the title compound (50 g, 131 mmol, 94% yield) as yellow oil used in the next step without further purification. LC-MS m/z 379.9 (M+H)⁺, 0.99 (ret. time).

### 29e) Methyl 1-(3-bromophenyl)-5-(2-(2,2-dibromovinyl)cyclopropyl)-1H-pyrazole-4-carboxylate

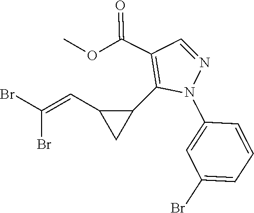 [SMILES: COC(=O)c1cnn(-c2cccc(Br)c2)c1C1CC1C=C(Br)Br]

To a solution of methyl 2-(2-(2,2-dibromovinyl)cyclopropanecarbonyl)-3-(dimethylamino)acrylate (50 g, 131 mmol) in Ethanol (300 mL) was added (3-bromophenyl)hydrazine hydrochloride (32.3 g, 144 mmol). To the resulting suspension was added TEA (36.6 mL, 262 mmol). The mixture turned to clear solution and it was stirred for 2.0 hours at room temperature. The mixture was filtered and the solid was washed with ethanol, dried to give the clean title compound as white powder (43.23 g, 86 mmol, 65.2% yield). LC-MS m/z 502.9 (M+H)⁺, 1.37 min (ret. time).

### 29f) Methyl 1-(3-bromophenyl)-5-(trans)-2-ethynylcyclopropyl)-1H-pyrazole-4-carboxylate

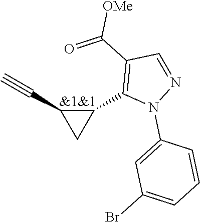 [SMILES: C#C[C@@H]1C[C@H]1c1c(C(=O)OC)cnn1-c1cccc(Br)c1]

To a solution of methyl 1-(3-bromophenyl)-5-(trans)-2-(2,2-dibromovinyl)cyclopropyl)-1H-pyrazole-4-carboxylate (1 g, 1.980 mmol) in dimethyl sulfoxide (9.90 ml) was added cesium carbonate (1.613 g, 4.95 mmol). Heated the resulting reaction mixture to 115° C. After 20 h, cooled to RT. Diluted with 20 mL of a mixture of ethyl acetate and hexanes (3:2 V:V), filtered through Celite, washed with 3:2 (V:V) ethyl acetate:hexanes. Diluted filtrate with 20 mL water. Separated layers, washed organics with 2×20 mL water, 2×20 mL brine. Dried organics over Na₂SO₄, filtered, and concentrated in vacuo to give a brown semi-solid. Purified by normal-phase CombiFlash ISCO (80 g Gold column, 0-20% EtOAc:Hexane) to give methyl 1-(3-bromophenyl)-5-(trans)-2-ethynylcyclopropyl)-1H-pyrazole-4-carboxylate as a clear, yellow oil (662.9 mg, 1.920 mmol, 97% yield). LC-MS m/z 344.9 (M+H)⁺, 1.07 min (ret. time). ¹H NMR (400 MHz, CHLOROFORM-d) δ ppm 1.11-1.21 (m, 1H) 1.34-1.43 (m, 1H) 1.51-1.65 (m, 1H) 1.95 (s, 1H) 2.26-2.38 (m, 1H) 3.90 (s, 3H) 7.36-7.45 (m, 1H) 7.52 (d, J=7.78 Hz, 1H) 7.61 (d, J=8.03 Hz, 1H) 7.76 (s, 1H) 8.04 (s, 1H).

### 29g) Methyl 1-(3-bromophenyl)-5-(trans)-2-(1-(2,2,2-trifluoroethyl)-1H-1,2,3-triazol-4-yl)cyclopropyl)-1H-pyrazole-4-carboxylate

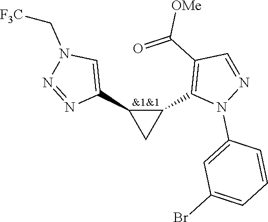 [SMILES: COC(=O)c1cnn(-c2cccc(Br)c2)c1[C@@H]1C[C@H]1c1cn(CC(F)(F)F)nn1]

To a solution of methyl 1-(3-bromophenyl)-5-(trans)-2-ethynylcyclopropyl)-1H-pyrazole-4-carboxylate (277.6 mg, 0.804 mmol) in tert-butanol (13.4 ml), water (3.4 μl) and N,N-dimethylformamide (3.4 ml) was added 2-azido-1,1,1-trifluoroethane (4.0 ml, 2.010 mmol, 0.5 M in MTBE), copper(I) iodide (30.6 mg, 0.161 mmol) and DIPEA (140 μl, 0.804 mmol). Heated to 70° C. After 2.5 h, filtered through Celite and washed with 50 mL EtOAc. Partitioned with 20 mL brine, separated layers. Back-extracted aqueous with 1×10 mL EtOAc. Washed combined organics with 4×20 mL brine, dried over Na₂SO₄, filtered, and concentrated in vacuo to give a pale yellow oil. Purified by normal-phase CombiFlash ISCO (24 g Gold column, 0-50% EtOAc:Hexanes) to give methyl 1-(3-bromophenyl)-5-(trans)-2-(1-(2,2,2-trifluoroethyl)-1H-1,2,3-triazol-4-yl)cyclopropyl)-1H-pyrazole-4-carboxylate as a white solid (188.9 mg, 0.402 mmol, 50% yield). LC-MS m/z 470.0 (M+H)⁺, 1.02 min (ret. time). ¹H NMR (400 MHz, CHLOROFORM-d) δ ppm 0.76-0.95 (m, 1H) 1.72-1.81 (m, 1H) 2.07 (s, 1H) 2.18-2.28 (m, 1H) 2.50-2.64 (m, 1H) 3.86 (s, 3H) 4.97 (d, J=8.53 Hz, 2H) 7.32 (s, 1H) 7.49 (s, 2H) 7.54-7.59 (m, 1H) 7.72 (s, 1H) 8.07 (s, 1H).

### 29h) Methyl 1-(3-(((4-methoxybenzyl)oxy)methyl)phenyl)-5-(trans)-2-(1-(2,2,2-trifluoroethyl)-1H-1,2,3-triazol-4-yl)cyclopropyl)-1H-pyrazole-4-carboxylate

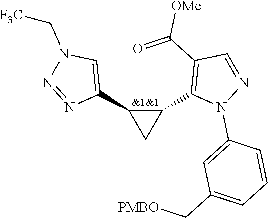

To a mixture of methyl 1-(3-bromophenyl)-5-(trans)-2-(1-(2,2,2-trifluoroethyl)-1H-1,2,3-triazol-4-yl)cyclopropyl)-1H-pyrazole-4-carboxylate (186.6 mg, 0.397 mmol), potassium (4-methoxy)benzyloxymethyl trifluoroborate (307 mg, 1.190 mmol), cesium carbonate (388 mg, 1.190 mmol), di(1-adamantyl)-n-butylphosphine (28.5 mg, 0.079 mmol) and palladium(II) acetate (8.91 mg, 0.040 mmol) under an atmosphere of nitrogen was added 1,4-dioxane (1653 μl) and water (331 μl). Warmed to 100° C. After 2.5 h, filtered through Celite, washed with 30 mL EtOAc, and partitioned with 15 mL saturated aqueous NaHCO₃. Separated layers, back-extracted aqueous with 3×10 mL EtOAc. Dried combined organics over Na₂SO₄, filtered, and concentrated in vacuo to give a yellow oil. Purified by normal-phase CombiFlash ISCO (24 g Gold column, 0-50% EtOAc:Hexane) to give a white solid (49.5 mg). LC-MS m/z 542.1 (M+H)⁺, 1.13 min (ret. time). Targeted compound was unable to be cleanly isolated and was carried forward without further purification.

### 29i) Methyl 1-(3-(hydroxymethyl)phenyl)-5-(trans)-2-(1-(2,2,2-trifluoroethyl)-1H-1,2,3-triazol-4-yl)cyclopropyl)-1H-pyrazole-4-carboxylate

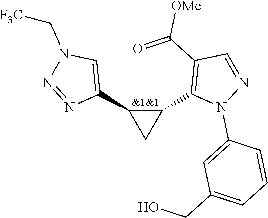 [SMILES: COC(=O)c1cnn(-c2cccc(CO)c2)c1[C@@H]1C[C@H]1c1cn(CC(F)(F)F)nn1]

Dissolved methyl 1-(3-(((4-methoxybenzyl)oxy)methyl)phenyl)-5-((1R,2R)-2-(1-(2,2,2-trifluoroethyl)-1H-1,2,3-triazol-4-yl)cyclopropyl)-1H-pyrazole-4-carboxylate (45.7 mg, 0.084 mmol) in dichloromethane (1990 μl) and added sequentially water (120 μl) and DDQ (22.99 mg, 0.101 mmol). After 90 min at RT, filtered through Celite and washed with 10 mL DCM. Partitioned with 3 mL water, separated layers. Extracted aqueous with 3×3 mL DCM. Dried over Na₂SO₄, filtered, and concentrated in vacuo to give an orange oil. Purified by reverse-phase HPLC (Mega Gilson, 10 min run, 10-80% CH₃CN:H₂O, acidic conditions) to give (following EtOAc/saturated aqueous NaHCO₃ workup of product-containing fractions) a red oil (28.1 mg). LC-MS m/z 422.1 (M+H)⁺, 0.76 min (ret. time). Product remained impure. Carried mixture forward.

### 29j) Methyl 1-(3-(((R)-4-ethyl-1,1-dioxido-3,4-dihydro-2H-benzo[b][1,4,5]oxathiazepin-2-yl)methyl)phenyl)-5-(trans)-2-(1-(2,2,2-trifluoroethyl)-1H-1,2,3-triazol-4-yl)cyclopropyl)-1H-pyrazole-4-carboxylate

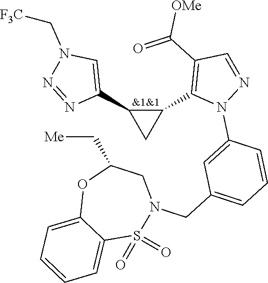 [SMILES: CC[C@@H]1CN(Cc2cccc(-n3ncc(C(=O)OC)c3[C@@H]3C[C@H]3c3cn(CC(F)(F)F)nn3)c2)S(=O)(=O)c2ccccc2O1]

To a solution of methyl 1-(3-(hydroxymethyl)phenyl)-5-(trans)-2-(1-(2,2,2-trifluoroethyl)-1H-1,2,3-triazol-4-yl)cyclopropyl)-1H-pyrazole-4-carboxylate (28.1 mg, 0.067 mmol) in tetrahydrofuran (333 μl) was added sequentially (R)-4-ethyl-3,4-dihydro-2H-benzo[b][1,4,5]oxathiazepine 1,1-dioxide (30.3 mg, 0.133 mmol), DTBAD (61.4 mg, 0.267 mmol) and tributylphosphine (66.6 μl, 0.267 mmol). After 30 min at RT, concentrated in vacuo to give a pale yellow oil. LC-MS m/z 631.1 (M+H)⁺, 1.16 min (ret. time). Carried forward without further purification.

### 29k) 1-(3-(((R)-4-Ethyl-1,1-dioxido-3,4-dihydro-2H-benzo[b][1,4,5]oxathiazepin-2-yl)methyl)phenyl)-5-(trans)-2-(1-(2,2,2-trifluoroethyl)-1H-1,2,3-triazol-4-yl)cyclopropyl)-1H-pyrazole-4-carboxylic acid

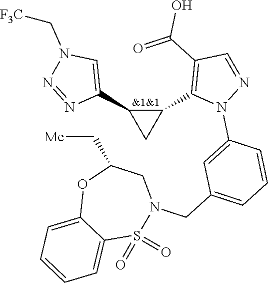 [SMILES: CC[C@@H]1CN(Cc2cccc(-n3ncc(C(=O)O)c3[C@@H]3C[C@H]3c3cn(CC(F)(F)F)nn3)c2)S(=O)(=O)c2ccccc2O1]

To a suspension of methyl 1-(3-(((R)-4-ethyl-1,1-dioxido-3,4-dihydro-2H-benzo[b][1,4,5]-oxathiazepin-2-yl)methyl)phenyl)-5-(trans)-2-(1-(2,2,2-trifluoroethyl)-1H-1,2,3-triazol-4-yl)cyclopropyl)-1H-pyrazole-4-carboxylate (42.1 mg, 0.067 mmol) in methanol (223 μl) was added an aqueous solution of NaOH (668 μl, 0.668 mmol, 1 M). Warmed to 70° C. After 2.5 h, cooled to RT, partitioned with 15 mL EtOAc and 5 mL H₂O, and separated layers. Back-extracted aqueous with 1×8 mL EtOAc. Acidified the aqueous layer to pH=2 with 1M aqueous HCl, partitioned with 10 mL EtOAc, and separated layers. Back-extracted aqueous with 3×5 mL EtOAc. Dried combined organics over Na₂SO₄, filtered, concentrated in vacuo to give an orange oil. Dissolved in 2 mL MeOH, purified by reverse-phase HPLC (10-70% CH₃CN:H₂O) to give (following concentration of product-containing fractions in vacuo) 1-(3-(((R)-4-ethyl-1,1-dioxido-3,4-dihydro-2H-benzo[b][1,4,5]oxathiazepin-2-yl)methyl)phenyl)-5-(trans)-2-(1-(2,2,2-trifluoroethyl)-1H-1,2,3-triazol-4-yl)cyclopropyl)-1H-pyrazole-4-carboxylic acid as a white solid (3.8 mg, 5.92 μmol, 9% yield). LC-MS m/z 617.1 (M+H)⁺, 1.08 min (ret. time). ¹H NMR (400 MHz, CHLOROFORM-d) δ ppm 1.14 (t, J=7.15 Hz, 3H) 1.34 (br. s., 1H) 1.57 (br. s., 1H) 1.69 (d, J=7.03 Hz, 1H) 1.73-1.83 (m, 1H) 2.36 (dd, J=11.67, 5.90 Hz, 1H) 2.51 (d, J=7.78 Hz, 1H) 3.18 (d, J=14.56 Hz, 1H) 3.75-3.89 (m, 1H) 3.95 (d, J=15.31 Hz, 1H) 4.04 (br. s., 1H) 4.49 (t, J=15.94 Hz, 1H) 4.96 (q, J=7.95 Hz, 2H) 7.23 (d, J=8.03 Hz, 1H) 7.39-7.59 (m, 6H) 7.87 (d, J=7.53 Hz, 1H) 8.13 (s, 1H) (one aromatic proton not visible—most likely overlapped by CHCl₃ signal).

### Example 30. 1-(3-(((S)-4-Methyl-1,1-dioxido-7-(trifluoromethyl)-4,5-dihydrobenzo[f][1,2]thiazepin-2(3H)-yl)methyl)phenyl)-5-(trans-2-(1-methyl-1H-pyrazol-4-yl)cyclopropyl)-1H-pyrazole-4-carboxylic acid

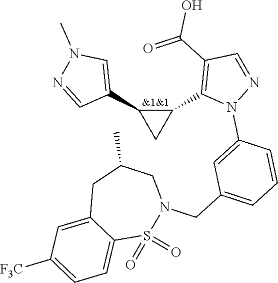 [SMILES: C[C@H]1Cc2cc(C(F)(F)F)ccc2S(=O)(=O)N(Cc2cccc(-n3ncc(C(=O)O)c3[C@@H]3C[C@H]3c3cnn(C)c3)c2)C1]

### 30a) (E)-tert-Butyl 3-(1-methyl-1H-pyrazole-4-yl)carbaldehyde

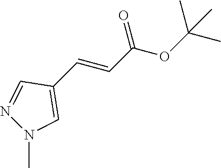

To a solution of tert-butyl 2-(diethoxyphosphoryl)acetate (12.60 g, 49.9 mmol) in tetrahydrofuran (THF) (30 mL) was added NaH 60% dispersion in mineral oil (2.179 g, 54.5 mmol) slowly. The mixture was stirred for 10 min at room temperature. A solution of 1-methyl-1H-pyrazole-4-carbaldehyde (5.0 g, 45.4 mmol) in tetrahydrofuran (THF) (30 mL) was added slowly. After addition, the reaction mixture was stirred for 20 min at room temperature. The LCMS showed a complete reaction. The mixture was diluted with water and extracted with ethyl acetate. The organic extract was washed with water and dried over anhydrous MgSO₄. It was filtered and the filtrate was concentrated to give the crude product which was then purified on the Combiflash column chromatography eluting with a gradient of 0 to 50% ethyl acetate in hexanes. The title compound was obtained as a clear colorless oil (9.0 g, 43.2 mmol, 95% yield). LC-MS m/z 208.9 (M+H)⁺, 0.93 min (ret. time).

### 30b) tert-Butyl (trans)-2-(1-methyl-1H-pyrazol-4-yl)cyclopropane-1-carboxylate

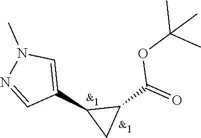 [SMILES: Cn1cc([C@@H]2C[C@H]2C(=O)OC(C)(C)C)cn1]

To a solution of trimethylsulfoxonium iodide (22.19 g, 101 mmol) in dimethyl sulfoxide (DMSO) (80 mL) was added NaH 60% dispersion in mineral oil (2.96 g, 73.9 mmol) at room temperature. The mixture was stirred for 1.0 hour at room temperature under N₂. A solution of tert-butyl (E)-3-(1-methyl-1H-pyrazol-4-yl)acrylate (7.0 g, 33.6 mmol) in tetrahydrofuran (THF) (80 mL) was then added slowly. After addition, the reaction mixture was stirred for 1.0 hour at room temperature, and then stirred for additional 1.0 hour at 50° C. The LCMS showed unreacted starting material. The reaction mixture was then stirred overnight at room temperature, after which the LCMS showed complete consumption of starting material. The mixture was diluted with brine/H₂O (1:1) and extracted with ethyl acetate. The organic extract was washed with brine and dried over anhydrous MgSO₄. It was filtered and the filtrate was concentrated to give the desired product as a clear colorless oil (6.3 g, 28.3 mmol, 84% yield). LC-MS m/z 222.9 (M+H)⁺, 0.94 min (ret. time).

### 30c) (trans)-2-(1-Methyl-1H-pyrazol-4-yl)cyclopropane-1-carboxylic acid

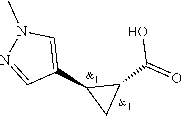

A mixture of tert-butyl (trans)-2-(1-methyl-1H-pyrazol-4-yl)cyclopropane-1-carboxylate (6.3 g, 28.3 mmol) and TFA (21.84 mL, 283 mmol) in dichloromethane (DCM) (20 mL) was stirred for 2.0 hours at room temperature, after which LCMS showed complete consumption of starting material. The mixture was concentrated in vacuo and the residue was diluted with water (40 mL) and extracted with ethyl acetate (40 mL). The aqueous layer was concentrated to give the desired product as clear colorless oil (3.76 g, 22.63 mmol, 80% yield). LC-MS m/z 166.8 (M+H)⁺, 0.53 min (ret. time).

### 30d) Methyl 3-(trans)-2-(1-methyl-1H-pyrazol-4-yl)cyclopropyl)-3-oxopropanoate

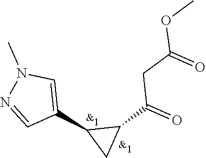 [SMILES: COC(=O)CC(=O)[C@@H]1C[C@H]1c1cnn(C)c1]

To a solution of (trans)-2-(1-methyl-1H-pyrazol-4-yl)cyclopropane-1-carboxylic acid (3.76 g, 22.63 mmol) in tetrahydrofuran (THF) (50 mL) was added CDI (5.50 g, 33.9 mmol). The mixture was stirred for 2 hours at room temperature. Methyl potassium malonate (10.60 g, 67.9 mmol) was added, followed by magnesium chloride (2.58 g, 27.2 mmol). The resulting reaction mixture was stirred overnight at room temperature. The mixture was neutralized with 6.0 N aqueous HCl, diluted with water, and extracted with ethyl acetate. The organic extract was washed with brine and dried over anhydrous magnesium sulfate. It was filtered and the filtrate was concentrated. The residue was dissolved in DCM and purified via CombiFlash column chromatography (80 g silica gel, solution load) eluting with a gradient of 0-5% methanol in dichloromethane. The title compound was obtained as clear light yellow oil (2.37 g, 10.66 mmol, 47.1% yield). LC-MS m/z 223.0 (M+H)⁺, 0.63 min (ret. time).

### 30e) Methyl 3-(dimethylamino)-2-(trans)-2-(1-methyl-1H-pyrazol-4-yl)cyclopropanecarbonyl)acrylate

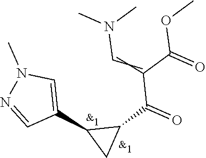 [SMILES: COC(=O)C(=CN(C)C)C(=O)[C@@H]1C[C@H]1c1cnn(C)c1]

A mixture of methyl 3-(trans)-2-(1-methyl-1H-pyrazol-4-yl)cyclopropyl)-3-oxopropanoate (2.35 g, 10.57 mmol) and N,N-dimethylformamide dimethylacetal (1.8 ml, 13.55 mmol) in 1,4-Dioxane (20 mL) was stirred for 18 hours at room temperature. The mixture was concentrated to give the title compound as light orange oil used in the next step without further purification (2.7 g, 9.74 mmol, 92% yield). LC-MS m/z 278.0 (M+H)⁺, 0.63 min (ret. time).

### 30f) 3-(4-(Methoxycarbonyl)-5-(trans)-2-(1-methyl-1H-pyrazol-4-yl)cyclopropyl)-1H-pyrazol-1-yl)benzoic acid

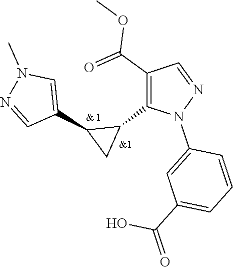 [SMILES: COC(=O)c1cnn(-c2cccc(C(=O)O)c2)c1[C@@H]1C[C@H]1c1cnn(C)c1]

To a solution of methyl 3-(dimethylamino)-2-(trans)-2-(1-methyl-1H-pyrazol-4-yl)cyclopropane-carbonyl)acrylate (1.0 g, 3.61 mmol) in ethanol (15 mL) was added 3-hydrazinylbenzoic acid hydrochloride (0.714 g, 3.79 mmol). To the resulting suspension was added TEA (1.256 mL, 9.01 mmol). The resulting clear solution was stirred for 1.0 hour at room temperature, after which LCMS showed a complete reaction. The mixture was concentrated and the residue was diluted with 30 mL aqueous 1.0 N HCl and extracted with ethyl acetate. The organic extract was concentrated to give the title compound as a yellow solid used in the next step without further purification (1.15 g, 3.14 mmol, 87% yield). LC-MS m/z 367.0 (M+H)⁺, 0.83 min (ret. time).

### 30g) Methyl 1-(3-(hydroxymethyl)phenyl)-5-(trans)-2-(1-methyl-1H-pyrazol-4-yl)cyclopropyl)-1H-pyrazole-4-carboxylate

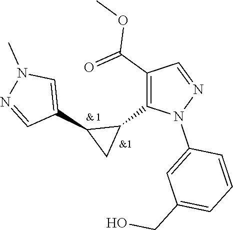 [SMILES: COC(=O)c1cnn(-c2cccc(CO)c2)c1[C@@H]1C[C@H]1c1cnn(C)c1]

To a solution of 3-(4-(methoxycarbonyl)-5-(trans)-2-(1-methyl-1H-pyrazol-4-yl)cyclopropyl)-1H-pyrazol-1-yl)benzoic acid (1.1 g, 3.00 mmol) in tetrahydrofuran (THF) (20 mL) was added CDI (1.461 g, 9.01 mmol). The mixture was stirred for 2 h at room temperature. The resulting solution was added slowly to sodium borohydride (0.568 g, 15.01 mmol) in water (5.0 mL). After addition, the mixture was stirred for 10 min at room temperature. The mixture was diluted with water and extracted with ethyl acetate. The organic extract was washed with water and dried over MgSO₄. It was filtered and the filtrate was concentrated. The crude product was purified via CombiFlash column chromatography eluting with a gradient of 100% hexanes to 70% EtOH/EtOAc (1:3, V:V). The title compound was obtained as a light brown oil (0.95 g, 2.70 mmol, 90% yield). LC-MS m/z 353.0 (M+H)⁺, 0.78 min (ret. time).

### 30h) Methyl 1-(3-(((S)-4-methyl-1,1-dioxido-7-(trifluoromethyl)-4,5-dihydrobenzo[f][1,2]thiazepin-2(3H)-yl)methyl)phenyl)-5-(trans-2-(1-methyl-1H-pyrazol-4-yl)cyclopropyl)-1H-pyrazole-4-carboxylate

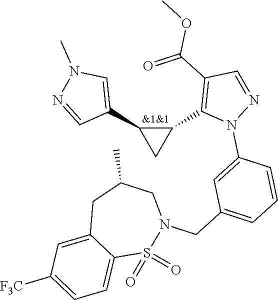 [SMILES: COC(=O)c1cnn(-c2cccc(CN3C[C@@H](C)Cc4cc(C(F)(F)F)ccc4S3(=O)=O)c2)c1[C@@H]1C[C@H]1c1cnn(C)c1]

To a solution of methyl 1-(3-(hydroxymethyl)phenyl)-5-(trans)-2-(1-methyl-1H-pyrazol-4-yl)cyclopropyl)-1H-pyrazole-4-carboxylate (60 mg, 0.170 mmol) and (S)-4-methyl-7-(trifluoromethyl)-2,3,4,5-tetrahydrobenzo[f][1,2]thiazepine 1,1-dioxide (52.3 mg, 0.187 mmol) in tetrahydrofuran (THF) (1.2 mL) was added trimethylphosphine (0.341 mL, 0.341 mmol), followed by DIAD (0.066 mL, 0.341 mmol). The reaction mixture was stirred for 4 h at room temperature, after which LCMS showed complete consumption of starting material. The mixture was diluted with water and extracted with ethyl acetate. The organic extract was dried over anhydrous magnesium sulfate. It was filtered and the filtrate was concentrated. The crude product was purified via CombiFlash column chromatography eluting with a gradient of 0-60% ethyl acetate in hexanes. The title compound was obtained as colorless wax (55 mg, 0.09 mmol, 52.6% yield). LC-MS m/z 614.3 (M+H)⁺, 1.27 min (ret. time).

### 30i) 1-(3-(((S)-4-Methyl-1,1-dioxido-7-(trifluoromethyl)-4,5-dihydrobenzo[f][1,2]thiazepin-2(3H)-yl)methyl)phenyl)-5-(trans-2-(1-methyl-1H-pyrazol-4-yl)cyclopropyl)-1H-pyrazole-4-carboxylic acid

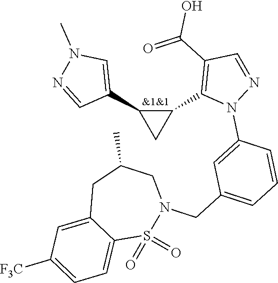 [SMILES: C[C@H]1Cc2cc(C(F)(F)F)ccc2S(=O)(=O)N(Cc2cccc(-n3ncc(C(=O)O)c3[C@@H]3C[C@H]3c3cnn(C)c3)c2)C1]

A mixture of methyl 1-(3-(((S)-4-methyl-1,1-dioxido-7-(trifluoromethyl)-4,5-dihydrobenzo[f][1,2]thiazepin-2(3H)-yl)methyl)phenyl)-5-(trans)-2-(1-methyl-1H-pyrazol-4-yl)cyclopropyl)-1H-pyrazole-4-carboxylate (55 mg, 0.090 mmol) and 6.0 N aqueous NaOH (0.3 mL, 1.800 mmol) in tetrahydrofuran (THF) (1.0 mL) and methanol (1.0 mL) was stirred for 3 days at room temperature, after which LCMS showed complete consumption of starting material. The mixture was concentrated and the residue was diluted with water (3 mL) and neutralized with 2.0 N aqueous HCl. The resulting precipitate was extracted with ethyl acetate. The organic extract was concentrated and the crude product was purified on the prep HPLC eluting with a gradient of 20-100% acetonitrile in water. The title compound was obtained as a white solid (41.5 mg, 0.066 mmol, 73.4% yield). LC-MS m/z 600.3 (M+H)⁺, 1.18 min (ret. time). ¹H NMR (400 MHz, DMSO-d₆) δ ppm 0.93 (br. s., 3H) 1.03 (m, 1H) 1.15 (m, 1H) 1.76-1.91 (m, 1H) 2.08 (m, 1H) 3.08 (m, 2H) 3.37 (m, 2H) 3.62 (m, 1H) 3.69 (s, 3H) 3.93 (d, J=10.54 Hz, 1H) 4.18 (dd, J=15.31, 6.27 Hz, 1H) 7.01 (d, J=7.28 Hz, 1H) 7.26 (d, J=15.06 Hz, 1H) 7.44 (br. s., 1H) 7.48 (br. s., 1H) 7.51 (d, J=4.77 Hz, 2H) 7.85 (d, J=7.78 Hz, 1H) 7.94 (s, 1H) 7.97 (s, 1H) 8.06 (t, J=6.40 Hz, 1H) 12.39 (br. s., 1H).

Examples in Table 8 were prepared in an analogous manner:

TABLE 8

| | | | | Retention | |
|---|---|---|---|---|---|
| | | | LCMS | Time | |
| Ex # | Structure | Name | [M + H]⁺ | (min) | ¹HNMR |
| Example 31 | 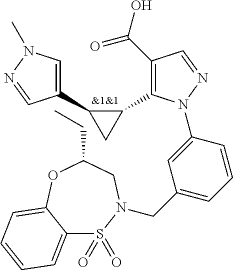 | 1-(3-(((R)-4-ethyl-1,1- dioxido-3,4-dihydro- 2H- benzo[b][1,4,5] oxathiazepin- 2-yl)methyl)-phenyl)- 5-(trans-2-(1-methyl- 1H-pyrazol-4- yl)cyclopropyl)-1H- pyrazole- 4-carboxylic acid | 548.3 | 1.02 | ¹H NMR (400 MHz, DMSO- d6) δ ppm 1.07 (m, 3H) 1.0- 1.2 (m, 2 H) 1.47-1.69 (m, 2 H) 1.86 (m, 1 H) 2.04-2.15 (m, 1 H) 3.11 (dd, J = 15.06, 7.28 Hz, 1 H) 3.70 (s, 3 H) 3.72-3.84 (m, 1 H) 3.93- 4.08 (m, 2 H) 4.32 (d, J = 15.06 Hz, 1 H) 7.03 (s, 1 H) 7.23-7.32 (m, 2 H) 7.36 (t, |
| | | | | | J = 7.65 Hz, 1 H) |
| | | | | | 7.48 (br. s., 1 H) |
| | | | | | 7.53 (br. s., 3 H) |
| | | | | | 7.65 (t, J = 7.65 |
| | | | | | Hz, 1 H) 7.79 |
| | | | | | (d, J = 6.27 Hz, 1 |
| | | | | | H) 7.97 (s, 1 H) |
| | | | | | 12.40 (s, 1 H) |
| Example 32 | 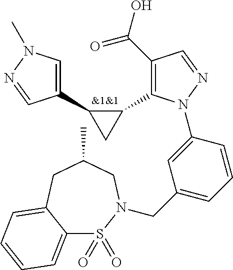 | 1-(3-(((S)-4-methyl-1,1- dioxido-4,5- dihydrobenzo[f][1,2] thiazepin-2(3H)-yl)methyl)- phenyl)-5-(trans-2-(1- methyl-1H-pyrazol-4- yl)cyclopropyl)- 1H-pyrazole-4- carboxylic acid | 532.3 | 1.03 | ¹H NMR (400 MHz, DMSO-d6) δ ppm 0.92 (br. s., 3 H) 1.04 (m, 1 H) 1.15 (m, 1 H) 1.75- 1.93 (m, 1 H) 1.98- 2.16 (m, 2 H) 2.75-3.07 (m, 2 H) 3.30- 3.40 (m, 1 H) 3.59 (m, 1 H) 3.85 (m, 1 H) 4.14 (dd, J = 14.68, 5.90 |
| | | | | | Hz, 1 H) 7.01 |
| | | | | | (d, J = 7.28 |
| | | | | | Hz, 1 H) 7.26 |
| | | | | | (d, J = 17.82 |
| | | | | | Hz, 1 H) 7.42- |
| | | | | | 7.54 (m, 6 |
| | | | | | H) 7.55- |
| | | | | | 7.62 (m, 1 H) |
| | | | | | 7.86 (m, 1 H) |
| | | | | | 7.97 (s, 1 H) |
| | | | | | 12.40 (br. s., |
| | | | | | 1 H) |
| Example 33 | 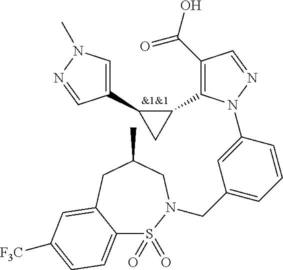 | 1-(3-(((R)-4-methyl-1,1- dioxido-7- (trifluoromethyl)-4,5- dihydro- benzo[f][1,2]thiazepin- 2(3H)-yl)methyl)phenyl)- 5-(trans-2-(1-methyl-1H- pyrazol-4-yl)cyclopropyl)- 1H-pyrazole-4-carboxylic acid | 600.3 | 1.13 | ¹H NMR (400 MHz, DMSO-d6) δ ppm 0.93 (br. s., 3 H) 1.04 (m, 1 H) 1.15 (m, 1 H) 1.73- 1.93 (m, 1 H) 2.08 (m, 2 H) 3.08 (m, 2 H) 3.4 (m, 1H) 3.62 (m, 1 H) 3.70 (br. s., 3 H) 3.93 (m, 1 H) 4.18 (dd, J = 15.06, 5.77 Hz, 1 H) |
| | | | | | 7.01 (d, |
| | | | | | J = 7.28 Hz, 1 |
| | | | | | H) 7.26 (d, |
| | | | | | J = 14.81 Hz, |
| | | | | | 1 H) 7.41- |
| | | | | | 7.58 (m, 4 H) |
| | | | | | 7.85 (d, |
| | | | | | J = 7.78 Hz, 1 |
| | | | | | H) 7.94 (s, 1 |
| | | | | | H) 7.97 (s, |
| | | | | | 1H) 8.03- |
| | | | | | 8.11 (m, 1 H) |
| | | | | | 12.39 (br. s., |
| | | | | | 1 H) |

### Example 34: 1-(3-((N-hexyl-[1,1′-biphenyl]-3-ylsulfonamido)methyl)phenyl)-5-((trans)-2-(1-methyl-1H-1,2,3-triazol-4-yl)cyclopropyl)-1H-pyrazole-4-carboxylic acid

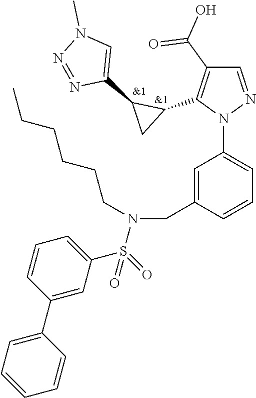 [SMILES: CCCCCCN(Cc1cccc(-n2ncc(C(=O)O)c2[C@@H]2C[C@H]2c2cn(C)nn2)c1)S(=O)(=O)c1cccc(-c2ccccc2)c1]

Anhydrous CH₃CN (1143 μl) was added to a mixture of [1,1′-biphenyl]-3-sulfonyl chloride (69.3 mg, 0.274 mmol) and hexan-1-amine (36.2 μl, 0.274 mmol), DIPEA (120 μl, 0.686 mmol) was then added to obtain a clear yellow solution. After stirring one minute, to the reaction was added NaH, 60 wt % in oil (36.6 mg, 0.914 mmol) followed by methyl 1-(3-(chloromethyl)phenyl)-5-((trans)-2-(1-methyl-1H-1,2,3-triazol-4-yl)cyclopropyl)-1H-pyrazole-4-carboxylate (85 mg, 0.229 mmol) to form a white slurry. The reaction was then heated in a microwave reactor to 140 C for 1 min. KOH 0.5 M aqueous (1372 μl, 0.686 mmol) was then added to the reaction, then the reaction heated in a microwave reactor to 140 C for 1 min, to obtain a homogeneous yellow solution which was then directly injected onto reverse-phase HPLC and purified, to obtain after evaporation 1-(3-((N-hexyl-[1,1′-biphenyl]-3-ylsulfonamido)methyl)phenyl)-5-((trans)-2-(1-methyl-1H-1,2,3-triazol-4-yl)cyclopropyl)-1H-pyrazole-4-carboxylic acid (47.4 mg, 32%). LC-MS m/z 639.2 (M+H)⁺, 1.28 (ret. time), acidic method. ¹H NMR (400 MHz, DMSO-d6): δ ppm 12.35 (br. s., 1H), 7.94-8.05 (m, 3H), 7.85 (s, 1H), 7.73 (m, 3H), 7.62 (s, 1H), 7.40-7.55 (m, 7H), 4.38 (m, 2H), 3.93 (s, 3H), 3.12 (m, 2H), 2.41 (m, 1H), 2.16 (m, 1H), 1.19-1.36 (m, 3H), 0.95-1.18 (m, 7H), 0.71 (t, J=6.65 Hz, 3H).

The examples in Table 9 were prepared in an analogous manner:

TABLE 9

| | | | | Retention | |
|---|---|---|---|---|---|
| | | | LCMS | Time | |
| Ex # | Structure | Name | [M + H]⁺ | (min) | ¹HNMR |
| Example 35 | 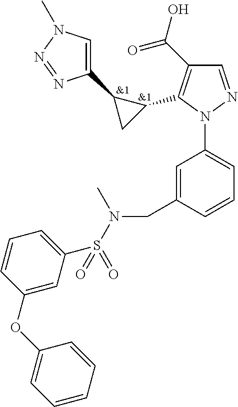 | 5-((trans)-2-(1-methyl- 1H-1,2,3-triazol-4- yl)cyclo-propyl)-1-(3-((N- methyl-3-phenoxy- phenyl-sulfonamido)- methyl)phenyl)-1H- pyrazole-4-carboxylic acid | 585.1 | 1.05 | ¹H NMR (400 MHz, DMSO-d6): δ ppm 12.37 (br. s., 1H), 7.98 (s, 1H), 7.63-7.71 (m, 1H), 7.56- 7.63 (m, 2H), 7.30-7.54 (m, 8H), 7.19- 7.27 (m, 1H), 7.12 (d. J = 7.78 Hz, 2H), 4.13 (m, 2H), 3.93 (s, 3H), 2.54 (s, 3H), 2.43 (m, 1H), 2.15 (m, 1H), 1.33 (m, 1H). 1.21 (m, 1H) |
| Example 36 | 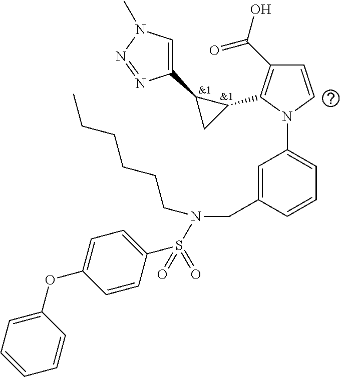 | 1-(3-((N-hexyl-4- phenoxyphenylsulfonamido) methyl)phenyl)-5- ((trans)-2-(1-methyl-1H- 1,2,3-triazol-4-yl)- cyclopropyl)-1H- pyrazole-4-carboxylic acid | 655.2 | 1.29 | ¹H NMR (400 MHz, DMSO-d6): δ ppm 12.37 (br. s., 1H), 7.98 (s, 1H), 7.56-7.68 (m, 3H), 7.36- 7.53 (m, 6H), 7.27-7.36 (m, 2H), 7.18- 7.26 (m, 1H), 7.09 (d, J = 8.03 Hz, 2H), 4.31 (m, 2H), 3.94 (s, 3H), 3.04 (t, J = 7.28 Hz, 2H), 2.41 (m, 1H), 2.18 (m, 1H), 1.32 (m, 1H), 0.96- 1.28 (m, 9H), 0.74 (t, |
| | | | | | J = 6.90 Hz, |
| | | | | | 3H) |
| Example 37 | 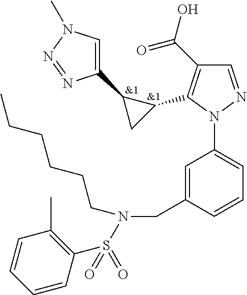 | 1-(3-((N-hexyl-2- methylphenylsulfonamido) methyl)phenyl)-5- ((trans)-2-(1-methyl-1H- 1,2,3-triazol-4-yl)cyclo- propyl)-1H-pyrazole-4- carboxylic acid | 577.2 | 1.21 | ¹H NMR (400 MHz, DMSO-d6): δ ppm 7.99 (s, 1H), 7.85 (d, J = 7.78 Hz, 1H), 7.62 (s, 1H), 7.33- 7.59 (m, 7H), 4.45 (m, 2H), 3.94 (s, 3H), 3.07 (t, J = 7.28 Hz, 2H), 2.55 (s, 3H), 2.41 (m, 1H), 2.17 (m, 1H), 0.92- 1.39 (m, |
| | | | | | 10H), 0.73 (t, |
| | | | | | J = 7.03 Hz, |
| | | | | | 3H) |
| Example 38 | 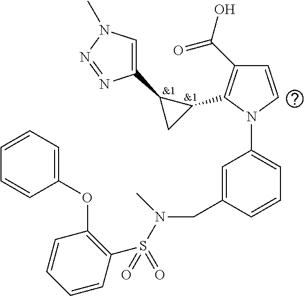 | 5-((trans)-2-(1-methyl- 1H-1,2,3-triazol-4- yl)cyclo-propyl)-1-(3-((N- methyl-2-phenoxyphenyl- sulfonamido)- methyl)phenyl)-1H- pyrazole-4-carboxylic acid | 585.1 | 1.00 | ¹H NMR (400 MHz, DMSO-d6): δ ppm 7.91- 8.02 (m, 2H), 7.56-7.66 (m, 2H), 7.35- 7.53 (m, 6H), 7.31 (t, J = 7.53 Hz, 1H), 7.19- 7.26 (m, 1H), 7.08 (d, J = 8.03 Hz, 2H), 6.96 (d, J = 8.28 Hz, 1H), 4.33 (m, 2H), 3.91 (s, 3H), 2.69 (s, |
| | | | | | 3H), 2.41 (m, |
| | | | | | 1H), 2.16 (m, |
| | | | | | 1H), 1.33 (m, |
| | | | | | 1H), 1.21 (m, |
| | | | | | 1H) |
| Example 39 | 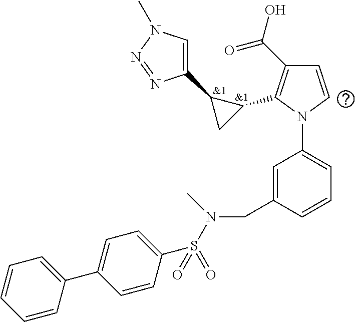 | 5-((trans)-2-(1-methyl- 1H-1,2,3-triazol-4- yl)cyclo-propyl)-1-(3-((N- methyl-[1,1′-biphenyl]-4- ylsulfonamido)- methyl)phenyl)-1H- pyrazole-4-carboxylic acid | 569.0 | 1.03 | ¹H NMR (400 MHz, DMSO-d6): δ ppm 7.89- 8.01 (m, 5H), 7.77 (m, 2H), 7.62 (s, 1H), 7.38-7.57 (m, 7H), 4.18 (m, 2H), 3.95 (s, 3H), 2.59 (s, 3H), 2.43 (m, 1H), 2.15 (m, 1H), 1.33 (m, 1H). 1.20 (m, 1H) |
| Example 40 | 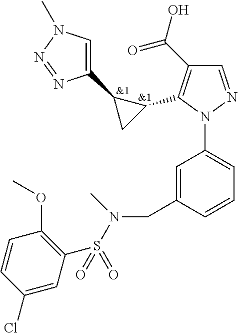 | 1-(3-((5-chloro-2- methoxy-N- methylphenyl- sulfonamido)- methyl)phenyl)-5- ((trans)-2-(1-methyl-1H- 1,2,3-triazol-4-yl)cyclo- propyl)-1H-pyrazole-4- carboxylic acid | 577.0 | 0.90 | ¹H NMR (400 MHz, DMSO-d6): δ ppm 7.99 (s, 1H), 7.75 (s, 1H), 7.69 (d, J = 8.78 Hz, 1H), 7.61 (s, 1H), 7.44- 7.53 (m, 2H), 7.34-7.43 (m, 2H), 7.30 (d, J = 8.53 Hz, 1H), 4.30 (d, J = 4.77 Hz, 2H), 3.93 (d, J = 7.53 Hz, 6H), 2.64 (s, 3H), 2.38- 2.46 (m, 1H), 2.10-2.20 (m, |
| | | | | | 1H), 1.30- |
| | | | | | 1.40 (m, 1H), |
| | | | | | 1.17-1.29 (m, |
| | | | | | 1H) |
| Example 41 | 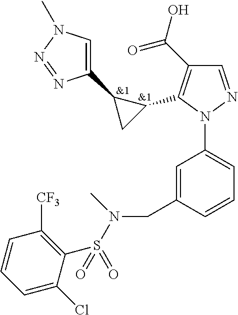 | 1-(3-((2-chloro-N-methyl- 6-(trifluoro- methyl)phenyl- sulfonamido)- methyl)phenyl)-5- ((trans)-2-(1-methyl-1H- 1,2,3-triazol-4-yl)cyclo- propyl)-1H-pyrazole-4- carboxylic acid | 595.0 | 0.95 | ¹H NMR (400 MHz, DMSO-d6): δ ppm 8.05 (d, J = 8.03 Hz, 2H), 8.00 (s, 1H), 7.80- 7.90 (m, 1H), 7.60 (s, 1H), 7.44-7.55 (m, 3H), 7.40 (d, J = 6.78 Hz, 1H), 4.47 (m, 2H), 3.94 (s, 3H), 2.66 (s, 3H), 2.39- 2.47 (m, 1H), 2.12-2.21 (m, 1H), 1.28- 1.41 (m, 1H), |
| | | | | | 1.16-1.28 (m, |
| | | | | | 1H) |
| Example 42 | 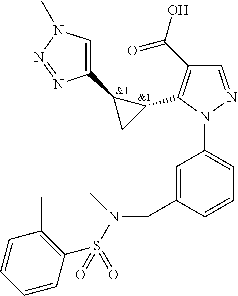 | 1-(3-((N,2- dimethylphenylsulfonamido) methyl)phenyl)-5- ((trans)-2-(1-methyl-1H- 1,2,3-triazol-4- yl)cyclopropyl)-1H- pyrazole-4-carboxylic acid | 507.0 | 0.87 | ¹H NMR (400 MHz, DMSO-d6): δ ppm 7.99 (s, 1H), 7.84 (d, J = 7.53 Hz, 1H), 7.34- 7.67 (m, 8H), 4.33 (m, 2H), 3.93 (s, 3H), 2.63 (s, 3H), 2.58 (s, 3H), 2.43 (m, 1H), 2.18 (m, 1H), 1.35 (m, 1H), 1.23 (m, 1H) |
| Example 43 | 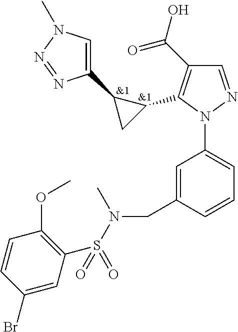 | 1-(3-((5-bromo-2- methoxy-N- methylphenylsulfonamido) methyl)phenyl)-5- ((trans)-2-(1-methyl- 1H-1,2,3-triazol-4- yl)cyclopropyl)-1H- pyrazole-4-carboxylic acid | 601.0 | 0.91 | ¹H NMR (400 MHz, DMSO-d6): δ ppm 7.99 (s, 1H), 7.87 (s, 1H), 7.81 (d, J = 8.78 Hz, 1H), 7.61 (s, 1H), 7.44-7.54 (m, 2H), 7.33-7.43 (m, 2H), 7.25 (d, J = 8.78 Hz, 1H), 4.21- 4.41 (m, 2H), 3.94 (s, 3H), 3.92 (s, 3H), 2.64 (s, 3H), 2.38- |
| | | | | | 2.47 (m, |
| | | | | | 1H), 2.10- |
| | | | | | 2.21 (m, |
| | | | | | 1H), 1.29- |
| | | | | | 1.44 (m, |
| | | | | | 1H), 1.11- |
| | | | | | 1.28 (m, 1H) |
| Example 44 | 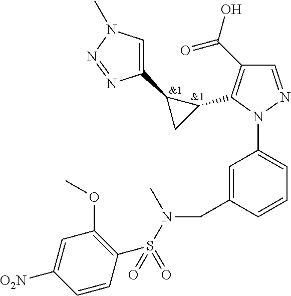 | 1-(3-((2-methoxy-N- methyl-4- nitrophenylsulfonamido) methyl)phenyl)-5- ((trans)-2-(1-methyl- 1H-1,2,3-triazol-4- yl)cyclopropyl)-1H- pyrazole-4-carboxylic acid | 568.0 | 0.84 | ¹H NMR (400 MHz, DMSO-d6): δ ppm 8.08 (d, J = 8.28 Hz, 1H), 7.89-8.01 (m, 3H), 7.63 (s, 1H), 7.45-7.54 (m, 2H), 7.33-7.41 (m, 2H), 4.34 (m, 2H), 4.05 (s, 3H), 3.95 (s, 3H), 2.70 (s, 3H), 2.35- 2.46 (m, |
| | | | | | 1H), 2.11- |
| | | | | | 2.21 (m, |
| | | | | | 1H), 1.29- |
| | | | | | 1.40 (m, |
| | | | | | 1H), 1.18- |
| | | | | | 1.28 (m, 1H) |
| Example 45 | 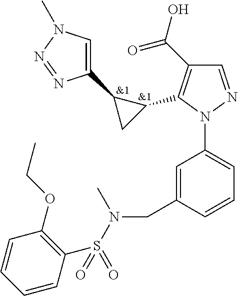 | 1-(3-((2-ethoxy-N- methylphenylsulfonamido) methyl)phenyl)-5- ((trans)-2-(1-methyl- 1H-1,2,3-triazol-4- yl)cyclopropyl)-1H- pyrazole-4-carboxylic acid | 537.1 | 0.86 | ¹H NMR (400 MHz, DMSO-d6): δ ppm 12.39 (m, 1H), 8.00 (s, 1H), 7.83 (d, J = 7.78 Hz, 1H), 7.57- 7.67 (m, 2H), 7.44- 7.52 (m, 2H), 7.35- 7.44 (m, 2H), 7.24 (d, J = 8.28 Hz, 1H), 7.10 (t, J = 7.53 Hz, 1H), 4.32 |
| | | | | | (m, 2H), |
| | | | | | 4.20 (m, |
| | | | | | 2H), 3.94 (s, |
| | | | | | 3H), 2.64 (s, |
| | | | | | 3H), 2.36- |
| | | | | | 2.47 (m, |
| | | | | | 1H), 2.12- |
| | | | | | 2.24 (m, |
| | | | | | 1H), 1.28- |
| | | | | | 1.44 (m, |
| | | | | | 4H), 1.18- |
| | | | | | 1.28 (m, 1H) |
| Example 46 | 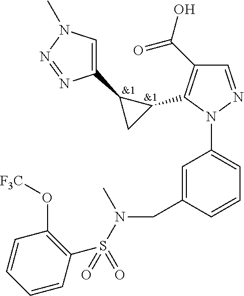 | 5-((trans)-2-(1-methyl- 1H-1,2,3-triazol-4- yl)cyclopropyl)-1-(3- ((N-methyl-2- (trifluoromethoxy) phenylsulfonamido) methyl)phenyl)-1H-pyrazole- 4-carboxylic acid | 577.1 | 0.95 | ¹H NMR (400 MHz, DMSO-d6): δ ppm 12.37 (br. s., 1H), 7.97-8.06 (m, 2H), 7.80-7.88 (m, 1H), 7.59-7.69 (m, 3H), 7.36-7.56 (m, 4H), 4.25-4.38 (m, 2H), 3.94 (s, 3H), 2.68 (s, 3H), 2.38-2.48 (m, 1H), |
| | | | | | 2.13-2.23 |
| | | | | | (m, 1H), |
| | | | | | 1.35 (m, |
| | | | | | 1H), 1.13- |
| | | | | | 1.27 (m, 1H) |
| Example 47 | 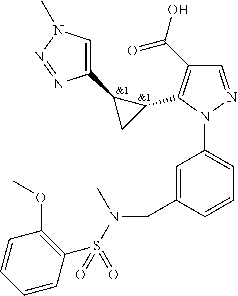 | 1-(3-((2-methoxy-N- methylphenylsulfonamido) methyl)phenyl)-5- ((trans)-2-(1-methyl- 1H-1,2,3-triazol-4- yl)cyclopropyl)-1H- pyrazole-4-carboxylic acid | 523.0 | 0.80 | ¹H NMR (400 MHz, DMSO-d6): δ ppm 7.99 (s, 1H), 7.81 (d, J = 7.53 Hz, 1H), 7.55-7.68 (m, 2H), 7.34-7.53 (m, 4H), 7.26 (d, J = 8.28 Hz, 1H), 7.11 (m, 1H), 4.27 (m, 2H), 3.93 (s, 3H), 3.91 (s, 3H), 2.61 (s, |
| | | | | | 3H), 2.42 |
| | | | | | (m, 1H), |
| | | | | | 2.16 (m, |
| | | | | | 1H), 1.34 |
| | | | | | (m, 1H), |
| | | | | | 1.24 (m, 1H) |
| Example 48 | 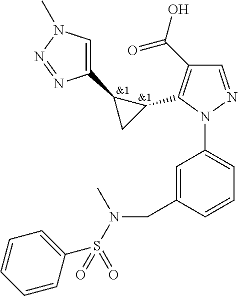 | 5-((trans)-2-(1-methyl- 1H-1,2,3-triazol-4- yl)cyclopropyl)-1-(3- ((N- methylphenylsulfonamido) methyl)phenyl)-1H- pyrazole-4-carboxylic acid | 493.0 | 0.81 | ¹H NMR (400 MHz, DMSO-d6): δ ppm 7.99 (s, 1H), 7.85 (d, J = 7.28 Hz, 2H), 7.59-7.77 (m, 4H), 7.36-7.54 (m, 4H), 4.05-4.19 (m, 2H), 3.94 (s, 3H), 2.54 (s, 3H), 2.38-2.47 (m, 1H), 2.10-2.20 |
| | | | | | (m, 1H), |
| | | | | | 1.33 (m, |
| | | | | | 1H), 1.16- |
| | | | | | 1.26 (m, 1H) |
| Example 49 | 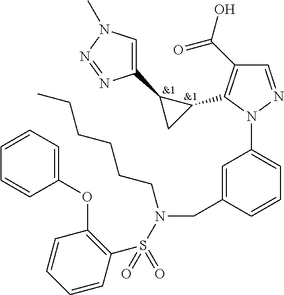 | 1-(3-((N-hexyl-2- phenoxyphenylsulfonamido) methyl)phenyl)- 5-((trans)-2-(1-methyl- 1H-1,2,3-triazol-4- yl)cyclopropyl)-1H- pyrazole-4-carboxylic acid | 655.2 | 1.26 | ¹H NMR (400 MHz, DMSO-d6): δ ppm 7.91- 8.00 (m, 2H), 7.55- 7.63 (m. 2H), 7.36- 7.52 (m, 6H), 7.19- 7.32 (m. 2H), 7.05 (d, J = 7.78 Hz, 2H), 6.93 (d, J = 8.28 Hz, 1H), 4.47 (m, 2H), 3.92 (s, 3H), 3.15 (t, |
| | | | | | J = 7.28 Hz, |
| | | | | | 2H), 2.39 |
| | | | | | (m, 1H), |
| | | | | | 2.17 (m, |
| | | | | | 1H), 1.19- |
| | | | | | 1.36 (m, |
| | | | | | 3H), 0.92- |
| | | | | | 1.19 (m, |
| | | | | | 7H), 0.72 (t, |
| | | | | | J = 6.90 Hz, |
| | | | | | 3H) |
| Example 50 | 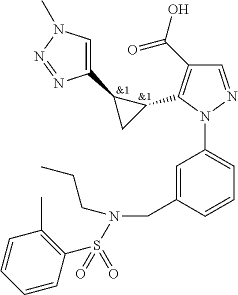 | 5-((trans)-2-(1-methyl- 1H-1,2,3-triazol-4- yl)cyclopropyl)-1-(3- ((2-methyl-N- propylphenylsulfonamido) methyl)phenyl)-1H- pyrazole-4-carboxylic acid | 535.1 | 1.01 | ¹H NMR (400 MHz, DMSO-d6): δ ppm 7.99 (s, 1H), 7.84 (d, J = 7.78 Hz, 1H), 7.62 (s, 1H), 7.32-7.57 (m, 7H), 4.32-4.54 (m, 2H), 3.94 (s, 3H), 3.06 (t, J = 7.40 Hz, 2H), 2.54 (s, 3H), 2.36- 2.45 (m, 1H), 2.10- |
| | | | | | 2.22 (m, |
| | | | | | 1H), 1.23- |
| | | | | | 1.40 (m, |
| | | | | | 3H), 1.10- |
| | | | | | 1.23 (m, |
| | | | | | 1H), 0.61 (t, |
| | | | | | J = 7.28 Hz, |
| | | | | | 3H) |
| Example 51 | 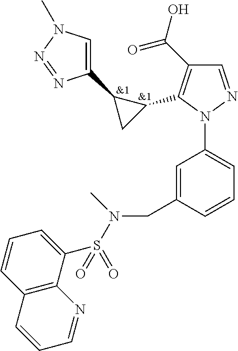 | 5-((trans)-2-(1-methyl- 1H-1,2,3-triazol-4- yl)cyclopropyl)-1-(3- ((N-methylquinoline-8- sulfonamido)methyl) phenyl)-1H-pyrazole-4- carboxylic acid | 544.1 | 0.79 | ¹H NMR (400 MHz, DMSO-d6): δ ppm 9.06- 9.17 (m, 1H), 8.54 (d, J = 8.28 Hz, 1H), 8.42 (d, J = 7.28 Hz, 1H), 8.31 (d, J = 8.03 Hz, 1H), 7.98 (s, 1H), 7.77 (t, J = 7.65 Hz, 1H), 7.71 (dd, J = 3.76, 7.78 Hz, 1H), 7.59 (s, 1H), 7.34- 7.51 (m, 4H), 4.50 (m, 2H), |
| | | | | | 3.92 (s, 3H), |
| | | | | | 2.72 (s, 3H), |
| | | | | | 2.40 (m, |
| | | | | | 1H), 2.14 |
| | | | | | (m, 1H), |
| | | | | | 1.34 (m, |
| | | | | | 1H), 1.21 |
| | | | | | (m, 1H) |
| Example 52 | 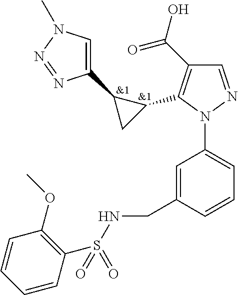 | 1-(3-((2- methoxyphenylsulfonamido) methyl)phenyl)- 5-((trans)-2-(1-methyl- 1H-1,2,3-triazol-4- yl)cyclopropyl)-1H- pyrazole-4-carboxylic acid | 509.0 | 0.71 | ¹H NMR (400 MHz, DMSO-d6): δ ppm 7.98 (s, 1H), 7.83-7.92 (m, 1H), 7.67-7.74 (m, 1H), 7.60 (s, 1H), 7.47-7.55 (m, 1H), 7.36 (s, 4H), 7.05-7.10 (m, 1H), 6.96-7.04 (m, 1H), 4.03 (m, 2H), 3.94 (s, |
| | | | | | 3H), 3.80 (s, |
| | | | | | 3H), 2.37 |
| | | | | | (m, 1H), |
| | | | | | 2.15 (m, |
| | | | | | 1H), 1.34 |
| | | | | | (m, 1H), |
| | | | | | 1.20 (m, 1H) |
| Example 53 | 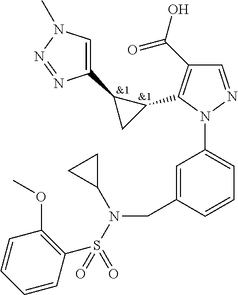 | 1-(3-((N-cyclopropyl-2- methoxyphenylsulfonamido) methyl)phenyl)- 5-((trans)-2-(1-methyl- 1H-1,2,3-triazol-4- yl)cyclopropyl)-1H- pyrazole-4-carboxylic acid | 549.1 | 0.87 | ¹H NMR (400 MHz, DMSO-d6): δ ppm 12.38 (br. s., 1H), 8.00 (s, 1H), 7.84 (d, J = 7.78 Hz, 1H), 7.57- 7.69 (m, 2H), 7.39- 7.51 (m, 4H), 7.26 (d, J = 8.28 Hz, 1H), 7.13 (t, J = 7.65 Hz, 1H), 4.50 (m, 2H), |
| | | | | | 3.94 (m, |
| | | | | | 3H), 2.43 |
| | | | | | (m, 1H), |
| | | | | | 2.19 (m, |
| | | | | | 2H), 1.36 |
| | | | | | (m, 1H), |
| | | | | | 1.22 (m, |
| | | | | | 1H), 0.42 |
| | | | | | (m, 4H) |

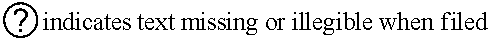

### Example 54: 1-(3-(((5-bromo-2-ethoxybenzyl)(methyl)amino)methyl)phenyl)-5-((trans)-2-(1-methyl-1H-1,2,3-triazol-4-yl)cyclopropyl)-1H-pyrazole-4-carboxylic acid trifluoroacetate

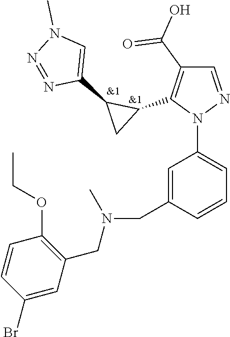 [SMILES: CCOc1ccc(Br)cc1CN(C)Cc1cccc(-n2ncc(C(=O)O)c2[C@@H]2C[C@H]2c2cn(C)nn2)c1]

Ethanol (800 μl) was added to a mixture of methyl 1-(3-(chloromethyl)phenyl)-5-((trans)-2-(1-methyl-1H-1,2,3-triazol-4-yl)cyclopropyl)-1H-pyrazole-4-carboxylate (65 mg, 0.175 mmol), 1-(5-bromo-2-ethoxyphenyl)-N-methylmethanamine, hydrochloride (98 mg, 0.350 mmol), and DIPEA (153 μl, 0.874 mmol). Reaction was then heated in a microwave reactor to 160 C for 1 min. NaOH 2 M aqueous (787 μl, 1.573 mmol) was then added to the reaction and the reaction was heated in a microwave reactor to 130 C for 1 min. The reaction was then directly injected onto reverse-phase HPLC to purify, to obtain after evaporation 1-(3-(((5-bromo-2-ethoxybenzyl)(methyl)amino)methyl)phenyl)-5-((trans)-2-(1-methyl-1H-1,2,3-triazol-4-yl)cyclopropyl)-1H-pyrazole-4-carboxylic acid trifluoroacetate (79.6 mg, 81%). LC-MS m/z 565.0 (M+H)⁺, 0.72 (ret. time), acidic method. ¹H NMR (400 MHz, DMSO-d6): δ ppm 9.65 (br. s., 1H), 8.02 (s, 1H), 7.81 (br. s., 1H), 7.51-7.76 (m, 6H), 7.05 (d, J=8.78 Hz, 1H), 3.99-4.60 (m, 6H), 2.41-2.73 (m, 7H), 2.18 (br. s., 1H), 1.31-1.42 (m, 1H), 1.12-1.30 (m, 4H).

The following example in Table 10 was prepared in an analogous manner:

TABLE 10

| | | | | Retention | |
|---|---|---|---|---|---|
| | | | LCMS | Time | |
| Ex # | Structure | Name | [M + H]⁺ | (min) | ¹HNMR |
| Example 55 | 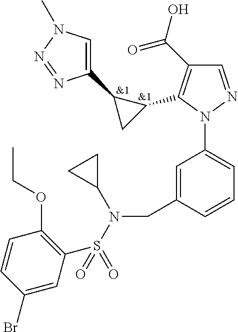 | 1-(3-(((5-bromo-2- ethoxybenzyl)cyclo- propyl)amino)methyl) phenyl)-5-((trans)-2-(1- methyl-1H-1,2,3- triazol-4- yl)cyclopropyl)-1H- pyrazole-4-carboxylic acid trifluoroacetate | 591.0 | 0.78 | ¹H NMR (400 MHz, DMSO- d6): δ ppm 9.26 (br. s., 1H), 8.00 (s, 1H), 7.16-7.91 (m, 7H), 7.00 (m, 1H), 4.40 (m, 3H), 3.89-4.10 (m, 6H), 2.47 (m, 1H), 2.20 (m, 1H), 1.35 (m, 1H), 1.17-1.28 (m, 5H), 0.51 (m, 4H) |

### Example 56. 1-(3-((N-Hexyl-2-methylphenylsulfonamido)methyl)phenyl)-5-(trans)-2-(1-methyl-1H-pyrazol-4-yl)cyclopropyl)-1H-pyrazole-4-carboxylic acid

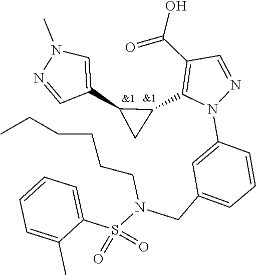 [SMILES: CCCCCCN(Cc1cccc(-n2ncc(C(=O)O)c2[C@@H]2C[C@H]2c2cnn(C)c2)c1)S(=O)(=O)c1ccccc1C]

### 56a) Methyl 1-(3-(chloromethyl)phenyl)-5-(trans)-2-(1-methyl-1H-pyrazol-4-yl)cyclopropyl)-1H-pyrazole-4-carboxylate

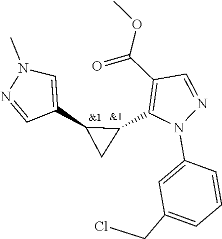 [SMILES: COC(=O)c1cnn(-c2cccc(CCl)c2)c1[C@@H]1C[C@H]1c1cnn(C)c1]

To a solution of methyl 1-(3-(hydroxymethyl)phenyl)-5-(trans)-2-(1-methyl-1H-pyrazol-4-yl)cyclopropyl)-1H-pyrazole-4-carboxylate (570 mg, 1.618 mmol) in dichloromethane (DCM) (10 mL) was added thionyl chloride (0.236 mL, 3.24 mmol). The reaction mixture was stirred overnight at room temperature. The mixture was concentrated and the residue was triturated with dichloromethane. It was filtered and washed with dichloromethane. The filtrate was washed with water and the organic layer was dried over anhydrous magnesium sulfate. It was subsequently concentrated and the crude product was purified via CombiFlash column chromatography eluting with a gradient of 0-80% ethyl acetate in hexanes. The title compound was obtained as colorless semi-solid (343 mg, 0.925 mmol, 57.2% yield). LC-MS m/z 371.1 (M+H)⁺, 1.0 min (ret. time).

### 56b) N-Hexyl-2-methylbenzenesulfonamide

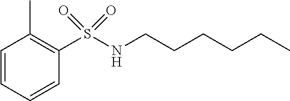 [SMILES: CCCCCCNS(=O)(=O)c1ccccc1C]

To a solution of 2-methylbenzene-1-sulfonyl chloride (0.169 mL, 1.049 mmol) in dichloromethane (DCM) (2.0 mL) was added TEA (0.292 mL, 2.098 mmol), followed by hexan-1-amine (0.194 mL, 1.469 mmol). The resulting cloudy mixture was stirred for 5 min at room temperature. The mixture was diluted with water and extracted with ethyl acetate. The organic extract was washed with water and dried over anhydrous magnesium sulfate. It was filtered and the filtrate was concentrated. The crude product was purified via CombiFlash column chromatography eluting with a gradient of 0-20% ethyl acetate in hexanes. The title compound was obtained as clear colorless oil (242 mg, 0.948 mmol, 90% yield). LC-MS m/z 256.0 (M+H)⁺, 1.23 min (ret. time).

### 56c) 1-(3-((N-Hexyl-2-methylphenylsulfonamido)methyl)phenyl)-5-(trans)-2-(1-methyl-1H-pyrazol-4-yl)cyclopropyl)-1H-pyrazole-4-carboxylic acid

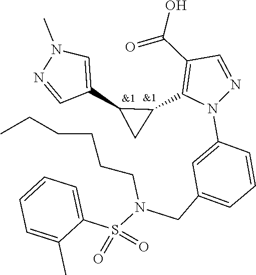 [SMILES: CCCCCCN(Cc1cccc(-n2ncc(C(=O)O)c2[C@@H]2C[C@H]2c2cnn(C)c2)c1)S(=O)(=O)c1ccccc1C]

A mixture of methyl 1-(3-(chloromethyl)phenyl)-5-(trans)-2-(1-methyl-1H-pyrazol-4-yl)cyclopropyl)-1H-pyrazole-4-carboxylate (40 mg, 0.108 mmol), N-hexyl-2-methylbenzenesulfonamide (25 mg, 0.098 mmol), and cesium carbonate (70.3 mg, 0.216 mmol) in N,N-dimethylformamide (DMF) (1.0 mL) was stirred for 2.0 hours at room temperature, after which LCMS showed complete consumption of starting material. The mixture was diluted with water and extracted with ethyl acetate. The organic extract was washed with water and concentrated to give the requisite intermediate. This intermediate was re-dissolved in tetrahydrofuran (THF) (1.0 mL) and methanol (1.0 mL). Aqueous NaOH (aq) (0.5 mL, 3.00 mmol, 6.0 N) was then added and the reaction mixture was stirred for 18 hours at room temperature. LCMS showed complete consumption of starting material. The mixture was concentrated and the residue was treated with water and neutralized with 2.0 HCl (aq). It was extracted with ethyl acetate and the organic extract was concentrated to give the crude product which was then purified on the prep HPLC eluting with a gradient of 20 to 100% acetonitrile in water (acidic conditions). However, the starting material sulfonamide was not separable from the product. Further purification was conducted on the prep HPLC eluting with a gradient of 10 to 60% acetonitrile (0.1% TFA) in water (0.1% TFA). The compound obtained was dissolved in ethyl acetate and neutralized with saturated NaHCO₃ (aq). The organic layer was separated and dried over anhydrous magnesium sulfate. It was filtered and the filtrate was concentrated to give the title compound as a white solid (12 mg, 0.02 mmol, 18.4% yield). LC-MS m/z 576.3 (M+H)⁺, 1.24 min (ret. time). ¹H NMR (400 MHz, DMSO-d₆) δ ppm 0.69-0.79 (m, 3H) 0.95-1.17 (m, 8H) 1.28 (m, 2H) 1.86 (m, 1H) 2.05-2.13 (m, 1H) 2.56 (s, 3H) 3.07 (t, J=7.28 Hz, 2H) 3.71 (s, 3H) 4.41-4.56 (m, 2H) 7.04 (s, 1H) 7.29 (s, 1H) 7.38-7.61 (m, 7H) 7.86 (d, J=7.78 Hz, 1H) 7.98 (s, 1H) 12.39 (br. s., 1H).

### Example 57. 1-(3-(((S)-4-Methyl-1,1-dioxido-7-(trifluoromethyl)-4,5-dihydrobenzo[f][1,2]thiazepin-2(3H)-yl)methyl)phenyl)-5-(trans-2-(3-methylisoxazol-5-yl)cyclopropyl)-1H-pyrazole-4-carboxylic acid

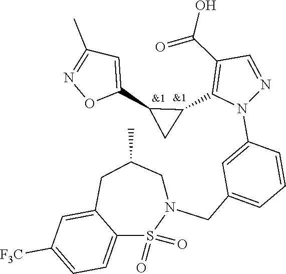 [SMILES: Cc1cc([C@@H]2C[C@H]2c2c(C(=O)O)cnn2-c2cccc(CN3C[C@@H](C)Cc4cc(C(F)(F)F)ccc4S3(=O)=O)c2)on1]

### 57a) Methyl 1-(3-bromophenyl)-5-(trans)-2-(3-methylisoxazol-5-yl)cyclopropyl)-1H-pyrazole-4-carboxylate

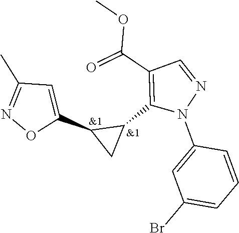 [SMILES: COC(=O)c1cnn(-c2cccc(Br)c2)c1[C@@H]1C[C@H]1c1cc(C)no1]

To a solution of acetaldehyde oxime (0.530 mL, 8.69 mmol) in N,N-Dimethylformamide (DMF) (4.0 mL) was added NCS (1207 mg, 9.04 mmol) and pyridine (0.070 mL, 0.869 mmol) at room temperature. After 1.0 h of stirring, rac-methyl 1-(3-bromophenyl)-5-(trans)-2-ethynylcyclopropyl)-1H-pyrazole-4-carboxylate (600 mg, 1.738 mmol) in N,N-Dimethylformamide (DMF) (4.0 mL) was added, and the mixture was stirred for 30 min, TEA (1.454 mL, 10.43 mmol) was added. The mixture was stirred overnight at 50° C. After cooled to the room temperature, the mixture was diluted with water and extracted with ethyl acetate. The organic extract was washed with water and dried over anhydrous magnesium sulfate. It was filtered and the filtrate was concentrated. The crude product was purified on the Combiflash eluting with a gradient of 0-30% ethyl acetate in hexanes. The title compound was obtained as white solid (566 mg, 1.407 mmol, 81% yield). LC-MS m/z 402.1 (M+H)⁺, 1.03 min (ret. time).

### 57b) 3-(4-(Methoxycarbonyl)-5-(trans)-2-(3-methylisoxazol-5-yl)cyclopropyl)-1H-pyrazol-1-yl)benzoic acid

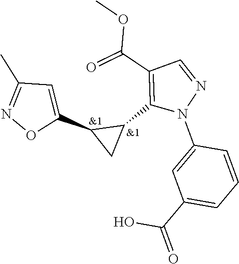 [SMILES: COC(=O)c1cnn(-c2cccc(C(=O)O)c2)c1[C@@H]1C[C@H]1c1cc(C)no1]

Palladium(II) acetate (18 mg, 0.080 mmol), xantphos (76 mg, 0.131 mmol), N-formylsaccharin (416 mg, 1.969 mmol), and potassium fluoride (238 mg, 4.10 mmol) were added to a microwave tube. The tube was then sealed and evacuated, backfilled with N₂ two times. A degassed solution of methyl 1-(3-bromophenyl)-5-(trans)-2-(3-methylisoxazol-5-yl)cyclopropyl)-1H-pyrazole-4-carboxylate (660 mg, 1.641 mmol) in anhydrous N,N-Dimethylformamide (DMF) (12 mL) was added and the mixture was stirred for 18 hours at 80° C. After cooled to the room temperature, TEA (0.572 mL, 4.10 mmol) and water (0.296 mL, 16.41 mmol) were added and the reaction mixture was stirred for 1.0 hour at room temperature. The mixture was diluted with water and extracted with ethyl acetate. The organic extract was washed with water and then 30 ml of saturated NaHCO₃ (aq) was added. The layers were separated and the aqueous layer was washed with ethyl acetate. Then the aqueous layer was acidified with 6.0 N HCl and extracted with ethyl acetate. The organic extract was washed with water and dried over anhydrous magnesium sulfate. It was filtered and the filtrate was concentrated to give the title compound as yellow solid (600 mg, 1.633 mmol, 100% yield) used in the next step without further purification. LC-MS m/z 368.1 (M+H)⁺, 0.88 min (ret. time).

### 57c) Methyl 1-(3-(hydroxymethyl)phenyl)-5-(trans)-2-(3-methylisoxazol-5-yl)cyclopropyl)-1H-pyrazole-4-carboxylate

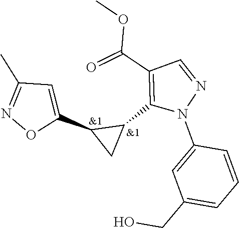 [SMILES: COC(=O)c1cnn(-c2cccc(CO)c2)c1[C@@H]1C[C@H]1c1cc(C)no1]

To a solution of rac-3-(4-(methoxycarbonyl)-5-(trans)-2-(3-methylisoxazol-5-yl)cyclopropyl)-1H-pyrazol-1-yl)benzoic acid (600 mg, 1.633 mmol) in Tetrahydrofuran (THF) (10 mL) was added CDI (795 mg, 4.90 mmol). The reaction mixture was stirred for 3.0 hours at room temperature. The mixture was added to another via containing sodium borohydride (309 mg, 8.17 mmol) and Water (5 mL). The reaction mixture was stirred for 20 min at room temperature.

The mixture was diluted with water and extracted with ethyl acetate. The organic extract was washed with water and dried over anhydrous magnesium sulfate. It was filtered and the filtrate was concentrated. The crude product was purified on the Combiflash eluting with a gradient of 0-75% ethyl acetate in hexanes. The title compound was obtained as clear colorless wax (250 mg, 0.707 mmol, 43.3% yield). LC-MS m/z 354.1 (M+H)⁺, 0.82 min (ret. time).

### 57d) Methyl 1-(3-(((S)-4-methyl-1,1-dioxido-7-(trifluoromethyl)-4,5-dihydrobenzo[f][1,2]thiazepin-2(3H)-yl)methyl)phenyl)-5-(trans-2-(3-methylisoxazol-5-yl)cyclopropyl)-1H-pyrazole-4-carboxylate

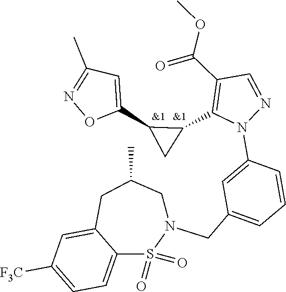 [SMILES: COC(=O)c1cnn(-c2cccc(CN3C[C@@H](C)Cc4cc(C(F)(F)F)ccc4S3(=O)=O)c2)c1[C@@H]1C[C@H]1c1cc(C)no1]

To a solution of rac-methyl 1-(3-(hydroxymethyl)phenyl)-5-(trans)-2-(3-methylisoxazol-5-yl)cyclopropyl)-1H-pyrazole-4-carboxylate (50 mg, 0.141 mmol) and (S)-4-methyl-7-(trifluoromethyl)-2,3,4,5-tetrahydrobenzo[f][1,2]thiazepine 1,1-dioxide (43.5 mg, 0.156 mmol) in Tetrahydrofuran (THF) (1.2 mL) was added trimethylphosphine (1.0 M in THF) (0.283 mL, 0.283 mmol), followed by DIAD (0.055 mL, 0.283 mmol). The reaction mixture was stirred for 3.0 hours at room temperature. The mixture was diluted with water and extracted with ethyl acetate. The organic extract was dried over anhydrous magnesium sulfate. It was filtered and the filtrate was concentrated. The crude product was purified on the Combiflash eluting with a gradient of 0-40% ethyl acetate in hexanes. The title compound was obtained as colorless wax (62 mg, 0.101 mmol, 71.3% yield). LC-MS m/z 615.3 (M+H)⁺, 1.35 min (ret. time).

### 57e) 1-(3-(((S)-4-methyl-1,1-dioxido-7-(trifluoromethyl)-4,5-dihydrobenzo[f][1,2]thiazepin-2(3H)-yl)methyl)phenyl)-5-(trans-2-(3-methylisoxazol-5-yl)cyclopropyl)-1H-pyrazole-4-carboxylic acid

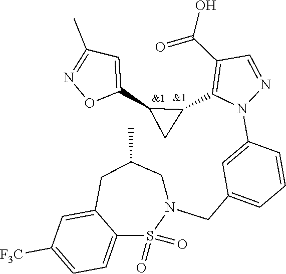 [SMILES: Cc1cc([C@@H]2C[C@H]2c2c(C(=O)O)cnn2-c2cccc(CN3C[C@@H](C)Cc4cc(C(F)(F)F)ccc4S3(=O)=O)c2)on1]

A mixture of methyl 1-(3-(((S)-4-methyl-1,1-dioxido-7-(trifluoromethyl)-4,5-dihydrobenzo[f][1,2]thiazepin-2(3H)-yl)methyl)phenyl)-5-(trans)-2-(3-methylisoxazol-5-yl)cyclopropyl)-1H-pyrazole-4-carboxylate (62 mg, 0.101 mmol) and 6.0 N NaOH (aq) (0.5 mL, 3.00 mmol) in Tetrahydrofuran (THF) (0.8 mL) and Methanol (0.8 mL) was stirred for 18 hours at room temperature. The mixture was concentrated and the residue was diluted with water (3 mL), neutralized with 2.0 N HCl (aq). The resulting precipitate was extracted with ethyl acetate. The organic extract was concentrated and the crude product was purified on the prep HPLC eluting with a gradient of 30-100% acetonitrile in water. The title compound was obtained as white solid (52 mg, 0.082 mmol, 82% yield). LC-MS m/z 600.3 (M+H)⁺, 1.13 min (ret. time). ¹H NMR (400 MHz, DMSO-d₆) δ ppm 0.94 (br. s., 3H) 1.26 (m, 1H) 1.41 (m, 1H) 2.12 (d, J=6.78 Hz, 3H) 2.16-2.32 (m, 1H) 2.56-2.70 (m, 1H) 3.08 (m, 2H) 3.34-3.46 (m, 2H) 3.64 (m, 1H) 3.83-4.02 (m, 1H) 4.19 (m, 1H) 5.86-5.97 (m, 1H) 7.43 (br. s., 1H) 7.46-7.56 (m, 3H) 7.85 (d, J=7.53 Hz, 1H) 7.94 (s, 1H) 8.00 (s, 1H) 8.06 (d, J=7.53 Hz, 1H) 12.46 (br. s., 1H).

1. A compound of Formula (I) 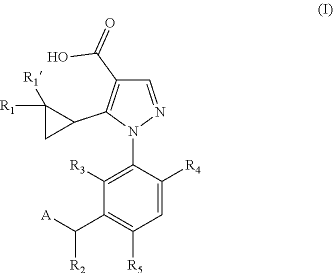
wherein:
R₁ is hydrogen, C₁₋₅alkyl, triazolyl, pyridyl, pyridazinyl, imidazolyl, pyrazolyl, isoxazolyl, halo, —NR₆—C(O)—R₇ or —C(O)R₇, and wherein the triazolyl, pyridyl, pyridazinyl, imidazolyl, pyrazolyl or isoxazolyl is unsubstituted or substituted by one or two substituents independently selected from —C₁₋₃alkyl, —CF₃ and halo;
R₁′ is hydrogen or halo;
R₂ is hydrogen, —C₁₋₅alkyl, —C₃₋₆cycloalkyl, or halo;
R₃ is hydrogen, —C₁₋₅alkyl, —C₃₋₆cycloalkyl, or halo;
or, when R₂ and R₃ are each —C₁₋₅alkyl, together they form a 5- to 6-membered cycloalkyl ring fused to the adjacent phenyl ring;
R₄ is hydrogen, —C₁₋₅alkyl, —C₃₋₆cycloalkyl, or halo;
R₅ is hydrogen, —C₁₋₅alkyl, —C₃₋₆cycloalkyl, or halo;
or, when R₂ and R₅ are each —C₁₋₅alkyl, together they form a 5- to 6-membered cycloalkyl ring fused to the adjacent phenyl ring;
R₆ and R₇ are independently hydrogen or —C₁₋₅alkyl;
A is 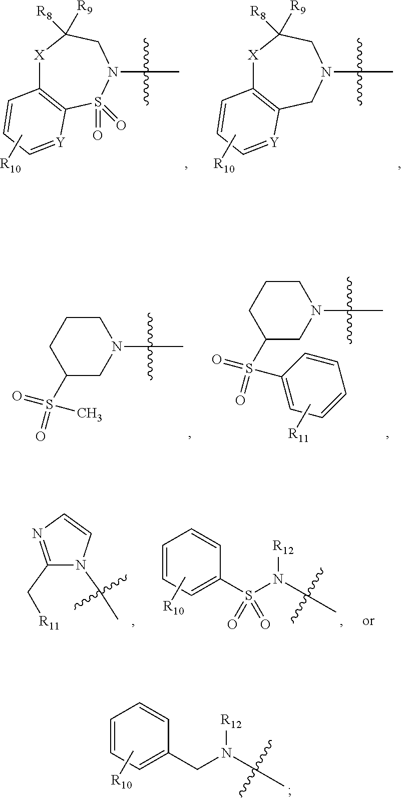
R₈ and R₉ are independently hydrogen or —C₁₋₅alkyl;
Each of R₁₀ is independently hydrogen, —C₁₋₅alkyl, —C₃₋₇cycloalkyl or halo;
R₁₁ is hydrogen or —C₅₋₈cycloalkyl;
R₁₂ is hydrogen, —C₁₋₆alkyl or —C₃₋₆cycloalkyl, wherein —C₁₋₆alkyl is unsubstituted or substituted with C₁₋₃alkyl;
X is CH₂ or O;
Y is CH or N;
or a pharmaceutically acceptable salt thereof.

2. The compound of claim 1 wherein:
R₁ is triazolyl, pyridyl, pyridazinyl, imidazolyl, pyrazolyl or isoxazolyl, and wherein the triazolyl, pyridyl, pyridazinyl, imidazolyl, pyrazolyl or isoxazolyl is unsubstituted or substituted by one or two substituents independently selected from —C₁₋₃alkyl, —CF₃ and halo;
R₁′ is hydrogen or halo;
R₂ is hydrogen, —C₁₋₅alkyl, —C₃₋₆cycloalkyl, or halo;
R₃ is hydrogen, —C₁₋₅alkyl, or halo;
R₄ is hydrogen, —C₁₋₅alkyl, or halo;
R₅ is hydrogen, —C₁₋₅alkyl, —C₃₋₆cycloalkyl, or halo;
A is 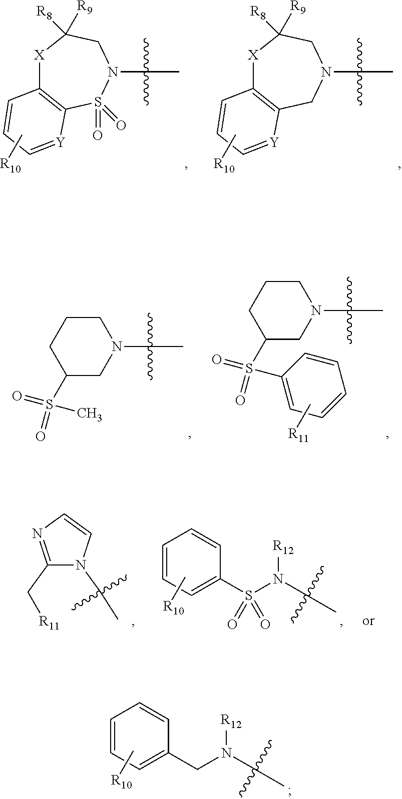
R₈ and R₉ are each hydrogen;
Each of R₁₀ is hydrogen;
R₁₁ is hydrogen;
R₁₂ is hydrogen or —C₁₋₆alkyl, wherein —C₁₋₆alkyl is unsubstituted or substituted with C₁₋₃alkyl;
X is CH₂ or O;
Y is CH or N;
or a pharmaceutically acceptable salt thereof.

3. The compound of claim 1 selected from:
1-(3-(((R)-4-ethyl-1,1-dioxido-3,4-dihydro-2H-benzo[b][1,4,5]oxathiazepin-2-yl)methyl)phenyl)-5-(trans)-2-(1-methyl-1H-1,2,3-triazol-4-yl)cyclopropyl)-1H-pyrazole-4-carboxylic acid;
1-(3-(((S)-4-Ethyl-1,1-dioxido-4,5-dihydrobenzo[f][1,2]thiazepin-2(3H)-yl)methyl)phenyl)-5-(trans)-2-(1-methyl-1H-1,2,3-triazol-4-yl)cyclopropyl)-1H-pyrazole-4-carboxylic acid.
1-(3-((8-fluoro-4,4-dimethyl-1,1-dioxido-3,4-dihydro-2H-benzo[b][1,4,5]oxathiazepin-2-yl)methyl)phenyl)-5-(trans)-2-(1-methyl-1H-1,2,3-triazol-4-yl)cyclopropyl)-1H-pyrazole-4-carboxylic acid;
1-(3-((R or S)-1-((S)-4-Methyl-1,1-dioxido-8-(trifluoromethyl)-4,5-dihydrobenzo[f][1,2]thiazepin-2(3H)-yl)ethyl)phenyl)-5-((1R,2R)-2-(1-methyl-1H-1,2,3-triazol-4-yl)cyclopropyl)-1H-pyrazole-4-carboxylic acid
1-(3-((4,4-Dimethyl-4,5-dihydro-1H-benzo[c]azepin-2(3H)-yl)methyl)phenyl)-5-(trans)-2-(1-methyl-1H-1,2,3-triazol-4-yl)cyclopropyl)-1H-pyrazole-4-carboxylic acid;
1-(3-((2,2-dimethyl-2,3-dihydrobenzo[f][1,4]oxazepin-4(5H)-yl)methyl)phenyl)-5-(trans)-2-(1-methyl-1H-1,2,3-triazol-4-yl)cyclopropyl)-1H-pyrazole-4-carboxylic acid;
1-(3-((2,2-dimethyl-2,3-dihydropyrido[2,3-f][1,4]oxazepin-4(5H)-yl)methyl)phenyl)-5-(trans)-2-(1-methyl-1H-1,2,3-triazol-4-yl)cyclopropyl)-1H-pyrazole-4-carboxylic acid;
1-(3-(((R)-4-ethyl-4,5-dihydro-1H-benzo[c]azepin-2(3H)-yl)methyl)phenyl)-5-(trans)-2-(1-methyl-1H-1,2,3-triazol-4-yl)cyclopropyl)-1H-pyrazole-4-carboxylic acid;
1-(3-(((S)-4-ethyl-4,5-dihydro-1H-benzo[c]azepin-2(3H)-yl)methyl)phenyl)-5-(trans)-2-(1-methyl-1H-1,2,3-triazol-4-yl)cyclopropyl)-1H-pyrazole-4-carboxylic acid;
1-(3-((7-bromo-2,2-dimethyl-2,3-dihydrobenzo[f][1,4]oxazepin-4(5H)-yl)methyl)phenyl)-5-(trans)-2-(1-methyl-1H-1,2,3-triazol-4-yl)cyclopropyl)-1H-pyrazole-4-carboxylic acid (trifluoroacetate);
1-(3-((5-ethyl-2,2-dimethyl-2,3-dihydrobenzo[f][1,4]oxazepin-4(5H)-yl)methyl)phenyl)-5-(trans)-2-(1-methyl-1H-1,2,3-triazol-4-yl)cyclopropyl)-1H-pyrazole-4-carboxylic acid;
Sodium 1-(3-(((R)-2-ethyl-2,3-dihydrobenzo[f][1,4]oxazepin-4(5H)-yl)methyl)phenyl)-5-(trans)-2-(1-methyl-1H-1,2,3-triazol-4-yl)cyclopropyl)-1H-pyrazole-4-carboxylate;
1-(3-((4-methyl-1,1-dioxido-4,5-dihydrobenzo[f][1,2]thiazepin-2(3H)-yl)methyl)phenyl)-5-((trans)-2-(1-methyl-1H-1,2,3-triazol-4-yl)cyclopropyl)-1H-pyrazole-4-carboxylic acid;
1-(3-((4-methyl-1,1-dioxido-8-(trifluoromethyl)-4,5-dihydrobenzo[f][1,2]thiazepin-2(3H)-yl)methyl)phenyl)-5-((trans)-2-(1-methyl-1H-1,2,3-triazol-4-yl)cyclopropyl)-1H-pyrazole-4-carboxylic acid;
1-(3-((8-bromo-4-methyl-1,1-dioxido-4,5-dihydrobenzo[f][1,2]thiazepin-2(3H)-yl)methyl)phenyl)-5-((trans)-2-(1-methyl-1H-1,2,3-triazol-4-yl)cyclopropyl)-1H-pyrazole-4-carboxylic acid;
1-(3-((4-methyl-1,1-dioxido-7-(trifluoromethyl)-4,5-dihydrobenzo[f][1,2]thiazepin-2(3H)-yl)methyl)phenyl)-5-((trans)-2-(1-methyl-1H-1,2,3-triazol-4-yl)cyclopropyl)-1H-pyrazole-4-carboxylic acid;
1-(3-((4-methyl-1,1-dioxido-7-(trifluoromethyl)-4,5-dihydrobenzo[f][1,2]thiazepin-2(3H)-yl)methyl)phenyl)-5-((trans)-2-(1-methyl-1H-1,2,3-triazol-4-yl)cyclopropyl)-1H-pyrazole-4-carboxylic acid;
1-(3-(((S)-4-ethyl-1,1-dioxido-8-(trifluoromethyl)-4,5-dihydrobenzo[f][1,2]thiazepin-2(3H)-yl)methyl)phenyl)-5-((1R,2R)-2-(1-methyl-1H-1,2,3-triazol-4-yl)cyclopropyl)-1H-pyrazole-4-carboxylic acid;
1-(3-(((S)-4-butyl-1,1-dioxido-8-(trifluoromethyl)-4,5-dihydrobenzo[f][1,2]thiazepin-2(3H)-yl)methyl)phenyl)-5-((1R,2R)-2-(1-methyl-1H-1,2,3-triazol-4-yl)cyclopropyl)-1H-pyrazole-4-carboxylic acid; 1-(3-(((S)-8-bromo-4-ethyl-1,1-dioxido-4,5-dihydrobenzo[f][1,2]thiazepin-2(3H)-yl)methyl)phenyl)-5-((1R,2R)-2-(1-methyl-1H-1,2,3-triazol-4-yl)cyclopropyl)-1H-pyrazole-4-carboxylic acid;
1-(3-((2-(Cycloheptylmethyl)-1H-imidazol-1-yl)methyl)phenyl)-5-(trans)-2-(1-methyl-1H-1,2,3-triazol-4-yl)cyclopropyl)-1H-pyrazole-4-carboxylic acid;
5-(trans)-2-(1-methyl-1H-1,2,3-triazol-4-yl)cyclopropyl)-1-(3-((2-(piperidin-1-ylmethyl)-1H-imidazol-1-yl)methyl)phenyl)-1H-pyrazole-4-carboxylic acid;
1-(3-(((R)-4-Ethyl-1,1-dioxido-3,4-dihydro-2H-benzo[b][1,4,5]oxathiazepin-2-yl)methyl)-4-methylphenyl)-5-(trans)-2-(1-methyl-1H-1,2,3-triazol-4-yl)cyclopropyl)-1H-pyrazole-4-carboxylic acid;
1-((R)-1-((S)-4-Methyl-1,1-dioxido-8-(trifluoromethyl)-4,5-dihydrobenzo[f][1,2]thiazepin-2(3H)-yl)-2,3-dihydro-1H-inden-4-yl)-5-((1R,2R)-2-(1-methyl-1H-1,2,3-triazol-4-yl)cyclopropyl)-1H-pyrazole-4-carboxylic acid;
1-((S)-1-((S)-4-Methyl-1,1-dioxido-8-(trifluoromethyl)-4,5-dihydrobenzo[f][1,2]thiazepin-2(3H)-yl)-2,3-dihydro-1H-inden-4-yl)-5-((1R,2R)-2-(1-methyl-1H-1,2,3-triazol-4-yl)cyclopropyl)-1H-pyrazole-4-carboxylic acid;
1-(3-(2-methoxy-N-methylphenylsulfonamido)-2,3-dihydro-1H-inden-5-yl)-5-((1R,2R)-2-(1-methyl-1H-1,2,3-triazol-4-yl)cyclopropyl)-1H-pyrazole-4-carboxylic acid;
1-(3-(((R)-2-ethyl-2,3-dihydrobenzo[f][1,4]oxazepin-4(5H)-yl)methyl)-4-methylphenyl)-5-(trans)-2-(1-methyl-1H-1,2,3-triazol-4-yl)cyclopropyl)-1H-pyrazole-4-carboxylic acid;
1-(3-(((R)-4-Ethyl-1,1-dioxido-3,4-dihydro-2H-benzo[b][1,4,5]oxathiazepin-2-yl)methyl)phenyl)-5-(trans)-2-(1-(2,2,2-trifluoroethyl)-1H-1,2,3-triazol-4-yl)cyclopropyl)-1H-pyrazole-4-carboxylic acid;
1-(3-(((S)-4-Methyl-1,1-dioxido-7-(trifluoromethyl)-4,5-dihydrobenzo[f][1,2]thiazepin-2(3H)-yl)methyl)phenyl)-5-(trans-2-(1-methyl-1H-pyrazol-4-yl)cyclopropyl)-1H-pyrazole-4-carboxylic acid;
1-(3-(((R)-4-ethyl-1,1-dioxido-3,4-dihydro-2H-benzo[b][1,4,5]oxathiazepin-2-yl)methyl)phenyl)-5-(trans-2-(1-methyl-1H-pyrazol-4-yl)cyclopropyl)-1H-pyrazole-4-carboxylic acid;
1-(3-(((S)-4-methyl-1,1-dioxido-4,5-dihydrobenzo[f][1,2]thiazepin-2(3H)-yl)methyl)phenyl)-5-(trans-2-(1-methyl-1H-pyrazol-4-yl)cyclopropyl)-1H-pyrazole-4-carboxylic acid;
1-(3-(((R)-4-methyl-1,1-dioxido-7-(trifluoromethyl)-4,5-dihydrobenzo[f][1,2]thiazepin-2(3H)-yl)methyl)phenyl)-5-(trans-2-(1-methyl-1H-pyrazol-4-yl)cyclopropyl)-1H-pyrazole-4-carboxylic acid;
1-(3-((N-hexyl-[1,1′-biphenyl]-3-ylsulfonamido)methyl)phenyl)-5-((trans)-2-(1-methyl-1H-1,2,3-triazol-4-yl)cyclopropyl)-1H-pyrazole-4-carboxylic acid;
5-((trans)-2-(1-methyl-1H-1,2,3-triazol-4-yl)cyclo-propyl)-1-(3-((N-methyl-3-phenoxy-phenyl-sulfonamido)-methyl)phenyl)-1H-pyrazole-4-carboxylic acid;
1-(3-((N-hexyl-4-phenoxyphenylsulfonamido)methyl)phenyl)-5-((trans)-2-(1-methyl-1H-1,2,3-triazol-4-yl)-cyclopropyl)-1H-pyrazole-4-carboxylic acid;
1-(3-((N-hexyl-2-methylphenylsulfonamido)methyl)phenyl)-5-((trans)-2-(1-methyl-1H-1,2,3-triazol-4-yl)cyclo-propyl)-1H-pyrazole-4-carboxylic acid;
5-((trans)-2-(1-methyl-1H-1,2,3-triazol-4-yl)cyclo-propyl)-1-(3-((N-methyl-2-phenoxyphenyl-sulfonamido)-methyl)phenyl)-1H-pyrazole-4-carboxylic acid;
5-((trans)-2-(1-methyl-1H-1,2,3-triazol-4-yl)cyclo-propyl)-1-(3-((N-methyl-[1,1′-biphenyl]-4-ylsulfonamido)-methyl)phenyl)-1H-pyrazole-4-carboxylic acid;
1-(3-((5-chloro-2-methoxy-N-methylphenyl-sulfonamido)-methyl)phenyl)-5-((trans)-2-(1-methyl-1H-1,2,3-triazol-4-yl)cyclo-propyl)-1H-pyrazole-4-carboxylic acid;
1-(3-((2-chloro-N-methyl-6-(trifluoro-methyl)phenyl-sulfonamido)-methyl)phenyl)-5-((trans)-2-(1-methyl-1H-1,2,3-triazol-4-yl)cyclo-propyl)-1H-pyrazole-4-carboxylic acid;
1-(3-((N,2-dimethylphenylsulfonamido)methyl)phenyl)-5-((trans)-2-(1-methyl-1H-1,2,3-triazol-4-yl)cyclopropyl)-1H-pyrazole-4-carboxylic acid;
1-(3-((5-bromo-2-methoxy-N-methylphenylsulfonamido)methyl)phenyl)-5-((trans)-2-(1-methyl-1H-1,2,3-triazol-4-yl)cyclopropyl)-1H-pyrazole-4-carboxylic acid;
1-(3-((2-methoxy-N-methyl-4-nitrophenylsulfonamido)methyl)phenyl)-5-((trans)-2-(1-methyl-1H-1,2,3-triazol-4-yl)cyclopropyl)-1H-pyrazole-4-carboxylic acid;
1-(3-((2-ethoxy-N-methylphenylsulfonamido)methyl)phenyl)-5-((trans)-2-(1-methyl-1H-1,2,3-triazol-4-yl)cyclopropyl)-1H-pyrazole-4-carboxylic acid;
5-((trans)-2-(1-methyl-1H-1,2,3-triazol-4-yl)cyclopropyl)-1-(3-((N-methyl-2-(trifluoromethoxy)phenylsulfonamido)methyl)phenyl)-1H-pyrazole-4-carboxylic acid;
1-(3-((2-methoxy-N-methylphenylsulfonamido)methyl)phenyl)-5-((trans)-2-(1-methyl-1H-1,2,3-triazol-4-yl)cyclopropyl)-1H-pyrazole-4-carboxylic acid;
5-((trans)-2-(1-methyl-1H-1,2,3-triazol-4-yl)cyclopropyl)-1-(3-((N-methylphenylsulfonamido)methyl)phenyl)-1H-pyrazole-4-carboxylic acid;
1-(3-((N-hexyl-2-phenoxyphenylsulfonamido)methyl)phenyl)-5-((trans)-2-(1-methyl-1H-1,2,3-triazol-4-yl)cyclopropyl)-1H-pyrazole-4-carboxylic acid;
5-((trans)-2-(1-methyl-1H-1,2,3-triazol-4-yl)cyclopropyl)-1-(3-((2-methyl-N-propylphenylsulfonamido)methyl)phenyl)-1H-pyrazole-4-carboxylic acid;
5-((trans)-2-(1-methyl-1H-1,2,3-triazol-4-yl)cyclopropyl)-1-(3-((N-methylquinoline-8-sulfonamido)methyl)phenyl)-1H-pyrazole-4-carboxylic acid;
1-(3-((2-methoxyphenylsulfonamido)methyl)phenyl)-5-((trans)-2-(1-methyl-1H-1,2,3-triazol-4-yl)cyclopropyl)-1H-pyrazole-4-carboxylic acid;
1-(3-((N-cyclopropyl-2-methoxyphenylsulfonamido)methyl)phenyl)-5-((trans)-2-(1-methyl-1H-1,2,3-triazol-4-yl)cyclopropyl)-1H-pyrazole-4-carboxylic acid; 1-(3-(((5-bromo-2-ethoxybenzyl)(methyl)amino)methyl)phenyl)-5-((trans)-2-(1-methyl-1H-1,2,3-triazol-4-yl)cyclopropyl)-1H-pyrazole-4-carboxylic acid trifluoroacetate; 1-(3-(((5-bromo-2-ethoxybenzyl)(cyclopropyl)amino)methyl)phenyl)-5-((trans)-2-(1-methyl-1H-1,2,3-triazol-4-yl)cyclopropyl)-1H-pyrazole-4-carboxylic acid trifluoroacetate; 1-(3-((N-Hexyl-2-methylphenylsulfonamido)methyl)phenyl)-5-(trans)-2-(1-methyl-1H-pyrazol-4-yl)cyclopropyl)-1H-pyrazole-4-carboxylic acid; and
1-(3-(((S)-4-Methyl-1,1-dioxido-7-(trifluoromethyl)-4,5-dihydrobenzo[f][1,2]thiazepin-2(3H)-yl)methyl)phenyl)-5-(trans-2-(3-methylisoxazol-5-yl)cyclopropyl)-1H-pyrazole-4-carboxylic acid.
or a pharmaceutically acceptable salt thereof.

4. A pharmaceutical composition comprising a compound of claim 1 and a pharmaceutically acceptable excipient.

5. A method of treating respiratory and non-respiratory disorders, including COPD, asthma, ALI, ARDS, fibrosis, chronic asthma and acute asthma, lung disease secondary to environmental exposures, acute lung infection, chronic lung infection, α1 antitrypsin disease, cystic fibrosis, autoimmune diseases, diabetic nephropathy, chronic kidney disease, sepsis-induced acute kidney injury, acute kidney injury (AKI), kidney disease or malfunction seen during kidney transplantation, Pulmonary Arterial Hypertension, atherosclerosis, hypertension, heart failure, Parkinson's disease (PD), Alzheimer's disease (AD), Friedreich's Ataxia (FA), amyotrophic lateral sclerosis (ALS), multiple sclerosis (MS), inflammatory bowel disease, colon cancer, neovascular (dry) AMD and neovascular (wet) AMD, eye injury, Fuchs Endothelial Corneal Dystrophy (FECD), uveitis or other inflammatory eye conditions, Non-alcoholic Steatohepatitis (NASH), toxin-induced liver disease (e.g., acetaminophen-induced hepatic disease), viral hepatitis, cirrhosis, psoriasis, dermatitis/topical effects of radiation, immunosuppression due to radiation exposure, Preeclampsia, and high altitude sickness, which comprises administering to a human in need thereof, a therapeutically effective amount of a compound of claim 1.

6. The method according to claim 5 wherein the compound is administered orally.

7. The method according to claim 5 wherein the compound is administered intravenously.

8. The method according to claim 5 wherein the compound is administered by inhalation.

9. The method according to claim 5 wherein the disease is COPD.

10. The method according to claim 5 wherein the disease is heart failure.

11-13. (canceled)
